(19) United States
(12) Patent Application Publication
(10) Pub. No.: US 2023/0111599 A1
(43) Pub. Date: Apr. 13, 2023

(54) VACCINES COMPRISING GLYCOENGINEERED BACTERIA

(71) Applicant: MALCISBO AG, Schlieren (CH)

(72) Inventor: Christine NEUPERT, Zurich (CH)

(21) Appl. No.: 17/908,829

(22) PCT Filed: Mar. 3, 2021
(86) PCT No.: PCT/EP2021/055298

§ 371 Date: Sep. 1, 2022

(30) Foreign Application Priority Data
Mar. 12, 2020 (EP) 20162625.6

(51) Int. Cl. A61K 39/102; A61P 31/04; C07K 14/195; C12N 15/52; C12N 15/70; C12N 15/74; C12N 9/10

(52) U.S. Cl. CPC A61K 39/102; A61P 31/04; C07K 14/195; C12N 15/52; C12N 15/70; C12N 15/74; C12N 9/1048; A61K 2039/521

(57) ABSTRACT

The present invention is directed to a gram-negative bacterial host cell for vaccine use comprising a heterologous functional Actinobacillus pleuropneumoniae (APR) rfb gene cluster producing an APR O-anti-gen bound to the lipid A-core of the bacterial host cell and located on the bacterial host outer surface, and wherein the endogenous rib gene cluster of the bacterial host cell is not functional. The invention further pertains to compositions comprising said host cells, in particular vaccines, and corresponding uses in the prophylaxis and/or therapy of Actinobacillus pleuropneumoniae (APR) infections.

### CROSS-REFERENCE TO RELATED APPLICATIONS

The present application is a National Stage Entry of PCT/EP2021/055298, filed 3 Mar. 2021, published as WO 2021/180532 A1, which claims the benefit of and priority to EP Application 20162625.6, filed 12 Mar. 2020, each of which is incorporated herein by reference in its entirety.

### INCORPORATION BY REFERENCE

In compliance with 37 C.F.R. § 1.52(e)(5), the sequence information contained in electronic file name: 50797PCT_sequence_ST25; size 245 KB; created on: 3 Mar. 2021; is hereby incorporated herein by reference in its entirety.

### FIELD OF THE INVENTION

The present invention is directed to a gram-negative bacterial host cell for vaccine use comprising a heterologous functional Actinobacillus pleuropneumoniae (APP) rfb gene cluster producing an APP O-antigen bound to the lipid A-core of the bacterial host cell and located on the bacterial host outer surface, and wherein the endogenous rfb gene cluster of the bacterial host cell is not functional. The invention further pertains to compositions comprising said host cells, in particular vaccines, and corresponding uses in the prophylaxis and/or therapy of Actinobacillus pleuropneumoniae (APP) infections.

### BACKGROUND OF THE INVENTION

Actinobacillus pleuropneumoniae (APP) is the major cause of porcine pleuropneumonia, a highly contagious respiratory disease in pigs responsible for major economic losses in the swine industry. Transmission occurs through aerosol or close contact with infected animals or asymptomatic carriers. To date, 18 serotypes of APP with different geographical distribution have been classified by their surface capsular polysaccharides (Bosse et al. 2018, Vet Microbiol., Vol. 220, 83-89). All serotypes are capable of causing disease, although differences in virulence exist. The existence of at least 18 serotypes makes it challenging to develop a broadly protective vaccine. The economic importance of the disease has stimulated intensive research in the last years in the APP vaccination field. However, as antibiotics are used to control the disease and antibiotic resistance has reached alarming levels all over the world, alternative solutions are needed.

Currently available vaccines against APP mainly consist of inactivated whole-cell bacterins, (chemically inactivated bacterial cells) or subunit vaccines based on outer membrane proteins. Some vaccines are based on or complemented with Apx toxins (ApxI-IV toxoids), a set of pore-forming cytolysins playing a central role in APP pathogenesis. To date, the protective effect induced by all commercialized vaccines is not satisfactory. Bacterin-based vaccines and subunit vaccines have been shown to provide limited protection against heterologous strains. Vaccines based on inactivated Apx toxins are effective in reducing the morbidity associated with infection, but they are unable to prevent colonisation of the lungs and their use poses a potential threat for inducing infection by asymptomatic carriers (Andresen et al. 1997, Acta Vet Scand, Vol. 38, 283-293, Antenucci et al 2017, Vet Res., Vol. 48:74, Antenucci et al 2018, Vet Res., 49:4, Haesebrouck et al. 2004, Vet Microbiol., Vol. 100, 255-268, Loera-Muro and Angulo 2018, Vet Microbiol. Vol. 217, 66-75, Ramjeet et al. 2008, Anim Health Res Rev, Vol. 9(1), 25-45).

Potential antigenic structures besides surface proteins for bacterial vaccine development are glycans presented on the surface of pathogenic bacteria. These glycans are one of the first contact points of an immune system of an infected host. Two prominent surface glycan structures of gram-negative bacteria are the extracellular capsular polysaccharides (CPS, in gram-positive and negative bacteria) and lipopolysaccharides (LPS, in gram-negative bacteria). Glycan-based vaccine development against bacterial diseases in the human field has mainly focused on CPS structures. Several bacterial diseases were addressed by isolating CPS structures and chemically conjugating these glycans to an immunostimulatory protein carrier. CPS of H. influenza type B conjugated to tetanus toxoid (product ActHIB) of Sanofi Pasteur and CPS of 4 Neisseria meningitidis serotypes conjugated to diphtheria toxoid (Menveo) of GSK Vaccines, CPS of 13 Streptococcus pneumoniae serotypes conjugated to diphtheria toxoid (Prevnar 13) of Pfizer represent three examples of the successful development of this type of CPS-based vaccine.

However, for veterinary vaccine development the isolation of surface glycans and subsequent chemical conjugation to a carrier structure leads to non-economical production costs.

The objective underlying the present invention is the provision of a safe and efficient vaccine that offers protection against many, most, and preferably essentially all serotypes of APP bacteria, which would allow for a significant reduction of antibiotic treatment in food production, and would reduce clinical outbreaks and losses during the fattening period of swine.

### SUMMARY OF THE INVENTION

This objective is solved by the provision of a gram-negative bacterial host cell for vaccine use comprising
(a) a heterologous functional Actinobacillus pleuropneumoniae (APP) rfb gene cluster, wherein the heterologous functional APP rfb gene cluster produces an APP O-antigen that is bound to the lipid A-core of the bacterial host cell and is located on the bacterial host outer surface, and wherein the endogenous rfb gene cluster of the bacterial host cell is not functional;
(b) optionally a heterologous promoter for regulating the transcription of the heterologous APP rfb gene cluster that is stronger than the endogenous promoter for the endogenous rfb gene cluster;
(c) optionally at least one further gene for functionally expressing an enzyme assisting the APP O-antigen synthesis;
(d) optionally at least one neutralizing epitope of Apx toxins, optionally at least one neutralizing epitope of Apx toxins I, II and III, optionally located on the bacterial host outer cell surface and/or secreted from the cell;
wherein optionally at least one of (a), (c) and (d) is codon-optimized for the bacterial host cell.

It was surprisingly found and clinically demonstrated that gram-negative bacterial host cells with a non-functional endogenous rfb gene cluster, i.e. without production of endogenous O-antigen, but comprising a heterologous functional Actinobacillus pleuropneumoniae (APP) rfb gene cluster and producing an APP O-antigen that is bound to the lipid A-core of the bacterial host cell and is located on the bacterial host outer surface will elicit an excellent immune response in swine that protects against APP infection.

The adjective term “heterologous”, as used herein, indicates that the so-termed matter, e.g. cell components such as genes, proteins, glycans, glycoproteins, metabolites, etc., is not naturally present in said cell by nature, i.e. it was artificially introduced and stems from a heterologous, i.e. not genetically identical organism.

The adjective term “endogenous”, as used herein, indicates that the so-termed matter, e.g. cell components such as genes, proteins, glycans, glycoproteins, metabolites, etc., is naturally and originally present in said cell by nature.

A person skilled in the art may routinely identify components and compounds of a cell as being heterologous or endogenous by known methods, e.g. by comparative molecular genetics and biochemical analysis. For example, a skilled person can routinely identify the APP rfb gene cluster and/or the APP O-antigen in a cell that is not APP as heterologous. As well, it is routine to demonstrate that an rfb gene cluster is non-functional or its endogenously absence or a non-functional gene structure in a gram-negative bacterium and/or the absence of the corresponding APP O-antigen in or on the cell or secreted from the cell of interest.

The term “non-functional”, as used herein, in particular, in the context of an rfb gene cluster in a bacterial host cell is meant to indicate the partial or full absence, structural or functional alteration or at least dysfunction of at least one gene, optionally all genes of the cluster, leading to the absence, malexpression and/or malfunction of at least one protein, optionally all proteins resulting from the gene cluster, and leading to essentially no production of O-antigen from the gene clusters expression products. For example, one or more of the genes of the rfb gene cluster may be altered and/or deleted and/or the cluster's gene regulation may be altered to render the expression of its genes dysfunctional, i.e. leading to physiologically irrelevant or no expression of proteins for O-antigen synthesis. The analysis of genes and proteins and assessing their functionality or the absence thereof can be achieved with routine techniques in molecular biology and biochemistry.

For achieving a physiologically effective immune response, the bacterial host cell will bind the APP O-antigen resulting from the expression of the heterologous functional APP rfb gene cluster to the lipid A-core of the bacterial host and transfer the conjugate to the outer surface of the bacterial host for display and contact with relevant immunity-related components in the environment, e.g. of a live and vaccinated swine.

The bacterial host cell of the present invention can be broadly selected from gram-negative bacterial cells because all gram-negative cells will express and comprise lipid A that is required for being bound to the heterologous O-antigen of the bacterial host cell of the invention. In a particular embodiment, for example, the bacterial host cell may be selected from the group consisting of Enterobacteriaceae, Burkholderiaceae, Pseudomonadaceae, Vibrionaceae, optionally Burkholderia thailandensis, Pseudomonas aeruginosa, Vibrio natriegens, Vibrio cholerae, Escherichia coli, optionally E. coli_5, Salmonella enterica, optionally Salmonella enterica subsp. enterica, optionally Salmonella enterica subsp. enterica selected from the group consisting of serovar Typhimurium, Enteritidis, Heidelberg, Gallinarum, Hadar, Agona, Kentucky and Infantis, and Salmonella enterica subsp. enterica serovar Typhimurium SL1344.

Of course, it is understood that the selected bacterial host cell must be suitable for pharmaceutical application, e.g. for vaccine use, either as dead or live vaccine.

In a further embodiment the bacterial host cell of the invention comprises a heterologous functional rfb gene cluster that may be selected from all known APP rfb gene clusters, in particular, the well-known APP1 to 18 rfb gene clusters. These gene clusters may be altered for use in the bacterial host cell of the invention, i.e. differ from the naturally existing gene clusters to the extent that the corresponding expressed heterologous APP O-antigen is functional, i.e. will elicit physiologically relevant immunity in swine against APP challenge and can still be bound to the lipid A core of the bacterial host cell.

In one embodiment the heterogeneous rfb gene cluster for practicing the invention is the APP2 or APP8 rfb gene cluster,
(i) comprising or consisting of SEQ ID NO: 1, SEQ ID NO: 3 or SEQ ID NO: 4;
(ii) having at least 70, 80, 90, 95 or 98% nucleic acid sequence identity to SEQ ID NO: 1, SEQ ID NO: 3 or SEQ ID NO: 4, optionally over the whole sequence;
(iii) hybridizing to the nucleic acid sequence of SEQ ID NO: 1, SEQ ID NO: 3 or SEQ ID NO: 4 under stringent conditions; and/or
(iv) is degenerated with respect to the nucleic acid sequence of any of (i) to (iii).

The term “% (percent) sequence identity” as known to the skilled artisan and used herein in the context of nucleic acids indicates the degree of relatedness among two or more nucleic acid molecules that is determined by agreement among the sequences. The percentage of “sequence identity” is the result of the percentage of identical regions in two or more sequences while taking into consideration the gaps and other sequence peculiarities.

The identity of related nucleic acid molecules can be determined with the assistance of known methods. In general, special computer programs are employed that use algorithms adapted to accommodate the specific needs of this task. Preferred methods for determining identity begin with the generation of the largest degree of identity among the sequences to be compared. Preferred computer programs for determining the identity among two nucleic acid sequences comprise, but are not limited to, BLASTN (Altschul et al., 1990, J. Mol. Biol., Vol. 215 p403-410) and LALIGN (Huang and Miller 1991, Adv. Appl. Math., Vol. 12, p337-357). The BLAST programs can be obtained from the National Center for Biotechnology Information (NCBI) and from other sources (BLAST handbook, Altschul et al., NCB NLM NIH Bethesda, Md. 20894).

The identity of related nucleic acid molecules, e.g. APP rfb gene clusters, can also be determined by their ability to hybridize to a specifically referenced nucleic acid sequence, optionally under stringent conditions. Next to common and/or standard protocols in the prior art for determining the ability to hybridize to a specifically referenced nucleic acid sequence under stringent conditions (e.g. Sambrook and Russell, (2001) Molecular cloning: A laboratory manual (3 volumes)), it is preferred to analyze and determine the ability to hybridize to a specifically referenced nucleic acid sequence under stringent conditions by com-paring the nucleotide sequences, which may be found in gene databases (e.g. NCBI) with alignment tools, such as e.g. the above-mentioned BLASTN (Altschul et al., 1990, J. Mol. Biol., Vol. 215 p403-410), LALIGN alignment tools and multiple alignment tools such as e.g. CLUSTALW (Sievers et al. 2011, Mol. Sys. Bio., Vol. 7, p539), MUSCLE (Edgar 2004, Nucl. Acids Res., Vol. 32, p1792-7) or T-COFFEE (Notredame et al. 2000, J. of Mol. Bio., Vol. 302(1), p205-17).

Most preferably, the ability of an APP rfb gene cluster for use in the present invention to hybridize to a specifically referenced nucleic acid, e.g. those listed in any of SEQ ID NOs 1, 3 and 4, is confirmed in a Southern blot assay under the following conditions: 6× sodium chloride/sodium citrate (SSC) at 45° C. followed by a wash in 0.2× SSC, 0.1% SDS at 65° C.

In a further embodiment the bacterial host cell produces an O-antigen of APP1 to 18, optionally an APP2 or APP8 O-antigen, wherein the APP rfb gene cluster optionally expresses at least one protein comprising or consisting of the amino acids of any one of SEQ ID NOs: 2, 50-61, or SEQ ID NO: 5, 62-72, or the at least one protein having at least 70, 80, 90, 95 or 98% amino acid sequence identity to these sequences.

The percentage identity of related amino acid molecules can be determined with the assistance of known methods. In general, special computer programs are employed that use algorithms adapted to accommodate the specific needs of this task. Preferred methods for determining identity begin with the generation of the largest degree of identity among the sequences to be compared. Preferred computer programs for determining the identity among two amino acid sequences comprise, but are not limited to, TBLASTN, BLASTP, BLASTX, TBLASTX (Altschul et al., 1990, J. Mol. Biol., Vol. 215 p403-410), or ClustalW (Larkin et al. 2007, Bioinformatics, Vol. 23, p2947-2948). The BLAST programs can be obtained from the National Center for Biotechnology Information (NCBI) and from other sources (BLAST handbook, Altschul et al., NCB NLM NIH Bethesda, MD 20894). The ClustalW program can be obtained from clustal.org.

The heterologous APP rfb gene clusters for use in the invention may be prepared synthetically by methods well-known to the skilled person, but also may be isolated from suitable DNA libraries and other publicly available sources of nucleic acids and subsequently may optionally be mutated. The preparation of such libraries or mutations is well-known to the person skilled in the art.

In one alternative embodiment the bacterial host cell of the present invention is one, wherein the endogenous rfb gene cluster of the bacterial host cell is non-functional and at least partially, optionally completely deleted.

It was found that the introduction of a heterologous promoter for regulating the transcription of the heterologous APP rfb gene cluster improves APP O-antigen expression if the heterologous promoter is stronger than the endogenous promotor for the APP rfb gene cluster. The heterologous promoter for the heterologous APP rfb gene cluster is optionally selected from the group consisting of kanamycin promoter, proD promoter, j23101 promoter, proC promoter, STER_RS05525 promoter, STER_RS01225 promoter, STER_RS04515 promoter, STER_RS05020 promoter, STER_RS06870 promoter, STER_RS00780 promoter, P32 promoter, optionally consisting of kanamycin promoter, proD promoter, j23101 promoter, STER_RS04515 promoter and P32 promoter, optionally consisting of kanamycin promoter and proD (Datsenko et al. 2000, PNAS, Vol. 97(12), p6640-6645, Davis et al. 2010, Nucleic acids research, Vol. 39(3), p1121-1141, Kong et al. 2019, ACS Synthetic Biology, Vol. 8, p1469-1472).

The term “stronger” in the context of comparing promoter efficiency, as used herein, is meant to indicate that the heterologous promoter will produce more transcription product, i.e. rfb gene cluster transcripts and translation products, i.e. rfb cluster expression products (enzymes), and more APP O-antigen than the naturally occurring endogenous and functional APP rfb promoter in the host cell.

It was found that some of the enzyme activities resulting from the APP rfb gene cluster may limit cellular production of APP O-antigen. In this regard it was demonstrated that cellular production of APP O-antigen in bacterial host cells of the present invention may be improved in efficacy by introducing at least one further gene for functionally expressing an enzyme assisting, for example involved in, or transforming intermediate products, etc., of the APP O-antigen synthesis. Optionally, the at least one further gene for functionally expressing an enzyme assisting the APP O-antigen synthesis is selected from the group consisting of the enzymes for nucleotide activated glycan biosynthesis, undecaprenylpyrophosphate glycosyltransferases, O-antigen glycosyltransferases, O-antigen polymerases, O-antigen chain length determinant protein, and N-glycan epimerases and combinations thereof, optionally selected from the group consisting of the gne gene and the wzy gene,
i. wherein the gne gene encodes an UDP-galactose/UDP-N-actetylgalacosamine epimerase, optionally an epimerase from Campylobacter jejuni, the gne gene optionally comprising or consisting of SEQ ID NO: 6, or having a nucleic acid sequence at least 70, 80, 90, 95 or 98% identical to SEQ ID NO: 6, optionally over the whole sequence, and/or hybridizing to the nucleic acid sequence of SEQ ID NO: 6 under stringent conditions;
ii. wherein the wzy gene encodes an O-antigen polymerase of APP, optionally of APP2, the wzy gene optionally comprising or consisting of SEQ ID NO: 7 or the codon-optimized wzy gene of SEQ ID NO:8 or having a nucleic acid sequence at least 70, 80, 90, 95 or 98% identical to SEQ ID NO: 7 or SEQ ID NO:8, optionally over the whole sequence, and/or hybridizing to the nucleic acid sequence of SEQ ID NO: 7 or SEQ ID NO:8 under stringent conditions.

Bacterial infections are often associated with the release of toxic compounds. APP bacteria typically secrete ApxI, ApxII, ApxIII and ApxIV toxins in various combinations dependent of the APP serotype. For improving symptoms of APP infections the bacterial host cell of the present invention may optionally comprise at least one neutralizing epitope of Apx toxins, optionally at least neutralizing epitopes of Apx toxins Ito III that are located on the bacterial host outer cell surface and bound to a membrane protein, optionally selected from the group consisting of cytolysin A, trimeric autotransporter adhesion, preferably AIDA-I and EaeA, and outer membrane proteins (OMP), preferably OmpA of E. coli (Xu et al. 2018, PlosOne, Vol. 13(1), Rutherford et al. 2006, Vol.188(11), Wentzel et al. 2001, J. Bacteriol., Vol. 183(24), 7273-7284, 2001, Georgiou et al. 1996, Protein Engineering, Vol. 9(2), 239-247).

Codon optimization by synonymous substitution is widely used for recombinant protein expression. Codon optimization refers to the adaption of the codon composition of a recombinant gene for improving protein expression without altering the resulting amino acid sequence. This is possible because most amino acids are encoded by more than one codon. Typically, codon optimization is adapted for the specific host organism (Burgess-Brown et al. 2008, Protein Expression & Purification Vol. 59, p94-102, Elena et al. 2014, Frontiers in Microbiology, Vol. 5 (21), 1-8).

In an alternative embodiment, the bacterial host cell of the present invention is one, wherein (a) the heterologous functional APP rfb gene cluster, (b) at least one further gene for functionally expressing an enzyme assisting the APP O-antigen synthesis, and/or (c) at least one neutralizing epitope of Apx toxins is codon-optimized for the bacterial host cell. Optionally, the heterologous functional APP rfb gene cluster is codon-optimized for the bacterial host cell.

In the following specific and non-limiting embodiments of the bacterial host cell of the present invention are presented for further illustrating the general inventive concept.

In one alternative embodiment, the bacterial host cell of the present invention is Escherichia coli, optionally E. coli_5, or Salmonella enterica, optionally Salmonella enterica subsp. enterica, optionally Salmonella enterica subsp. enterica serovar Typhimurium, optionally Salmonella enterica subsp. enterica serovar Typhimurium SL1344, wherein
(a) the heterologous functional APP rfb gene cluster is selected from APP1 to 18 rfb gene clusters, optionally is the APP2 or APP8 rfb gene cluster;
(b) the heterologous promoter for regulating the transcription of the heterologous APP rfb gene cluster is the kanamycin or proD promoter;
(c) the at least one further gene for functionally expressing an enzyme assisting the APP O-antigen synthesis is the wzy gene, optionally a codon optimized wzy, and/or gne gene, both genes optionally integrated into the genome of the bacterial host cell or located on a plasmid;
(d) and optionally comprising at least one of neutralizing epitopes of Apx toxins I, II and III, optionally bound to a membrane protein, optionally bound to cytolysin A of E. coli, or secreted from the host cell;
wherein (i) the APP2 or the APP8 rfb gene cluster, (ii) the gne gene and/or (iii) the wzy gene, optionally the APP2 rfb gene cluster and the wzy gene are codon-optimized for the bacterial host cell Escherichia coli, optionally E. coli-5, or Salmonella enterica, optionally Salmonella enterica subsp. enterica, optionally Salmonella enterica subsp. enterica serovar Typhimurium.

In another alternative embodiment, optionally of the above, the bacterial host cell of the present invention is Salmonella enterica subsp. enterica serovar Typhimurium, optionally Salmonella enterica subsp. enterica serovar Typhimurium strain SL1344, wherein
(a) the codon optimized heterologous functional APP rfb gene cluster is the APP2 rfb gene cluster, optionally (i) comprising or consisting of SEQ ID NO: 3; (ii) having at least 70, 80, 90, 95 or 98% nucleic acid sequence identity to SEQ ID NO: 1 or SEQ ID NO: 3, optionally over the whole sequence; (iii) hybridizing to the nucleic acid sequence of SEQ ID NO: 1 or SEQ ID NO: 3 under stringent conditions; and/or (iv) is degenerated with respect to the nucleic acid sequence of any of (i) to (iii), vand the endogenous rfb gene cluster of the bacterial host cell is at least partially or completely deleted;
(b) the optional heterologous promoter for regulating the transcription of the heterologous

APP2 rfb gene cluster is the kanamycin promoter;
(c) the at least one further gene for functionally expressing an enzyme assisting the APP O-antigen synthesis is the gne gene and/or the wzy gene, optionally integrated into the genome of the bacterial host cell;
   i. wherein the gne gene, optionally the gne gene of Campylobacter jejuni, optionally comprises or consists of SEQ ID NO: 6 or has a nucleic acid sequence at least 70, 80, 90, 95 or 98% identical to SEQ ID NO: 6, optionally over the whole sequence, and/or hybridizes to the nucleic acid sequence of SEQ ID NO: 6 under stringent conditions;
   ii. wherein the wzy gene, optionally comprises or consists of SEQ ID NO: 7 or SEQ ID NO: 8, or has a nucleic acid sequence at least 70, 80, 90, 95 or 98% identical to SEQ ID NO: 7 or SEQ ID NO: 8, optionally over the whole sequence, and/or hybridizing to the nucleic acid sequence of SEQ ID NO: 7 or SEQ ID NO: 8 under stringent conditions;
(d) and optionally comprising at least 2 neutralizing epitopes of Apx toxins I, II and III, optionally of at least Apx toxins II and III, optionally bound to a membrane protein, optionally cytolysin A of E. coli.

In a further alternative embodiment, the bacterial host cell of the present invention is E. coli, optionally E. coli_5, wherein
(a)the heterologous functional APP rfb gene cluster is the APP2 rfb gene cluster, optionally
   (i) comprising or consisting of SEQ ID NO: 3; (ii) having at least 70, 80, 90, 95 or 98% nucleic acid sequence identity to SEQ ID NO: 1 or SEQ ID NO: 3, optionally over the whole sequence; (iii) hybridizing to the nucleic acid sequence of SEQ ID NO: 1 or SEQ ID NO: 3 under stringent conditions; and/or (iv) is degenerated with respect to the nucleic acid sequence of any of (i) to (iii), and the endogenous rfb gene cluster of the bacterial host cell is at least partially or completely deleted;
(b) the heterologous promoter for regulating the transcription of the heterologous APP2 rfb gene cluster is the kanamycin or the proD promoter, optionally the kanamycin promoter;
(c) the at least one further gene for functionally expressing an enzyme assisting the APP O-antigen synthesis is the gne gene, optionally integrated into the genome of the bacterial host cell or located on a plasmid,
   wherein the gne gene, optionally of Campylobacter jejuni, optionally comprises or consists of SEQ ID NO: 6 or has a nucleic acid sequence at least 70, 80, 90, 95 or 98% identical to SEQ ID NO: 6, optionally over the whole sequence, and/or hybridizes to the nucleic acid sequence of SEQ ID NO: 6 under stringent conditions;
(d) and optionally comprising at least one of neutralizing epitopes of Apx toxins I, II and III, optionally of at least Apx toxins II and III, optionally bound to a membrane protein, optionally bound to cytolysin A of E. coli, or secreted from the host cell; wherein the APP2 rfb gene cluster is optionally codon-optimized for E. coli.
In another embodiment of the present invention, the bacterial host cell is Salmonella enterica subsp. enterica serovar Typhimurium, optionally Salmonella enterica subsp. enterica serovar Typhimurium strain SL1344 or Escherichia coli, optionally E. coli_5, wherein
(a) the heterologous functional APP rfb gene cluster is the APP8 rfb gene cluster, optionally codon-optimized, optionally (i) comprising or consisting of SEQ ID NO: 4; (ii) having at least 70, 80, 90, 95 or 98% nucleic acid sequence identity to SEQ ID NO: 4, optionally over the whole sequence; (iii) hybridizing to the nucleic acid sequence of SEQ ID NO: 4 under stringent conditions; and/or (iv) is degenerated with respect to the nucleic acid sequence of any of (i) to (iii),
(b) the optional heterologous promoter for regulating the transcription of the heterologous APP2 rfb gene cluster is the kanamycin or proD promoter, optionally the kanamycin promoter;
(c) the at least one further gene for functionally expressing an enzyme of the APP O-antigen synthesis is the wzy and/or gne gene, optionally codon optimized, optionally both genes integrated into the genome of the bacterial host cell;
   i. wherein the gne gene, optionally of Campylobacter jejuni, optionally comprises or consists of SEQ ID NO: 6 or has a nucleic acid sequence at least 70, 80, 90, 95 or 98% identical to SEQ ID NO: 6, optionally over the whole sequence, and/or hybridizes to the nucleic acid sequence of SEQ ID NO: 6 under stringent conditions;
   ii. wherein the wzy gene, optionally codon-optimized, optionally comprises or consists of SEQ ID NO: 7 or SEQ ID NO: 8, or has a nucleic acid sequence at least 70, 80, 90, 95 or 98% identical to SEQ ID NO: 7 or SEQ ID NO: 8, optionally over the whole sequence, and/or hybridizing to the nucleic acid sequence of SEQ ID NO: 7 or SEQ ID NO: 8 under stringent conditions;
(d) and optionally comprises at least 2 neutralizing epitopes of Apx toxins I, II and III, optionally at least Apx toxins II and III, optionally bound to a membrane protein, optionally cytolysin A of E. coli.

The bacterial host cells of the present invention may be administered to swine in live or inactivated form.

The above-described bacterial host cells of the invention are highly immunogenic and produce immune responses against APP infections. Furthermore, once prepared they can be easily propagated and mass-produced. They can be administered live or inactivated, for example, as live or dead vaccines, live vaccines allowing for prolonged propagation and sustained immune stimulus in the host as well as full immune responses without adjuvants.

Therefore, the present invention also relates to the medical use of live or dead bacterial host cells of the present invention, in particular for preparing a medicament for the prophylaxis and/or therapy of APP infections, preferably a vaccine.

Preferably, the medicament is useful for the prevention and/or treatment of APP, in particular APP1-18 infections, preferably APP infections in swine.

A further aspect of the present invention relates to a composition, optionally a pharmaceutical composition comprising at least one bacterial host cell of the present invention as described herein, and a physiologically acceptable excipient.

In one embodiment, the composition or pharmaceutical composition of the present invention comprises bacterial host cells expressing at least two different O-antigens, optionally O-antigens from APP1 to APP18, optionally selected from the group consisting of APP1, 2, 4, 5, 6, 7, 8, 9, 10, 12, 14, 16, 17 and 18, optionally combinations of O-antigens selected from the group consisting of APP1, 2, 5, 7, 8 10, 12, 14 and 18.

In a further embodiment the bacterial host cell or the composition of the invention is for use in the prophylaxis and/or therapy of Actinobacillus pleuropneumoniae (APP) infections, optionally of APP2 infections in a mammal, optionally in swine (Sus) or domestic swine (Sus scrofa domesticus).

In another embodiment the invention comprises feed or drinking water for animals, in particular swine livestock, and a physiologically acceptable excipient and/or food stuff. For example, such compositions such as vaccines or feed would greatly reduce APP colonisation of swine livestock and consequently decrease the chance of infection spreading.

In another aspect the present invention is directed to a method of treatment comprising the step of administering a physiologically effective amount of a bacterial host cell or a composition, food or feed of the present invention to a mammalian subject in need thereof for the treatment of APP infections, optionally for the prophylaxis of APP infections in swine.

For therapeutic and/or prophylactic use the compositions, pharmaceutical compositions, food or feed of the invention may be administered in any conventional dosage form in any conventional manner.

Routes of administration include, but are not limited to intranasal, oral, oronasal, transcutanous, conjunctive, in ovo, subcutaneous, intradermal, intramuscular, sublingual, transdermal, or conjunctival. For example, application devices and methods include syringes, atomization and nebulizing devices, sprays (coarse sprays, spray on feed), and drinking water. Inhalation implies inhalation of liquids or powder. Application routes may be combined, e.g. intranasal and oral application.

The preferred modes of administration are intranasally, orally, oronasally, sublingually, subcutaneously, intradermally, transcutanously, conjunctivally and intramuscularly, intranasally and orally being most preferred.

The bacterial host cell of the invention may be administered alone or in combination with adjuvants that enhance stability and/or immunogenicity of the bacteria, facilitate administration of pharmaceutical compositions containing them, provide increased dissolution or dispersion, increase propagative activity, provide adjunct therapy, and the like, including other active ingredients.

Pharmaceutical dosage forms of the bacterial host cells, for example, E. coli and Salmonella enterica subsp. Enterica, optionally serovar Typhimurium, as described herein, include pharmaceutically acceptable carriers and/or adjuvants known to those of ordinary skill in the art. These carriers and adjuvants include, for example, water or buffered solutions with or without detergents and or salts, metallic salts (for example aluminium based), saponins, oils (mineral and non-mineral oils), oil emulsions, bacterial derivatives, cytokines, iscoms, liposomes, micorparticles, vitamines (for example alpha tocopherole), dextran, carbomer, micro-emulsions, synthetic oligodeoxynucleotides and other immunostimulating compounds, alone or in combination. Preferred dosage forms include solutions, suspensions, emulsions, powders, tablets, capsules, and transdermal patches. Methods for preparing dosage forms are well known, see, for example, Ansel et al. 1990, Pharmaceutical Dosage Forms and Drug Delivery Systems, 5th ed., ISBN: 978-0812112559 and, in particular, Pastoret 1999, Acad. Sci. Paris/Elsevier SAS, Vol. 322, p967-972.

For example, vaccination with the bacterial host cells and compositions of the present invention may be performed as single vaccination, or with a boost vaccination, possibly followed by re-vaccinations after defined periods. The vaccine may consist of live bacteria, applied in a dose of, for example, between 10E4 to 10E6 cfu (colony forming units), or it may consist of inactivated bacteria, applied in a dose of, for example, between 10E6 to 10E11 cfu (colony forming units), with or without adjuvant(s).

The following Figures and Examples serve to illustrate the invention and are not intended to limit the scope of the invention as described in the appended claims.

### BRIEF DESCRIPTION OF THE DRAWINGS

FIG. 1 is a schematic drawing of the APP rfb cluster identified from sequenced APP2 strain. The rfb cluster of APP2 with a length of 12928 bp consists of 13 genes encoding putative glycosyltransferases, glycan modifying enzymes, chain length determinant protein, O-antigen transporter, acetyltransferase and hypothetical protein (with homology to O-antigen polymerizing enzyme wzy). Positions of genes and cluster size is given in base pairs. h.P.—hypotethical protein

FIG. 2: is a schematic drawing of the O-antigen biosynthesis cluster of APP2 with flanking gene erpA and an additional downstream sequence was used to be integrated into SL1344 and E. coli_5. The complete fragment has a length of 13758 bp. Positions of genes and cluster size is given in base pairs. h.P.—hypotethical protein

FIG. 3 is a schematic drawing of the APP2 O-antigen biosynthesis cluster located on pDOC plasmid with flanking homologous host cell regions of the rfb cluster. The 13758 bp fragment (see FIG. 2) containing the APP2 rfb cluster and its flanking regions was modified for integration in an antisense orientation to the endogenous rfb cluster into SL1344 and E. coli_5. For this a kanamycin resistance cassette flanked by FRT sites was fused downstream. The fusion construct is flanked upstream and downstream by homologous regions flanking the rfb cluster of either SL1344 or E. coli_5 (including the galF gene in antisense direction). The whole construct is flanked by I-Scel restriction endonuclease recognition sites.

FIG. 4: is a schematic drawing of the codon optimized APP2 O-antigen biosynthesis cluster located on the pDOC plasmid with flanking homologous regions of the endogenous rfb cluster. To integrate the codon optimized rfb cluster of APP2 into SL1344 and E. coli_5 the original integration sites at the endogenous rfb cluster were kept identical. A codon optimized (for E. coli expression) APP2 rfb cluster was synthesized which encoded at its 5′ region the kanamycin resistance cassette (flanked by FRT sites). This results in the transcription regulated by the promoter of the kanamycin gene. The whole construct is flanked by I-Scel restriction endonuclease recognition sites.

FIG. 5A, 5B, 5C, and 5D are photographs of a Western blot (5A, 5C) directed against the APP2 LPS and a silver staining (5B, 5D) of the gel for the verification of the extrachromosomal expression of wzy and gne in SL1344 expressing the APP2 0-antigen biosynthesis cluster (5A, 5B) and the extrachormosomal expression of gne in E. coli_5 expressing the APP2 O-antigen biosynthesis cluster (5C, 5D). Wzy and/or gne were encoded on plasmids under the control of an arabinose inducible promoter in SL1344 and E. coli_5 cells expressing the APP2 rfb cluster (with or without codon optimization) downstream of the kanamycin resistance cassette. Cells were induced with 0.1% (SL1344 cells) or 0.2% (E. coli_5) arabinose and overnight cultures analysed for APP2 LPS expression by proteinase K treatment of cells and analysis of digested extract via SDS-PAGE. On the left side the molecular marker bands are indicated in kDa. Lane 1: APP2 P1875, lane 2: SL1344, lane 3: SL1344 Δrfb, lane 4: SL1344 Δrfb::kanR-APP2.LPS pMLBAD-gne pEC415-wzy, lane 5: SL1344 Δrfb::kanR-APP2.LPS(cod.opt.) pMLBAD pEC415, lane 6: SL1344 Δrfb::kanR-APP2.LPS(cod.opt.) pMLBAD-gne, lane 7: SL1344 Δrfb::kanR-APP2.LPS(cod.opt.) pEC415-wzy, lane 8: SL1344 Δrfb::kanR-APP2.LPS(cod.opt.) pMLBAD-gne pEC415-wzy, lane 9: E. coli_5, lane 10: E. coli_5 Δrfb, lane 11: E. coli_5 Δrfb::kanR-APP2.LPS(cod.opt.), lane 12: E. coli_5 Δrfb::kanR-APP2.LPS(cod.opt.) pMLBAD, lane 13: E. coli_5 Δrfb::kanR-APP2.LPS(cod.opt.) pMLBAD-gne.

FIGS. 6A and 6B are schematic representations of overlap PCR to generate fragment rfaL-Ωgne/cat (6A) and rfaL-Ωgne-wzy(cod.opt.)/cat (6B) to be integrated into E. coli_5 and SL1344 derivatives. (6A) To generate rfaL-Ωgne/cat individual fragments for gne and cat with overlapping regions were amplified. With an overlap PCR the two fragments were fused and the 5′ and 3′ ends were elongated to encode homologous regions with the target integration sites in the genome of E. coli_5 and SL1344. The same principle was followed by generating the integration construct rfaL-Ωgne-wzy(cod.opt.)/cat (6B) except that an additional fragment encoding the codon optimized wzy was generated before combining the 3 fragments by overlap PCR and integrating homologous recombination sequences at the 5 and 3′ end.

FIG. 7 is a schematic representation of the cat/kP integration construct. To integrate the kanamycin promoter (kP) upstream of the codon optimized APP2 rfb cluster a 1414 bp construct was synthesized containing a chloramphenicol resistance cassette with flanking FRT sites followed by the 373 bp promoter region of the kanamycin resistance cassette (encoded on pKD4).

FIGS. 8A, 8B, and 8C show a comparison of the APP2 O-antigen expression levels in SL1344 and E. coli_5 expressing the APP2 O-antigen biosynthesis cluster with or without further glycoengineering (KanR and Kp control of APP2 rfb cluster expression, wzy and/or gne integration). Saturated overnight cultures of cells listed in FIG. 8A were analysed for APP2 O-antigen expression by proteinase K treatment and analysis of digested cell extract via SDS-PAGE. FIG. 8B is photograph of a silver staining directed against the APP2 LPS and FIG. 8C a Western blot using rabbit serum reactive with APP2 LPS of the gel. On the left side, the molecular marker bands are indicated in kDa.

FIG. 9 is a schematic drawing of the APP rfb cluster identified from sequenced APP8 strain. The rfb cluster of APP8 with a length of 13598 bp consists of 13 genes (potential functions of the individual genes are listed in the table). Positions of genes and cluster size is given in base pairs.

FIG. 10 is a schematic drawing of the codon optimized APP8 O-antigen biosynthesis cluster located on the pDOC_SL1344_Δrfb::cat-kP-APP8.LPS(cod.opt.) with flanking homologous regions of the endogenous rfb cluster (including galF of SL1344). Upstream of the codon optimized rfb cluster of APP8 a chloramphenicol resistance cassette (flanked by FRT sites) followed by the kP (kanamycin) promoter are located. The whole construct is flanked by I-Scel restriction endonuclease recognition sites.

FIGS. 11A and 11B show the APP8 O-antigen expression level in SL1344 encoding the codon optimized APP8 rfb cluster under the control of the kP promoter. Saturated overnight cultures of SL1344 (lane 1), SL1344 Δrfb (lane 2), APP8 (lane 3), APP3 (lane 4) and SL1344 Δrfb::cat-kP-APP8.LPS(cod.opt.) (lane 5) were analysed for APP8 O-antigen expression by proteinase K treatment and analysis of digested cell extract via SDS-PAGE. (11A) is photograph of a silver staining directed against the LPS and (11B) a Western blot using pig serum reactive with APP3 LPS of the gel. The molecular marker bands (M) are indicated in kDa.

FIGS. 12A and 12B demonstrate the expression and purification HIS10-ApxII(439-801aa). HIS10-ApxII(439-801aa) was expressed in BL21 cells encoding pMLBAD-H1510-ApxII(439-801aa) after arabinose induction. After 4 h incubation cells were harvested and broken via lysozyme treatment followed by sonication-freeze-thawing cycles. Proteins were purified under denaturing conditions by Ni-NTA binding and gravity flow. Elution (E) from the Ni-NTA beads was achieved by 5 times 1 ml denaturing buffer containing 0.5 M imidazole. 7.5 μl of each elution fraction (lane 1: E1, Ian2 2: E2, lane 3: E3, lane 4: E4, lane 5: E5) were loaded on for SDS-PAGE and analysed via Coomassie staining (12A) and Western blot directed against HIS epitopes (12B). On the left and right side, the molecular marker bands are indicated in kDa.

FIGS. 13A and 13B demonstrate the expression and purification of ApxIII(27-245aa)-HIS9. ApxIII(27-245aa)-HIS9 in BL21 cells encoding pMLBAD-ApxIII(27-245aa)-HIS9 after arabinose induction. After 4 h incubation cells were harvested and broken via lysozyme treatment followed by sonication-freeze-thawing cycles. Proteins were purified by Ni-NTA binding and gravity flow. The buffer was exchanged to PBS in the pooled elution fractions. 7.5 μl of the pooled and dialysed sample was used for SDS-PAGE and analysed via Coomassie staining (13A) and Western blot directed against HIS epitope (13B). On the left side, the molecular marker bands are indicated in kDa.

FIG. 14 is a schematic representation of synthesized ClyA-ApxI(628-845aa)-ApxII(612-801aa)-ApxIII(626-860aa)-HIS6 with flanking homologous recombination sites, synthetic promoter and 3′ chloramphenicol resistance cassette.

FIGS. 15A, 15B, and 15C demonstrate the expression of ClyA-ApxI(aa626-845)-ApxII(aa612-801)-ApxIII(aa626-860)-HIS6 in SL1344 with improved APP2 O-antigen expression. The strains analyzed are shown with their loading pattern indicated in (15A). Overnight cultures in liquid medium were diluted to 0.05 OD600 and grown until logarithmical growth (OD₆₀₀˜1). Cells were harvested, cooked in 1×Lämmli, loaded on a gradient Bis Tris gel for SDS-PAGE and analysed via Coomassie staining (15B) and Western blot directed against HIS epitope (15C). On the left and right side, the molecular marker bands are indicated in kDa.

FIG. 16 is an overview of the immunization schedule. Pigs were immunized with inactivated test materials twice at study day (SD) 0 and SD 14 and euthanized 2 weeks after the last immunization (SD 28). Blood was sampled weekly for serum collection. The animals were daily monitored for clinical signs.

FIG. 17 shows serum IgG mediated absorbance at timepoints SD 0 and SD 28 of all animals tested against purified LPS of APP serotype 2 (APP2) and 7 (APP7). The individual animals are shown according to their ear tags. The OD at 450 nm was recorded of single measurements. The individual diagrams are sorted for the applied antigen and its application (oral & nasal vs. injection).

FIG. 18 shows BALF IgA mediated absorbance at timepoints SD 28 of all animals tested against purified LPS of APP serotype 2 (APP2), 1 (APP1), 5 (APPS) and 7 (APP7). The individual animals are shown according to their ear tags. The OD at 450 nm was recorded of duplicate measurements (error bars are indicated). The individual diagrams are sorted for the applied antigen and its application (oral & nasal vs. injection).

FIG. 19 shows serum IgG mediated absorbance at timepoints SD 0 and SD 28 and BALF IgA mediated absorbance at timepoint SD 28 of all animals of group 2 and 7, two animals of group 3 and 10 and one animal of group 11 were tested against purified ApxII(439-801aa) and AcrA-HIS6 protein (as negative control). The individual animals are according to their ear tags. The OD at 450 nm was recorded of single measurements. The individual diagrams are sorted for the applied antigen and its application (oral & nasal vs. injection).

FIG. 20 is a schematic overview of the immunization schedule. Pigs were immunized with live, recombinant bacteria twice at study day (SD) 0 and SD 14 and euthanized 2 weeks after the last immunization (SD 28). Blood was sampled weekly for serum collection. The animals were daily monitored for clinical signs.

FIG. 21 is an overview of the vaccination and challenge schedule. Pigs were vaccinated twice at SD 0 and SD21, followed by a challenge with APP2 bacteria at SD 42. The pigs were euthanized on day 48 (SD 48). At indicated timepoints sera were collected. The animals were regularly monitored for clinical signs (at least twice daily during the 6 days after challenge).

FIG. 22 shows lesion scoring according to Hannan et al. 1982. Group 1: live SL1344 Δrfb::kanR-APP2.LPS(cod.opt.) rfaL-Ωgne-wzy(cod. opt.)/cat, purified HIS10-APXII(439-801aa), purified APXIII(27-245aa)-HIS9. Group 2: inactivated SL1344 Δrfb::kanR-APP2.LPS pEC415-wzy pMLBAD-gne, purified HIS10-APXII(439-801aa), purified ApxIII(27-245aa)-HIS9. Group 3: purified HIS10-APXII(439-801aa), purified ApxIII(27-245aa)-HIS9. Group 4: non-vaccinated control group. Statistics were performed by Dunnett's multiple comparison test. Statistic significance shown as (*) P Value 0.01.

FIG. 23 shows the survival rate (%) over 6 days post challenge sorted by groups with day 0 being the day of challenge and day 6 the end of the study. Group 1: inactivated SL1344 Δrfb::kanR-APP2.LPS pEC415-wzy pMLBAD-gne, purified HIS10-APXII(439-801aa), purified APXIII(27-245aa)-HIS9; Group 2: commercial vaccine Porcilis® APP. Group 3: non-vaccinated control group.

FIG. 24 shows lesion scoring according to Hannan et al. 1982. Group 1: inactivated SL1344 Δrfb::kanR-APP2.LPS pEC415-wzy pMLBAD-gne, purified HIS10-APXII(439-801aa), purified ApxIII(27-245aa)-HIS9. Group 2: commercial vaccine Porcilis® APP. Group 3: non-vaccinated control group. Statistics were performed by Dunnett's multiple comparison test. Statistic significance shown as (**) P-value<0.01.

FIG. 25 shows APP2 resolution evaluated of all animals and sorted by groups. The bacteriology score values are set as 0 =no APP2 bacteria, 1=<20 CFU (colony forming units) APP2 bacteria, 2=<200 CFU APP2 bacteria, 3 =>200 CFU APP2 bacteria reisolated.

### DETAILED DESCRIPTION OF THE INVENTION

In the following the present invention will by described by representative examples, none of which are to be interpreted as limiting the scope of the claims as appended.

### Examples

### Materials and Methods

The bacterial strains used in the experimental examples and their sources are listed in Table 1.

TABLE 1
Bacteria

| Background | Genotype | Resistance | Source/reference |
|---|---|---|---|
| APP | Serotype 2 strain P1875 | | Provided by V. Perreten, |
| | | | University of Bern |
| APP | Serotype 2 strain HK361 | | NCTC 10976 |
| APP | Serotype 1 strain S4074 | — | ATCC 27088 |
| APP | Serotype 3 strain ORG1224 | — | Provided by V. Perreten, |
| | | | University of Bern |
| APP | Serotype 5 strain K17 | — | APP reference strain |
| | | | provided by V. Perreten, |
| | | | University of Bern |
| APP | Serotype 7 WF83 | — | APP reference strain |
| | | | provided by V. Perreten, |
| | | | University of Bern |
| APP | Serotype 8 strain MIDG2331 | — | Provided by V. Perreten, |
| | | | University of Bern |
| SL1344 | his- (histidine auxotroph) | Strep | Hoiseth et al. 1981, Nature, |
| | | | Vol. 291(5812), p238-239 |
| SL1344 | his-, Δrfb::kanR | Kan, Strep | This study |
| SL1344 | his-, Δrfb | Strep | This study |
| SL1344 | his-, Δrfb::APP2.LPS/kanR | Kan, Strep | This study |
| SL1344 | his-, Δrfb::APP2.LPS | Strep | This study |
| SL1344 | his-, Δrfb:: kanR -APP2.LPS | Strep, Kan, Cm | This study |
| SL1344 | his-, Δrfb::kanR -APP2.LPS(cod. opt.) | Kan, Strep | This study |
| SL1344 | his-, Δrfb::kanR -APP2.LPS rfaL-Ωgne/cat | Kan, Strep, Cm | This study |
| SL1344 | his-, Δrfb::kanR -APP2.LPS rfaL-Ωgne- | Strep, Kan, Cm | This study |
| | wzy(cod. opt.)/cat | | |
| SL1344 | his-, Δrfb::kanR -APP2.LPS(cod. opt.) | Strep, Kan, Cm | This study |
| | rfaL-Ωgne-wzy(cod. opt.)/cat | | |
| SL1344 | his-, Δrfb::APP2.LPS(cod. opt.) | Strep | This study |
| | rfaL-Ωgne-wzy(cod. opt.) | | |
| SL1344 | his-, Δrfb::cat/kP-APP2.LPS rfaL- | Strep, Cm | This study |
| | Ωgne-wzy(cod. opt.) | | |
| SL1344 | his-, Δrfb::kP-APP2.LPS(cod. opt.) | Strep | This study |
| | rfaL-Ωgne-wzy(cod. opt.) | | |
| SL1344 | his-, Δrfb rfaK-ΩproD-ClyA- | Strep | This study |
| | ApxI(aa626-845)-ApxII(aa612-801)- | | |
| | ApxIII(aa626-860)-HIS6-rfaL | | |
| SL1344 | his-, Δrfb::kP-APP2.LPS(cod. opt.) | Strep | This study |
| | rfaL-Ωgne-wzy(cod. opt) pliC-ΩproD- | | |
| | ClyA-ApxI(aa626-845)-ApxII(aa612-801)- | | |
| | ApxIII(aa626-860)-HIS6-pagC | | |
| SL1344 | his-, Δrfb::cat-kP-APP8.LPS(cod. opt.) | Strep | This study |
| E. coli_5 | | Amp, Strep | Provided by Roger Stephan, |
| | | | University of Zurich |
| E. coli_5 | Δrfb::kanR | Kan, Amp, | This study |
| | | Strep | |
| E. coli_5 | Δrfb | Amp, Strep | This study |
| E. coli_5 | Δrfb:APP2.LPS/kanR | Kan, Amp, | This study |
| | | Strep | |
| E. coli_5 | Δrfb::APP2.LPS | Amp, Strep | This study |
| E. coli_5 | Δrfb::kanR -APP2.LPS | Amp, Strep, | This study |
| | | Kan | |
| E. coli_5 | Δrfb::kanR -APP2.LPS(cod. opt.) | Kan, Amp, | This study |
| | | Strep | |
| E. coli_5 | Δrfb::kanR -APP2.LPS(cod. opt.) | Amp, Strep, | This study |
| | rfaL-Ωgne/cat | Kan, Cm | |
| E. coli_5 | Δrfb::kanR-APP2.LPS(cod. opt.) | Amp, Strep, | This study |
| | rfaL-Ωgne-wzy(cod. opt.)/cat | Kan, Cm | |

The plasmids used in the experimental examples and their sources are listed in Table 2.

TABLE 2
Plasmids

| | | |
|---|---|---|
| pMLBAD | Tmp | Lefebre et al. 2002, |
| | | Appl. Environ. Microbiol., |
| | | Vol. 68(12), p5956-64 |
| pEC415 | Amp | Schulz et al. 1998, Science, |
| | | Vol. 181, p1197-1199 |
| pMLBAD-HIS10-APXII(439-801aa) | Tmp | This study |
| pMLBAD-HIS10-APXIII-APP2 | Tmp | This study |
| pMLBAD-APXIII(27-245aa)-HIS9 | Tmp | This study |
| pMLBAD-gne-HA | Tmp | This study |
| pEC415-wzy | Amp | This study |
| pDOC | Amp | Lee et al. 2009, BMC |
| | | Mircobiol., Vol. 9(252) |
| pDOC_E.coli_5_Δrfb::APP2.LPS/kanR | Amp, Kan | This study |
| pDOC_SL1344_Δrfb::APP2.LPS/kanR | Kan, Amp | This study |
| pDOC_E.coli_5_Δrfb::kanR-APP2.LPS(cod. opt.) | Amp, Kan | This study |
| pDOC_SL1344_Δrfb::kanR-APP2.LPS(cod. opt.) | Amp, Kan | This study |
| pDOC_SL1344_Δrfb::cat-kP-APP8.LPS(cod. opt.) | Amp, Cm | This study |
| pACBSCE | Cm | Lee et al. 2009, BMC |
| | | Mircobiol., Vol. 9(252) |
| pKD46 | Amp | Datsenko et al. 2000, PNAS, |
| | | Vol. 97(12), P6640-6645 |
| PLAMBDA46 | Gent | This study |
| pCP20 | Amp, Cm | Cherepanov et al. 1995, |
| | | Gene, Vol. 158, p9-14 |
| pCP20-GentR | Gent, Cm | This study |
| pKD3 | Amp, Cm | Datsenko et al. 2000, PNAS, |
| | | Vol. 97(12), P6640-6645 |
| pKD4 | Amp, Kan | Datsenko et al. 2000, PNAS, |
| | | Vol. 97(12), P6640-6645 |

The plasmids' oligonucleotides used in the experimental examples and their nucleic acid sequences are listed in Table 3.

TABLE 3

| OLIGO name | Sequence |
|---|---|
| Ec_SL/Kan_f | CAAATTCCGGTTAAAAAAAG |
| W | ACCGCTTGTTTGAGAGTGAT |
| | AATCGCAAACAAGCGGTCTT |
| | TTTTGATCAA |
| | AATATTATTACACGTCTTGA |
| | GCGATTG |
| | (SEQ ID NO: 9) |
| Ec_SL/Kan_re | GATGAAGAGCAAAGATTGGG |
| V | AGATAATGTGAGAAATCTTT |
| | AGATTCAAACTAAGCTGAGA |
| | AGAAAAAG |
| | GTCCATATGAATATCCTCCT |
| | TAG |
| | (SEQ ID NO: 10) |
| E.c._5_Δrfb | GTAATGTTAATGAAAGCATA |
| fw | TAAGAAATTTTCAAATGAAT |
| | AAAGAAACTGTTTCAGTTAT |
| | TATTACACGT |
| | CTTGAGCGATTG |
| | (SEQ ID NO: 11) |
| E.c._5_Δrfb | GAGCATGTAATCTTCTGATA |
| rev | AAAATCATTTGTACGATATT |
| | TTCAGTTACATACTATGCGT |
| | AGGTCCATATG |
| | AATATCCTCCTTAG |
| | (SEQ ID NO: 12) |
| SL1344_Δrfb | GAGCAATTAATTTTTATTGG |
| fw | CAAATTAAATACCACATTAA |
| | ATACGCCTTATGGAATAGAA |
| | AAATTACACG |
| | TCTTGAGCGATTG |
| | (SEQ ID NO: 13) |
| SL1344_Δrfb | GCGTTCAGATTTTACGCAGG |
| rev | CTAATTTATACAATTATTAT |
| | TCAGTACTTCTCGGTAAGCG |
| | GTCCATATGAA |
| | TATCCTCCTTAG |
| | (SEQ ID NO: 14) |
| Ec_SL/Kan_ | CAGGGCTAGCGCTAATTACC |
| fw_elo | AATTTATTGTTTAGCTTAGG |
| | AATTTTTTTAGGTTAGTTGC |
| | AAATTCCGGTT |
| | AAAAAAAGACCGCTTGTTTG |
| | A |
| | (SEQ ID NO: 15) |
| Ec_SL/Kan_ | CAATATTAGCTTATGTATTA |
| rev_elo | TATTAGAAGGCCTACAGATA |
| | AGCAAAAAATATTATTGATG |
| | AAGAGCAAA |
| | GATTGGGAGATAATGTGAGA |
| | AAT |
| | (SEQ ID NO: 16) |
| BamHI-Fw | CGGGAATTCAAGCTTGGATC |
| KanR-Fw | CC |
| | (SEQ ID NO: 17) |
| XhoI 3′rspU- | GACGCTAGCATATGAGCTCG |
| Rev | AG |
| | (SEQ ID NO: 18) |
| BamHI-SL- | GTTTCATCAGTAATGGGACA |
| gnd Fw ext | GAAAGGTACC |
| | (SEQ ID NO: 19) |
| XhoI-SL-GalF | CACACTCGAGCAATTGACCG |
| Rev ext | GTTTTTCTATTCCATAAGGC |
| | (SEQ ID NO: 20) |
| 5′ NdeI_wzy | CAGGTACCATATGAACTCCT |
| | TAGTATATAGAATAGATATT |
| | AGAACA |
| | (SEQ ID NO: 21) |
| 3′ EcoRI_wzy | CTTATCAGAATTCATTTTTT |
| | ACATTCCAAATAGCGTACAA |
| | (SEQ ID NO: 22) |
| 3′ | GTACCGAGCTCGAATTCTTG |
| EcoRI_pEC41 | AAGACGAAAGG |
| 5fw | (SEQ ID NO: 23) |
| 5′ | CACTGCAATCGCGATAGCTG |
| NdeI_pEC415 | TCTTTTTCATATGT |
| rev | (SEQ ID NO: 24) |
| 3′-gne- | GAATAGGAACTAAGGAGGAT |
| cat_overlap | ATTCATATGGACCTTAAGCG |
| | TAATCTGGAACATCGTATGG |
| | G |
| | (SEQ ID NO: 25) |
| 5′- | ATTGCTCAAATTGGTATCAT |
| gne_SI1344rf | TACCGGTTTTLTGCTGGCGC |
| aL | TAAGAAATAGATAATGAAAA |
| | TTCTTATTAG |
| | CGGTGGTGCA |
| | (SEQ ID NO: 26) |
| 3′- | AAAAACTGGTTTGATAAGTG |
| cat_SI1344 | ATTGAGTCCTGATGATGGAA |
| rfaL | AACGCGCTGATACCGTAATT |
| | GTGTAGGCT |
| | GGAGCTGCTTC |
| | (SEQ ID NO: 27) |
| 5′-elo- | TTTTATCTTTCGTCGGTTTT |
| gne_SH344rf | TATAFCGTTCGTGGCAATTT |
| aL | TGAACAGGTCGATATTGCTC |
| | AAATTGGTATC |
| | ATTACCGGT |
| | (SEQ ID NO: 28) |
| 3′-elo- | TTTCAAAATACAGTTGGGAA |
| cat_SI1344 | AATGTAGCGCAGCGTTTCGA |
| rfaL | GGAACAAATGAAAAACTGGT |
| | TTGATAAGT |
| | GATTGAGTCCT |
| | (SEQ ID NO: 29) |
| 5′-cat-P2new | GGTCCATATGAATATCCTCC |
| | TTAGTTCCTATTC |
| | (SEQ ID NO: 30) |
| 5′ gne | CCCATACGATGTTCCAGATT |
| overlap_wzy | ACGCTTAATGAATTCTTTAG |
| (co) | TGTATCGCATTGACATCC |
| | (SEQ ID NO: 31) |
| 3′ wzy | GAATAGGAACTAAGGAGGAT |
| (co)- | ATTCATATGGACCTCATTTT |
| cat_overlap | TTACACTCTAAATAACGCAC |
| | AATATTGG |
| | (SEQ ID NO: 32) |
| 3′ gne_wzy | GGATGTCAATGCGATACACT |
| (co) overlap | AAAGAATTCATTAAGCGTAA |
| | TCTGGAACATCGTATGGG |
| | (SEQ ID NO: 33) |
| 5′ XmaI-XhoI- | AAAAAACCCGGGCTCGAGAT |
| APXIIIne-HIS- | GGATGTAACTAAAAATGGTT |
| fw | TGCAATATGGG |
| | (SEQ ID NO: 34) |
| 3′ APXIIIne- | AAAAAAAAGCTTTTAGTGGT |
| HIS-HindIII- | GATGATGATGGTGATGGT |
| rv | (SEQ ID NO: 35) |
| FW_ | CTAATTAGTAACCACTTTTA |
| pliCint | AGCATGGTTAATCCTATTTT |
| | GAAAAAGCAAAATCCCTGGT |
| | GTTTTCAAAAT |
| | A |
| | (SEQ ID NO: 36) |
| REV_ | GATTCACTCTGAAAAATTTT |
| pagCint | CCTGGAATTAATCACAATGT |
| | CAGGTCGATATTGCTCAAAT |
| | TGGTATCATTA |
| | (SEQ ID NO: 37) |
| eloFW_ | CGTAACGTTAAAGAATATGT |
| pliCint | GAATCACTACCGTAGTATAA |
| | TGGCTAATTAGTAACCACTT |
| | TTAAGCATGG |
| | TTAATC |
| | (SEQ ID NO: 38) |
| eloREV_ | GATAAGCAGGAAGGAAAATC |
| pagCint | TGGTGTAAATAACGCCAGAT |
| | CTCACAAGATTCACTCTGAA |
| | AAATTTTCC |
| | TGGAATTAAT |
| | (SEQ ID NO: 39) |
| FW_rfaK_ | CTATTTATATGGCGCTATCA |
| rfaL | TCAGGGAAACAG |
| | (SEQ ID NO: 40) |
| REV_rfaL_ | GACAGTATAATTAATGATAT |
| rfaK | TAACCGTGCGCTTG |
| | (SEQ ID NO: 41) |

### Methods—Growth of Bacterial Strains

The bacterial strains and plasmids listed in above table 1 and 2 were grown in Luria-Bertani (LB) medium (10 g/L tryptone, 5 g/L yeast extract, 5 g/L NaCl), TB medium (12 g/L tryptone, 24 g/L yeast extract, 0.017 M KH₂PO₄, 0.072 M K₂HPO₄) or BHI+NAD (37 g/L brain heart infusion broth (BHI; exact composition see Sigma Aldrich cat. Nr. 53286) supplemented with 2 mg/L β-Nicotinamide adenine dinucleotide (NAD)).

Agar plates were supplemented with 1.5% (w/v) agar. Antibiotics were used in the following final concentrations: Ampicillin (Amp) 100 μg/ml, kanamycin (Kan) 50 μg/ml, chloramphenicol (Cm) 25 μg/ml, streptomycin (Strep), trimethoprim (Tmp) 10 μg/ml, gentamycin (gent) 15 μg/ml.

### Example 1

### 1) Generation of a vaccine strain Expressing The APP2 O-Antigen Biosynthesis Cluster

1.1) Integration of the APP2 O-antigen Biosynthesis Cluster in E. coli and SL1344

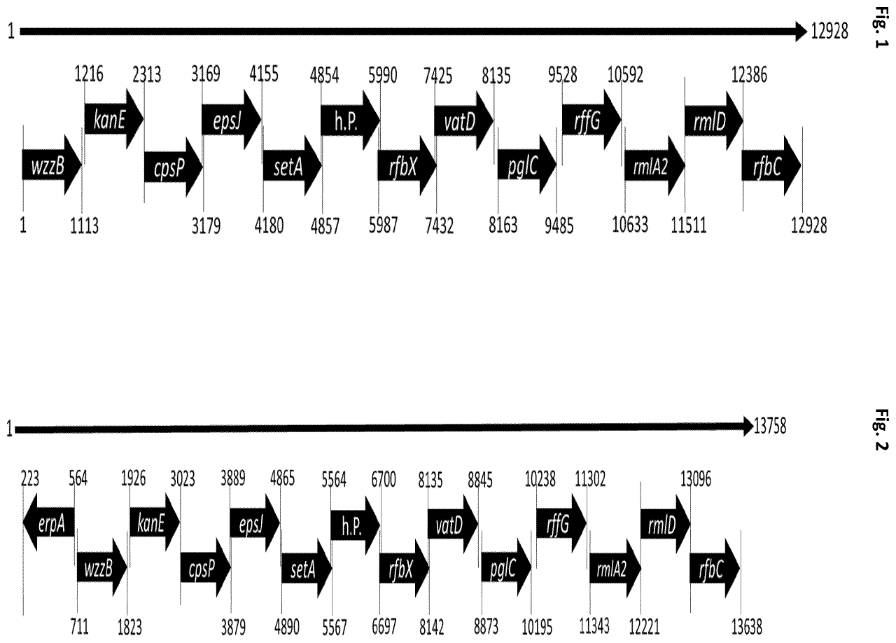

In a first step an Actinobacillus pleuropneumoniae serotype 2 (APP2) strain (P1875) was sequenced and the O-antigen biosynthesis cluster (rfb cluster) identified according to Xu et al. 2010, J. Bacteriol., Vol. 192(21), p5625-5636. It was shown that the rfb cluster of all APP serotypes used in this publication were located between the erpA and rpsU genes. FIG. 1, Table 4 and Seq. 1 show the genetic organization and annotated genes/proteins for the sequenced APP2 strain of this study.

TABLE 4
rfb locus of APP2 flanked by erpA and rpsU with its
respective annotated genes and proteins/functions.

| Annotated gene | Predicted protein/function |
|---|---|
| wzzB | chain length determinant protein |
| kanE | alpha-D-kanosaminyltransferase |
| cpsP | glycosyl transferase family 2 |
| epsJ | putative glycosyltransferase EpsJ |
| setA | subversion of eukaryotic traffic protein A |
| hypothetical | hypothetical protein |
| gene | |
| rfbX | putative O-antigen transporter |
| vatD | streptogramin A acetyltransferase |
| pglC | undecaprenyl phosphate N,N′- |
| | diacetylbacillosamine 1-phosphate transferase |
| rffG | dTDP-glucose 4,6-dehydratase 2 |
| rmlA2 | glucose-1-phosphate thymidylyltransferase 2 |
| rmlD | dTDP-4-dehydrorhamnose reductase |
| rfbC | dTDP-4-dehydrorhamnose 3,5-epimerase |

As vaccine strains, two vector bacteria derived from Salmonella enterica subsp. enterica Typhimurium and Escherichia coli (see strain list) were selected. Salmonella enterica subsp. enterica serovar Typhimurium SL1344 parental strain was isolated from infected cattle (Hoiseth et al. 1981, Nature, Vol. 291(5812), p238-239). The strain SL1344 is the genetic marked version of the parental strain. The second strain, Escherichia coli strain 5 (E. coli_5) was isolated from tonsils of healthy pigs in Switzerland. It was shown to be PCR negative for virulence factors stx, eae, LT and ST. Both strains were sequenced and the endogenous O-antigen biosynthesis cluster (rfb cluster) identified between genes galF and gnd. Furthermore, both strains were genetically modified not to express endogenous O-antigen and instead a gene cluster encoding the O-antigen biosynthetic pathway of APP2 inserted at this position (Seq. 42, FIG. 2).

The 13758 bp fragment (FIG. 2) containing the APP2 rfb cluster and its flanking regions was modified for integration in an antisense orientation to the endogenous rfb cluster into SL1344 and E. coli_5. For this a kanamycin resistance cassette flanked by FRT sites was fused downstream. The fusion construct is flanked upstream and downstream by homologous regions flanking the rfb cluster of either SL1344 or E. coli_5 (including the galF gene in antisense direction).

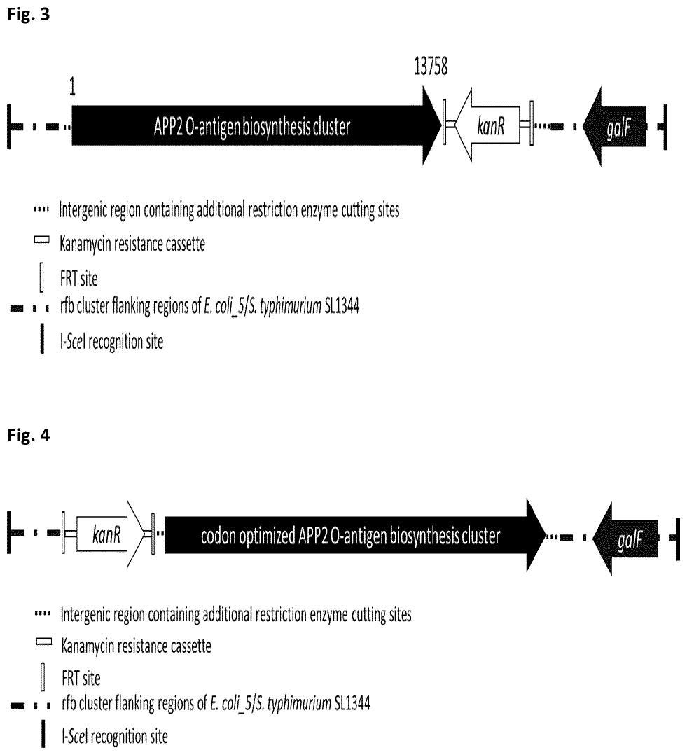

In detail, to integrate the APP2 rfb cluster (Seq. 1) into E. coli_5 and SL1344, shuttle vector plasmids pDOC_E.coli_5_Δrfb::APP2.LPS/kanR (for E. coli_5) and pDOC_SL1344_Δrfb::APP2.LPS/kanR (for SL1344) based on the pDOC system of Lee et al. 2009 were generated/synthesized (by CRO). The identified APP2 rfb operon (gene wzzB to rfbC) was elongated on the 5′ and 3′ end to include the flanking upstream pro-moter region (containing also the erpA gene) and downstream the terminator region (containing the 3′ region of rpsU gene) of Sequence 1 (see FIG. 2). This sequence was fused at the 3′ end with a kanamycin resistance marker gene (kanR) with 5′ and 3′ flanking FRT recognition sites (palindromic flippase recognition sites). From the sequencing results of E. coli_5 and SL1344 the flanking regions of the endogenous rfb cluster were retrieved and used as flanking regions to the APP2 rfb cluster and kanR resistance cassette as following. An 800bp fragment right after the stop of the last gene of the endogenous rfb cluster of E. coli_5) and SL1344 was chosen as the upstream homologous recombination sequence located before the APP2 rfb cluster. Regions of 1252bp (E. coli_5) or 1399bp (5L1344) were chosen as homologous recombination sequences downstream of the kanR. These regions contain the entire galF gene and all nucleotides up to the +1 position before the start codon of first gene of the cluster. The whole integration cluster was flanked by I-Secl restriction endonuclease cleavage sites. The integration cassette was designed to integrate in an antisense direction of the endogenous rfb cluster. A general schematic representation of the constructs is shown in FIG. 3. For the integration of the APP2 rfb operon on the shuttle vector further genes were necessary. As plasmid selection marker an ampicillin resistance cassette was used. The sucB gene (involved in sucrose conversion which in presence of sucrose generates a toxic metabolite) was integrated for vector plasmid counterselection.

The shuttle vector plasmids (pDOC_E.coli_5_Δrfb::APP2.LPS/kanR in E. coli_5 and pDOC_SL1344_Δrfb::APP2.LPS/kanR in SL1344) together with the helper plasmid pACBSCE which encodes the arabinose inducible λ-recombination system, and carries the I-Scel restriction enzyme as well as I-Scel cleavage sites, were transformed into target cells. After transformation the expressed I-Scel enzyme cleaved the shuttle vector plasmid at the I-Scel restriction endonuclease cleavage sites, linearizing the above described modified APP2 rfb operon with the kanamycin resistance gene flanked by endogenous homologous sequences. The λ-recombination system recognized the homologous flanking regions at the respective sites in the genome of the recipient cell and exchanged the endogenous DNA with the donor DNA resulting in the integration of the APP2 O-antigen biosynthesis cluster with a kanamycin resistance cassette in either E. coli_5 or SL1344 (resulting in strains E. coli_5 Δrfb::APP2.LPS/kanR and SL1344Δrfb::APP2.LPS/kanR). Cells selected on kanamycin and sucrose containing medium were positively tested by PCR for the presence of the foreign DNA and the absence of shuttle vector and helper plasmid by lack of growth on selective medium.

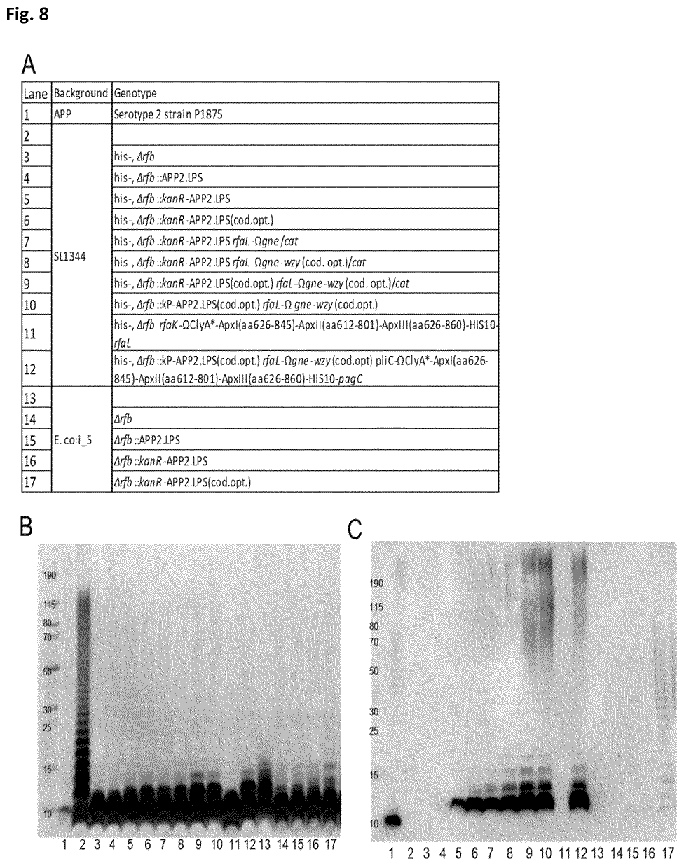

To “out-recombine” the kanamycin resistance marker, a temperature sensitive plasmid pCP20 (Cherepanov et al. 1995, Gene, Vol. 158, p9-14) for SL1344 derivatives or pCP20-Gent for E. coli_5 derivatives (downstream of the chloramphenicol resistance gene a gentamycin resistance gene was integrated), encoding for the flippase, which recognized the palindromic FRT sites, was introduced in the cells. After the “flip out” event only one FRT site remained in the genome. With increasing cultivation temperature, the positive clones were counter-selected against the flippase encoding plasmid. A final PCR verified the absence of all helper plasmids, absence of kanamycin resistance marker and the introduction of the APP2 rfb cluster construct (resulting in strains E. coli_5 Δrfb::APP2.LPS and SL1344 Δrfb::APP2.LPS). The expression of APP2 0-antigen displayed on the surface of E. coli_5 and SL1344 was verified by SDS-PAGE and immunoblotting (FIG. 8). Procedure of analysis and results are described under 1.6.

As control, the endogenous rfb cluster was deleted in the wild type cells E. coli_5 and SL1344. For this a knock-out cassette was generated by PCR using pKD4 as template (Datsenko et al. 2000, PNAS, Vol. 97(12), p6640-6645) and oligonucleotides E.c._5_Δrfb fw/E.c._5_Δrfb rev for E. coli_5 rfb and SL1344_Δrfb fw/ SL1344_Δrfb fw for SL1344 rfb cluster deletion. The resulting PCR product encodes the kanamycin resistance cassette flanked by FRT sites and in addition contains 21-24bp overlap sequences with the endogenous 5′ (before the start of the first gene of rfb cluster→wfgD (E. coli_5) or rfbB (SL1344)) and 3′ (after stop codon of last gene of rfb cluster→pg/H (E. coli_5) or rfbP (SL1344)) needed for homologous recombination. The DNA fragment (E. coli_5 1759bp, SL1344 1623bp) and a temperature sensitive helper plasmid encoding λ-recombination system pKD46 (Datsenko et al. 2000, PNAS, Vol. 97(12), p6640-6645) for SL1344 and pLAMBDA46 (the β-lactamase gene was exchanged with a gentamycin resistance gene) for E. coli_5 were introduced. The λ-recombination system recognized the homologous flanking regions at the respective sites before the start of the first gene and after the stop codon of the last gene in the rfb cluster and deleted the endogenous rfb gene cluster by integrating the kanamycin resistance cassette flanked by the FRT sites. The resulting strains E. coli_5 Δrfb::kanR and SL1344 Δrfb::kanR were further manipulated to lose the antibiotic marker. To “out-recombine” the kanamycin resistance marker, the flip out technique established by Cherepanov et al. 1995, Gene, Vol. 158, p9-14 was used as described above. The final E. coli_5 Δrfb and SL1344 Δrfb strains were verified by PCR and analyzed for the loss of O-antigen expression by immunoblotting (FIG. 8). Procedure of analysis and results are described under 1.6.

### 1.2) Improving the Expression of the APP2 O-antigen in E. coli_5 and SL1344 by introducing the kanR resistance marker upstream of the APP2 rfb cluster

To improve the APP2 O-antigen presentation in the glycoengineered E. coli_5 and SL1344 strains the promoter region of the rfb cluster was exchanged with a kanamycin resistance gene of which the kanamycin promoter is exploited to induce transcription of downstream genes. For this a PCR fragment was generated using oligonucleotide Ec_SL/Kan_fw and Ec_SL/Kan_rev and pKD4 as template amplifying the encoded kanamycin promoter and resistance gene with the 5′ and 3′ encoded FRT sites. Furthermore, the oligonucleotides introduced homologous recombination sequences homologous to the erpA gene before the start codon and after the stop codon. The resulting product with a size of 1644bp was used together with oligonucleotides Ec_SL/Kan_fw_elo and Ec_SL/Kan_rev_elo for a second PCR reaction to elongate the homologous recombination sequences for an increase in integration efficiency. This DNA fragment and a temperature sensitive helper plasmid encoding λ-recombination system pKD46 (Datsenko et al. 2000, PNAS, Vol. 97(12), p6640-6645) for SL1344 Δrfb::APP2.LPS and pLAMBDA46 for E. coli_5 Δrfb::APP2LPS strain were introduced. The λ-recombination system recognized the homologous flanking regions at the respective sites at the start and stop codon of erpA and “out-recombined” the erpA gene and integrated the kanamycin promoter and resistance gene flanked by the FRT sites into the respective site. The introduced kanamycin resistance gene allowed the selection of positive clones (successful integration). These positive candidates were verified for the deletion of erpA and the integration of the PCR fragment by PCR. The temperature sensitive helper plasmid was lost from the cells by increasing the growth temperature for several rounds of incubations. The O-antigen presentation in the final strains E. coli_5 Δrfb::kanR-APP2.LPS and SL1344 Δrfb::kanR-APP2.LPS was tested by immunoblotting (FIG. 8). Procedure of analysis and results are described under 1.6.

### 1.3) Improving the Expression of the APP2 O-antigen in E. coli_5 and SL1344 by Codon Optimization of the APP2 rfb Cluster

To improve the APP2 O-antigen cluster protein/enzyme expression and ultimately increase the APP2 O-antigen presentation on lipid A, the nucleotide triplet codons of the gene coding sequences could be optimized for SL1344 and E. coli_5. This results in the decrease of usage of unwanted triplets which might be in favour in A. pleuropneumoniae but unfavourable in SL1344 and E. coli_5. In this study the codon optimization was done by CRO and done for E. coli expression levels. If the same holds true for Salmonella enterica subsp. enterica serovar Typhimurium SL1344 needed to be shown by integration of the same codon optimized APP2 rfb cluster into SL1344 endogenous rfb cluster.

To integrate the codon optimized rfb cluster of APP2 into SL1344 and E. coli_5 the original integration sites at the endogenous rfb cluster where kept identical. A codon optimized (for E. coli expression) APP2 rfb cluster was synthesized which encoded the kanamycin resistance cassette (flanked by FRT sites) at its 5′ end. This results in the transcription of the APP2 rfb cluster (codon optimized) regulated by the promoter of the kanamycin gene. In detail, a new integration cassette was designed (FIG. 4) combining the kanamycin resistance gene integration upstream of the APP2 rfb cluster (Seq. 1 and 7) and the codon optimization of the APP2 rfb cluster (CRO which performed the synthesis applied the codon optimization for E. coli expression of the plasmid). The resulting plasmid (Seq. 43; pDOC_E.coli_5_Δrfb::kanR-APP2.LPS(cod.opt.)) was used for the manipulation of E. coli_5 and for generating the plasmid pDOC_SL1344_Δrfb::kanR-APP2.LPS(cod.opt.) (Seq. 44) which was used to genetically manipulate SL1344 cells. Oligonucleotides BamHI-Fw KanR-Fw/Xhol 3′rspU-Rev were used to amplify a 14972 bp fragment from plasmid pDOC_E.coli_5_Δrfb::Kan-APP2.LPS(cod.opt.) containing the codon optimized APP2 rfb cluster with the upstream integrated kanamycin resistance cassette flanked by FRT sites. In addition, new restriction cleavage sites were integrated at the 5′ (BamHI) and 3′ (Xhol) end by PCR. A second PCR was performed to generate a fragment containing the pDOC backbone encoding the homologous recombination sequences for the integration of the cassette in the rfb cluster of SL1344. As template the pDOC_SL1344_Δrfb::APP2.LPS/kanR was used and an 8113bp fragment with introduced BamHI and Xhol cleavage sites was generated by using primer BamHI-SL-gnd Fw ext/Xhol-SL-GalF Rev ext. After restriction digest with BamHI and Xhol of the PCR products both fragments were ligated. The sequence was confirmed by sequencing (pDOC_SL1344_Δrfb::kanR-APP2.LPS(cod.opt.)) and the plasmid further used for replacing the rfb cluster of SL1344 with the codon optimized APP2 rfb cluster downstream of the kanamycin resistance cassette. To integrate the kanamycin resistance cassette and the codon optimized APP2 rfb cluster into E. coli_5 and SL1344 the plasmids pDOC_E.coli_5_Δrfb::kanR-APP2.LPS(cod.opt.) and pDOC_SL1344_Δrfb::kanR-APP2.LPS(cod.opt.) were transformed into E. coli_5 or SL1344 respectively. The procedure was followed as described above and by Lee et al. 2009. The final strains E. coli_5 Δrfb::kanR-APP2.LPS(cod.opt.) and SL1344 Δrfb::kanR-APP2.LPS(codon optimized.) were verified by PCR and the O-antigen expression tested by immunoblotting (FIG. 8). Procedure of analysis and results are described under 1.6.

### 1.4) Improving the Expression of the APP2 O-antigen in SL1344 Expressing the APP2 rfb Cluster by Introducing gne and wzy

As seen in the generated strains expressing the APP2 rfb cluster the typical “ladder” pattern of the lipopolysaccharide was only weakly present or not detectable (FIG. 8). Instead a strong accumulation of O-antigen as single subunit oligosaccharide was observed on lipid A. This indicated that the O-antigen polymerase (Wzy) might not efficiently assemble the LPS glycan. The APP2 O-antigen specific wzy was chosen to express Wzy encoded on a plasmid. wzy was annotated by Xu et al. 2010, J. Bacteriol., Vol. 192(21), p5625-5636 as the sixth gene in the APP2 cluster and corresponds to a hypothetical protein located between setA and rfbX in Seq. 1 and FIG. 1. Blasting the hypothetical protein sequence against the NCBI protein database identified the Wzy of Lactococcus lactis (locus AZY91860) with 29% identity, a polysaccharide polymerase of Oenococcus oeni (locus WP_071436899) with 22% identity and a polysaccharide polymerase Streptococcus sp. DD12 (locus KXR76217) with 24% identity. Based on these findings it was assumed that the hypothetical protein might represent the O-antigen polymerase Wzy of the APP2 rfb cluster. Oligonucleotides 5′ Ndel_wzy/3′ EcoRl_wzy were used to amplify the potential wzy gene from plasmid pDOC_E.coli_5_Δrfb::APP2.LPS/kanR with 5′ Ndel and 3′ EcoRI restriction cleavage sites (fragment size of the PCR product was 1138bp). For expression of wzy, the vector pEC415 with an arabinose inducible promoter was chosen. For cloning wzy into pEC415 the vector was linearized, amplified and modified by PCR using oligonucleotides 3′ EcoRl_pEC415fw/ 5′ Ndel_pEC415rev (fragment of 5419bp). Again, EcoRI and Ndel cleavage sites were introduced at the fragment ends. PCR generated EcoRl-wzy-Ndel and EcoRl-pEC415-Ndel fragments were digested with the respective restriction endonucleases and ligated. After confirmation of the sequence (pEC415-wzy) the plasmid was further used for testing in cells expressing the APP2 rfb cluster.

A second potential limiting factor for O-antigen biosynthesis could be the availability and/or transfer of the glycans. LPS structural data for certain APP serotypes were published by Perry et al. 1990, identifying galactose at the reducing end of the APP2 O-antigen pentasaccharide [→2)-α-D-Galp-(1→3)-γ-D-Glcp-(1→4)-α-D-Glcp(6-(Ac)₀₋₆₅)-(1→4)-β-D-GalpNAc-(1→2)-α-L-Rhap-(1→]ₙ. One key enzyme in the generation of galactose in the bacterial cells is the conversion of galactose from glucose as substrate. This needs the action of an epimerase. The UDP-GlcNAc/Glc 4-epimerase gne of Campylobacter jejuni was identified to provide galactose and N-acetylgalactosamine in the bacterial cell for cell-surface carbohydrates (Bernatchez et al. 2005). The sequence of gne of C. jejuni 81116 (locus C8J_1070) was fused with a C-terminal HA (hemagglutinin) tag and 5′ EcoRI and 3′ Xbal restriction cleavage sites. This fragment was synthesized and afterwards cloned into the EcoRI/Xbal cleavage sites of pMLBAD to be under the control of an arabinose inducible promoter (pMLBAD-gne-HA).

The plasmid-based expression of wzy and/or gne was tested in SL1344 and E. coli_5 expressing the APP2 O-antigen on its surface. SL1344 and its derivatives (Δrfb, Δrfb::kanR-APP2.LPS pMLBAD-gne pEC415-wzy, Δrfb::kanR-APP2.LPS(cod.opt.) pMLBAD pEC415, Δrfb::kanR-APP2.LPS(cod.opt.) pMLBAD-gne, Δrfb::kanR-APP2.LPS(cod.opt.) pEC415-wzy and Δrfb::kanR-APP2.LPS(cod.opt.) pMLBAD-gne pEC415-wzy) as well as E. coli_5 and its derivatives (Δrfb, Δrfb::kanR-APP2.LPS(cod.opt.), Δrfb::kanR-APP2.LPS(cod.opt.) pMLBAD, Δrfb::kanR-APP2.LPS(cod.opt.) pMLBAD-gne) were grown in LB medium supplemented with antibiotics (according to table 1 and 2) until an OD₆₀₀ of 0.6 shaking at 37° C. To induce the wzy and/or gne expression arabinose was added to a final concentration of 0.1% (for SL1344 and its derivatives) or 0.2% (for E. coli_5 and its derivatives). After another 5 h of incubation at 37° C. (shaking) arabinose was added again to 0.1% (for SL1344 and its derivatives) or 0.2% (for E. coli_5 and its derivatives) and the cultures were incubated for about 16 h at 37° C. (shaking). Stationary cells were harvested and further processed. As a control for the APP2 O-antigen presentation on lipid A, APP2 P1875 strain was grown in BHI +NAD at 37° C. with slow shaking (110 rpm) to a stationary phase after which the cells were harvested. For APP2 O-antigen analysis, cells were re-suspended in 1×Lämmli buffer (1 OD₆₀₀ cells/100 μ1×Lämmli buffer). The samples were incubated at 95° C. for 5 min. 12 μg proteinase K per OD₆₀₀ equivalent cells (stock 20 mg/ml in 10 mM Tris-HCl pH 7.5, 20 mM CaCl₂, 50% glycerol) was added and the samples were incubated for 1 h at 60° C. Afterwards proteinase K treated samples (0.1 OD₆₀₀ cell equivalent) were loaded on 4-12% Bis-Tris gels, and molecules were separated by size in MES buffer. The gels were further processed for immunoblotting and silver staining. To analyze the LPS synthesis via immunoblot, LPS was transferred from the gel onto PVDF membranes. The membrane was incubated in blocking solution (PBS pH 7.5/0.05% Tween/0.1% casein) shaking for 2 h at room temperature. Then, the membrane was incubated shaking overnight at 4° C. in antibody binding solution (PBS pH 7.5/0.05% Tween/0.05% casein) containing a 1:2000 dilution of a rabbit serum reactive against APP2 LPS (rabbit immunized with proteinase K treated extracts of APP2 P1875). The immunoblot was washed 3 times for 5 min with an excess of PBS 0.05% Tween buffer pH 7.5. Afterwards the membrane was incubated for 1 h shaking at room temperature in antibody binding solution with secondary goat anti rabbit IgG-HRP antibody (BETHYL Cat# A120-401P) in a 1:2000 dilution. The membrane was washed 4 times for 5 min with an excess of PBS 0.05% Tween buffer pH 7.5. The antibody binding was visualized with overlaying the membrane with ECL solution (GE healthcare #RPN2105) and light signal detection with Stella 8300 (Raytest). For silver staining the protocol described by Tsai et al. 1982, Anal. Biochem., Vol. 119(1), p115-9. was used. Briefly, the gel was fixed overnight in 40% EtOH/5% acetic acid at room temperature. After, the gel was treated for 10 min with 0.7% periodic acid in 40% EtOH/5% acetic acid, followed by 3 times 15 min washes with ddH₂O. The gel was stained with staining solution (0.187 N sodium hydroxide, 0.2 N ammonium hydroxide, 0.667% silver nitrate) and again extensively washed 3 times 10 min with ddH₂O. The LPS on the gel was visualized by using developing solution (0.25 mg/ml citric acid monohydrate, 0.0185% formaldehyde solution).

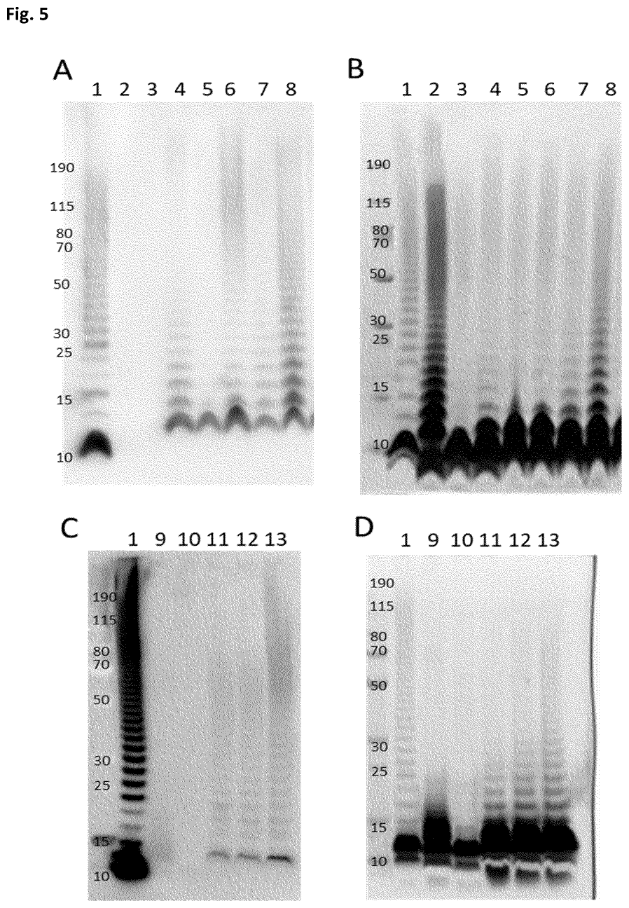

Analyzing the immunoblot of the membrane treated with the rabbit serum against the LPS of APP2 demonstrated a strong staining of the lipid A fraction at around 10 kDa, as well as a ladder pattern migrating between ˜12.5 and 190 kDa (O-antigen polymerization on lipid A) of strain APP2 P1875 (FIG. 5A, C lane 1). No recognition by the rabbit serum was observed for SL1344 and SL1344 Δrfb (FIG. 5A lane 2, 3) or E. coli_5 and E. coli_5 Δrfb. In the silver staining the LPS of SL1344 could be detected, which disappears in the SL1344 Δrfb indicating the successful deletion of the endogenous O-antigen biosynthesis (FIG. 5 B lanes 2, 3). LPS of E. coli_5 was only visible at lower molecular weight and disappeared in the E. coli_5 Δrfb cells (FIG. 5 D lanes 9 and 10). The integration of the codon-optimized APP2 rfb cluster downstream of the kanamycin resistance cassette in the rfb cluster of SL1344 (FIG. 5A lane 5) resulted in a band between 10 and 15 kDa which likely corresponded to lipid A with a single O-antigen attached. When gne or wzy were overexpressed in these cells (FIG. 5A lanes 6, 7) the before described ladder pattern appears and can be further enhanced if both proteins are overexpressed in the SL1344 Δrfb::kanR-APP2.LPS(cod.opt.) (FIG. 5A, B lane 8). Also, these cells show a stronger display than cells without codon optimized APP2 rfb cluster with the same expression setup (FIG. 5A lane 4). Thus, it has been demonstrated that gne and wzy expression enhances the APP2 LPS expression in SL1344 Δrfb::kanR-APP2.LPS (cod.opt.) cells. Also, for E. coli_5 cells expressing the codon optimized APP2 rfb cluster the ladder pattern for the APP2 O-antigen could be further improved by overexpression of gne (FIG. 5 C, D comparing lane 12 and 13)

### 1.5) Integration of gne and Codon Optimized wzy into SL1344 Expressing the APP2 0-Antigen

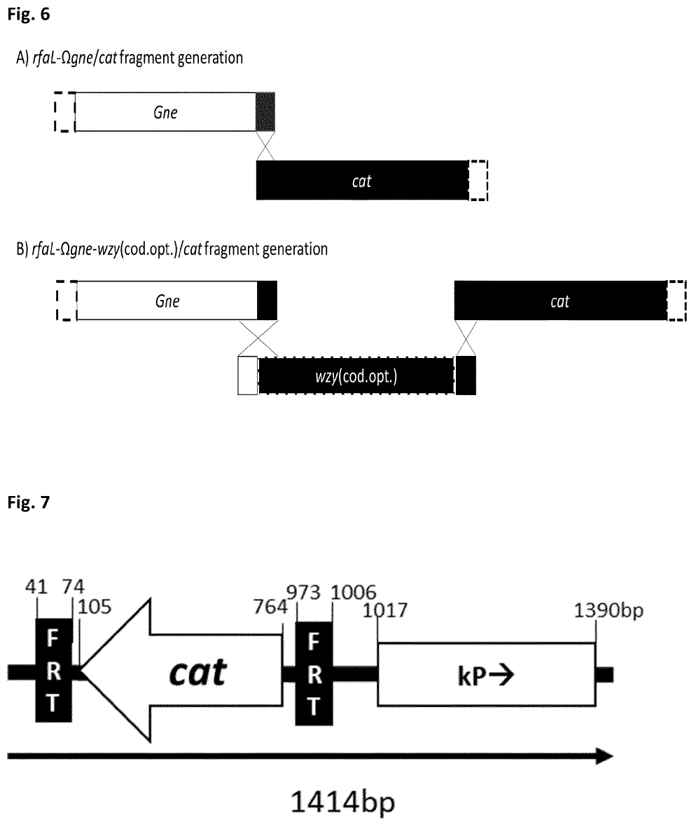

Having seen an improvement of APP2 O-antigen synthesis after introducing gne and/or wzy on plasmid into genetically modified 5L1344, these genes were integrated into the genome of strains expressing the APP2 rfb cluster. To compare the expression levels of APP2 LPS, gne alone and gne fused to the codon-optimized wzy (wzy(cod.opt.) of APP2, Seq. 8) were integrated. As integration site, the downstream area of the O-antigen ligase rfaL was chosen to use the rfaL promoter to transcribe as well the integrated gne and wzy(cod.opt.). The general design (FIG. 6) is based on the generation of two (FIG. 6A) to three (FIG. 6 B) individual PCR fragments encoding for each of the two enzymes (gne, wzy-cod.opt.) and the antibiotic resistance cassette cat (chloramphenicol resistance cassette) flanked by FRT sites.

To generate rfaL-Ωgne/cat (FIG. 6A) individual fragments for gne and cat with overlapping regions were amplified. With an overlap PCR the two fragments were fused and the 5′ and 3′ ends were elongated to encode homologous regions with the target integration sites in the genome of SL1344. The same principle was followed by generating the integration construct rfaL-Ωgne-wzy(cod.opt.)/cat (FIG. 6 B) except that an additional fragment encoding the codon optimized wzy was generated before combining the 3 fragments by overlap PCR and adding homologous recombination sequences at the 5′ and 3′ end. The 5′ and 3′ ends of the fusion constructs were further elongated to include the homologous regions which can recombine with the targeted genomic integration site downstream of the rfaL stop codon (method adjusted from Bryskin et al. 2010, Biotechniques, Vol. 48(6), p463-465). The template and oligonucleotide information for the individual PCRs and the overlap PCRs to generate the substrates for manipulating SL1344 are listed in Table 5.

TABLE 5

| | | PCR1 | | | overlap/elongation PCR2 | | |
|---|---|---|---|---|---|---|---|
| Fusion construct | Fragment | Template | Oligo 1 | Oligo 2 | Template | Oligo 1 | Oligo 2 |
| rfaL-gne/cat | gne | pMLBAD-gne-HA | 5′-gne_Sl1344rfaL | 3′-gne-cat_overlap | SL1344 PCR1 | 5′-ela- | 3′-elo- |
| | cat | pkD3 | 5′-cat-P2new | 3′-cat_Sl1344faL | gne/PCR1 cat | gne_Sl1344rf | cat_Sl1344rfaL |
| rfaL-gne- | gne | pMLBAD-gne-HA | 5′-gne_Sl1344rfaL | 3′-gne_wzy (co) | SL1344 PCR1 | 5′-elo- | 3′-elo- |
| wzy(cod.opt.)/cat | | | | overlap | gne/PCR1 | gne_ | cat_Sl1344rfaL |
| | wzy(cod. | pDOC_E.coli_5_Δrfb::kanR | 5′ gne overlap_wzy | 3′ wzy (co)- | wzy(cod.opt.) | Sl1344rfaL | |
| | opt.) | APP2.LPS(cod.opt.) | (co) | cat_overlap | PCR1cat | | |
| | cat | pKD3 | 5′-cat-P2new | 3-cat_Sl1344rfaL | | | |

The fusion constructs resulting from overlap PCR were transformed together with the temperature sensitive helper plasmid encoding λ-recombination system pLAMBDA46 (modified from Datsenko et al. 2000, PNAS, Vol. 97(12), p6640-6645) for SL1344 Δrfb::kanR-APP2.LPS, SL1344 Δrfb::kanR-APP2.LPS(cod.opt.). The λ-recombination system recognized the homologous flanking regions of the fusion constructs and recombined them in the genome downstream after the stop codon of rfaL. The introduced chloramphenicol resistance gene (cat) allowed the selection of positive clones (successful integration). These positive candidates were verified for the integration of the PCR fragment by PCR. The temperature sensitive helper plasmid was lost from the cells by increasing the growth temperature for several rounds of incubations. The O-antigen presentation in the final strains SL1344 Δrfb::kanR-APP2.LPS rfaL-Ωgne/cat, SL1344 Δrfb::kanR-APP2.LPS rfaL-Ωgne-wzy(cod. opt.)/cat, SL1344 Δrfb::kanR-APP2.LPS(cod.opt.) rfaL-Ωgne/cat and SL1344 Δrfb::kanR-APP2.LPS(cod.opt.) rfaL-Ωgne-wzy(cod. opt.)/cat was tested by immunoblotting (FIG. 8). Procedure of analysis and results are described under 1.6.

### 1.6) Exchanging the Kanamycin Resistance Cassette with Only the Kanamycin Promoter to “Drive” the Expression of the APP2 rfb Cluster

For vaccines based on whole cell bacteria, it is recommended to have no antibiotic resistance. Therefore, the kanamycin resistance—which was introduced to enhance the APP2 rfb expression—needed to be deleted from the genome. But the advantage of increased transcription by the kanamycin promoter may be used in future bacterial vaccine strains. In a first step, the kanamycin and chloramphenicol resistance cassettes were “flipped out” by introducing the temperature sensitive plasmid pCP20 (Cherepanov et al. 1995, Gene, Vol. 158, p9-14) into SL1344 Δrfb::kanR-APP2.LPS(cod.opt.) rfaL-Ωgne-wzy(cod. opt.)/cat cells. After “flip out” event of the kanamycin and chloramphenicol resistance cassettes at each location only one FRT site remained in the genome. With increasing cultivation temperature, the positive clones were counter selected against the flippase encoding plasmid. By PCR the absence of pCP20 and the absence of kanamycin as well as chloramphenicol resistance marker was verified. To integrate the kanamycin promoter (kP) upstream of the codon optimized APP2 rfb cluster a 1414 bp construct was synthesized containing a chloramphenicol resistance cassette with flanking FRT sites followed by the 373 bp promoter region of the kanamycin resistance cassette (FIG. 7, Seq. 45). To introduce sequence 22 upstream of the APP2 rfb cluster a PCR fragment was generated using oligonucleotide Ec_SL/Kan_fw and Ec_SL/Kan_rev and sequence 24 as template amplifying the kP integration cassette and adding homologous recombination sequences for the upstream region of the rfb cluster. The resulting product was used together with oligonucleotide Ec_SL/Kan_fw_elo and Ec_SL/Kan_rev_elo for a second PCR reaction to elongate the homologous recombination sequences for an increase in integration efficiency (fragment size 1671 bp). This DNA fragment and a temperature sensitive helper plasmid encoding λ-recombination system pKD46 (Datsenko et al. 2000, PNAS, Vol. 97(12), p6640-6645) for SL1344 Δrfb::APP2.LPS(cod.opt.) rfaL-Ωgne-wzy(cod. opt.) were introduced. The λ-recombination system recognized the homologous flanking regions upstream of the rfb cluster and integrated the chloramphenicol resistance gene with the flanking FRT sites fused to the kanamycin promoter into the respective site. The introduced chloramphenicol resistance gene allowed the selection of positive clones (successful integration). These positive candidates were verified for the integration of the PCR fragment. The temperature sensitive helper plasmid was lost from the cells by increasing the growth temperature for several rounds of incubations. To remove the chloramphenicol resistance cassette the generated strain SL1344 Δrfb::cat/kP-APP2.LPS(cod.opt.) rfaL-Ωgne-wzy(cod. opt.) was transformed with pCP20 and the marker flipped out as described above. The resulting strain SL1344 Δrfb::kP-APP2.LPS(cod.opt.) rfaL-Ωgne-wzy(cod. opt.) was analyzed for its APP2 O-antigen generation and compared to its parental strain (FIG. 8).

Wild type SL1344 and E. coli_5 as well as genetically modified cells lacking the endogenous O-antigen biosynthesis (E. coli_5 Δrfb and SL1344 Δrfb) or expressing the APP2 rfb cluster (E. coli_5 Δrfb::APP2.LPS, Δrfb::kanR-APP2.LPS, Δrfb::kanR-APP2.LPS(cod.opt.) and SL1344 Δrfb::APP2.LPS, Δrfb::kanR-APP2.LPS, Δrfb::kanR-APP2.LPS(cod.opt.), Δrfb::kanR-APP2.LPS rfaL-Ωgne/cat, Δrfb::kanR-APP2.LPS rfaL-Ωgne-wzy(cod. opt.)/cat, Δrfb::kanR-APP2.LPS(cod. opt.) rfaL-Ωgne-wzy(cod. opt.)/cat, Δrfb::kP-APP2.LPS(cod.opt.) rfaL-Ωgne-wzy(cod.opt.), Δrfb rfaK-ΩClyA-ApxI(aa626-845)-ApxII(aa612-801)-ApxIII(aa626-860)-HIS6-rfaL, Δrfb::kP-APP2.LPS(cod.opt.) rfaL-Ωgne-wzy(cod.opt.) pliC-ΩClyA-ApxI(aa626-845)-ApxII(aa612-801)-ApxIII(aa626-860)-HIS6-pagC) were grown to saturation (OD₆₀₀ >2) in LB medium, shaking at 37° C. For further processing cells were harvested. As a control for the APP2 O-antigen presentation on lipid A, APP2 P1875 strain was grown in BHI+NAD to a stationary phase at 37° C. with slow shaking (110 rpm). Cells were harvested and used for further processing. For APP2 O-antigen analysis, cells were re-suspended in 1×Lämmli buffer (1 OD₆₀₀ cells/100 μl 1×Lämmli buffer). The samples were incubated at 95° C. for 5 min. 12 μg proteinase K per OD₆₀₀ equivalent cells (stock 20 mg/ml in 10 mM Tris-HCl pH 7.5, 20 mM CaCl₂, 50% glycerol) were added and the samples were incubated for 1 h at 60° C. Afterwards proteinase K treated samples (0.1 OD₆₀₀ cell equivalent) were loaded on 4-12% Bis-Tris gels, and molecules were separated by size in MES buffer. The gels were further processed for Immunoblotting and Silver staining. To analyze the LPS synthesis via immunoblot, LPS was transferred from the gel onto PVDF membrane. The membrane was incubated in blocking solution (PBS pH 7.5/0.05% Tween/0.1% casein) shaking for 2 h at room temperature. After, the membrane was incubated shaking overnight at 4° C. in antibody binding solution (PBS pH 7.5/0.05% Tween/0.05% casein) containing a rabbit serum reactive against APP2 LPS in a in a 1:2000 dilution. The immunoblot was washed 3 times for 5 min with an excess of PBS 0.05% Tween buffer pH 7.5. Afterwards the membrane was incubated for 1 h shaking at room temperature in antibody binding solution with secondary goat anti rabbit IgG-HRP antibody (BETHYL Cat# A120-401P) in a 1:2000 dilution. The membrane was washed 4 times for 5 min with an excess of PBS 0.05% Tween buffer pH 7.5. Afterwards the specific antibody binding was visualized by adding ECL solution (GE healthcare #RPN2105) to the membrane and recording the light signal detected with Stella 8300 (Raytest). For silver staining the protocol described by Tsai et al. 1982, Anal. Biochem., Vol. 119(1), p115-9. was used. Briefly, the gel was fixed overnight in 40% EtOH/5% acetic acid at room temperature. After that the gel was treated for 10 min with 0.7% periodic acid in 40% EtOH/5% acetic acid followed by 3 times 15 min washes with ddH₂O. The gel was stained with staining solution (0.187 N sodium hydroxide, 0.2 N ammonium hydroxide, 0.667% silver nitrate) and again extensively washed for 3 times 10 min with ddH₂O. The LPS on the gel was visualized by adding developing solution (0.25 mg/ml citric acid monohydrate, 0.0185% formaldehyde solution).

Immunoblotting by using the rabbit serum against the LPS of APP2 showed a strong staining of the lipid A fraction at around 10 kDa and a ladder pattern migrating between ˜12.5 and 190 kDa (O-antigen polymerization on lipid A) of strain APP2 P1875 (FIG. 8 C lane 1). No recognition by the rabbit serum could be seen for SL1344 and SL1344 Δrfb (FIG. 8 C, lane 2, 3), SL1344 Δrfb rfaK-ΩClyA-ApxI(aa626-845)-ApxII(aa612-801)-ApxIII(aa626-860)-H156-rfaL (FIG. 8 C, lane 11) or E. coli_5 and E. coli_5 Δrfb (FIG. 8 C lanes 13, 14). In the silver staining the LPS of SL1344 (ranging from 10 kDa to >115 kDa) and E. coli_5 (10 kDa—15 kDa) could be detected, which disappears in the SL1344 Δrfb and E. coli_5 Δrfb indicating the successful deletion of the endogenous O-antigen biosynthesis (FIG. 8C lane 2, 3, 13, 14). The integration of the APP2 rfb cluster resulted in the appearance of a very faint band between 10 and 15 kDa which likely corresponds to lipid A with a single O-antigen attached (FIG. 8 C, lane 4, 15). This signal could be enhanced when the APP2 O-antigen biosynthesis was located downstream of the kanamycin resistance cassette (FIG. 8C, lane 5, 16). The codon optimization of the APP2 rfb cluster combined with the upstream kanamycin resistance cassette further improved the APP2 O-antigen biosynthesis (FIG. 8C, lanes 6, 17). Especially for E. coli_5 this resulted in the appearance of the polymerized O-antigen chain (ladder pattern, FIG. 8 C lane 17). The ladder pattern at the lower molecular weight can also be detected in the silver staining analysis (FIG. 8B, lane 17). After introduction of gne or gne and wzy into SL1344 Δrfb::kanR-APP2.LPS (FIG. 8 C, lanes 7, 8) a stepwise increase in APP2 O-antigen expression with bands appearing at higher molecular weight was observed which indicates polymerization of the O-antigen on lipid A. Genomic expression of gne and codon optimized wzy in SL1344 expressing the codon optimized APP2 rfb cluster either downstream of the kanamycin resistance cassette (FIG. 8C, lane 9) or the kanamycin promoter only (FIG. 8C, lane 10) increased the O-antigen polymerization and presentation on lipid A to an amount that even at high molecular weight (>190 kDa) the glycan structure can be detected. In both cell lines also low molecular weight bands can be detected in the silver staining analysis (FIG. 8B, lane 9, 10). The final glycoengineered SL1344 presenting in addition the ClyA-ApxI(aa626-845)-ApxII(aa612-801)-ApxIII(aa626-860)-HIS6 (FIG. 8 C, lane 12) on the cell surface shows the same strength of APP2 O-antigen expression as its parental strain SL1344 Δrfb::kP-APP2.LPS(cod.opt.) rfaL-Ωgne-wzy(cod.opt.).

### 1.7) Integration of the Codon Optimized APP8 0-Antigen Biosynthesis Cluster Controlled by the kP Promoter in SL1344

To prove that the described transfer of heterologous O-antigen into host bacteria can be applied to various O-antigen types, the rfb cluster of Actinobacillus pleuropneumoniae serotype 8 (APP8) strain (MIDG2331) was genomically integrated into SL1344 and tested for the APP8 O-antigen presentation on lipid A.

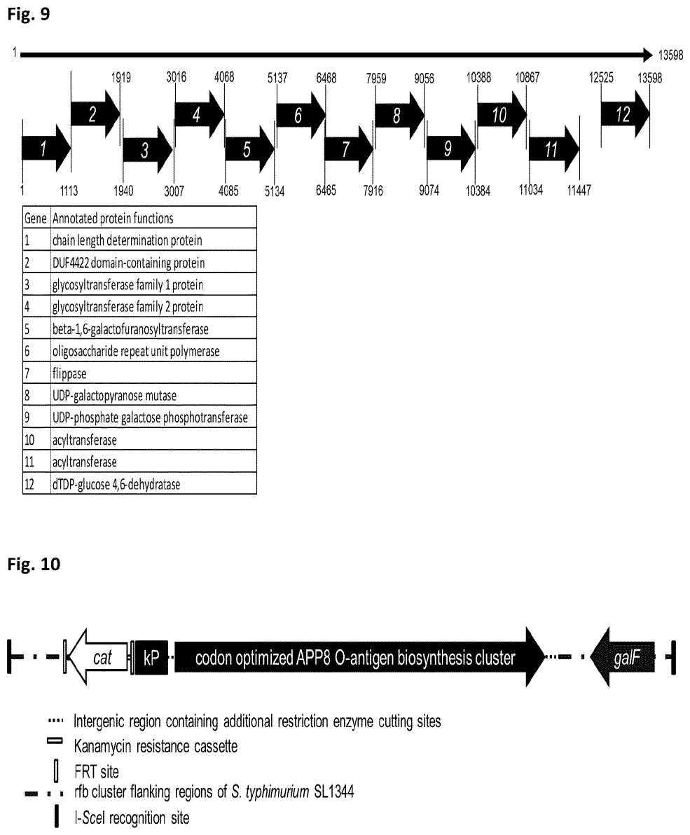

The codon optimized (for E. coli expression which has been shown for the APP2 rfb cluster to work as well for SL1344) 13598 bp fragment (FIG. 9, Seq. 4) containing the APP8 rfb cluster was modified for integration in an antisense orientation to the endogenous rfb cluster into SL1344. Upstream of the APP8 rfb cluster a chloramphenicol resistance cassette flanked by FRT sites followed by the 373 bp promoter region of the kanamycin resistance cassette was fused. The construct was flanked upstream and downstream by homologous regions flanking the rfb cluster of either SL1344 (including the galF gene in antisense direction) flanked by I-Scel restriction endonuclease recognition sites (Seq. 46, FIG. 10). To integrate the codon optimized APP8 rfb cluster controlled by the kP into SL1344, shuttle vector plasmids pDOC_SL1344_Δrfb::cat-kP-APP8.LPS(cod.opt.) based on the pDOC system of Lee et al. 2009 was generated/synthesized (by CRO).

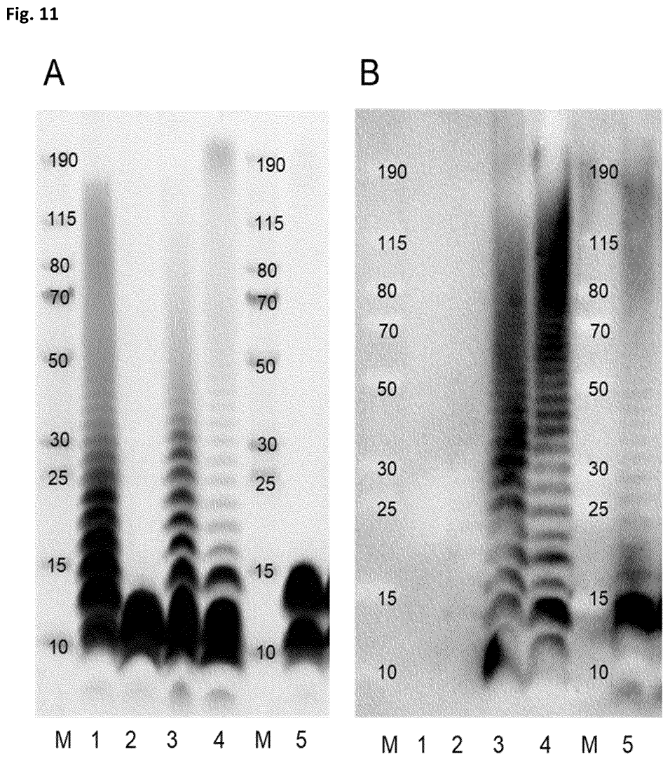

The shuttle vector plasmids (pDOC_SL1344_Δrfb::cat-kP-APP8.LPS(cod.opt.) together with the helper plasmid pACBSCE, were transformed into SL1344. The procedure was followed as described above and by Lee et al. 2019. The final strains SL1344 Δrfb::cat-kP-APP8.LPS(cod.opt.) were verified by PCR and the O-antigen expression tested by immunoblotting (FIG. 11).

Wild type SL1344 as well as genetically modified cells lacking the endogenous O-antigen biosynthesis (SL1344 Δrfb) or expressing the codon optimized APP8 rfb cluster under the control of the kP promoter (SL1344 Δrfb::cat-kP-APP8.LPS(cod.opt.)) were grown to saturation (OD₆₀₀>2) in LB medium, shaking at 37° C. For further processing cells were harvested. As a control for the APP8 O-antigen presentation on lipid A, APP8 (MIDG2331) and APP3 (ORG1224) strain were grown in BHI+NAD to a stationary phase at 37° C. with slow shaking (110 rpm). Cells were harvested and used for further processing. For APP8 O-antigen analysis, cells were re-suspended in 1×Lämmli buffer (1 OD₆₀₀ cells/100 μl 1×Lämmli buffer). The samples were incubated at 95° C. for 5 min. 12 μg proteinase K per OD₆₀₀ equivalent cells (stock 20 mg/ml in 10 mM Tris-HCl pH 7.5, 20 mM CaCl₂, 50% glycerol) were added and the samples were incubated for 1 h at 60° C. Afterwards proteinase K treated samples (0.1 OD₆₀₀ cell equivalent) were loaded on 4-12% Bis-Tris gels, and molecules were separated by size in MES buffer. The gels were further processed for Immunoblotting and Silver staining. To analyze the LPS synthesis via immunoblot, LPS was transferred from the gel onto PVDF membrane. The membrane was incubated in blocking solution (PBS pH 7.5/0.05% Tween/0.1% casein) shaking for 2 h at room temperature. After, the membrane was incubated shaking overnight at 4° C. in antibody binding solution (PBS pH 7.5/0.05% Tween/0.05% casein) containing a pig serum reactive against APP3 LPS in a in a 1:500 dilution. The immunoblot was washed 3 times for 5 min with an excess of PBS 0.05% Tween buffer pH 7.5. Afterwards the membrane was incubated for 1 h shaking at room temperature in antibody binding solution with secondary pig anti-IgG-HRP (BETHYL Cat#A100-105P) in a 1:2000 dilution. The membrane was washed 4 times for 5 min with an excess of PBS 0.05% Tween buffer pH 7.5. Afterwards the specific antibody binding was visualized by adding ECL solution (GE healthcare #RPN2105) to the membrane and recording the light signal detected with Stella 8300 (Raytest). For silver staining the protocol described by Tsai et al. 1982, Anal. Biochem., Vol. 119(1), p115-9. was used. Briefly, the gel was fixed overnight in 40% EtOH/5% acetic acid at room temperature. After that the gel was treated for 10 min with 0.7% periodic acid in 40% EtOH/5% acetic acid followed by 3 times 15 min washes with ddH₂O. The gel was stained with staining solution (0.187 N sodium hydroxide, 0.2 N ammonium hydroxide, 0.667% silver nitrate) and again extensively washed for 3 times 10 min with ddH₂O. The LPS on the gel was visualized by adding developing solution (0.25 mg/ml citric acid monohydrate, 0.0185% formaldehyde solution).

A rabbit serum reactive against serotype 3 was used since the O-antigen structures of APP3 and APP8 are identical (Perry et al. 1990, Serodiagnosis and Immunotherapy in Infectious Disease, Vol. 4(4), p299-308).

Immunoblotting by using the rabbit serum against the LPS of APP3 showed a strong staining of the lipid A fraction at around 10 kDa and a ladder pattern migrating between ˜12.5 and 190 kDa (O-antigen polymerization on lipid A) of strain APP8 and 3 (FIG. 11 B lane 3, 4) proving the cross reactivity of the pig serum with both serotype O-antigens. No recognition by the rabbit serum could be seen for SL1344 and SL1344 Δrfb (FIG. 11B, lane 1, 2). In the Silver staining the LPS of SL1344 (ranging from 10 kDa to >115 kDa) could be detected, which disappears in the SL1344 Δrfb (FIG. 11A, lane 1, 2). The integration of the codon optimized APP8 rfb cluster under the control of the kP promoter resulted in the appearance of a strong band between 10 and 15 kDa which likely corresponds to lipid A with a single O-antigen attached (FIG. 11B, lane 5). Furthermore, a faint ladder pattern ranging from 15 to 190 kDa reactive with the APP3 reactive pig serum and indicating the O-antigen polymerization could be detected.

It can be concluded that the heterologous APP8 rfb cluster (codon optimized and under the control of the kP promoter) could be successfully transferred into the heterologous host SL1344 and resulted in the presentation of APP8 O-antigen on lipid A.

### Example 2

### 2) Cloning of Neutralizing Epitopes of Apx Toxins of APP as Potential Vaccination Candidates

2.1) Generation of plasmids encoding soluble neutralizing epitopes of ApxII and ApxIII

For an effective vaccine against APP infection inactivated toxins (Apx toxoids) are considered to be present in the final vaccine formulation. These pore-forming Apx toxin belong to the major virulence factors of APP and do contribute strongly to the pathogenesis of infection (Bosse et al. 2002, Microbes Infect., Vol. 4(2), p225-235). Four identified Apx toxins (ApxI, II, III and IV) are encoded in their pretoxin structural forms in the apx operon which furthermore encodes the activator gene and secretion-apparatus-encoding genes. The four toxins are expressed in various combinations in the different APP serotypes. APP serotype 2 encodes for ApxII, III and IV (Beck et al. 1994, J. Clin. Microbiol., Vol. 32(11), p2749-2754). Here truncated versions of ApxII and III were generated, expressed in E. coli and purified. In recent publications neutralizing epitopes of ApxII and III were identified which could induce an immune response, and antibodies generated against these epitopes were protective in vitro. Kim et al. 2010 described an N-terminally HIS10 tagged truncated ApxII from APP2 containing amino acid 439-801aa. The Sequence of RTX toxin IIA (ApxII) was retrieved from the database (GenBank: AF363362.1), synthesized and used for generating the truncated HIS10 tagged ApxII(439-801aa) (Seq. 47) encoded on the pMLBAD vector pMLBAD-HIS10-ApxII(439-801aa)). The sequence of ApxIII was retrieved from the database (GenBank: AF363363.1) and used as template for synthesis. In a previous study, it was shown that the N-terminal domain of ApxIII was recognized by a convalescent pig serum, having cytotoxicity neutralizing activity and preventing in vitro neutrophil apoptosis induced by ApxIII (Seah et al. 2004, Vaccine, Vol. 22(11-12), p1494-1497). The N-terminal domain of ApxIII (amino acid 27-245) was synthesized with a C-terminal HIS10 tag (Seq. 48) and used as a template to introduce a start codon and 5′ Xmal/3′ HindIII restriction endonuclease cleavage sites by using primer 5′ Xmal-Xhol-APXIIIne-HIS-fw/3′ APXIIIne-HIS-HindIII-ry in a PCR reaction. The resulting fragment was digested with the respective enzymes and ligated into Xmal/HindIII treated pMLBAD vector (pMLBAD-ApxIII (27-245aa)-H159; in the course of the cloning one HIS epitope was lost). Both plasmids were introduced into E. coli BL21 for protein expression testing and purification for pig vaccination trials. To purify HIS10-ApxII(439-801aa) an overnight culture of BL21 pMLBAD-HIS10-ApxII (439-801aa) grown at 37° C. shaking in LB+Tmp (10 μg/ml) was diluted to 0.1 OD₆₀₀ in 1 L LB+Tmp and incubated shaking at 37° C. until the OD600 was around 0.8-0.9. Arabinose (0.2% final concentration) was added to induce the protein expression. Cells were incubated for another 4 h at 37° C. on a shaker and then harvested by centrifugation (5000×g/4° C./10 min and discarding the supernatant). For cell lysis, 20 ml 0.1 mg/ml lysozyme in lx PBS were added. Cells were broken by sonication-freeze-thaw rounds. Briefly, cell pellets were sonicated with an amplitude of 85%, frozen in liquid nitrogen for 1 min and thawed for 10 min at 37° C. (shaking). This procedure was repeated 3 times. The cell suspension was centrifuged for 5 min at 4° C. with 10000×g. It was noticed that during the before described procedure HIS10-ApxII(439-801aa) aggregates which results in the protein to be found in the pellet fraction after centrifugation. Around 20 ml denaturing buffer (6 M guanidinium hydrochloride, 0.1 M TrisHCl pH 8.0) were added to the pellet and the material was centrifuged again for 20 min at 10000×g at 4° C. 4 ml equilibrated Nickel-NTA resin was added to the supernatant and the material was incubated for 1 h on a rotation wheel at 4° C. Afterwards the suspension was loaded on a gravity flow column. The resin was washed 3 times with 10 ml 1×PBS and proteins eluted with 5 times 1 ml denaturing buffer containing 0.5 M imidazole. To analyze the collected material 60 μl of each elution fraction were mixed with 20 μl 4×Lämmli buffer. The samples were cooked for 5 min at 95° C. and 10 μl were loaded on 4-12% Bis-Tris gels, and molecules were separated by size in MES buffer. The gels were further processed for Coomassie staining (FIG. 12A) and immunoblotting using a HIS specific antibody (FIG. 12B). For Coomassie staining the gel was overlaid with Coomassie staining solution (3 mM Coomassie brilliant blue R 250, 40% ethanol, 10% acetic acid) and incubated shaking overnight at room temperature. The gel was placed repeatedly in Coomassie destain solution (30% ethanol, 10% acetic acid) until the desired destain of the gel and stain of proteins was observed. To detect purified HIS10-ApxII(439-801aa) via immunoblot, proteins were transferred from the gel onto PVDF membrane. The membrane was incubated in blocking solution (PBS pH 7.5/0.05% Tween/0.1% casein) shaking for 2 h at room temperature. After, the membrane was incubated shaking overnight at 4° C. in antibody binding solution (PBS pH 7.5/0.05% Tween/0.05% casein) containing a Tetra-HIS antibody (Qiagen #34670) in a 1:2000 dilution. The membrane was washed 3 times for 5 min with an excess of PBS 0.05% Tween buffer pH 7.5. Afterwards, the membrane was incubated for 1 h shaking at room temperature in antibody binding solution with secondary goat anti mouse IgG-HRP antibody (BETHYL Cat# A90-116P) in a 1:2000 dilution. The membrane was washed 4 times for 5 min with an excess of PBS 0.05% Tween buffer pH7.5. Afterwards the specific antibody binding was visualized by adding ECL solution (GE healthcare #RPN2105) to the membrane and recording the light signal detected with Stella 8300 (Raytest).

With the applied method of protein expression and purification, the HIS10-ApxII(439-801aa) could be purified in high quantities via binding to Nickel-NTA resin and elution using gravity flow (FIG. 12). The highest protein quantities are found in elution fraction 3-5. Besides the purified HIS10-ApxII(439-801aa) at around 41 kDa certain impurities can be detected at higher and lower molecular weight (FIG. 12A) which are also recognized by the HIS antibody (FIG. 12 B). With this experiment a successful expression and purification of the truncated HIS10-ApxII(439-801aa) could be shown.

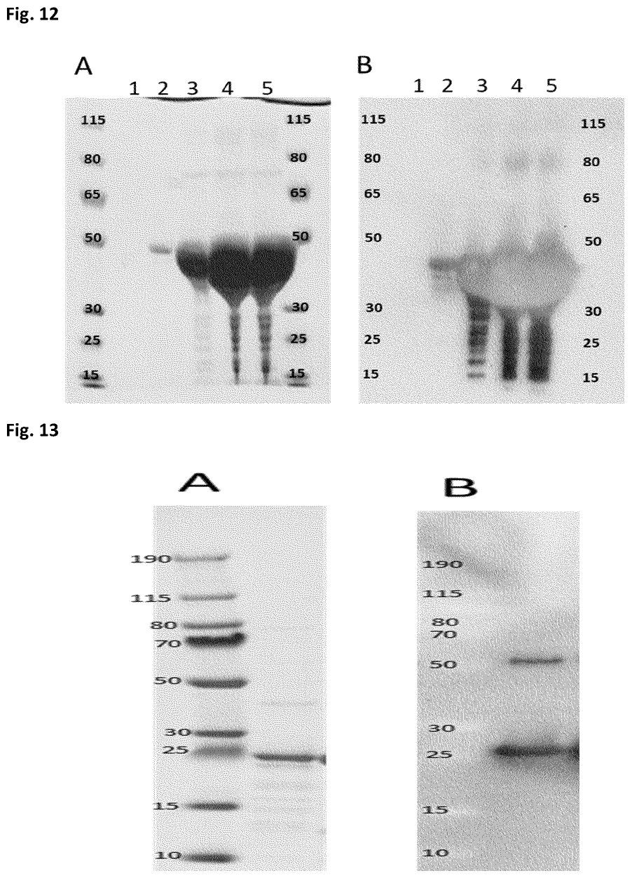

To purify ApxIII(27-245aa)-HISS an overnight culture of BL21 pMLBAD-ApxIII(27-245aa)-HIS9 grown at 37° C. shaking in TB+Tmp (10 μg/ml) was diluted to 0.1 OD₆₀₀ in 1 L TB+Tmp and incubated shaking at 37° C. until the OD₆₀₀ was around 0.6-0.8. Arabinose (0.2% final concentration) was added to induce the protein expression. Cells were incubated for another 4 h at 37° C. on a shaker and then harvested by centrifugation. A similar procedure was followed as described for HIS10-ApxII(439-801aa). Briefly, cell lysis was done in a final volume of 70 ml (30 mM Tris HCl pH 7.5, 300 mM NaCl, 20% sucrose, 1 mM EDTA, 1 g/l lysozyme, 1× protease inhibitor cocktail). Cells were lysed by 3 rounds of sonication-freeze-thaw rounds (as described above). The cell suspension was centrifuged for 15 min at 4° C. at 5525×g. The supernatant was centrifuged again for 1 h at 20000×g at 4° C. 0.3 ml Nickel-NTA resin equilibrated in binding buffer (30 mM Tris HCl pH 7.5, 300 mM NaCl) was added to the supernatant and the material was incubated for 1 h on a rotation wheel at 4° C. Afterwards, the suspension was loaded on a gravity flow column. The resin was washed 2 times with 3 ml binding buffer and 1 time with 3 ml washing buffer (30 mM Tris HCl pH 7.5, 300 mM NaCl, 10 mM imidazole). Elution was done by adding 4 times 0.3 ml elution buffer 1 (3 0mM Tris HCl pH 7.5, 300 mM NaCl, 200 mM imidazole), 2 times 0.5 ml elution buffer 2 (30 mM Tris HCl pH 7.5, 300 mM NaCl, 500 mM imidazole) and 3 times 0.5 ml elution buffer 3 (30 mM Tris HCl pH 7.5, 300 mM NaCl, 1M imidazole). To analyze the cell preparation and protein purification all fractions were tested by immunoblot against the HIS epitope and Coomassie staining and it was decided to pool elution fraction 4-8 and exchange the buffer by dialysis in PBS. To analyze the pooled and dialyzed sample, 60 μl of material were mixed with 20 μl 4×Lämmli buffer. The samples were cooked for 5 min at 95° C. and 10 μl were loaded on 4-12% Bis-Tris gels, and molecules were separated by size in MES buffer. The immunoblot using a HIS specific antibody and the Coomassie staining were performed as described above (FIG. 13). A HIS specific signal was identified around 25 kDa consistent with the calculated molecular weight of ApxIII(27-245aa)-HIS9 (FIG. 13B) which could also be detected on the Coomassie stained gel (FIG. 13A). Some impurities at lower and higher molecular weight are as well detected. A successful expression and purification of the truncated ApxIII(27-245aa)-HIS9 was demonstrated (FIG. 10).

### 2.2) Genomic Integration Of Neutralizing Epitopes of ApxI, II and III as fusion Constructs to be Expressed on Cell Surfaces of Either E. coli_5 or SL1344, Both Expressing the APP2 O-antigen

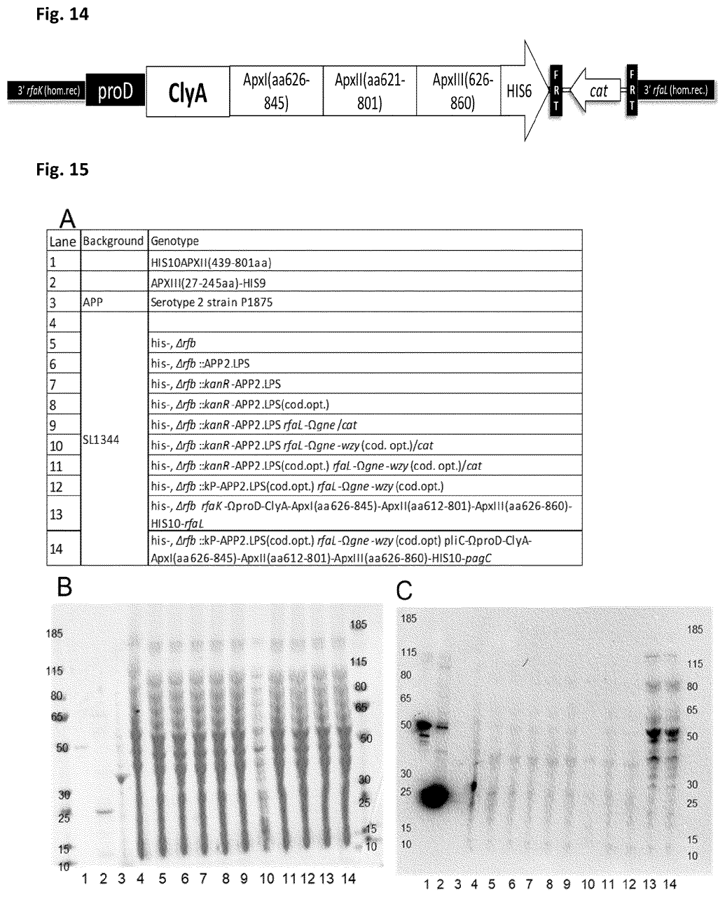

For generating a live Salmonella vaccine strain presenting the APP2 LPS and the neutralizing epitopes of Apx toxins on its surface, the corresponding genes were genomically integrated to result in a surface presentation of the neutralizing epitopes. Xu et al. 2018, PlosOne, Vol. 13(1) described a fusion construct consisting of ClyA, a pore-forming hemolytic protein expressed on the cell surface which induces specific immune responses linked to truncated ApxI (628-845aa), truncated ApxII (612-801aa) and truncated ApxIII (626-860aa) with a C-terminal HIS6 tag. In vaccination and challenge studies of mice the group showed elevated immunoglobulin and cytokine levels as well as an increased survival rate after the challenge with different APP serotypes. The sequence of the construct is publicly available and was modified as follows. With an algorithm provided by the synthesizing company the codon usage of ClyA-ApxI(628-845aa)-ApxII(612-801aa)-ApxIII(626-860aa)-HIS6 was optimized for E. coli expression systems. For a strong transcription rate the sequence of the synthetic promoter proD (Davis et al. 2010, Nucleic acids research, Vol. 39(3), p1121-1141) was integrated before the start codon. To select for successful integration into the genome a chloramphenicol resistance cassette (cat) with flanking FRT sites (based on pKD3; Datsenko et al. 2000, PNAS, Vol. 97(12), p6640-6645) was added after the stop codon of the fusion construct. For homologous integration into the genome, upstream and downstream of the construct homologous sequences to the intergenic rfaK-rfaL region were chosen (addition 213bp 5′ of proD promoter, 250bp after the chloramphenicol resistance cassette). The complete synthesized construct is schematically represented in FIG. 14 (Seq. 49). To integrate the fusion construct proD-ClyA-ApxI(628-845aa)-ApxII(612-801aa)-ApxIII(626-860aa)-HIS6/cat into 5L1344 Δrfb::kP-APP2.LPS rfaL-Ωgne-wzy(cod.opt.) the flanking region of Sequence 32 was changed by PCR using oligonucleotides FW_pliCint/REV_pagCint followed by an elongation of the 5′ and 3′ end with oligonucleotides eloFW_pliCint/eloREV_pagCint. This procedure created an 83bp homologous region with the downstream region of pliC in SL1344 and 87bp homologous region with the downstream region of pagC in SL1344. With this modification the fusion construct proD-ClyA-ApxI(628-845aa)-ApxII(612-801aa)-ApxII(626-860aa)-HIS6/cat could be integrated into the intergenic region between pliC and pagC. The final construct was transformed together with the temperature sensitive helper plasmid encoding γ-recombination system pKD46 (Datsenko et al. 2000, PNAS, Vol. 97(12), p6640-6645) into SL1344 Δrfb::kP-APP2.LPS rfab-Δgne-wzy(cod.opt.). The λ-recombination system recognized the homologous flanking regions at the respective genomic sites and recombined the fusion construct proD-ClyA-ApxI(628-845aa)-ApxII(612-801aa)-ApxIII(626-860aa)-HIS6/cat into the before mentioned integration site (SL1344 Δrfb::kP-APP2.LPS rfaL-Ωgne-wzy(cod.opt.) pliC-ΩproD-ClyA-ApxI(aa626-845)-ApxII(aa612-801)-ApxIII(aa626-860)-HIS6-cat/pagC). To generate a control strain lacking the APP2 rfb cluster but expressing the proD-ClyA-ApxI(628-845aa)-ApxII(612-801aa)-ApxIII(626-860aa)-HIS6 fusion construct, Seq. 32 was amplified using oligonucleotides FW-rfaK_rfaL/REV_rfaL_rfaK generating a 4513bp fragment containing the integration construct with flanking homologous regions for integration at the intergenic region between rfaK and rfaL of SL1344. This fragment was transformed together with the pKD46 into SL1344 Δrfb and the procedure was followed as described above. The construct proD-ClyA-ApxI(628-845aa)-ApxII(612-801aa)-ApxIII(626-860aa)-HIS6/cat was integrated into the intergenic region between the genes rfaK and rfaL (SL1344 Δrfb rfaK-ΩproD-ClyA-ApxI(aa626-845)-ApxII(aa612-801)-ApxIII(aa626-860)-HIS6-rfaLfrat). The introduced antibiotic resistance cassette in both strains allowed the selection of positive clones (successful integration). These positive candidates were verified for the integration of the PCR fragment by PCR. The temperature sensitive helper plasmid was lost from the cells by increasing the growth temperature for several rounds of incubations. To remove the chloramphenicol resistance cassette the generated strains SL1344 Δrfb::kP-APP2.LPS rfaL-Ωgne-wzy(cod.opt.) pliC-ΩproDClyA-ApxI(aa626-845)-ApxII(aa612-801)-ApxIII(aa626-860)-HIS6-cat/pagC and SL1344 Δrfb rfaK-ΩproD-ClyA-ApxI(aa626-845)-ApxII(aa612-801)-ApxIII(aa626-860)-HIS6-cat/rfaL were transformed with the temperature sensitive plasmid pCP20 (Cherepanov et al. 1995, Gene, Vol. 158, p9-14), encoding for the flippase, which recognizes the palindromic FRT sites. After “flip out” event an FRT remained in the genome. Again, with increasing cultivation temperature the positive clones were counter selected against the flippase encoding plasmid. A final PCR verified the absence of all helper plasmids, absence of chloramphenicol resistance marker and the integration of proD-ClyA-ApxI(628-845aa)-ApxII(612-801aa)-ApxIII(626-860aa)-HIS6. The resulting strains SL1344 Δrfb::kP-APP2.LPS rfaL-Ωgne-wzy(cod.opt.) pliC-ΩproD-ClyA-ApxI(aa626-845)-ApxII(aa612-801)-ApxIII(aa626-860)-HIS6-pagC and SL1344 Δrfb rfaK-ΩproD-ClyA-ApxI(aa626-845)-ApxII(aa612-801)-ApxIII(aa626-860)-HIS6-rfaL were analyzed for their APP2 O-antigen generation as well as ClyA-ApxI(628-845aa)-ApxII(612-801aa)-ApxIII(626-860aa)-HIS6 expression. To verify the expression of ClyA-ApxI(628-845aa)-ApxII(612-801aa)-ApxIII(626-860aa)-HIS6, SL1344 Δrfb::kP-APP2.LPS rfaL-Ωgne-wzy(cod. opt.) and SL1344 Δrfb whole cell extracts were analyzed by immunoblot and Coomassie staining.

Overnight cultures of wild type SL1344, genetically modified cells lacking the endogenous O-antigen biosynthesis (SL1344 Arfb), expressing the APP2 rfb cluster (SL1344 Δrfb::APP2.LPS, Δrfb::KanR-APP2.LPS, Δrfb::KanR-APP2.LPS(cod.opt.), Δrfb::KanR-APP2.LPS rfaL-Ωgne/cat, Δrfb::KanR-APP2.LPS rfaL-Ωgne-wzy(cod. opt.)/cat, Δrfb::KanR-APP2.LPS(cod. opt.) rfaL-Ωgne-wzy(cod. opt.)/cat, Δrfb::kP-APP2.LPS(cod.opt.) rfaL-Q gne-wzy(cod.opt.) or cells with or without integrated improved APP2 rfb cluster expressing ClyA-ApxI(aa626-845)-ApxII(aa612-801)-ApxIII(aa626-860)-HIS6-pagC (SL1344 Δrfb rfaK-ΩproD-ClyA-ApxI(aa626-845)-ApxII(aa612-801)-ApxIII(aa626-860)-HIS6-rfaL, Δrfb::kP-APP2.LPS(cod.opt.) rfaL-Ωgne-wzy(cod.opt.) pliC-ΩproD-ClyA-ApxI(aa626-845)-ApxII(aa612-801)-ApxIII(aa626-860)-HIS6-pagn were diluted to an OD₆₀₀/ml of 0.05 in LB medium and grown at 37° C. on a shaker to a logarithmical growth phase (OD₆₀₀˜1). In addition, APP2 P1875 strain was grown BHI+NAD to a stationary phase at 37° C. with slow shaking (110 rpm). SL1344 derivatives and APP2 cells were harvested and cooked in 1×Lämmli (1 OD600 cell equivalent/100 μl 1×Lämmli) for 5 min at 95° C. 0.1 OD₆₀₀ cell equivalents were loaded on a on 4-12% Bis-Tris gels. As controls purified 2.5 μg HIS10-APXII(439-801aa) and 5 μg ApxIII(27-245aa)-HISS in 1×Lämmli were loaded on the gel. Proteins and whole cell extracts were separated by size in MOPS buffer (loading scheme in FIG. 15A). The gels were further processed for Coomassie staining (FIG. 15 B) and Western blotting to detect the HIS epitope (FIG. 15C). For Coomassie staining the gel was overlaid with Coomassie staining solution (3 mM Coomassie brilliant blue R 250, 40% ethanol, 10% acetic acid) and incubated shaking overnight at room temperature. The gel was placed repeatedly in Coomassie destain solution (30% ethanol, 10% acetic acid) until the desired destain of the gel and stain of proteins was observed. To detect ClyA-ApxI(aa626-845)-ApxII(aa612-801)-ApxIII(aa626-860)-HIS6, HIS10-APXII(439-801aa) and ApxIII(27-245aa)-H159 via immunoblot, proteins were transferred from the gel onto PVDF membrane. The membrane was incubated in blocking solution (PBS pH 7.5/0.05% Tween/0.1% casein) shaking for 2 h at room temperature. Then, the membrane was incubated shaking overnight at 4° C. in antibody binding solution (PBS pH 7.5/0.05% Tween/0.05% casein) containing a Tetra-HIS antibody in a 1:2000 dilution (Qiagen #34670). The immunoblot was washed 3 times for 5 min with an excess of PBS 0.05% Tween buffer pH 7.5. After-wards, the membrane was incubated for 1 h shaking at room temperature in antibody binding solution with secondary goat anti mouse IgG-HRP antibody (BETHYL Cat# A90-116P) in a 1:2000 dilution. The membrane was washed 4 times for 5 min with an excess of PBS 0.05% Tween buffer pH 7.5. Then the specific antibody binding was visualized by adding ECL solution (GE healthcare #RPN2105) to the membrane and recording the light signal detected with Stella 8300 (Raytest). Same amounts of cell equivalents were loaded when comparing SL1344 and its derivatives on a Coomassie stained gel (FIG. 15B, lanes 4-14) except SL1344 Δrfb::Kan-APP2.LPS rfaL-Ωgne-wzy(cod. opt.)/cat (potential loading error, FIG. 15B, lane 10). The Coomassie analysis of an APP2 whole cell extract revealed less overall protein staining which also differs from SL1344 cells. The purified HIS10-APXII(439-801aa) was seen as a faint band at around 50 kDa (FIG. 15B, lane 1) which is recognized by the used HIS antibody in the Western blot analysis (FIG. 15C, lane 1). Also, APXIII(27-245aa)-H159 was detectable at around 25 kDa (FIG. 15B, lane 2). In the HIS immunoblot a highly intensive signal was monitored for this protein (FIG. 15C, lane 2). Cells expressing the ClyA-ApxI(aa626-845)-ApxII(aa612-801)-ApxIII(aa626-860)-H156 (FIG. 15C, lane 13, 14) showed a signal in the HIS immunoblot above 115 kDa which is higher than the calculated molecular weight of around 104 kDa. This could be due to the SDS-PAGE parameters resulting in a changed running pattern for this transmembrane protein. Also, further bands below 115 kDa were detected which could be due to proteolytic cleavage of the fusion construct. The detected signals were absent in all other tested control cells lacking the fusion construct.

### Example 3—Clinical Studies

### 3.1) Immunization Study in Piglets with Inactivated Vaccine Strains Presenting the Recombinant APP2 O-antigen and Inactivated APP2 Bacteria

The goals of this first immunization trial was to test safety and immunogenicity of APP2 LPS after intranasal/oral or intramuscular immunization of pigs with inactivated E. coli_5 and SL1344 encoding the APP2 rfb cluster (for trial layout see Table 6).

Bacterial whole cell vaccines were prepared as following: Group 1 and 6 contained SL1344 Δrfb::kanR-APP2.LPS pMLBAD-gne pEC415-wzy) in which wzy and gne expression needed to be induced (both genes under the control of an arabinose inducible promoter) before preparing the strains for immunization. Due to that the cells were cultivated in LB with the respective antibiotics (see Table 2) and 0.01% arabinose at 37° C. shaking at 180 rpm until an OD₆₀₀ of around 0.6. Cells were induced with 0.1% arabinose. After 6 hours of incubation, cells were harvested and re-suspended in PBS buffer. Of these cell suspensions the OD600 was determined (1 OD600 corresponded to 4.1×10E8 cfu). Cells of group 4 and 8 consisted of SL1344 Δrfb and E. coli_5 Δrfb, which were used in a 1:1 mixture in the individual immunezations. These cells were cultured in LB medium until the cultures reached OD₆₀₀ above 2 and harvested. APP serotype 2 cells (Group 5 and 9) were inoculated in BHI +NAD to a stationary phase at 37° C. with slow shaking (110 rpm) and harvested and resuspended in PBS buffer. Of the cell suspensions the OD₆₀₀ was determined. Due to the culturing conditions the OD₆₀₀ to cfu conversion rate varies from the descrybed above (1 OD₆₀₀ corresponded to 4.1×10E5 cfu for APP2). Glycoengineered SL1344 as well as the SL1344 Δrfb, E. coli_5 Δrfb and the APP2 cells (in PBS buffer) were heat inactivated by incubating the material for 90 min at 80° C. shaking at 600 rpm. Before applying the material, the cells were tested for complete inactivation.

Protein based materials were prepared as follows: N-terminally HIS10 tagged neutralizing epitope of ApxII from APP2 (439-801aa) was expressed and purified from E. coli (BL21 pMLBAD-HIS10-ApxII(439-801aa)) via Ni-NTA sepharose binding and imidazole elution over FPLC. The purified protein was dialyzed in PBS. Protein concentration was determined.

To prepare the materials for intranasal and oral immunization (Groups 1-5) 2 doses of 1 ml per animal per immunization were prepared. Each dose contained 10E11 (Group 1, 4) or 10E8 (Group 5) cfu in PBS, 400 μg HIS10-ApxII(439-801aa) (Group 2) or PBS only (Group 3) and was mixed with Montanide IMS1313 (Seppic) according to supplier information to reach a concentration of 25%.

For the intramuscular immunization, 0.5 ml total volume per animal were prepared. Each dose contained 0.375 ml inactivated cells (Group 6, 8 with 10E8 cfu, Group 9 with 10E5 cfu in PBS) or 400 μg HIS10-ApxII(439-801aa) (Group 7) and 0.125 ml Montanide ISA 25 (Seppic) to reach a final concentration of 25% adjuvant. The mixture was homogenized according to supplier recommendations. Group 10 was adjuvant control (0.375 ml PBS buffer mixed with 0.125 ml Montanide ISA 25 and homogenized) and Group 11 was kept as an untreated control and did not receive any antigen.

Table 6 provides an overview of immunization groups 1-11 with applied inactivated cells, protein, adjuvant only or nothing for intranasal and oral or intramuscular application. The amount of antigen and the dose volumes are given. For intra nasal and oral vaccination 3 applications were prepared to be given into each nostril (0.5 ml per nostril) and oral (1 ml).

TABLE 6

| Group | Bacteria/proteins immunized | Adjuvant | Application | Amount antigen | Dose volume |
|---|---|---|---|---|---|
| 1 | SL1344 Δ rfb::kanR -APP2.LPS | Montanide | intra nasal | 2 × 10E11 cfu | 0.5 ml per nostril, |
| | pMLBAD-gene pEC415-wzy | IMS 1313 | & oral | (10E11 cfu in 1.0 ml) | 1.0 ml oral |
| 2 | purified HIS10-ApxII(439-801aa) | | | 2 × 400 ug | |
| | | | | (400 ug in 1.0 ml) | |
| 3 | Adjuvant control | | | | |
| 4 | SL1344 Δ rfb/E. coli_5 Δrfb | | | 2 × 10E11 cfu | |
| | | | | (10E11 cfu in [symbol] 1.0 ml) | |
| 5 | APP serotype 2 strain P1875 | | | 2 × 10E8 cfu | |
| | | | | (10E8 cfu in [symbol] 1.0 ml) | |
| 6 | SL1344 Δ rfb::konR -APP2.LPS | Montanide | intra | 10E8 cfu | 0.5 ml |
| | pMLBAD-gene pEC415-wzy | ISA 25 | muscular | | |
| 7 | purified HIS10-ApxII(439-801aa) | | | 400 ug | |
| 8 | SL1344 Δ rfb/E. coli_5 Δrfb | | | 10E8 cfu | |
| 9 | APP serotype 2 strain P1875 | | | 10E5 cfu | |
| 10 | Adjuvant control | | | | |
| 11 | Neg control | n.a. | n.a. | n.a. | n.a. |

[symbol]

indicates data missing or illegible when filed

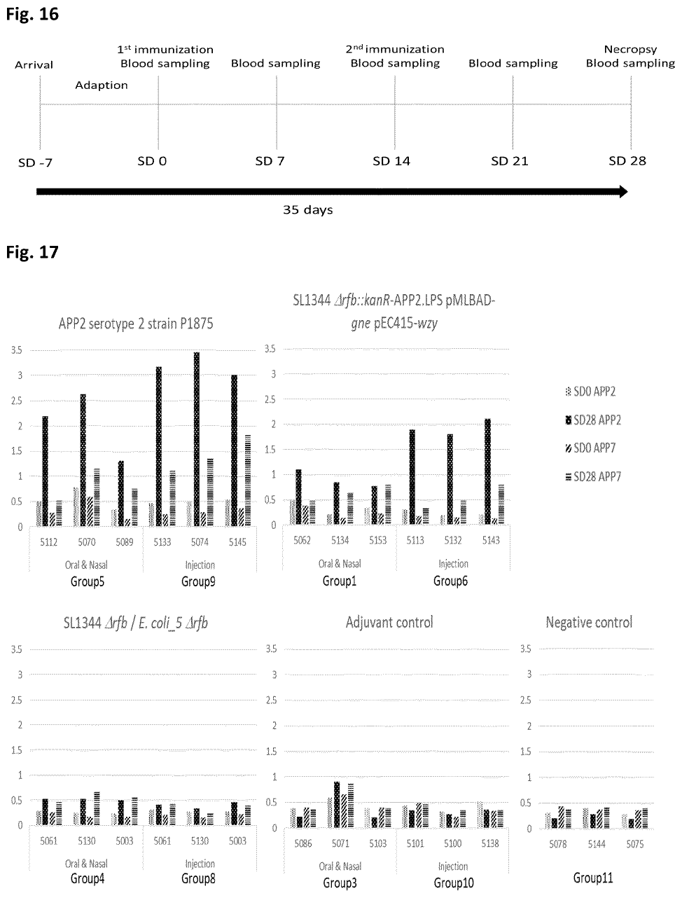

For the study, weaned pigs of approximately 4 weeks of age at study day (SD) 0 of a commercial breed of pigs (Swiss Landrace×Large White) were used. The pigs came from a confirmed APP-free holding and all animals were negative for antibodies against APP by ELISA at SD -7. Three animals per group (randomized) were immunized twice at SD 0 and SD 14 (FIG. 16).

Intranasal administration was performed using MAD Nasal Intranasal Mucosal Atomization Device MAD 100 with 3 ml syringe (Teleflex). For each animal two syringes/MAD devices were filled with each 1 ml of air and 0.5 ml of the relevant antigen/adjuvant mixture. Each pig received 0.5 ml per nostril.

Oral administration was performed using MAD Nasal Intranasal Mucosal Atomization Device MAD 100 OS with 3 ml syringe (Teleflex). For each animal one syringe/MAD device was filled with 1 ml of air and 1 ml of the relevant antigen/adjuvant mixture. The materials were administered on the tonsils.

For intramuscular administration each animal received 0.5 ml of the antigen/adjuvant mixture by intramuscular injection to the right side of the neck.

Blood was taken weekly to isolate the serum and the animals were examined at least daily for clinical signs. In comparison to the untreated control, there was a transient increase of rectal temperature up to 41.0° C. from 4 to 10 h after intramuscular injection of adjuvants as well as the vaccine containing adjuvants suggesting a non-specific rise of body temperature in response to adjuvants.

After euthanasia at SD28 the lungs were removed and BALF (bronchoalveolar lavage fluid) was collected. For each set of lungs, a 500 ml volume of PBS was flushed into the trachea, the lungs were gently inverted for 5 to 10 seconds and the fluid recovered.

IgG and IgA responses in serum and BALF towards LPS were analyzed by ELISA. Phenol/chloroform extracted LPS (followed instruction manual of iNtRON Biotechnology #17141, processing 10 OD₆₀₀ bacteria per reaction) of APP2 and 7 for sera analysis and APP1, 2, 5a and 7 for BALF analysis were coated (0.05 OD₆₀₀ equivalent in 100 μl coating buffer (PBS buffer pH 7.5)/well) in a MaxiSorb 96 well plate. The plates were incubated overnight at 4° C. on a shaker. Plates were washed one time with 200 μl washing solution (PBS pH 7.5/0.05% Tween/0.05% casein) per well and 150 μl blocking buffer (PBS pH 7.5/0.05%Tween/0.1% casein) per well was added. The plates were incubated shaking for 1 h at room temperature. The blocking buffer was removed and pig sera from SD 0 (preimmune), and 28 (2 weeks after 2nd immunization/euthanasia date) were added in a 1:500 dilution (in washing solution). BALF from SD 28 was added in a 1:2 dilution (in washing solution). After an incubation period of 1 h, shaking at room temperature, the plates were washed 3 times with 200 μl washing solution per well and secondary pig anti-IgG-HRP (BETHYL Cat#A100-105P) for sera or pig anti-IgA-HRP (BETHYL Cat# A100-102P) antibody for BALF was added in a 1:1000 dilution in washing solution (total 100 μl per well). After an incubation period of 1 h on a shaker at room temperature, the plates were washed 4 times with 200 μl washing solution per well. Development of the plates was done by adding 110 μl developing solution per well (per plate 1.5 mg 3,3′,5,5′-Tetramethylbenzidine dihydrochloride was dissolved in 1.5 ml 100% DMSO and then 13.5 ml 0.05 M phosphate-citrate buffer was added; 2 μl of fresh 30% H₂O₂ was added prior usage). The reaction was stopped by adding 110 μl stop reagent BioFX after appropriate color development was observed. The absorbance was measured at 450 nm by using Tecan Infinite M Nano reader.

To analyze the immune response towards ApxII, purified N-terminally HIS10 tagged neutralizing epitope of ApxII expressed in BL21 pMLBAD-HIS10-ApxII(439-801aa) as well as purified AcrA-HIS6 protein (HIS6 tagged C. jejuni which was used as negative control) purified from E. coli were used. 500 ng of each protein were coated per well (in 100 μl coating buffer) in 96-well plates (TPP, 92096) and probed against pig sera and BALF. Procedure was done as described above. For the ELISA to detect specific ApxII antibody generation all animals of groups 2 and 7, two animals of groups 3 and 10 and one animal of group 11 were tested.

Serum IgG analysis (FIG. 17) revealed specific anti-APP2 LPS responses at SD 28 in both animals immunized with heat inactivated APP2 (mucosally and intramuscularly immunized animals, groups 5 and 9) and in pigs immunized with glycoengineered SL1344 (SL1344 Δrfb::ΔonR-APP2.LPS pMLBAD-gne pEC415-wzy, mucosally and intramuscularly immunized animals, groups 1 and 6). No IgG responses were detectable before immunizations at SD 0. The IgG level in the sera of animals receiving SL1344 Δrfb and E. coli_5 Δrfb (group 4, 8), adjuvant (group 3, 10) or negative control (group 11) were in the background level of the performed ELISA.

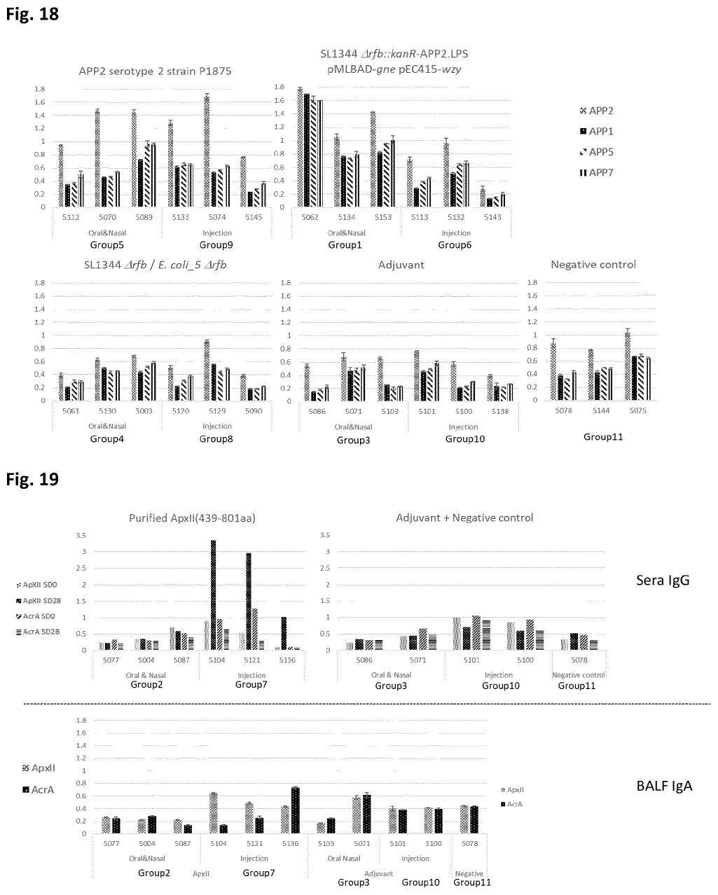

For analysis of IgA responses towards APP2 LPS, BALF was used at a 1:2 dilution. The control groups immunized with SL1344 Δrfb and E. coli_5 Arfb, adjuvant and the non-immunized group showed a high background level (FIG. 18). In addition, cross-reactivities against APP1, APPS and APP7 LPS were detected in all animals. In all animals immunized with inactivated APP2, both by mucosal and intramuscular routes, responses towards APP2 clearly exceeded those towards APP1, 5 and 7, indicating a specific IgA response towards APP2 LPS. When analyzing the group 1 and 6 (application of glycoengineered SL1344 - SL1344 Δrfb::kanR-APP2.LPS pMLBAD-gne pEC415-wzy), It appears that mucosally immunized animals showed a higher IgA titer towards APP2 LPS than immunized intramuscularly. Moreover, one individual animal (pig 5062) showed a similar level of recognition of all 4 tested APP LPS serotypes.

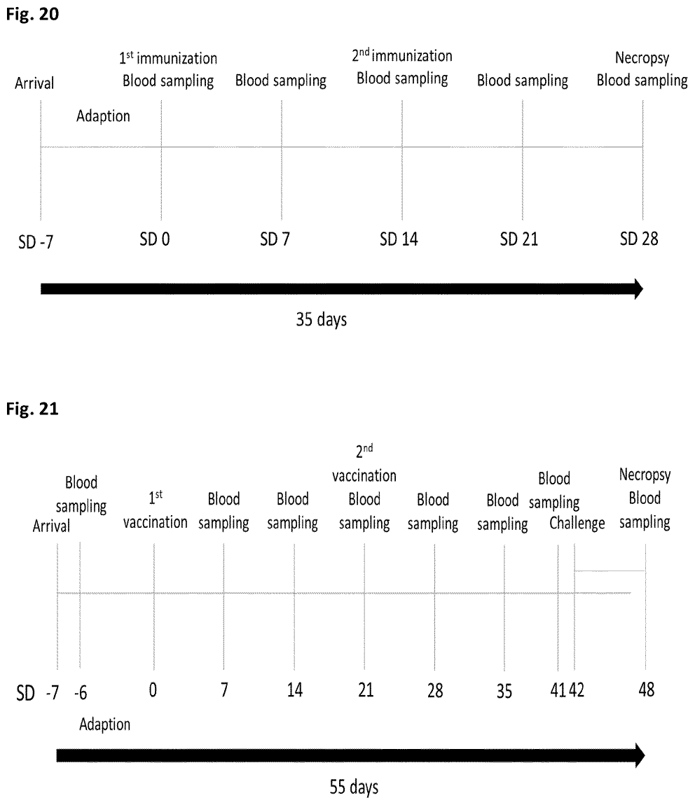

Animals immunized with purified HIS10-ApxII(439-801aa) (FIG. 19) developed elevated serum IgG towards ApxII at SD 28 after intramuscular, but not after mucosal immunization.

No specific IgA towards ApxII were detectable in BALF of pigs immunized with purified HIS10-ApxII(439-801aa). Animal 5136 showed also elevated BALF IgA for AcrA-HIS6 suggesting rather the development of HIS antibodies and not towards ApxII (both modified proteins AcrA and ApxII have only the HIS tag in common and no further homologies). In addition, animals of the control groups (adjuvant and non-immunized control groups) unspecifically recognized HIS10-ApxII(429-801aa) and AcrA-HIS6.

In conclusion, inactivated antigens were safe in pigs. The observed transient increase of body temperature was most likely mainly attributable to adjuvant reactions. Glycoengineered SL1344 (SL1344 Δrfb::kanR-APP2.LPS pMLBAD-gne pEC415-wzy) was immunogenic and specific serum IgG response towards APP2 LPS was detectable in all 6 pigs, specific IgA towards APP2 LPS was found in 4 out of 6 pigs. Immunization with purified HIS10-ApxII(439-801aa) initiated the formation of specific IgG in serum of intramuscularly immunized pigs (not after mucosal immunization). No specific IgA towards ApxII were detectable in BALF.

### 3.2) Immunization Study with Improved Live, Recombinant APP Vaccine Strains in Piglets

In the second immunization trial safety and immunogenicity of live SL1344 encoding the APP2 rfb cluster with improved APP2 O-antigen presentation on the cell surface was tested in pigs (for trial layout see Table 7). Group 1 with SL1344 Δrfb::kanR-APP2.LPS(cod.opt.) rfaL-Ωgne-wzy(cod. opt.)/cat and group 2 was kept as untreated control (no antigen applied).

For this experiment, live bacteria were prepared as following. Bacteria were cultured in LB medium shaking at 37° C. until the cultures reached OD₆₀₀ above 2 and harvested. The OD₆₀₀ was determined to estimate the cfu/ml (1 OD₆₀₀ equals 4.1×10E8 cfu). The cells were washed with sterile PBS and resuspended in PBS to a cell concentration of 1×10E8 cfu/ml. Cells were kept on ice until applied to the animals.

Table 7 provides an overview of immunization groups 1 with applied live cells (intranasal and oral) or untreated control group 2. The amount of antigen and the dose volumes are given. For intranasal and oral vaccination 2 applications (2 times 1 ml) were prepared and given into each nostril (each nostril 0.5 ml) and oral (1 ml).

TABLE 7

| Group | Test material | Application | Amount antigen | Dose volume |
|---|---|---|---|---|
| 1 | SL1344 Δ rfb::kanR -APP2.LPS(cod. opt.) | intra nasal | 2 × 10E8 cfu | 0.5 ml per nostril, |
| | rfaL-Ωgne-wzy (cod. opt.)/cat | & oral | (10E8 cfu in 1.0 ml) | 1.0 ml oral |
| 2 | untreated control | n.a. | n.a. | n.a. |

n.a.—not applied

For the study, Danebreed (a commercial cross of landrace and large white) with Duroc sires pigs of approximately 4-5 weeks of age at SD 0 were used. 6 animals per group (randomized) were immunized twice at SD 0 and SD 14 (FIG. 20). The pigs came from a confirmed APP-free holding and all animals were negative for antibodies against APP by ELISA at SD-7. Intranasal administration of live bacteria in PBS was performed using MAD Nasal Intranasal Mucosal Atomization Device MAD 100 with 3 ml syringe (Teleflex). For each animal two syringes/MAD devices were filled with each 1 ml of air and 0.5 ml of the relevant live cell/PBS mixture. Each pig received 0.5 ml per nostril. Oral administration was performed using MAD Nasal Intranasal Mucosal Atomization Device MAD 100 OS with 3 ml syringe (Teleflex). For each animal one syringe/MAD device was filled with 1 ml of air and 1 ml of the relevant live cell/PBS mixture. The materials were administered on the tonsils. Blood was taken weekly to isolate the serum and the animals were examined for clinical signs.

After euthanasia at SD 28, the lung was removed and a BALF was collected. For each set of lungs, a 500 ml volume of PBS was flushed into the trachea, the lungs gently inverted for 5 to 10 seconds and the fluid recovered (BALF). Persistence of the bacterial strain used for immunization was tested after euthanasia. Swab samples were collected from the tonsils, lung (left and right diaphragmatic lobes) and tracheabronchial lymph nodes of each animal in group 1 and 2. Each swab was aseptically streaked onto a sterile LB agar plate containing Kanamycin and incubated at 37° C. for 16 to 24 hours.

All animals remained in good health throughout the study. No abnormal clinical signs were observed in any animal. After necropsies, no lung abnormalities were observed. No lesions or other pathologies were noted in the lungs of any animal.

Bacterial colonies were recovered from two animals in group 1. One animal (number 341609) had a total of 366 colonies recovered from the tonsils, but no other tissues. The second animal (number 341618) had a single colony present from the tonsils and tracheobronchial lymph nodes, but no other tissues. No bacteria could be isolated from animals of the non-immunized control group.

The sera and BALF immune responses directed against APP2 LPS were analyzed by immunoblot. APP2 P1875 strain was grown in BHI+NAD to a stationary phase at 37° C. with slow shaking (110 rpm). Cells were harvested and used for further processing. For APP2 0-antigen analysis, cells were resuspendded in 1×Lämmli buffer (1 OD₆₀₀ cells/100 μl 1×Lämmli buffer). The sample was incubated at 95° C. for 5 min. 12 μg proteinase K per OD₆₀₀ equivalent cells (stock 20 mg/ml in 10 mM Tris-HCl pH 7.5, 20 mM CaCl₂, 50% glycerol) were added and the sample was incubated for 1 h at 60° C. Afterwards proteinase K treated sample (0.1 OD₆₀₀ cell equivalent) was loaded on 4-12% Bis-Tris gels, and molecules were separated by size in MES buffer. The gels were further processed for immunoblotting. LPS was transferred from the gel onto PVDF membranes. The membranes were incubated in blocking solution (PBS pH 7.5/0.05%Tween/0.1% casein) shaking for 2 h at room temperature. After, the membranes were incubated shaking overnight at 4° C. in antibody binding solution (PBS pH 7.5/0.05% Tween/0.05% casein) containing pig sera of 6 animals of group 1 immunized with SL1344 Δrfb::kanR-APP2.LPS(cod.opt.) rfaL-Ωgne-wzy(cod. opt.)/cat and 2 animals of group 2 (untreated control) at SDO and 28 in a 1:500 dilution and BALF at SD28 in a 1:2 dilution. The immunoblot were washed 3 times for 5 min with an excess of PBS 0.05% Tween buffer pH 7.5. Afterwards the membrane was incubated for 1 h shaking at room temperature in antibody binding solution with secondary pig anti-IgG-HRP antibody (BETHYL Cat#A100-105P) or pig anti-IgA-HRP (BETHYL Cat# A100-102P) in a 1:2000 dilution. The membranes were washed 4 times for 5 min with an excess of PBS 0.05% Tween buffer pH 7.5. Afterwards the specific antibody binding was visualized by adding ECL solution (GE healthcare #RPN2105) to the membrane and recording the light signal detected with Stella 8300 (Raytest).

TABLE 8

| | | Detected | |
|---|---|---|---|
| Group | Test material | immune response | Response |
| 1 | SL1344 Drfb::kanR-APP2.LPS(cod. opt.) | Systemic IgG | 6 out of 6 animals |
| | rfaL Ωgne-wzy(cod. opt.)/cat | | |
| | | Mucosal IgA | 5 out of 6 animals |
| 2 | Untreated control | Systemic IgG | 0 out of 2 animals |
| | | Mucosal IgA | 0 out of 2 animals |

Evaluating the specific immune responses against APP2 LPS (Table 8) showed for 6 out of 6 animals immunized with live SL1344 Δrfb::kanR-APP2.LPS(cod.opt.) rfaL-Ωgne-wzy(cod. opt.)/cat (group 1) a clear APP2 LPS recognition by sera IgG at SD 28, which was absent at SD 0. 2 tested animals of group 2 (untreated control) did not show any significant increase in serum IgG directed specifically towards APP2 LPS.

Analyzing IgA against APP LPS in the BALF shows for 5 out of 6 animals an elevated specific recognition of APP2 O-antigen in group 1. Both tested animals of the untreated control group 1 showed no IgA response against APP2.

In conclusion, live glycoengineered SL1344 was safe and colonization could be confirmed in 2 pigs after euthanasia. In addition, the recombinant APP2 O-antigen was immunogenic, both systemic IgG and mucosal IgA responses were induced.

### 3.3) Efficacy of Glycoengineered APP Vaccine Candidates Against APP Serotype 2 in Piglets

The aim of this study was to examine the efficacy of preventing an APP2 infection in pigs immunized with the developed glycoengineered SL1344 vaccine strain displaying the APP2 O-antigen on its surface (for group overview see Table 9).

Vaccine strain SL1344 Δrfb::kanR-APP2.LPS(cod.opt.) rfaL-Ωgne-wzy(cod. opt.)/cat was used as live vaccine (group 1), the second strain (SL1344 Δrfb::kanR-APP2.LPS pEC415-wzy pMLBAD-gne with induced wzy and gne expression) was used as inactivated vaccine (group 2). Both were applied in combination with neutralizing epitopes of ApxII and III. Vaccines were administered by oral and intranasal routes, neutralizing epitopes of Apx toxins by intramuscular application at SD 0 and 21 (FIG. 21). These groups were compared to animals vaccinated with either inactivated APP2 in combination with the injection of the 2 Apx neutralizing epitopes (group 3), Apx neutralizing epitopes alone (group 4) or non-vaccinated animals (group 5). Pigs were challenged with an infectious dose of APP serotype 2 (HK 361, NCTC 10976) at SD 42 and euthanized at SD 48. The animals were regularly monitored for clinical signs (at least twice daily during the 6 days after challenge) and rectal temperature measurements were performed either once or twice daily. Online, telemetric measurement of body temperature (AniPill from Body Cap) was used in 50% of the animals by subcutaneous located temperature probes.

For this experiment bacteria and proteins were prepared as follows. To prepare 5L1344 Δrfb::kanR-APP2.LPS(cod.opt.) rfaL-Ωgne-wzy(cod. opt.)/cat for live vaccination (group 1) the cells were inoculated in LB medium at 37° C. shaking for around 24 h. On the day of vaccination, the bacterial culture grown to stationary phase (OD₆₀₀>2) was cooled on ice for approximately 10 min. The culture was centrifuged for 15 minutes at 4100× g at 4° C. The pelleted cells were carefully re-suspended in sterile pre-cooled PBS buffer. The suspension was centrifuged again for 15 minutes at 4100× g at 4° C., the supernatant removed, and the resulting cell pellet resuspended in pre-chilled PBS. The optical density at 600 nm (OD₆₀₀) was determined (1 OD₆₀₀ corresponded to 4.1×10E8 cfu). Each vaccine dose applied to pigs contained 1×10E8 cfu in 1 ml volume.

To prepare SL1344 Δrfb::kanR-APP2.LPS pEC415-wzy pMLBAD-gne for inactivation and vaccination (group 2) the cells were inoculated in LB medium at 37° C. shaking overnight. The following day, the OD₆₀₀was determined, and cells diluted to 0.075 OD₆₀₀/ml in LB medium supplemented with Amp, Tmp and 0.01% arabinose. The culture was incubated at 37° C. shaking. At an OD₆₀₀ of 0.6 arabinose was added to 0.1% final concentration to induce the expression of the plasmid-encoded proteins (gne and wzy). The culture was incubated further at 37° C. shaking. Again, after 6 h arabinose was added to 0.1%. The culture was further incubated at 37° C. shaking for 10-12 h. The next day the OD₆₀₀ was determined, and the culture was centrifuged for 10 min at 7,000×g at 4° C. The supernatant was removed, then the pelleted cells were carefully resuspended with PBS. The optical density at OD₆₀₀ was determined (1 OD₆₀₀ corresponded to 4.1×10E8 cfu). For heat inactivation the cell suspension was incubated for 90 min at 80° C. in a water bath. Afterwards the suspension was frozen and stored at −80° C. until the day of vaccination. Before applying the material, the cells were tested for complete inactivation.

Protein based vaccines were prepared as follows. N-terminally HIS10-tagged neutralizing epitope of ApxII(439-801aa) (group 1-4) was expressed and purified from E. coli (BL21 pMLBAD-HIS10-ApxII(439-801aa)) via Ni-NTA sepharose binding and imidazole elution over FPLC. The purified protein was dialyzed in PBS. Protein concentration was determined. C-terminally HIS9 tagged neutralizing epitope of ApxIII(27-245aa) (group 1-4) was expressed and purified from E. coli (BL21 pMLBAD-APXIII(27-245aa)-H159) via Ni-NTA sepharose binding and imidazole elution. The purified protein was dialyzed in PBS. Protein concentration was determined.

To prepare the live vaccine (group 1) for intranasal and oral application 2 doses of 1 ml per animal were prepared. Each vaccine dose applied contained 1×10E8 cfu in 1 ml volume.

To prepare the inactivated vaccines for intranasal and oral vaccination (Groups 2 and 3) 2 doses of 1 ml per animal were prepared. Each dose contained 10E11 (Group 2) and 10E8 (Group 3) cfu in PBS and was mixed with the adjuvant Montanide IMS1313 (Seppic) according to supplier information to reach a concentration of 25%.

For the intramuscular vaccination of HIS10-APXII(439-801aa) and ApxIII(27-245aa)-HIS9 (group 1-3), 0.5 ml total volume per antigen and per animal were prepared. Each dose contained 0.375 ml 400 μg protein antigen and 0.125 ml Montanide ISA 28 (Seppic) to reach a final concentration of 25%. The mixture was homogenized according to supplier recommendations. Group 4 was kept as untreated control (no antigens applied).

Challenge material was prepared as follows. Two days prior to challenge infection the APP2 strain HK361 was cultured on HIS+V culture plates and grown overnight at 37° C. and 5% CO₂. The following day, cultures were prepared by transferring one colony to 10 new HIS+V culture plates and incubated for 6 h. After, all colonies of each plate were transferred to tubes filled with PBS and stored overnight in the fridge. The cfu cell/PBS solution were determined by diluting and plating the cells on HIS+V culture plates. The next morning cfu's were determined and the solution was diluted to obtain the challenge concentration of 10E6 cfu/ml.

TABLE 9

| | No. | | | | | |
|---|---|---|---|---|---|---|
| Group | animals | Test materials | Adjuvant | Route | Amount (Volume) | Admin. day |
| 1 | 7^{a} | Live SL1344 Δrfb::kanR- | none | oral & nasal | 10E8 cfu (1 ml) oral & 10E8 | SD 0 & SD 21 |
| | | APP2.LPS(cod.opt.) rfaL - | | | cfu (1 ml) i.n. | |
| | | Ωgne-wzy(cod. opt.)/cat | | | | |
| | | Purified HIS10-APXII(439- | MontanidelSA 28 | intramuscular | 400 μg HIS10-APXII(439- | |
| | | 801aa) | | | 801aa) (0.5 ml)/400 μg | |
| | | Purified APXIII(27-245aa)-HIS9 | | | APXIII(27-245aa)-HIS9 | |
| | | | | | (0.5 ml) | |
| 2 | 7^{b} | Inactivated SL1344 | Montanide IMS | oral & nasal | 10E11 cfu, 0.583 g (1 ml) oral | |
| | | Δrfb::kanR-APP2.LPS | 1313 | | & 10E11 cfu, 0.583 g (1 ml) | |
| | | pEC415-wzy pMLBAD-gne | | | i.n. | |
| | | Purified HIS10-APXII(439- | MontanideISA 28 | intramuscular | 400 μg HIS10-APXII(439- | |
| | | 801aa) | | | 801aa) (0.5 ml)/400 μg | |
| | | Purified APXIII(27-245aa)-HIS9 | | | APXIII(27-245aa)-HIS9 | |
| | | | | | (0.5 ml) | |
| 3 | 8 | Purified HIS10-APXII(439- | MontanideISA 28 | intramuscular | 400 μg HIS10-APXII(439- | SD 0 & SD 21 |
| | | 801aa) | | | 801aa) (0.5 ml)/400 μg | |
| | | Purified APXIII(27-245aa)-HIS9 | | | APXIII(27-245aa)-HIS9 | |
| | | | | | (0.5 ml) | |
| 4 | 8 | Non-vaccinated control group | | | | |

^{a}One pig of group 1 died unexpectedly before the study start during the accommodation phase (day −5) most likely due to sudden heart failure.
^{b}One pig of group 2 died unexpectedly on SD 5. The pig was found dead without prior disease signs. It is most likely not related to the vaccination. A comparable picture was found in the pig of group 1, which died prior to the first vaccination.

For the study, pigs of approximately 5 weeks of age of a commercial breed of pigs (TOPIGS-Norsvin, TN70, Z-line) were used. The pigs came from confirmed APP-free holdings and all animals were negative for antibodies against APP by ELISA at SD -7. 7-8 animals per group (randomized) were vaccinated 2 times in an interval of 3 weeks (SD 0, SD 21; FIG. 21). Groups 1 and 2 contained only 7 animals each due to two animals dying before study start (group 1) or at SD 5 (group 2) without any detectable lesions at necropsy.

Intranasal administration was performed using MADgic Laryngotracheal Mucosal Atomization Device MAD 600 with 3 ml syringe (Teleflex). For each animal two syringes/MAD devices were filled with each 1 ml of air and 0.5 ml of the relevant antigen/adjuvant mixture. Each pig received 0.5 ml per nostril.

Oral administration was performed using Nasal Intranasal Mucosal Atomization Device MAD 100 OS with 3 ml syringe (Teleflex). For each animal one syringe/MAD device was filled with 1 ml of air and 1 ml of the relevant antigen/adjuvant mixture. The materials were administered on the tonsils.

For intramuscular administration each animal received 0.5 ml of each antigen/adjuvant mixture by intramuscular injection to the neck.

The challenge was performed on day 42 by intranasal inoculation of 2 ml with 10E6 cfu/ml of APP2 strain HK361 by the MAD Nasal™ Intranasal Mucosal Atomization Device)(Teleflex®.

Blood was taken at SD -6, 7, 14, 21, 28, 35, 41 and 48 (FIG. 21) to isolate the serum and the animals were examined for clinical signs.

At SD 48 days the animals were euthanized, and necropsy was performed. Lesions of the lungs and pleura were scored according to Hannan et al. 1982, Res. Vet. Sci., Vol. 33(1), p76-88. Scoring was based on the percentage of each lobe (7 locations) that is affected by typical APP lesions (Table 10). Both the dorsal and ventral surfaces of each lung were palpated and visually examined, but a single value was arrived at for each lobe based on an average of the entire surface area. Tukey-Kramer's All Pairs Simultaneous Confidence Intervals of Mean Difference and P-Value was used as statistic evaluation method.

TABLE 10

| Lesion scoring | Description |
|---|---|
| 0 | 0% area affected by lesions |
| 1 | 1-15% area affected by lesions |
| 2 | 16-30% area affected by lesions |
| 3 | 31-45% area affected by lesions |
| 4 | 46-60% area affected by lesions |
| 5 | Peracute lungs (swelling, haemorrhage and consolidation |
| | of a large part [>50%] or the whole of the lung |
| | volume, often with fibrin deposition on the surface) |

To test for the presence or absence of the challenge APP2 strain, from all groups tissue specimens from the lung was sampled for bacteriological analysis. For re-isolation of APP2 bacteria, lung tissue samples were emerged in cooking water for 7 sec and thereafter disrupted in a stomacher to achieve a suspension, of which 100 μl were plated on HIS+V plates and incubated overnight at 37° C. and 5% CO₂. Colonies confirmed by MALDI-TOF.

No pig showed abnormal local or systemic reactions after vaccinations. There was a moderate increase of body temperatures in all vaccinated pigs 0.5 days after both vaccinations, which returned to normal values 1 day after vaccinations.

Body temperature was measured twice daily after challenge. There was an increase of rectal temperature starting one day after challenge. In general, temperature rise was lower in vaccinated groups compared to the non-vaccinated control group. This difference was most pronounced at 4 days post challenge, when average temperatures of all vaccinated groups were statistically significantly lower compared to the unvaccinated control group (Table 11).

TABLE 11
Statistically significant (*) lower increase of body temperature
in vaccinated groups (group 1 to 3) compared to the non-vaccinated
control group (group 4). Group 1: live SL1344 Δrfb::kcanR-
APP2.LPS(cod. opt.) rfaL-Ωgne-wzy(cod. opt.)/cat, purified
HIS10-APXII(439-801aa), purified APXIII(27-245aa)-HIS9. Group
2: inactivated SL1344 Δrfb::kanR-APP2.LPS pEC415-wzy pMLBAD-
gne, purified HIS10-APXII(439-801aa), purified APXIII(27-245aa)-
HIS9. Group 3: purified HIS10-APXII(439-801aa), purified APXIII(27-
245aa)-HIS9. Group 4: non-vaccinated control group. Dunnett's
multiple comparison test.

| | | |
|---|---|---|
| Mean | 95,00% | |
| difference | confidence | |
| of rectal | interval of | Adjusted P |
| temperatures | difference | Value |

4 days post challenge, morning measurement

| | | | |
|---|---|---|---|
| Group 4 versus Group 1 | 0.7071 * | 0.2709 to 1.143 | 0.0003 |
| Group 4 versus Group 2 | 0.7214 * | 0.2852 to 1.158 | 0.0002 |
| Group 4 versus Group 3 | 0.5625 * | 0.1410 to 0.9840 | 0.0042 |

4 days post challenge, afternoon measurement

| | | | |
|---|---|---|---|
| Group 4 versus Group 1 | 0.6661 * | 0.2298 to 1.102 | 0.0008 |
| Group 4 versus Group 2 | 0.7946 * | 0.3584 to 1.231 | <0.0001 |
| Group 4 versus Group 3 | 0.7000 * | 0.2785 to 1.121 | 0.0002 |

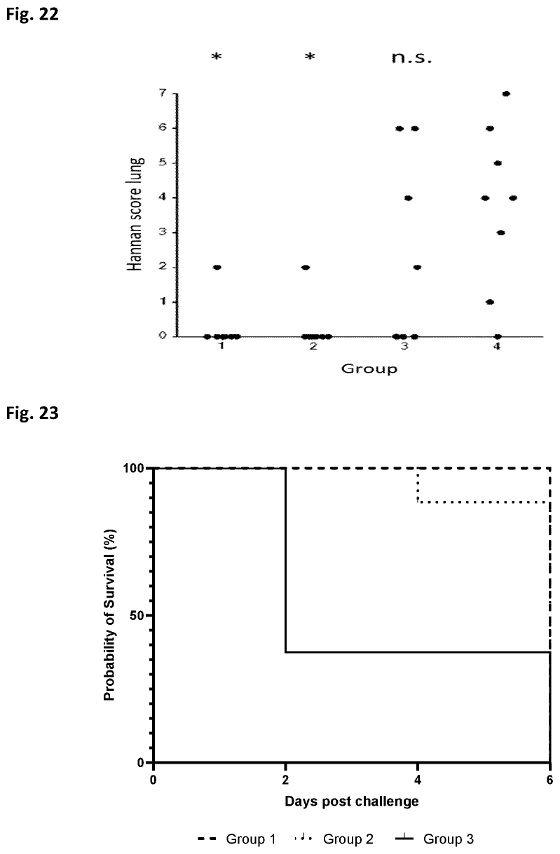

Evaluating the lung lesions (see FIG. 22, Table 12) lung scoring of individual animals revealed animals in the negative control group (group 4) showing lung damages from Hannan score 0 — 7 (mean at 3.75). Animals vaccinated only with Apx toxins (group 3) showed no significant reduction in lung scores compared to the unvaccinated control group, although the mean value in this group dropped to 2.25.

6 out of 7 animals of group 1 vaccinated with inactivated APP2 and Apx neutralizing epitopes had no detectable lung lesions and one animal showed lung lesions with a score of 2. With a mean lung lesion score of 0.286 the lung lesions were significantly (P =0.00369) less developed than in the control animals (group 4).

TABLE 12
Tukey-Kramer's All Pairs Simultaneous Confidence Intervals of Mean
Difference and P-Value of all groups compared to negative control
group 4. Shown are the mean, the pairwise differences between the
means of the Hannan lung scoring of each group as well as the multiple
comparison P-value (group 4 against groups 1-3). This is the significance
level at which the difference becomes significant using the Tukey-
Kramer multiple comparison procedure. Statistic significant lung
scoring compared to group 4 when P-value < 0.05.

| | | | Mean | |
|---|---|---|---|---|
| Group | Animals | Mean | difference | P-value |
| 4 | 8 | 3.75 | | |
| 1 | 7 | 0.285714 | 3.464 | 0.00369 |
| 2 | 7 | 0.285714 | 3.464 | 0.00369 |
| 3 | 8 | 2.25 | 1.5 | 0.42721 |

The semi-quantitative results of the re-isolation of the challenge strain (Table 13) showed that in all animals of group 4 high numbers of APP2 bacteria could be re-isolated. No statistical evaluation could be done with the data recorded, but a tendency was observed that lung lesion scores positively correlated with the numbers of re-isolated bacteria: In groups 1 only the one pig with detectable lesions revealed high numbers of re-isolated challenge bacteria. In 2 more animals of this group low amount of APP2 could be re-isolated from lung areas without visible lesions. Although groups 1 and 2 did not differ regarding their lung lesion scores (in both groups 6 out of 7 pigs were without lesions), in group 2 the one pig with detectable lesions and five lesion-free pigs were positive in reisolation of low to high numbers of challenge bacteria (6 out of 7 animals APP2 challenge strain positive). For the 4 animals with lung lesions of group 3 high amounts of APP2 could be isolated from the lungs either from areas with lesions or from regions without visible lesions.

TABLE 13
Comparison of lung lesions scores and culture scores.

| | | Lung | Re-isolation of APP2 bacteria from lung | | | |
|---|---|---|---|---|---|---|
| | | lesion | Total culture | Score in lung | Score outside | |
| Group | Animal | score | score ^{a} | lesions ^{b} | of lesions ^{c} | |
| 1 | 233 | 2 | +++ | +++ | − | |
| 1 | 234 | 0 | + | − | + | (17/4) |
| 1 | 235 | 0 | + | − | + | (10/0) |
| 1 | 232 | 0 | − | − | − | |
| 1 | 236 | 0 | − | − | − | |
| 1 | 237 | 0 | − | − | − | |
| 1 | 238 | 0 | − | − | − | |
| 2 | 244 | 2 | +++ | +++ | + | (0/13) |
| 2 | 243 | 0 | +++ | − | +++ | (1000/0) |
| 2 | 246 | 0 | ++ | − | ++ | (250/150) |
| 2 | 239 | 0 | + | − | + | (1/0) |
| 2 | 240 | 0 | + | − | + | (2/2) |
| 2 | 245 | 0 | + | − | + | (2/2) |
| 2 | 241 | 0 | − | − | − | (0/0) |
| 3 | 265 | 6 | +++ | +++ | + | (3/0) |
| 3 | 266 | 6 | +++ | +++ | + | (1/0) |
| 3 | 261 | 4 | +++ | − | +++ | (0/1000) |
| 3 | 262 | 2 | +++ | ++ | +++ | (>1000/0) |
| 3 | 259 | 0 | − | − | − | (0/0) |
| 3 | 260 | 0 | − | − | − | (0/0) |
| 3 | 263 | 0 | − | − | − | (0/0) |
| 3 | 264 | 0 | − | − | − | (0/0) |
| 4 | 268 | 7 | +++ | +++ | +++ | (1000/16) |
| 4 | 269 | 6 | +++ | +++ | +++ | (>1000/80) |
| 4 | 257 | 5 | +++ | − | +++ | (2/>1000) |
| 4 | 256 | 4 | +++ | +++ | + | (0/1) |
| 4 | 267 | 4 | +++ | +++ | + | (0/2) |
| 4 | 255 | 3 | +++ | +++ | + | (0/12) |
| 4 | 258 | 1 | +++ | +++ | + | (21/0) |
| 4 | 270 | 0 | ++ | − | ++ | (0/250) |

^{a} The total challenge strain reisolation culture score is derived from the highest score determined (either in lung lesions or outside of visible lesions).
^{a} Samples were taken from visible lung lesions.
^{b} Samples were taken from two defined lung locations outside of visible lesions. Scoring see above. Total numbers of cfu isolated at 2 locations are given in brackets.
Scoring: − (no growth), + (<50 cfu), ++ (50 to 500 cfu), +++ (>500 cfu).

In summary, the glycoengineered candidate vaccine was highly efficacious and significantly reduced lung lesions in vaccinated animals compared to untreated animals. The experiments prove that a recombinant bacterial vaccine presenting the heterologous APP O-antigen on the surface, optionally in combination with neutralizing epitopes of ApxII and III, can almost completely prevent lung lesion development and strongly reduce colonization of lung with APP challenge bacteria.

### 3.4) Efficacy of Glycoengineered APP Vaccine Candidates Against APP Serotype 2 in Piglets Administered by Oral and Nasal Route

The aim of this study was to reproduce the efficacy of inactivated APP2 vaccine strain (SL1344 Δrfb::kanR-APP2.LPS pEC415-wzy pMLBAD-gne with induced wzy and gne expression) when applied orally and intranasally in combination with intramuscularly injected neutralizing epitopes of ApxII and III (Group 1). Furthermore, the efficacy was compared to animals treated with the commercial vaccine Porcilis° APP (Group 2). The procedure was comparable to the experiment described in chapter 3.3. Vaccines were administered at SD 0 and 21. Both groups were compared to non-vaccinated animals (group 3). Pigs were challenged with an infectious dose of APP serotype 2 (HK 361, NCTC 10976) at SD 42 and euthanized at SD 48. The animals were regularly monitored for clinical signs (at least twice daily during the 6 days after challenge) and rectal temperature measurements were performed either once or twice daily.

For this experiment bacteria and proteins as well as the challenge material were prepared as described in chapter 3.3 above.

TABLE 14

| | No. | | | | | |
|---|---|---|---|---|---|---|
| Group | animals | Test materials | Adjuvant | Route | Amount (Volume) | Admin. day |
| 1 | 8 | Inactivated SL1344 Δrfb::kanR-APP2.LPS | Montanide IMS 1313 | oral & nasal | 10E11 cfu (1 ml) oral & 1 | SD 0 & SD 21 |
| | | pEC415-wzy pMLBAD-gne | | | 10E1 cfu, (1 ml) i.n. | |
| | | Purified HIS10-APXII(439-801aa) | MontanideISA 28 | intramuscular | 400 μg HIS10-APXII(439- | |
| | | Purified APXHI(27-245aa)-HIS9 | | | 801aa) (0.5 ml)/400 μg | |
| | | | | | APXIII(27-245aa)-HIS9 | |
| | | | | | (0.5 ml) | |
| 2 | 8 | Commercial APP vaccine (Porcilis APP) | a-Tocopherol | intramuscular | According to manufacturer | |
| | | | | | manual | |
| 3 | 8 | Non-vaccinated control group | | oral & nasal | Phosphate buffered saline | |

For the study pigs of approximately 5 weeks of age of a commercial breed of pigs (TOPIGS-Norsvin, TN70, Z-line) were used. The pigs came from confirmed APP-free holdings and all animals were negative for antibodies against APP prior study day 1. 8 animals per group (randomized) were vaccinated 2 times in an interval of 3 weeks (SD 0, SD 21). Intranasal and oral administration of the glycoengineered vaccine as well as the intramuscular injection of the Apx antigens (group 1) was performed as described in chapter 3.3 above. 2 ml Porcilis° APP were injected intramuscular in animals of group 2.

The challenge was performed on day 42 by intranasal inoculation of 2 ml with 10E6 cfu/ml of APP2 strain HK361 by the MAD Nasal™ Intranasal Mucosal Atomization Device (Teleflex®).

Blood was taken at SD -1, 7, 14, 20, 28, 35, 41 and 48 to isolate the serum and the animals were examined for clinical signs.

At SD 48 the animals were euthanized, and necropsy was performed. Lesions of the lungs and pleura were scored according to Hannan et al. 1982, Res. Vet. Sci., Vol. 33(1), p76-88. Scoring was based on the percentage of each lobe (7 locations) that is affected by typical APP lesions (Table 10). Both the dorsal and ventral surfaces of each lung were palpated and visually examined, but a single value was arrived at for each lobe based on an average of the entire surface area. Dunnett's multiple comparison test and P-Value was used as statistic evaluation method.

To test for the presence or absence of the challenge APP2 strain, from all groups tissue specimens from the lung was sampled for bacteriological analysis. For re-isolation of APP2 bacteria, lung tissue samples were emerged in cooking water for 7 sec and thereafter disrupted in a stomacher to achieve a suspension, of which 100 μl were plated on HIS+V plates and incubated overnight at 37° C. and 5% CO₂. Colonies were confirmed by MALDI-TOF. The bacterial scoring values were translated in 0 =no APP2 bacteria isolated, 1=<20 CFU (colony forming units) APP2 bacteria isolated, 2=<200 CFU APP2 bacteria isolated and 3=>200 CFU APP2 bacteria isolated.

No pig showed abnormal local or systemic reactions after vaccinations. There was a moderate increase of body temperatures in all vaccinated pigs 0.5 days after both vaccinations, which returned to normal values 1 day after vaccinations.

Body temperature was measured twice daily after challenge. There was a moderate increase of rectal temperature starting one day after challenge in the control group 3. In general, temperature rise was lower in vaccinated groups compared to the non-vaccinated control group.

Due to the symptoms caused by the infection animals reaching the human endpoint criteria were euthanized and clinically evaluated. FIG. 23 shows the graphs of the probability of survival for animals of all 3 groups. At day 2 post challenge, 5 out of 8 animals of the control group 4 had to be euthanized due to increased sickness rates. At day 4, 1 animal of the commercial vaccine group 2 had to be euthanized. None of the animals of the glycoengineered vaccine group 1 showed clinical symptoms and all animals survived until the end of the study on day 6 post challenge.

Evaluating the lung lesions (see FIG. 24, Table 15), lung scoring of individual animals revealed animals in the negative control group (group 3) showing lung damages from Hannan score 0 — 8 (mean at 3.75). Animals vaccinated with the commercial vaccine (group 2) showed not a significant but a tendency in reduction in lung scores with a P value slightly above 0.05 compared to the unvaccinated control group.

None of the animals of group 1 vaccinated with inactivated APP2 and Apx neutralizing epitopes had detectable lung lesions. The vaccination group 1 was significantly different from the control group 3 (P =0.007).

TABLE 15
Dunnett's multiple comparison and P-Value of all groups
compared to negative control group 3. Shown are the mean,
the pairwise differences between the means of the Hannan
lung scoring of each group as well as the multiple comparison
P-value (group 3 against groups 1 and 2). Statistic significant
lung scoring compared to group 3 when P-value < 0.05.

| | | | Mean | |
|---|---|---|---|---|
| Group | Animals | Mean | Difference | P-value |
| 3 | 8 | 3.75 | | |
| 1 | 8 | 0 | 3.75 | 0.007 |
| 2 | 8 | 1.125 | 2.625 | 0.0527 |

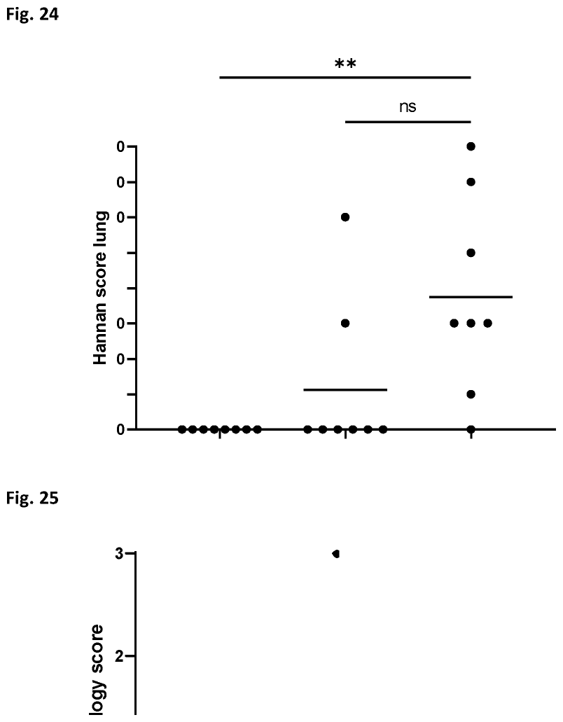

The semi-quantitative results of the re-isolation of the challenge strain (FIG. 25) showed that in all animals but one of group 3 high numbers of APP2 bacteria could be re-isolated. In the commercial vaccine group 2, APP2 bacteria could be reisolated from 4 out of 8 animals with 2 animals having high numbers of bacteria present. In contrast, none of the animals vaccinated with the glycoengineered vaccine in group 1 was positive for APP2 bacteria reisolation.

In summary, the results of the previous study could be confirmed. The glycoengineered candidate vaccine was highly efficacious and resulted in no APP2 bacteria reisolation 6 days post challenge and no lung lesions development in vaccinated animals compared to untreated animals. Furthermore, the efficacy was higher than observed for animals treated with a commercial APP vaccine.

1.-20. (canceled).

21. A gram-negative bacterial host cell suitable for vaccines, the bacterial host cell comprising
(a) a heterologous functional Actinobacillus pleuropneumoniae (APP) rfb gene cluster, wherein the heterologous functional APP rfb gene cluster produces an APP O-antigen that is bound to the lipid A-core of the bacterial host cell and is located on the bacterial host outer surface, and wherein the endogenous rfb gene cluster of the bacterial host cell is not functional.

22. The bacterial host cell according to claim 1, further comprising at least one of:
(b) a heterologous promoter for regulating the transcription of the heterologous APP rfb gene cluster that is stronger than the endogenous promoter for the endogenous rfb gene cluster;
(c) at least one further gene for functionally expressing an enzyme assisting the APP O-antigen synthesis; or
(d) at least one neutralizing epitope of Apx toxins.

23. The bacterial host cell according to claim 21, wherein the bacterial host cell is selected from the group consisting of Enterobacteriaceae, Burkholderiaceae, Pseudomonadaceae, Vibrionaceae, optionally Burkholderia thailandensis, Pseudomonas aeruginosa, Vibrio natriegens, Vibrio cholerae, Escherichia coli, optionally E. coli_5, Salmonella enterica, optionally Salmonella enterica subsp. enterica, optionally Salmonella enterica subsp. enterica selected from the group consisting of serovar Typhimurium, Enteritidis, Heidelberg, Gallinarum, Hadar, Agona, Kentucky and Infantis, and Salmonella enterica subsp. enterica serovar Typhimurium SL1344.

24. The bacterial host cell according to claim 21, wherein the heterologous rfb gene cluster is selected from the APP1 to 18 rfb gene clusters,
(i) comprising or consisting of SEQ ID NO: 1, SEQ ID NO: 3 or SEQ ID NO: 4;
(ii) having at least 70, 80, 90, 95 or 98% nucleic acid sequence identity to SEQ ID NO: 1, SEQ ID NO: 3 or SEQ ID NO: 4, optionally over the whole sequence;
(iii) hybridizing to the nucleic acid sequence of SEQ ID NO: 1, SEQ ID NO: 3 or SEQ ID NO: 4 under stringent conditions; and/or
(iv) is degenerated with respect to the nucleic acid sequence of any of (i) to (iii).

25. The bacterial host cell according to claim 22, wherein the heterologous rfb gene cluster is selected from the APP1 to 18 rfb gene clusters,
(i) comprising or consisting of SEQ ID NO: 1, SEQ ID NO: 3 or SEQ ID NO: 4;
(ii) having at least 70, 80, 90, 95 or 98% nucleic acid sequence identity to SEQ ID NO: 1, SEQ ID NO: 3 or SEQ ID NO: 4, optionally over the whole sequence;
(iii) hybridizing to the nucleic acid sequence of SEQ ID NO: 1, SEQ ID NO: 3 or SEQ ID NO: 4 under stringent conditions; and/or
(iv) is degenerated with respect to the nucleic acid sequence of any of (i) to (iii).

26. The bacterial host cell according to claim 21, wherein the heterologous functional APP rfb gene cluster produces an O-antigen of APP1 to 18.

27. The bacterial host cell according to claim 21, wherein the endogenous rfb gene cluster of the bacterial host cell is at least partially or completely deleted.

28. The bacterial host cell according to claim 22, wherein the heterologous promoter for regulating the transcription of the heterologous APP rfb gene cluster is a promoter selected from the group consisting of kanamycin promoter, proD promoter, j23101 promoter, proC promoter, STER_RS05525 promoter, STER_RS01225 promoter, STER_RS04515 promoter, STER_RS05020 promoter, STER_RS06870 promoter, STER_RS00780 promoter, and P32 promoter.

29. The bacterial host cell according to claim 22, wherein at least one further gene for functionally expressing an enzyme assisting the APP O-antigen synthesis is selected from the group consisting of the enzymes for nucleotide activated glycan biosynthesis, undecaprenylpyrophosphate glycosyltransferases, O-antigen glycosyltransferases, O-antigen polymerases, O-antigen chain length determinant protein, N-glycan epimerases, and combinations thereof.

30. The bacterial host cell according to claim 22, wherein the at least one neutralizing epitope of Apx toxins:
is at least one neutralizing epitope of Apx toxins I, II and III;
is located on the bacterial host outer cell surface and/or secreted from the cell; and/or
is bound to a membrane protein.

31. The bacterial host cell according to claim 21, wherein
(a) the heterologous functional APP rfb gene cluster,
(b) the at least one further gene for functionally expressing an enzyme assisting the APP O-antigen synthesis, and/or
(c) the at least one neutralizing epitope of Apx toxins, is codon-optimized for the bacterial host cell.

32. The bacterial host cell according to claim 22, wherein
(a) the heterologous functional APP rfb gene cluster,
(b) the at least one further gene for functionally expressing an enzyme assisting the APP O-antigen synthesis, and/or
(c) the at least one neutralizing epitope of Apx toxins is codon-optimized for the bacterial host cell.

33. The bacterial host cell according to claim 31, wherein the heterologous functional APP rfb gene cluster (a) is codon-optimized for the bacterial host cell.

34. The bacterial host cell according to claim 21, wherein the bacterial host is Escherichia coli or Salmonella enterica, wherein
(a) the heterologous functional APP rfb gene cluster is selected from APP1 to 18 rfb gene clusters;
(b) the heterologous promoter for regulating the transcription of the heterologous APP rfb gene cluster is the kanamycin or proD promoter;
(c) the at least one further gene for functionally expressing an enzyme assisting the APP O-antigen synthesis is the wzy gene, and/or the gne gene;
wherein (i) the APP1 to 18 rfb gene cluster, (ii) the gne gene and/or (iii) the wzy gene are codon-optimized for the bacterial host cell Escherichia coli or Salmonella enterica.

35. The bacterial host cell according to claim 34, wherein the bacterial host is Salmonella enterica subsp. enterica serovar Typhimurium, wherein
(a) the codon optimized heterologous functional APP rfb gene cluster is the APP2 rfb gene cluster;
(b) the heterologous promoter for regulating the transcription of the heterologous APP2 rfb gene cluster is the kanamycin promoter; and
(c) the at least one further gene for functionally expressing an enzyme assisting the APP O-antigen synthesis is the gne gene and/or the wzy gene.

36. The bacterial host cell according to claim 34, wherein the bacterial host is E. coli, wherein
(a) the heterologous functional APP rfb gene cluster is the APP2 rfb gene cluster
(b) the heterologous promoter for regulating the transcription of the heterologous APP2 rfb gene cluster is the kanamycin or the proD promoter; and
(c) the at least one further gene for functionally expressing an enzyme assisting the APP O-antigen synthesis is the gne gene.

37. The bacterial host cell according to claim 34, wherein the bacterial host is Salmonella enterica subsp. enterica serovar Typhimurium or Escherichia coli, wherein
(a) the heterologous functional APP rfb gene cluster is the APP8 rfb gene cluster,
(b) the heterologous promoter for regulating the transcription of the heterologous APP2 rfb gene cluster is the kanamycin or proD promoter; and
(c) the at least one further gene for functionally expressing an enzyme of the APP O-antigen synthesis is the wzy and/or gne gene.

38. The bacterial host cell according to claim 34, wherein the bacterial host is E. coli_5, Salmonella enterica subsp. Enterica, Salmonella enterica subsp. enterica serovar Typhimurium, or Salmonella enterica subsp. enterica serovar Typhimurium SL1344.

39. The bacterial host cell according to claim 34, wherein the heterologous functional APP rfb gene cluster is the APP2 or APP8 rfb gene cluster.

40. The bacterial host cell according to claim 34, wherein the wzy gene is a codon optimized wzy gene, and/or both the wzy and the gne genes are integrated into the genome of the bacterial host cell or located on a plasmid.

41. The bacterial host cell according to claim 34, comprising at least one of neutralizing epitopes of Apx toxins I, II and III, at least one of neutralizing epitopes of Apx toxins I, II and III bound to a membrane protein, or at least one of neutralizing epitopes of Apx toxins I, II and III bound to cytolysin A of E. coli, or secreted from the host cell.

42. The bacterial host cell according to claim 34, the APP2 rfb gene cluster and the wzy gene, are codon-optimized for the bacterial host cell Escherichia coli or Salmonella enterica.

43. The bacterial host cell according to claim 34, wherein the bacterial host is Salmonella enterica subsp. enterica serovar Typhimurium, Salmonella enterica subsp. enterica serovar Typhimurium strain SL1344, E. coli or E. coli_5 and at least one of:
the codon optimized heterologous functional APP rfb gene cluster is the APP2 rfb gene cluster, (i) comprising or consisting of SEQ ID NO: 3; (ii) having at least 70, 80, 90, 95 or 98% nucleic acid sequence identity to SEQ ID NO: 1 or SEQ ID NO:
3; (iii) hybridizing to the nucleic acid sequence of SEQ ID NO: 1 or SEQ ID NO: 3 under stringent conditions; and/or (iv) is degenerated with respect to the nucleic acid sequence of any of (i) to (iii), and the endogenous rfb gene cluster of the bacterial host cell is at least partially or completely deleted;
the at least one further gene for functionally expressing an enzyme assisting the APP O-antigen synthesis is the gne gene and/or the wzy gene is integrated into the genome of the bacterial host cell;
i. wherein the gne gene comprises or consists of SEQ ID NO: 6 or has a nucleic acid sequence at least 70, 80, 90, 95 or 98% identical to SEQ ID NO: 6, and/or hybridizes to the nucleic acid sequence of SEQ ID NO: 6 under stringent conditions;
ii. wherein the wzy gene comprises or consists of SEQ ID NO: 7 or SEQ ID NO: 8, or has a nucleic acid sequence at least 70, 80, 90, 95 or 98% identical to SEQ ID NO: 7 or SEQ ID NO: 8, and/or hybridizes to the nucleic acid sequence of SEQ ID NO: 7 or SEQ ID NO: 8 under stringent conditions; and/or
the bacterial host cell comprises at least 2 neutralizing epitopes of Apx toxins I, II and III.

44. The bacterial host cell according to claim 21, wherein the bacterial host is live or inactivated.

45. The bacterial host cell according to claim 22, wherein the at least one neutralizing epitope of Apx toxins is a neutralizing epitope of Apx toxins I, II and III.

46. The bacterial host cell according to claim 22, wherein the at least one neutralizing epitope of Apx toxins is located on the bacterial host outer cell surface and/or is secreted from the cell.

47. The bacterial host cell according to claim 1, wherein (a) is codon-optimized for the bacterial host cell.

48. The bacterial host cell according to claim 22, wherein at least one of (a), (c) and (d) is codon-optimized for the bacterial host cell.

49. The bacterial host cell according to claim 21, wherein the heterologous rfb gene cluster is selected from the APP1 to 18 rfb gene clusters.

50. The bacterial host cell according to claim 21, wherein the heterologous rfb gene cluster is the APP2 or APP8 rfb gene cluster.

51. The bacterial host cell according to claim 21, wherein the heterologous functional APP rfb gene cluster produces an O-antigen of APP1 to 18.

52. The bacterial host cell according to claim 21, wherein the heterologous functional APP rfb gene cluster produces an O-antigen of APP2 or APP8.

53. The bacterial host cell according to claim 21, wherein the APP rfb gene cluster expresses at least one protein comprising or consisting of the amino acids of any one of SEQ ID NOs: 2, 50-61, or SEQ ID NO: 5, 62-72, or the at least one protein having at least 70, 80, 90, 95 or 98% amino acid sequence identity to these sequences.

54. The bacterial host cell according to claim 22, wherein the at least one further gene for functionally expressing an enzyme assisting the APP O-antigen synthesis is selected from the group consisting of the gne gene and the wzy gene.

55. The bacterial host cell according to claim 54, wherein
i) the gne gene encodes an UDP-galactose/UDP-N-actetylgalacosamine epimerase, and/or
ii) the wzy gene encodes an O-antigen polymerase of APP,

56. The bacterial host cell according to claim 54, wherein
i) the gne gene encodes an epimerase from Campylobacter jejuni, and/or
ii) the wzy gene encodes an O-antigen polymerase of APP2.

57. The bacterial host cell according to claim 54, wherein
i) the gne gene encodes an epimerase from Campylobacter jejuni; and/or
ii) the wzy gene comprises or consists of SEQ ID NO: 7 or the codon optimized wzy of SEQ ID NO:8 or having a nucleic acid sequence at least 70, 80, 90, 95 or 98% identical to SEQ ID NO: 7 or SEQ ID NO:8, and/or hybridizing to the nucleic acid sequence of SEQ ID NO: 7 or SEQ ID NO:8 under stringent conditions.

58. The bacterial host cell according to claim 54, wherein the gne gene comprises or consists of SEQ ID NO: 6, or having a nucleic acid sequence at least 70, 80, 90, 95 or 98% identical to SEQ ID NO: 6, and/or hybridizing to the nucleic acid sequence of SEQ ID NO: 6 under stringent conditions.

59. The bacterial host cell according to claim 22, wherein the at least one neutralizing epitope of Apx toxins is/are located on the bacterial host outer cell surface and bound to a membrane protein selected from the group consisting of cytolysin A, trimeric autotransporter adhesion, AIDA-I, EaeA , outer membrane proteins (OMP), and OmpA of E. coli.

60. A pharmaceutical composition comprising at least one bacterial host cell according to claim 1.

61. The pharmaceutical composition of claim 60, comprising bacterial host cells expressing at least two different O-Antigens from APP.

62. A method of treatment comprising the step of administering a physiologically effective amount of a bacterial host cell according to claim 1 to a mammalian subject in need thereof for the treatment and/or prophylaxis of APP infections.


SEQUENCE LISTING (72 sequences)

SEQ ID NO: 1 - DNA, 12928 bp, Artificial Sequence
  Identified APP2 O-antigen biosynthesis cluster of sequenced APP2 strain
atgcttgaacaagcatcaaaccaaactaacgaggaaatcgatctgattgaattaattcgcgtgctttggaagaaaaaattattaattgctattgttacctttatttttactgcattggcagcagtttatgcttttaccgcgaaagagaaatggacatctcaagcagaggtaattgcgcctagagtgacagatatttccgaatatttatcgttacgaaaagaatataatttaattatgggttctgagttcaaagaaaatgatattcgtaatgaactaaatgagcttttttctcgttatgtgctttcttatgacgaagcaatctctttctttaagaccacagatacttataaaaaacttgcagaaaaagaaaatgaagcaggtttacaaagagcagcttctgaatttacaacggaatcattgaaggtgataaaaccggatgcgaaaaaagaccttaatgctttaggcagtaagattgctatttcatctgagactgctttatctgcacaaacagagttaaatgattttattcgtcatattagtgatatttcttttaattttagtaaaaatgaatttatttattgggtaaaagagagcatatctagcctaaattacgagaaagaagtgatagagcaagatcagagtattcaacgaaaagttcagattcaaaacttagaagccgcacttgatatggcgaaaaaagcaggaattaaagagtatagttctgcattgtcatcaaatagttctgtggctaatctcgcggtaagtgatacaaaaattccgttatcagattctaaattagcagatggaacctatttatttatgcttggtgagaaaaatctacaagcacaattagatattgcgaaaaccaaggaaattgtttattcgccaagatattatcagattcaagagcaacttttaaaattaaatactttattacctaaagtagagaaagtaactgggcaaacttatagttatgtatcttcgccgacatatcctgttatcaaagatgcacctaaaaaaggaattattttagttataggtttcttggtaggattgttactgagttcattcactgttttgatttctgtattagtgagaaataagaagagctaattatgaggtatgaagaggaatattaacctctttatgtgccttttgatgatccaatttactgaagtgcttagactatttaggtaataaaatatagggataataatgaagcataagatattacattttagccaagtgcttggcggtgtcggtcgatatttagagctatatgataagtacatcaataaagattcatttgaaaatatatatatattacctataggtgattgggaggcggcagaagctcaggataaacggtatatattaaatattgaacaatccttttcaccaattaaattgatctctaatgttataaaaattagaaatatcttaaaaaaagaaaagccagatatcttttatctacatagtacttttgctggcgttattgggcggttagctgctattggtatgaggtgtaaagtaatttataaccctcatggttggtcatttaagatgaatgtatctaggctaaaacaaactttttataagattatcgaaggtggtttagttttcttaactgataagtttgttttaatttcgaaatcggagtatgaggcggcacgttcaattggtgtttcagagaagaaatgttgtctcatatataacggtattgaaacgataaaaaaaacagatatagcaatcattcctaaattagatgataaatatatcattggaatgataggacgtattagtgagcaaaaaaatccaatgttttttgcccagtttgccaaagagattattaaacaataccctaatacttatttcattttggttggtgacggtgagcaacgagaatcgttagaagactatttagaacgtaataacttgaatgatgttttttatattacgggttgggtaactaatcccgaaagctacttaaatttatttgatcaagcagtattattctcaaaatgggaagggctatgtctttcagtctgtgagtatatgttatatgaaaagcctatattagtaagtaatattggtggtattaacgatcttattcagaatgaagttaatggttttactattgttgaaggtgatcttaaggatgcggttaacaaatctaatagattaagaaatgagcctaaaactgtagctaagtttattgaagcctcaaacatacttattcaagagaaatttaatgctcaaaaaatggtaaatagtttagaaaaactttttatcaaattatcagagaataaataatgaaagaaaaatttagttgcattgttgtttgttataaccccgataattcggtgcttgacaatctcaaaaactatattagttatgtgggaaaagtaatcgtagttgataattcagatgtggataattctcaattattttcctcactttcagaatacttaatttatataccattgtataaaaatgtgggtattgcctatgcactaaatataggagtagaaaagtccaaagaattaggatatgaatatatcattactatggatcaagatagttcttttgctactaatctagtggatgtatattcacattatataagtaattatcctatagatcagataggagcattatccccagtttatattacggacaggggatttaatcgaacaagtaaagaagaatttaaacaaataaaaattactatgcaatcaggttctatgttctttactgataaatttgatgtaatcggtcgctttgataatgatctgttcttagatgtagttgattgggaatatttctttagaatttatacgttaggatataaaacgattcaatgtaataaagcaatgctgaaacatgctccagcggaaacgctaacgttatttaaaataaaaggaaaaacaattggtgttggagtcgcttctccattaaggtattactatcaaattagaaatctactttggtgtgttttacataaaaagagtttttttatgataaaaacaatagcttataaatttattaagattctatttttgtttaataataaaaagcaatatttatcatttgcttatatggctattaaagacgcctttaataatcgtttaggggcatatgatacactttatttagagaaatctcgtaatgaaaaatgatttaccattaattagtattattattcctatctataacgtgaagccttatcttgaaaaatgtgtaaatagtgtattatcacaatcatatcctaatcttgaaattattctagttgatgacggtgcaactgatggttctgctcaagtgtgtgatgatttttctgaaaagtatgcaaatattcaggtaattcataagaaaaatggtgggctatcctcagcaagaaatgctggaattgaagctatgaagggagaatacgtatttttcttagatagtgatgactggattgctaatgatgcaatttctcaattatatgatgatatggtggaatataatgcagatataacagggattagtttttatcaagcatattcagacggtaatttagtattaaatacacatcttattgaaaaacaaatgctttcaaagaaagaggctttacgtactttcctatttaataattaccttactccttgttcctgtggaaaactttataaagcaagtctatggaaagatataagatttccggagggacgattatttgaagatcagcttactacttataaagttatcgagttagcaaatacaattatttttaatcctgctgcaaagtatttttattttaaaagaataggatctatcggtcattctgctttttctgaaaaaacatatgacctttatgaggctgttaatgaacaatataatgaaataactaagcatcatcctgatattgaatctgatttggcggttgctaaaattacttgggaaattgtatttattaatatgatgctcaattcaaattattcagatcaagcgatagttgataaaacacgagtttttgcaagaaaacgtattttagatgtagtgaaatgtgagtttatccctaatttacgaaaatttcagattactttatttgcatataattttagtttatataaagttttatatgcaagatataaaaagaaaaatccattatcttaattattgattttatcggttgttacaatgatacctaaaaaaattcattattgttggtttggtggaaatccattacctaaaagtgtgaaaaaatgtattaaatcttggaaaaaatactgtccagattacgagattattgagtggaatgaatctaattacaatgtgcataagaacctttttataaaagaagcttatgagaaaaaaaagtttgcatttgtttcagattatgctcgtttagatgtggttcattctgaaggtgggatttatttagatactgatgttgagttgataaaacctatagatgatttattagctcatagttgttttttagcatctgaatctattgatgatgttaatacagggctaggttttggggctgaaaaaggacattggtttatcgcagaaaatatgagtgtctatgaaaatatgtactttaatatggaaaatattattacctgtgtagagattactactaaattattaatagaaagaggtttttctgctagtgataaaattcaaaatatagatgatattttcatttatccaactgagtatttttgcccattaaattataaaacccacgagttgcatataacacagaatacttattctatacatcactatgatgcaacttggcaaagccctcttatgaaatttaaaacaaaaattaagtatatattgtgtttagccggaataataaaatgaactccttagtatatagaatagatattagaacacttattttttctattttttattttacttttttagtatcggattttttattattagctcaagatggcactattacaaaagatatcatcaaatgggttaaattattctcattattgccattgctcttattaatatttaaattgcctttgaatctcttgattttaggtttttttactataatgataagtgctttttattctatttatacgggagattcgtttttattatatatatgtttgctgatgtctttttcttataaagttaattttaactttttattcaagataggattatatcttacttcaattctagttgttctaatactaacttatttcttttttgaatattttctgattggtgacagtcattttgtatatgatgcgacctattggtttaaacgttatacatttaattttgataatcctaatgcatttcctatgagaatattcgttttttttatattttatatattgcatgtaggtaagctgcgactttttgatacatttctatttgttatactatttggaatagttttctatttttcaaattctagaactgcattttatatttttattttgtgtgtccttactattcattttaaccaagtttttaatgtgctaaataatacttttgttaaattactaattaataattcaattatatttataactattttttcaatttggtcggctatatattatcaagattattattcctatttagaaccgattaacaaaattttatctaaaagaatatactttgctaatgaggcttataagagtttaggatttgaattttaccctaggaatattaaatggtggatagaagaatctgattggcatattatagataatggatatgtatatttatttatttctggtggtcttttagtaggaaatttatttatattttctataacttggcttatgtatagactaaataaatttaacctaagtaatgaggcaatattattaatgttttctatgttatatcttttatctgagagtcattttataaatatattttacaatatacctattttattattagctattttcattaataaaactaatattgtacgctatttggaatgtaaaaaatgaataaaaaccttgtaaataatagtattatgagttttttgcttacaatatctaactttatttttccattaattacttttacttatgcggcaagaattttgcaacctgataatatgggaaagtttgcattttctctatcggttgtagattatctatctctatttgctacatttggtgttgtaggttatggtgttagagcttgtgcagaagtaagaaacaataaagaagaactaactaaaacggtacaagaaattttatttattaatatttttttagctattattgcctatcttgtgatatttcttctaattagctatcagcatgcatttagagaagatactttgttattcttaattatgtcttcttgtattatctttaatgtgataggaatagaatggttatataaaagtctcgatgaatatagatacattacagtaagaagtattctattaaaaataatttcattaataatgattttatgttttgttaaagaaaaggatgattatccactttttgcattgttttttgttctaccaatttgtctatcttcgttgttaaatattataaattcaagaaaaatattgctttttaaattatttaaacttgatttatcaaagcatataaaaccaatgtttgttttatttttagtgacattatcttatacattatatgctaatgttaatgatgtgctattagctactgtaactaatacagaacaagttggttactatagtgttgctttcaaaataaaagctgcattattagctttcattactagtacaagtatggtttttttacctcgattaacagagtatattaaaaataatcaagatattgaatttattgacttattaagaaagtcttttgatctggttttttttctagctgtgccaataacattatttttctttttatacgctaaagaaacaatatttttattgtttggtgagaaatataataagtcaagtttattattgcaaaccatgatatggtctgttttttttggtggtttaaataatatattaagtgtacaaatgttattgcctttaaaaaaagataatcagttcttaatttctattttaagtggtggatgtatatctttagttgtgaattttatcttcttgagggagcttcaatcattaagtacatcaatttcagttctagttgcagaagttgttatactgattattcaattagttattctaagaaaatatattgtaagaatttttaataatttaaatcctttaaaggtgataatgtcggtttttttttctatatggtttgttaatttaatttatgccaattttattgctctaggtaatagtttcttagagtatattatttctatttttatattttcattattttatgtgtttttacttttttttagtaaagaaagatttgttcatgatgtgtttttttatataaggagtaaatttgattaatttattaattagtattctagctaaaattctttctaggatttctaaactgattttgaatataaaaaaacggaaggaatacaaacgagttggctctatagttgattcaaagaatatagatttgagttttatttgtggtaactattgtagagtagggagagatactgtaattgagaaaaatgttattatggggagattatcttacattaattcagatatgggaaaaacatatattggtagtaatgtaaagattggtagtttatgctcaatttcctcaggtgtaataattgctcctgtaaatcattacctaaattatgtgacaacgcacccattactttataattcctattatagtagcattttaaatattaattctaatctgttatctcaacaagaattagatgcaaatgtatcaacagtgattggtaatgatgtatggattggagctaatgtgattataaagagaggagtaactataggagatggagcggttattggtgcaggtagtattataacaaaagatattccttcttatgcagtagtagcaggagttccagctaaaattattaaatatcgtttttcaaaagatgtaatagaaagcctgaaagatagtaagaatgtttgggaattatctacctcagaattagaagagaatttttctcatttatatgatgttgagaaatatcttaatagatttaagttgtaggattaatttttagtctaggattttagtatgagtaagaaaaatatagttgcacaaactttattactttgcttagatttattactaattagtatggcaatctttttagctgtatttattagaaataatattttaccgaatattatgttatttgagcctgtatcatatatagagtatctagtatacccatttccttatgtaatcattgttacattgtttatgtggtttgggctatatacaagaagatatgatttatggcaggagtcattatttattataaaagtatgttttatttcttttattattatctttgcaacattagcattgggtaagaatatagaatattattctagagctgttttattattatctcttttcttatcagtgatatttttaccaataggtcgttattttttgaaaaaaagcttgtttagactgggtctttgggaaaggaaagtaaagtttattggcaatttaaataagaatgaaattgggatttttaattctcctcatgtaggatatgtgttatctaaagatgatacatatgatgttatatttatatctagtggtgataagagtgtatcagaattaaatgatttaattgaaagtaataaattattgaatcgtgaggttctatttatccctgtgttaaatcaatatgattttactcaatctgttttgtacaataattttagtacaaggctaaatctatttacgttagaaaataaattacttggaaagcaaaataaaattttgaagtatttactagattatgtactagtattatctactttacctttttggggggggctgattttacttattagtataaaattaaaattagaagatcctaaagggaaaatatttttcttacaaaagagattaggtcaagagggtaagatattctattgttataaatttagaacaatggtttcagaccagagctttatgcaacaatggcttattgataatccagaagaaagagattattacgctgtgtatcataagtatattaatgatcctagaattactaaattcggacattttttgcgaagaacatctttagatgagttaccccaattatttaatgtacttaaaggggatatgagtttagttggaaatagaccttatatggttgaggaacaacaaaaaatgaaagatgctgccagtattattttgatgtcaaaaccaggagtaacaggtttatggcaagtaagtgggcggagtgacgtttcatttgaagaacgtttacaaattgattcttggtatattaaaaattggtctatttggaatgatattgttattttattcaaaacagttggtgttgtattaagaaaagatggagcatcttagtaataatgtaattacattaaattattatagatagggattattatgaaaaaaattttagtcaccggtggtgcaggttttattggctctgcggttgtacgtcatattataaatgatacacaagatagtgttgtaaatgttgataaacttacctatgcgggtaatttagaatcgttattaatggtagaaaatagccctcgttacgtatttgagcaagtagatatttgtaatcgtgcggaacttgatcgcgtatttgcccaacatcagcctgatgcagttatgcacttagccgcagaaagccatgttgaccgttcaatcgatgggccggctgcttttatcgaaacaaatattgtcggtacttacactttgctcgaagctgctcgctattattggaatagtttagatgctgataaaaaatcattattccgttttcatcatatttctacggatgaggtatatggtgatttggaaggtacagaagatttgtttacggaaacgacgccgtattctccgtctagcccatattcggcttctaaagcgtcaagtgatcatttagtccgtgcttggcttcgtacttatggattacctacgattgtgaccaattgttcgaataactatggtccgttccattttcctgaaaaattaattcctttaatgattttaaatgctttagagggtaaaccattaccagtttatggtaatgggcaacaaatccgtgactggttatttgtagaagatcatgctagagcattatacaaagtggtaacggaaggtaaggtgggagaaacttataatataggtggacataatgaaaaagctaatattgatgttgttcgtactatttgtagtttattagaagagcttgtaccaaataaaccggcgggtgtgcataaatatgaggatttaattacctacgttacagatcgtccagggcatgatgttcgttatgcaattgatgcaacaaaaattggacgagaattaggttggaagccacaagaaacatttgaaacaggtattcgtaaaacagtcgaatggtatttaaataatacagagtggtggagtcgtgtattagacggttcttacaatcgtgagcgtttaggttcaaattaatattattacaagcgatccaatttttaataaggtttacaatatgaaaggtattattcttgcaggtggctcaggtactcgtctttacccgattactcgtggcgtgtcaaaacagctcttaccggtatacgataaaccaatgatttattatcctttatcagtacttatgcttgcaggtatccgagaagtcttaattattacaacaccggaggataatgagagctttaaacgtttattaggcgacggttctgatttcggtatccaactttcctatgctattcaacctagtccagatggcttagctcaagcatttttaattggtgaagagtttatcggtcaggacagtgtatgtttggttctaggtgataatatcttctacggtcagcattttactcaatctttacaagaggctgtaaaatcggtagaaacgaaaggtgcgactgtatttggttatcaagtgaaagatccggaacgttttggtgtggtagagtttgatgacaatttccgtgcattgtctattgaggaaaaaccgattcaacccaaatctaattgggcggtaaccgggttatatttctatgataaccgagtagtagaatttgcaaaacaagtaaaaccctctgcacgtggcgaattagagattaccactcttaatgagatgtatcttaatgatggttcacttaatgtacaattattagggcgaggctttgcttggttagataccggcacacatgatagcttacatgatgcggcagcatttgtgaaaacagtacaaaatctacagaatttacaggtagcatgcttagaggaaattgcctatcgtaacggttggttatcacttgagcaacttgaagcattaacaaaaccgatggcgaaaaatgaatacggtcaatatttgttacgtttaacaaaaggaacaaaataatggcacgtttcttaattacgggagcgaagggacaggttggatattgtcttactaagcaattacagagcaaagcagatgtcttagcagtagatcgtgatgagcttgatataacaaatcgtgatgctgtatttaaagttgtcagagagtttcatcctgatgttattattaatgctgccgcacatactgctgtagatcgggctgagagtgaaatcgaactatcggaagcgattaacgtgaaaggcccacaatatcttgcagaagcagccaatgagattgatgcaatcattttacatatttcaacggattatgtctttgaagggacaggttctggagaatataaagaaaatgatgaacctaatccacaaggcgtatacggcaaaacaaaacttgccggagagatagcagttcaacaggcaaataaaaggcatatcattttgcgtactgcttgggtatttggtgaacatggtaataactttgttaaaacgatgctccgtttagcaaaagaaagagaatctttgggaattgtgagtgatcaatttggcggacctacctatgcaggggatattgcgagtagcctgattcatatagcaaatatcattcttaatagtaagatagatgtatttggtgtttaccattttactggcaagccttatgtaagttgggccgattttgctaagaaaatttttgatgaagctgtttcgcaaaaggtattagaaaaagcaccgcttgttaattttattgctacaagtaattatccaacatcagcaaaacgaccggcaaattctcgcttagatttaactaaaattgatgaggtttttggtattaaaccgagtaattggcaacaagcattaaaaaatattaaggcatatgcgtaatgaagattattgaaacaaatattccggatgtaaagcttttagaacctcaagtatttggtgatgaacgcggtttttttatggaaatttttcgagatgaatggttcagacaatatgtcgctgatcgtactttcgttcaagaaaatcattcaaaatctattaagggagttttgagaggcttacattatcaaactgaaaatacacaaggcaagttagtgcgtgtagtgcaggggtctgtgtttgatgtagcggtagatttacgtaaaagttctccgacttttggacaatgggttggggaagtattatccgctgaaaataaacgtcaactttgggtccctgaaggatttgctcacggtttttatgtattgacagaaaccgctgaatttacctataaatgcacagattactataatccaaaagcggaacattcattgatttggaatgatccgacagtagcgattaattggaatcttggtggcgcgcctagtttatcagcaaaggatttagctggtaaggtgttaaatgaagctgttttatttgaatag

SEQ ID NO: 2 - protein, 370 aa, Artificial Sequence
  amino acid sequence of rfb (APP2) cluster gene wzzB
MLEQASNQTNEEIDLIELIRVLWKKKLLIAIVTFIFTALAAVYAFTAKEKWTSQAEVIAPRVTDISEYLSLRKEYNLIMGSEFKENDIRNELNELFSRYVLSYDEAISFFKTTDTYKKLAEKENEAGLQRAASEFTTESLKVIKPDAKKDLNALGSKIAISSETALSAQTELNDFIRHISDISFNFSKNEFIYWVKESISSLNYEKEVIEQDQSIQRKVQIQNLEAALDMAKKAGIKEYSSALSSNSSVANLAVSDTKIPLSDSKLADGTYLFMLGEKNLQAQLDIAKTKEIVYSPRYYQIQEQLLKLNTLLPKVEKVTGQTYSYVSSPTYPVIKDAPKKGIILVIGFLVGLLLSSFTVLISVLVRNKKS

SEQ ID NO: 3 - DNA, 12928 bp, Artificial Sequence
  nucleotide sequence of codon optimized rfb cluster
atgctggaacaagctagcaatcaaaccaacgaagaaattgatctgattgagctgattcgcgtgctgtggaagaagaagctgctgattgccatcgttaccttcatctttacagctttagccgcagtttatgcctttacagcaaaagagaaatggaccagtcaagctgaagtgattgccccgcgcgtgaccgacattagtgaatatttatctttacgtaaagaatacaatctgattatgggcagtgaatttaaagaaaatgatattcgcaatgaattaaacgaattattcagccgctacgtgctgagctatgatgaagccatctccttttttaaaaccaccgatacctacaagaagctggccgagaaagaaaacgaggctggtctgcagcgcgcagccagtgaattcaccaccgaatctttaaaggtgattaagccggacgccaagaaagatttaaatgctttaggcagcaagatcgccattagcagcgaaaccgctttaagtgcccagaccgagctgaacgactttattcgccacatcagcgatattagcttcaattttagcaaaaatgaatttatttattgggtgaaggagagcatcagctctttaaactatgaaaaagaagtgatcgaacaagatcagagcatccagcgcaaagttcagatccagaatctggaggccgcactggacatggccaaaaaggccggcatcaaagagtacagcagcgcactgagcagcaacagcagcgtggcaaatttagcagtgagcgacaccaagattccgctgagcgacagtaaactggcagatggcacctatttattcatgctgggcgagaagaatttacaagcccaactggatatcgccaagaccaaagagatcgtgtacagcccgcgctattaccagatccaagaacagctgctgaagctgaatactttactgccgaaagttgagaaggtgaccggccagacatatagttacgtgagcagcccgacctatccggtgatcaaagacgccccgaagaaaggcatcattctggttatcggctttttagtgggtttactgctgagcagctttaccgtgctgatcagcgtgctggtgcgcaacaaaaagagttaattatgaggtatgaagaggaatattaacctctttatgtgccttttgatgatccaatttactgaagtgcttagactatttaggtaataaaatatagggataataatgaaacataagattttacattttagtcaagttctgggcggtgtgggccgctatctggaactgtatgacaaatacatcaacaaagatagctttgaaaacatttatattttaccgattggtgattgggaagccgcagaagcccaagataaacgttacattctgaatattgaacagagctttagcccgattaaactgattagtaatgttattaagattcgtaacattctgaaaaaagaaaaaccggatatcttttatttacacagcaccttcgccggtgttattggccgtttagcagccattggcatgcgctgcaaagtgatctacaacccgcatggctggagcttcaagatgaatgtgagccgtttaaagcagaccttctataagatcattgagggcggtttagtgtttttaacagataaattcgtgctgatcagcaagagcgaatacgaagcagcccgcagcatcggcgttagcgagaaaaaatgctgtttaatttacaatggcattgaaaccattaaaaagaccgatattgcaattattccgaagctggatgataaatatatcattggcatgattggccgcatcagcgagcagaaaaacccgatgtttttcgcccagttcgccaaggagatcattaagcagtacccgaacacctactttattttagttggcgatggtgagcagcgcgagagtctggaggattatttagaacgcaataacttaaacgacgtgttttatatcaccggctgggtgaccaacccggagagctatctgaatctgttcgaccaagctgttctgttcagtaaatgggaaggtttatgtttaagcgtgtgcgagtatatgctgtacgagaagccgattttagtgagcaacattggtggcatcaacgatttaatccagaacgaggttaacggcttcaccatcgttgagggcgatttaaaggatgccgtgaacaagagtaaccgtttacgcaatgaaccgaaaaccgtggccaagttcatcgaagccagcaacattttaattcaagaaaagttcaacgcacagaaaatggtgaatagcttagaaaaactgttcatcaaactgagcgaaaataaataatgaaggaaaaatttagttgcatcgtggtgtgttacaacccggacaatagcgtgctggataatttaaaaaactatattagttatgtgggtaaagtgattgtggtggataacagtgatgtggacaacagccagctgttcagctctttaagcgagtatctgatctacatcccgctgtacaaaaacgtgggcatcgcctatgctttaaacatcggtgtggagaagagcaaggaactgggttatgagtatattattaccatggaccaagatagcagcttcgccacaaatctggtggatgtgtacagccattacatcagcaactacccgatcgatcagattggcgctttaagcccggtgtatattaccgaccgcggtttcaaccgtaccagcaaagaagaatttaaacagattaagatcaccatgcagagcggcagcatgttcttcaccgacaaattcgatgtgatcggccgctttgacaacgatttatttttagacgtggtggactgggaatactttttccgcatttatactttaggttataaaacaattcagtgcaataaagccatgctgaaacacgccccggccgaaactttaactttatttaaaattaaaggtaaaaccattggtgtgggcgtggcaagcccgctgcgctattactatcagattcgcaatctgctgtggtgcgtgctgcacaagaaaagcttcttcatgattaagaccattgcctataagtttatcaagattctgtttctgtttaataataaaaaacagtatttaagcttcgcatacatggccatcaaggacgccttcaataaccgtttaggcgcctatgatacactgtatctggagaaaagccgtaatgaaaaatgatctgccgctgatcagcatcatcatcccgatctataacgtgaaaccgtatttagaaaagtgcgtgaacagcgtgctgagccagagctatccgaatctggagattattctggtggatgacggtgccaccgatggcagcgcacaagtttgcgatgattttagcgaaaaatatgcaaatattcaagttattcataagaaaaatggtggtttaagcagcgcacgtaatgccggtattgaggccatgaaaggcgagtacgtgttctttctggatagcgacgactggatcgcaaatgacgccatcagccagctgtacgatgatatggtggagtacaacgccgacatcaccggcatcagcttttaccaagcttatagcgacggtaatttagtgctgaacacccatctgatcgagaagcagatgctgagcaagaaagaggcactgcgtacctttttattcaataattatttaaccccgtgtagctgcggcaagctgtataaagcctctttatggaaggacatccgctttccggaaggtcgtttatttgaagatcagctgaccacctataaagttatcgaactggccaacaccatcatcttcaatccggccgccaaatacttttatttcaaacgtatcggcagcatcggccacagcgccttcagcgagaaaacctatgatttatatgaggcagtgaatgaacagtacaacgagatcaccaaacaccacccggatatcgagagtgatctggccgtggccaaaattacttgggaaattgtgtttattaatatgatgctgaacagtaactacagcgatcaagctatcgtggacaaaacccgcgtgtttgcacgcaaacgtattttagatgtggtgaagtgcgagttcatcccgaatttacgcaagtttcagatcactttatttgcctacaatttcagtctgtataaagttctgtatgcccgctataagaagaaaaatccgctgagttaattattgattttatcggttgttacaatgattccgaagaaaattcattattgctggttcggcggcaatccgctgccgaaaagtgtgaagaagtgcattaaaagttggaaaaaatattgtccggattatgaaattattgaatggaatgagagcaattacaatgtgcataaaaatttatttattaaagaggcctacgagaagaagaagttcgccttcgtgagcgattacgcccgtttagatgtggtgcacagtgaaggtggcatctatctggacaccgatgtggagctgatcaaaccgatcgatgatttactggcccatagctgctttctggccagcgaaagcatcgatgacgtgaataccggtttaggctttggtgccgaaaaaggccactggttcatcgccgagaacatgagcgtgtatgaaaatatgtactttaatatggaaaatattatcacttgtgtggagatcaccaccaaactgctgatcgaacgcggctttagcgccagcgataaaattcagaatattgatgatatttttatttatccgaccgaatatttttgcccgctgaactacaaaacccacgaactgcacatcacccagaacacctacagcatccaccactatgatgccacttggcagagcccgctgatgaaattcaaaaccaagatcaagtacattctgtgtttagccggcattattaaatgaattctttagtgtatcgcattgacatccgcactttaatttttagcatcttttattttacctttctggtgagcgattttttactgctggcccaagatggcacaatcaccaaggacatcatcaagtgggtgaagctgttttctttactgccgctgctgctgctgatcttcaagctgccgctgaatttactgattctgggcttttttaccattatgattagtgccttctacagcatctataccggtgacagctttttactgtacatctgtttactgatgagctttagctacaaagttaattttaattttttatttaagattggtttatatctgaccagtattttagtggtgctgattctgacatattttttctttgagtactttctgatcggcgacagccactttgtgtacgacgccacctactggttcaaacgctacacctttaactttgataacccgaacgcatttccgatgcgcatctttgtcttttttattttttacattctgcatgtgggcaaactgcgtttattcgacacctttctgttcgttattctgtttggtatcgttttttactttagcaacagccgtacagccttttacatttttattctgtgtgttctgaccattcattttaatcaagtgtttaatgttctgaataatacctttgttaaactgctgattaacaatagcattatctttatcaccatttttagcatttggagcgcaatctattatcaagattactattcttatctggaaccgattaataaaattttaagcaaacgtatttattttgcaaacgaagcctataagtctttaggcttcgagttctacccgcgcaatatcaagtggtggatcgaggagagcgactggcatattatcgacaatggctatgtttatttatttatcagcggcggtttactggtgggcaacttatttatcttttctattacttggctgatgtatcgtctgaataaatttaatttaagcaacgaggccattttactgatgtttagcatgctgtatttactgagcgagagccattttatcaatattttttataacatcccgattttactgctggcaatttttattaataaaaccaatattgtgcgttatttagagtgtaaaaaatgaataagaatctggtgaataatagcattatgagctttttactgaccatcagcaacttcatcttcccgctgatcaccttcacctacgccgcacgtattctgcagccggacaacatgggtaagttcgcctttagcttaagcgtggttgattatttatctttatttgccacctttggtgtggtgggttatggcgtgcgtgcttgtgccgaagttcgcaataataaggaagaactgaccaaaacagtgcaagaaattctgtttattaatatctttttagcaattattgcatatctggttatttttctgctgatcagttatcagcatgcctttcgcgaggacacactgctgtttctgatcatgagtagctgcatcattttcaacgttatcggcattgagtggctgtataaatctttagatgagtaccgctacatcaccgtgcgcagcattttactgaagattattagcttaatcatgattctgtgctttgtgaaggagaaagacgattatccgctgttcgctttattctttgtgctgccgatctgtttaagcagcttactgaacattattaacagccgcaaaattttactgtttaaactgtttaagctggacttaagcaaacatattaaaccgatgtttgtgctgtttctggttactttaagttacactttatacgccaacgtgaatgatgtgctgctggccacagtgaccaacaccgagcaagttggctactatagtgtggcatttaaaatcaaggccgctttactggcatttatcaccagcaccagcatggtgtttctgccgcgcttaaccgagtatatcaaaaacaatcaagatatcgaatttattgatctgttacgtaaaagctttgatctggtgttctttttagccgtgcctattactttattcttttttctgtatgccaaagagaccatctttttactgtttggtgaaaaatataacaagagctctttactgctgcagaccatgatctggagcgttttcttcggcggtttaaataacattttaagcgttcagatgctgctgccgctgaaaaaggacaatcagtttttaatcagtattttaagcggcggctgcatttctttagtggtgaatttcatctttttacgcgaattacagagtctgagtaccagtatcagcgttctggtggccgaagtggtgattttaatcatccagctggtgattttacgcaagtacatcgttcgtatcttcaataatttaaatccgctgaaagttattatgagcgtcttttttagcatttggtttgtgaatttaatctatgccaactttatcgctttaggcaacagctttctggagtatattattagtatttttatcttctctttattctatgtgtttctgctgtttttcagcaaagaacgctttgtgcatgatgtgttcttttatattcgcagcaaatttgattaatctgctgatcagcattctggccaaaattttaagccgcattagcaaactgattttaaatatcaagaagcgtaaagagtacaagcgcgtgggcagtattgtggatagcaaaaatattgatttaagctttatctgcggcaattattgccgcgtgggccgtgataccgtgattgagaagaacgtgatcatgggccgtttaagctacatcaacagcgacatgggcaagacctatatcggcagcaatgtgaagatcggctctttatgtagcatcagcagcggcgtgatcattgccccggtgaaccactatttaaactatgtgaccacccacccgctgctgtataacagctactacagcagcattctgaacatcaacagcaatttactgagccagcaagaactggacgcaaacgtgagcaccgtgattggcaatgatgtgtggatcggtgccaacgtgatcatcaaacgcggcgtgaccattggcgatggtgcagtgatcggcgctggtagcattatcaccaaagacatcccgagttacgcagtggttgccggcgtgccggccaaaatcatcaaatatcgctttagcaaagatgtgatcgagtctttaaaggacagcaagaatgtgtgggaactgagcaccagcgaactggaggaaaactttagccatttatacgacgtggaaaaatatctgaaccgttttaaactgtaagattaatttttagtctaggattttagtatgagtaaaaagaacatcgtggcacagactttactgctgtgtctggatttactgctgatcagcatggccatctttctggcagtgtttattcgtaataatattttaccgaacatcatgctgttcgagccggtgagctatatcgagtatttagtttatcctttcccgtatgttattattgtgactttattcatgtggtttggtttatacacacgtcgctacgatctgtggcaagaatctttatttatcatcaaagtgtgctttattagttttattatcatttttgcaactttagccttaggtaaaaatattgaatactacagccgtgccgtgctgctgctgtctttatttttaagcgtgatttttctgccgatcggccgttattttctgaagaaatctctgtttcgtttaggtttatgggaacgcaaagtgaaattcattggcaatctgaataaaaacgaaattggcatcttcaacagcccgcacgtgggttacgtgctgagcaaggacgacacctacgacgtgatcttcatcagcagcggcgataagagcgttagcgaactgaacgatttaatcgagagcaataaactgctgaaccgcgaggttctgttcatcccggttctgaaccagtatgacttcacccagagtgttctgtacaataatttcagcacccgtttaaatttatttacactggagaacaaattactgggcaaacagaataaaattttaaagtatttactggactacgttctggtgctgagtacactgccgttctggggcggtctgattttactgattagcattaagctgaaactggaagatccgaaaggcaaaattttctttttacagaagcgtctgggccaagaaggcaaaatcttctattgttataagtttcgtaccatggtgagcgaccaaagcttcatgcagcagtggctgatcgataacccggaggaacgcgactactatgccgtgtaccataagtatattaatgacccgcgcatcacaaaattcggccattttctgcgccgtaccagtctggatgaactgccgcagctgtttaatgtgctgaagggcgatatgtctttagttggcaatcgcccgtatatggttgaagagcagcagaagatgaaggacgccgccagcatcattctgatgagtaaaccgggcgttaccggtttatggcaagttagtggtcgtagcgatgtgagctttgaagagcgtttacagattgacagctggtatatcaaaaattggagcatttggaacgatattgttattctgttcaagaccgtgggcgtggtgctgcgtaaagatggcgccagttaataataatgtaattacattaaattattatagatagggattattatgaagaaaattctggttactggtggcgctggttttattggcagtgccgtggttcgccatatcatcaacgatacccaagatagcgtggtgaacgtggataaactgacatacgccggcaatctggagtctttactgatggtggaaaatagcccgcgctacgtgttcgaacaagttgacatttgcaatcgcgccgaactggaccgtgtttttgcccagcatcagccggatgccgttatgcatctggccgcagaaagccacgttgatcgcagcatcgatggcccggccgccttcatcgagaccaatatcgtgggcacatatactttactggaggccgcccgctattattggaatagtctggacgccgacaaaaagtctttatttcgcttccaccacattagcaccgatgaggtgtatggcgatttagaaggcaccgaggatttatttaccgaaaccaccccgtatagcccgagcagcccgtacagcgcaagcaaagcaagcagcgatcatctggtgcgcgcttggctgcgcacatatggtttaccgaccatcgtgaccaactgcagcaacaactacggcccgttccattttccggagaaactgatcccgctgatgattttaaatgctttagaaggtaaaccgctgccggtgtatggtaacggccagcagattcgtgattggctgttcgtggaggatcacgcccgtgctttatataaggttgtgaccgaaggcaaagtgggcgagacctacaatattggtggccacaacgagaaggccaacatcgacgttgtgcgcacaatttgctctttactggaggaactggttccgaataaaccggccggcgtgcataagtatgaagatttaatcacatatgtgaccgaccgccccggtcacgatgttcgttacgccattgatgccaccaagatcggtcgcgaactgggttggaaacctcaagaaaccttcgaaaccggcatccgtaaaaccgtggaatggtatttaaacaataccgagtggtggagccgtgtgctggatggtagctacaatcgcgaacgtttaggcagcaactaatattattacaagcgatccaatttttaataaggtttacaatatgaaaggcattattctggccggcggtagcggtacccgtttatatccgattacacgcggtgtgagcaaacagctgctgccggtgtatgataagccgatgatctattatccgttaagcgtgctgatgctggccggcatccgtgaggtgctgattattaccaccccggaggacaacgagagctttaaacgtctgctgggcgatggcagcgatttcggcattcagctgagttacgccattcaaccgagcccggatggtctggcacaagcttttctgatcggtgaagagttcatcggccaagatagcgtgtgtttagtgctgggcgacaacattttttacggtcagcatttcacccagagtctgcaagaggccgttaagagcgttgagaccaaaggtgccaccgtgtttggctaccaagttaaagatccggaacgctttggcgtggtggagtttgacgataacttccgcgctttaagtatcgaggagaaaccgatccagcctaaaagcaactgggccgtgaccggtctgtacttctacgacaaccgtgtggtggaattcgccaaacaagttaagccgagtgcacgcggcgagttagagattaccactttaaacgaaatgtatttaaacgatggcagtctgaacgtgcagctgctgggccgcggttttgcatggctggataccggtacccacgatagtctgcacgacgccgcagcctttgtgaaaaccgttcagaatttacagaatctgcaagttgcttgtttagaagaaatcgcctatcgtaacggctggctgagcttagagcagctggaggctttaaccaaaccgatggcaaagaacgagtatggccagtatctgctgcgtttaaccaaaggcaccaaataatggcacgttttttaatcaccggcgcaaaaggccaagttggttattgtttaaccaagcagctgcagagcaaagccgatgttctggccgtggatcgcgatgaactggacatcacaaaccgcgatgccgtgtttaaagtggtgcgcgaattccacccggacgtgattatcaatgccgccgcccataccgcagtggatcgtgcagaaagcgagatcgaactgagcgaagccatcaacgttaagggtccgcagtatctggccgaggcagcaaacgagatcgacgccatcattttacacattagcacagactacgtgttcgagggcaccggcagcggcgaatataaagagaatgatgaaccgaacccgcaaggtgtgtacggcaaaaccaaactggccggcgaaatcgcagttcagcaagctaacaagcgccatatcattctgcgcaccgcttgggttttcggcgaacacggcaacaacttcgtgaaaacaatgctgcgtttagccaaagaacgcgagagcttaggcattgtgagcgatcagttcggtggtccgacctatgccggtgacatcgccagctctttaattcatattgccaacatcatcttaaacagtaaaattgatgtgttcggcgtgtaccatttcaccggtaagccgtatgtgagctgggccgatttcgccaaaaagatcttcgacgaggccgttagccagaaggttctggaaaaagccccgctggtgaatttcatcgccaccagcaactatccgaccagcgccaaacgcccggcaaacagccgtttagatttaaccaaaatcgacgaggtgtttggcatcaagccgagcaattggcagcaagctttaaagaatatcaaagcctatgcctaatgaaaattatcgaaaccaacatcccggatgtgaaactgctggaaccgcaagtttttggcgacgagcgcggctttttcatggagatcttccgcgacgagtggtttcgccagtacgtggcagatcgcacctttgttcaagaaaaccacagcaagagcatcaagggtgtgctgcgcggtctgcactatcagaccgaaaacacccaaggtaaactggtgcgtgtggtgcaaggtagcgtgtttgacgtggccgtggatctgcgcaaaagcagcccgacctttggtcagtgggtgggtgaagtgctgagcgccgaaaataaacgtcagctgtgggtgccggaaggcttcgcccatggtttctatgtgctgaccgagaccgcagagtttacctacaagtgcaccgactactataacccgaaagccgagcattctttaatctggaacgatccgaccgtggccattaactggaatctgggtggtgccccgtctttaagtgccaaagatctggccggcaaagtgctgaacgaagcagtgctgtttgaataa

SEQ ID NO: 4 - DNA, 13598 bp, Artificial Sequence
  nucleotide sequence codon optimized rfb APP8
atggcgacgccagagcaagtgagcaaccagaccaacgaggagattgacctcatcgagctggttcgcgtgctgtggaagaaaaagctcctcatcgccatcgtgacgtgcatcttcaccgccctcgcggccgtttatgccttcaccgccaaagagaagtggacgagccagaccgaagttatcgcgccacgtgtgaccgacattagcgaatatctgagtctgcgcaaggagtacaacctcatcatcggcagcgagttcaaagaaaacgaaatccgcaacgagctcagcgagctcttcagtcgctatgtgctgagttacgacgaagccatcgccttcttcaagacgacggacacgtacaagaagctggccgaaaccgagaatgaagtgggcctccagcgcgcggttgccgaattcacgacggagagtctgaaggtgatcaagccagacgccaagaaagacccgaatgcgctgggcagtaagattgcgatcagcttcgataccgcgctgagtgcccagaccacgctgaacgacttcatctgccacatcagcgacaccagtttcaacttcagtaagaatgagttcatttattggattaaggagagcattagcagtctgaattacgagaaagaggttatcgaacaagatcagagcatccaacgcaaggtgcagatccagaatctggagacggccctcgatatggcgaagaaggcgggcatcaaagagtacagcagcgccctcagcagtaatagtagcgtggccaatctggcggtgagcgataccaaaatcccgctgagcgatagcaagctggcggacggtacctatctgttcatgctgggcgagaagaatctgcaagcgcagctcgacatcgcgaaaacgaaggagatcgtgtacagcccgcgctactaccagatccaagaacagctgctgaagctgaataccctcctcccgaaagtggaaaaggttaccggccagagcttcagctatatcagcagcccagagctcccgattaagcgcgattggccaaagcgctttattctgctcctcattggcgcggtgattggtggcgttctgagcagtctgtgggtgatcggcaaacaaatcttcggccagaaataattatgaacaaggacatcaagattctgatcgcgacgcataagcagcacttcatgccgagcgacgaaatgtatctgccgctccacgtgggcaagctgggtaaagccgatctgggttaccaaggcgacgacagcggcgacaacatcagcatcaaaaacccaaatttttgcgagctgaccggtctgtactgggcgtggaaaaatctgccgaacgattatctgggtctgatccattatcgccgcttcttcagcgtgaagaaccgcgcggaacgcaaaaacaatccgctggagacgctgtatctgaccaacgaagaagccaaccagctgctgagtcagtacgatgtgatcgttccgagcaagcgcaactactacatcgagacgctgtacagccattacgccaatacgctgcacgccgaacatctggacgttacccgcgaaatcatcgcggaaaagtgcagcgagtacctcgccagctttgacgcggtgatcaaacagcgcagcggctacatgttcaacatgttcatcatgagcaaagcgctggtgaacgactactgcagctggctgttcccgattctgtttgaactggagaagcgtatcccaacggaccagtacagcgccttccatgcccgtttctacggccgcgtgagcgaactgctgttcaacgtgtggctgaaacagtacagccagagcaacccactgaaggtgaaggccatcccgtttgtgtatggcgagaagatcaactggctgaagaagggtaccgcgtttctggttgcgaaattcttcggcaaaaaatatgagaaaagcttctaagttattatagggacaaataaatgaagcgcattctggtttacggcatgaccgacaacttcggtggcatggaggcctacattcataacatctatcagcatctggataaaacccaaatccagtttgacttcgtgtgtgacttcccgaaaatgacgctgagcgactactatctggacaacggttgcaagatccacttcatcccgccgaaaaaccaaggtctgtttaagagtctgtgggcgatgtggaaagtgatcaaggagaacaactatgatgttatctatttcaacatcatgaacgcgggctacgtgctcaacatgctgccggcctttctgctgggtaagaagatcatcgcgcacagccacaatgcggacaccgacaaaaagaagctgcactacggtctgcgtctgctgctgaacatcgtgacgaagatcaagctcgcgtgcagtaaggaggccggtttcttcatgttcggcaaggaagaaaatttcagcatcattaataatgcgatcaacctcgaccgctatctgtatagcgaggagaaataccgcgacctccgccacaaactgggctggggcgataagaaggttattctgtacgtggcccgcatgaatcaccagaagaatccgctgttcgcgctgtatatcatgcgcgaactgaagcagagcatgccgaatgccgttctggtgtacgtgggtacgggtgagctgaaggaacaagttcagcagtacattctggacaacaacctcgacaacgtgattctgctgggtctgcgcaacgatgtgaacgagctcatgatcgcggccgatctgtttattctgccgagtctgtttgagggtctgccgattgttgccgttgaggcgcaagccgcgggtctcccaatcattctgagcgaaaacatcagcatcgaggcgaaactcgtgaacagcacctacttcctcccgattaacgacgtttttctgtgggttaataaaatcaaaaagattctggagatcagtggcaacaagcgctttagtgaccagctggcgctgagcaaagcgggttacaacatcgagagcgtggtgaagaacatccagaagattctcgtgaattaaggtttggtatgaataacaccaagatcagtctgatcttcgcgtgctataacgtgagccagtatctggacaatctgttccagctgctgacgaaccagccgtaccaaaatattgaaatcattttcgtggaggactgtgccacggacgatacgaaggcgaaactccagagcttcaacgatccgcgcgtgaagctgctgtgcaacgagaaaaatatcggtgcggccgaaagccgtaaccgtggcatccagatcgttaccggcgaatacatctggttcccggatccagacgatctgtttgacgaactgctgctgaccaaggtgaacaccatcatccagaaaaaccgcccggatgtgatcagcatcggcatgcaagaacgctacgagatcaacggcaagacggactacacgaaggacatcatcagccgctacgacggcctcattaccggcgacttcaccgatgttttcgtggatctggaggaaagctttctgtttggctacacgaataacaagttttacaaggccaacatcatccataagtaccgcattctgaacgagcaccaagcgctgaaggaagatttcgaattcaatatcaaggtgtttaaacaagttagtaacttctatctgctgaatgaaccgctctatttctacatgaagcgcaacaacggcagtctgaccagcaaattcgtgccggactatttccgcatccacatgcagaccctcgccagcttcaaaagtctgatcgaggtgaaggccaccatcaacgacaacgtgaaccgtctgctggtgaaccgcttcgttcgctactgcctcagcgcgatcgagcgcaacagcagtctgaaaagcggcatgagctttctggagcagaaccaatggattaaggaaaatatctttaatcaagaaaaatacaacgagtatctgctgctgagcgatctggtgaacaagaaacagaagctgttttactttctgatcaagtatcgcatcggttttctgctcgtgacggccgcgaacatcgtgaaactggtgaaggcgaagttcccgattctgttcgtgaagctgaagggttaattaactggattttaaaatgaagaagtaccagatcgtggagctgagtaccgaacacaaccatgcgggcagcaaggccgtgcaagatgtgtatgagatcgcgctcagcatgggttacaaggcgaatgtggttcgcacggccaccagtgtggatagtctgctggccaaaattctgcgccaagttatcttcttcatcgactggctgaagatctacttcagcatcgagagtaacagcatcgtgctgatccagaacccgtactaccacaaacagctcatccgtaactggattctgaatcgtctgaagcgcattaaaaaagtgaagtttatcagtctggttcacgacgtggaagagctgcgcaagagtctgtacaacaactactataaaaacgagttcgagaccatgctgagtctggcggacagcatcatcgtgcacaatgataagatgaaaagctttttcatcaaaaagggctacagcgaggacaaactcatcagtctgggcatcttcgactatctgcagaagagcgtggacaaaaagcgcgtgagcttcgaacgtgcgatcagcgtggcgggcaacctcgatatcaagaagagcagctatattgcgcagctcggcagcctcccggcgatcaaagcgcatctgtacggtccgaacttcgaacatagtctggaggcgttcccgaacatcgaataccacggtagcttcccggccacggaaatcccgcagaaactcgtgagcggttttggtctggtgtgggacggccagagcattgaaacgtgcaccggcgacttcggcgagtacctccagtacaataacccgcacaagctgagcctctatctgagcagtggcatgccggttgtgatctgggacaaagccgccgaggccgatttcgtgaagaaacacaacgtgggtctgtgcgtgagcagtctgagcgagctccaagacaagctcaacgtgatgaccgagcaagaatttgaagaaatggtgaacaacgtggaaaaacagaccgcgtgcctcatcagcggcgagtacaccaaaaaggcgatcagcgaggcggaacgtgtgatctaagaatgttcctctatctgctggtgttcagtctgctgctgattctgatcttcaatctgctcatcgtgaatctggactacatgcacccgagcatcctctttgttgtgccatttctggtgtttggcgtgacgagcattctgggcgaggaggcgtataagatcatcttccacgaggagacgctgctggtgatcgttagcagcgcgctgatcttcaccttcatcacgctgctgagccagaccgtgtacaaaagcaaagagaatctgaacttcccgctgaccgagatcatcatcagtaagaaagtgacgctgttttttattgtgttcttcatcgtgacccagctggcgttcatcaagtatctggaggccattagtctggcccacttcggttacagcggcagtctgggtgagatgatcagtctgtacgacgtgatgacgaagttctggaccgagatcttcagcgaactcaacgtgccgatcccgctgctctaccgtatcggcaatccaatcacgcaaggcttcggctatctgattgtgtatattttcatccacaactacgttgccaccaagcgcatcgataagctgcatctgctgatcattctgctgctgtgtctgaacatcattctcaacggcagccgcagtccgatcttccgcatcgttacgatgatgctgatcaccttttatgtgctgtataacaagcagaacaacgtgcgtcgcggcaacatcaagtttctgctgaagagtctgctgatcgtgatcttcagcggcaccttcttcattgcgctgctgagtctgatgggccgtgaaaacgatctggacatgttccattacatttttatctacgttggtgcgccgctggtgaacctcgataactatctggcgtttcgtccggatggtagctacgccaccatctttggcgagcaaacgtttcgcggtctgtacgcctatatcgcgaagatcatcagcgatgagagtctgatcttcccgacgatcgatcagttcacgttcagcaacaacggtctggagatcggtaacgtgtataccaccttctatagcttcatctacgatttcgagtacgtgggcttcatcccgctgattctgattatcgcgctgtactacgtgttcacgtatcagcgcctcaagacgcgcgccatcaagaccaataaagtgcatttcagtctgttcatctatgcctacctcttcaacgacctcatcatgctggccttcagtaatcgcttctacaccacggtgctggacatcggcttcatcaagattgttatcttcagctatatctgccacctcctctttgtgcaccgcagcaagatcaaaggcaccgtatgaacgttaaaagtgtgaaatttaatttcattatgaatctgattctgaccgttagcaactttctgttcccgctggtgacgttcccatacgttagtcgcattctgcagccagaaggtaccggtaaagtggcctttgcgattagcgtggttagctacttcagcatcttcgcgagtctgggtgtggccacctatggcgttcgtgcgtgtgcgcaagttcgcgacaataaagatctgctgagtcgtacggtgcatgagctgctgttcatcaacatcatcgccacgatcattgtgtacgtttgctttctgctggtggtggcgtttaccccacgctttagcgcggaaaaagagctgttctgggcgacgagcatctttattctgttcaccatcattggcatcgagtggctctacaagggtctggagaagtaccagtacatcacgatccgcacgatcatcttcaagctcattgcgctggtgctcgtgtttgtgttcatcaagacgaaggatgactacgtgatcttcgccgtgatcagtgtgtttgcgatcgttggcagcggcatcttcaacctctttaacagtcgcaagctgattaactaccatctgtacgaggattacgagttccgcaagcatttcaagccaatgtttctgctgtttctcacgacgctcagcatcgccatctacaccagtgtggatgaagcgattctgggtctgctgacgagtccgcaagatgtgggctactataacgcggccatgaaggttaagggcattctgtttacgctgatcaccagtctgggcattgtgctgctgccgcgtctgagctattatgttgagaacaatatgacggatgaattccatgccgccctcaagaagagcatgaacttcatcatcgtgatcgccgttccagtggtgatcttcttcatgctgttcgccaaggagattattctgctgctggccggcgaaagttatatcaacgccattctgccgctgcagattattgtgtgggcgctgctgctcagcgccattaccaacattctgggcatccagattctgctgccgctcaagaaggataaagagctgctgatcagcgtgctgctcgcggccattgtggacattgtggccaatctgattctggttccgcaactcgccagcgttggtaccgccatcagcgttgtgatggccgaactcaccgtgctggtggtgcagctggttatcctccgcaagtacatctggatcctcttcagcaatctccagttcgtgcgcatcggtctgagcatcgttttcagcatcgtgctgagcctcagcatctatcagtggaacatcacgaacagcatcatgctcacgtttctgatcatgggcttcatcttcttcacgacctacttcattctgctgctgattctgaaggagaacttcatgatgtacgtgtaccagaccatccagcacaagattctgaaataaattatatagtgttatcacataacgtatccttggagaatagaaatgaaatatgattatctgatcgtgggcgccggtctgtttggcagcatctttgcgcgcgaggccaccaagcgtggcaagaaatgtctggttatcgagaagcgcgatcacatcggtggcaactgctacacgcagaacgtggaaggcatcaacgttcacaaatacggtgcgcacatcttccacaccagcaacaaggtggtttgggactacatccagcagttcgccgagttcaatcgctttaccaacagcccggtggcccgctataaggacgaactgtacagcctcccgttcaacatgctcaccttcaacaagatgtggggcgttatcacgccgcaagaagccgaagcgaaaatcaaggagcagatcgcgaaggagaacatcacggatccgaagaatctcgaggagcaagccatcagtctggttggtcgcgatatctacgagaagctcatcaagggctataccgagaagcagtggggccgtaagtgtacggagctgccagccttcatcatcaagcgtctgccagttcgctacacgtacgacaacaactacttctacgacacctatcaaggcatcccgatcggtggctacaccggcatctttgaacgcatgctcgagggcatcgaggtgaaactgggcgttgacttcttcgcggaacgcgaacattacgagagtctggccgagaagatcgtgttcaccggtatgattgacgaatattttggttaccagttcggcaaactggaataccgcagtctgcgcttcgacaacgaagtgctgaacatcccgaactaccaaggcaatgcggtggtgaactatacggaagccgaggtgccatatacgcgcatcatcgagcataagcatttcgagtacggcacccagccgaaaaccgtgatcacgcgcgaacacagcaaggagtacgaagaaggcgacgagccgtattacccgatcaacgacgcccgcaacaacgaactgtacgccaagtacaaggcgctggccgacgcgaccccaaacgttattttcggtggccgtctggcccagtataagtacttcgacatgcacaatatcatcgccgaggcgctggagtgcgttaaggtgcacttttaatataagggagtaacgctatgaataagatcatcgcgaagatcagtctgatcctcgtggatatcgtggccatcttcgttagcattctgatcgccgtgagtctgcgtaaaattctgggtctgctcttcacgctgccggagatcgactacagctacatcttcttcgcgtatgtgtatctgattctgattctgatgatgacgtacctcggcgcgtataccaaacgctacgacttttggcacgaaagccgtctgatcgtgcgcggcagctttctcagtctgctgattctgctgagtgccctcgcgctgggccaaaacgcggaatactatagccgcagcacgctcgtgctgatctttctctgctgcgccatcgtgctgccgatcgccaagattttcaccaaaaaaattctgttcaaactgggtatctggcagctgccggcgaaggtgatcagcgagaacgaccagttcaaaaacgagctcttcgaagaccagtatctgggctatgtgaaggcgaaacacagcgagcacaagattatcttcatcgacggcgcgaatctgggcaaagatcgtctgaaccagatcatcgaggacaacatcaagaatagccgtgagatcatcttcaccccggttctgaatggctacgacttcagccatagctacatttataacatcttcaacacgcgcaccaacattttcacgctggagaacgagctgctgagcaaaagcaaccgcatcttcaaactgctgatggactatattctggtgctgggtagtgccgtgttctgggtgccggtgctggtgctcatcgcgttctggatcaagaaggaggatccgaaaggcgaggtgttctttctgcagcgtcgcctcggcgtgaatggcaaggaattcatgtgctacaaattccgcagcatgtacagcgaccagagcttcatgcaagaatggctggagaaaaatccggaggaggccgcgtactaccgcatctaccataagtatatgaacgatccgcgcatcaccaaaatcggcgcgttcctccgcaaaaccagtctggacgaactgccgcagctgatcaacgtgctgcgtggtgagatgagtctcgttggtccgcgcccgtacatggttatcgagaagaaggacatcggcaaaaaagccccactggtgctcgcggttaagccgggcattacgggcatgtggcaagttagcggccgcagtgatgtgaacttcgacagccgcgtggagatggatgtgtggtatatgaaaaattggagtctgtggaatgacatcgtgattctgatcaaaacggtgcaagccgtgttcaagcgcgacggtgcctattaaagtatgatcaccagcatccagtacctccgtggcatcgccgcgctgttcgtggtgctgttccacatgaagtggatgctcaacaatgtgtacgtggagaagaacctcggcgacatcttcttcatcagcggcaacttcggcgtggatctgttcttcgtgatcagcggcttcgtgatctgtctgagcacggaacgcgaaacgctgcacccggtgaaggagtttttcatccgccgcttcttccgcatctacccactgctgctgctgagcgtttgcaccatctacattctgggcgacttcaagatccacgagctgatcctcagcatgatcccaatccatctggactacagcagcccgagcccggtgttcggctacaacattctggttagcgcgtggaccatcacctacgagattagcttctacatcatcctcgtgctgagtctgatgatcaaccatcgcttccgctgcgaactgaccattctgttctaattatcattaatatagtttcaaactattattattttggtgaatatagcctatcactagatagagagataccccttgataaaaggggacatttttttgttatgttctcatcatcaatgttattaacatttatttatgggattttaatatatataaaattacaaattttatgaaaagcattatcatcctcgacaagtacttcctctacagcattctgctggtggtgatcagcttcgtgttcatcaaacacccgatcttcgacggccacggtgtgctgaaatggggctttctgagcttcatcattctgctgattctgctcatcatcgagaacacctacggcatcgccaaaagcaactttctgttctggctgggcgaaatcagctacagtctgtatctgacgcacatcattatcctcgaattcattctgaagcacatcaccccggagatctggaacaacccgaatctgggcatgagcaagatcctcttctacctcgccatcagcatcagcttcagctatctggtgtatctgctggtggagaagccgttcatcaacctcggcaagaagctgatcacgaagctgtaaatattaatggatgattttatgaagtcacgaaatctcgaacctacaaaaacgcatctgatctatttagatatactaaatatttttgcttgcattgctgtacttttttacatcacaatggtattgtacattggtataacgtaaatgaattggcttggaaacaagccttattttttgaagtggctttttattgggctgttcctattttctttatgctcaccggcgccacgctgttcgaataccgcaaccgctacagcacgaagcagtttttcatcaagcgcatccagcgcgccgtgttcccgtttctgagctgcagcctcattctgctgggctatagcttttacagcggcatgatcgaggcctttagcatccgcgacagcatcagtgccatcttcaacaccaaggacatcccgttcattgaaatctattggttttttatccatctctttagtctgtacatggtgatcccggtgctcagtctgctgaaagataactaccgcattctgtgctatattgtgggcgccatgtttctgacccacagtctgtttccggtgatctttgacttcttcaagctgcactacaactggagcatcattttcccgatggcgggctacagcatctatctggttctgggctatctgctgagtaaggtgaaactggaaaagaaatatcagatcatcatttacattctgggcattctgagcgtgctgctccgctacttttatacctacgtgagcagtctggaggccaaccagctcgatcgcacgctgttcagctacatgcaattccacaccgtgtttctggcggtggcgatcttcattttcgtgaaggaattcttcagcggtgtgaaactgttcaacgccaaggtgctggcggtgttcagcagctgtagtctgggcatctatctgatccacaagctcgtgatggactacgaactcaagtttctgggcatcagcgaggacaatctctactggcgctttttcggcgccttcatgacgtacggcgcgtgcctcgtgatcgtgctgtttgttaagcgcatcccgtatctgcgcgccatctttccgtaaagatattataaatatgaaaattctgatcaccggtggcgccggttttatcggcagcgccgtgatccgctatatcatccagcatacccaagatagcgtggtgaatgtggacaaactgacctacgccggcaatctggcgagtctggaaagcgtgagcaatagcagccgctaccactttgagcaagcggatatttgcgacagcacccgcatcagtcagatcttctgcaagtaccagccggatgttgtgatgcatctggccgccgagagccacgttgatcgcagcattgatggtccggcggcgttcatgcagacgaacatcatcggcacctataccctcctcgaagccagccgccagtattggctcagtctgccgctggaacgcaagcaaaccttccgcttccagcacatcagtacggacgaggtgtatggcgatctcaacgatagcaacgagctgttcagcgagaacacggcctatagcccgagcagcccatatagcgccagcaaggccgccagcgatcatctcgttcgtgcgtggtttcgtacctatggtctgccgacgctggtgaccaactgcagcaataactatggcccgttccagttcccggagaaactgatcccgctgatgattctgaacgccattagtggcaaaccgctgccgatctatggcaatggtctgcagatccgcgactggctgttcgttgaagaccacgccatcgcgctgtatcaagttctctgtcgcggcaaagtgggcgaaacgtacaacatcggtggccacaatgagaagaccaatatcgaggtggtgcaagcgatctgccgtctgctggacgaactggtgccgaataaaccgagcggcatcgagcagtatgaagaactcgtgacctacgtggccgatcgcccgggccatgatgttcgctacgccatcgacgcgagcaaaatcgagaatcagctgggttggacgccgaaagaaaccttcgaaagcggtctccgcaagaccgtggagtggtatctgaataaccagaagtggtggcagagcgttctggatggcagttactgcggtgagcgtctgggtctgagtctgaaaagctactaa

SEQ ID NO: 5 - protein, 370 aa, Artificial Sequence
  amino acid sequence rfb APP8 Chain length determination protein
MATPEQVSNQTNEEIDLIELVRVLWKKKLLIAIVTCIFTALAAVYAFTAKEKWTSQTEVIAPRVTDISEYLSLRKEYNLIIGSEFKENEIRNELSELFSRYVLSYDEAIAFFKTTDTYKKLAETENEVGLQRAVAEFTTESLKVIKPDAKKDPNALGSKIAISFDTALSAQTTLNDFICHISDTSFNFSKNEFIYWIKESISSLNYEKEVIEQDQSIQRKVQIQNLETALDMAKKAGIKEYSSALSSNSSVANLAVSDTKIPLSDSKLADGTYLFMLGEKNLQAQLDIAKTKEIVYSPRYYQIQEQLLKLNTLLPKVEKVTGQSFSYISSPELPIKRDWPKRFILLLIGAVIGGVLSSLWVIGKQIFGQK

SEQ ID NO: 6 - DNA, 1014 bp, Artificial Sequence
  nucleotide sequence of gne-HA
atgaaaattcttattagcggtggtgcaggttatataggttctcatactttaagacaatttttaaaaacagatcatgaaatttgtgttttagataatctttctaagggttctaaaatcgcaatagaagatttgcaaaaaataagaacttttaaattttttgaacaagatttaagtgattttcaaggcgtaaaagcattgtttgagagagaaaaatttgacgctattgtgcattttgcagcgagcattgaagtttttgaaagtatgcaaaaccctttaaagtattatatgaataacactgttaatacgacaaatctcatcgaaacttgtttgcaaactggagtgaataaatttatattttcttcaacggcagccacttatggcgaaccacaaactcccgttgtgagcgaaacaagtcctttagcacctattaatccttatgggcgtagtaagcttatgagcgaagaggttttgcgtgatgcaagtatggcaaatcctgaatttaagcattgtattttaagatattttaatgttgcaggtgcttgcatggattatactttaggacaacgctatccaaaagcgactttgcttataaaagttgcagctgaatgtgccgcaggaaaacgtaataaacttttcatatttggcgatgattatgatacaaaagatggcacttgcataagagattttatccatgtggatgatatttcaagtgcgcatttatcggctttggattatttaaaagagaatgaaagcaatgtttttaatgtaggttatggacatggttttagcgtaaaagaagtgattgaagcgatgaaaaaagttagcggagtggattttaaagtagaacttgccccacgccgtgcgggtgatcctagtgtattgatttctgatgcaagtaaaatcagaaatcttacttcttggcagcctaaatatgatgatttagggcttatttgtaaatctgcttttgattgggaaaaacagtgctacccatacgatgttccagattacgcttaa

SEQ ID NO: 7 - DNA, 1137 bp, Artificial Sequence
  nucleotide sequence of wzy (APP2)
atgaactccttagtatatagaatagatattagaacacttattttttctattttttattttacttttttagtatcggattttttattattagctcaagatggcactattacaaaagatatcatcaaatgggttaaattattctcattattgccattgctcttattaatatttaaattgcctttgaatctcttgattttaggtttttttactataatgataagtgctttttattctatttatacgggagattcgtttttattatatatatgtttgctgatgtctttttcttataaagttaattttaactttttattcaagataggattatatcttacttcaattctagttgttctaatactaacttatttcttttttgaatattttctgattggtgacagtcattttgtatatgatgcgacctattggtttaaacgttatacatttaattttgataatcctaatgcatttcctatgagaatattcgttttttttatattttatatattgcatgtaggtaagctgcgactttttgatacatttctatttgttatactatttggaatagttttctatttttcaaattctagaactgcattttatatttttattttgtgtgtccttactattcattttaaccaagtttttaatgtgctaaataatacttttgttaaattactaattaataattcaattatatttataactattttttcaatttggtcggctatatattatcaagattattattcctatttagaaccgattaacaaaattttatctaaaagaatatactttgctaatgaggcttataagagtttaggatttgaattttaccctaggaatattaaatggtggatagaagaatctgattggcatattatagataatggatatgtatatttatttatttctggtggtcttttagtaggaaatttatttatattttctataacttggcttatgtatagactaaataaatttaacctaagtaatgaggcaatattattaatgttttctatgttatatcttttatctgagagtcattttataaatatattttacaatatacctattttattattagctattttcattaataaaactaatattgtacgctatttggaatgtaaaaaatga

SEQ ID NO: 8 - DNA, 1137 bp, Artificial Sequence
  nucleotide sequence of codon optimized wzy (APP2)
atgaattctttagtgtatcgcattgacatccgcactttaatttttagcatcttttattttacctttctggtgagcgattttttactgctggcccaagatggcacaatcaccaaggacatcatcaagtgggtgaagctgttttctttactgccgctgctgctgctgatcttcaagctgccgctgaatttactgattctgggcttttttaccattatgattagtgccttctacagcatctataccggtgacagctttttactgtacatctgtttactgatgagctttagctacaaagttaattttaattttttatttaagattggtttatatctgaccagtattttagtggtgctgattctgacatattttttctttgagtactttctgatcggcgacagccactttgtgtacgacgccacctactggttcaaacgctacacctttaactttgataacccgaacgcatttccgatgcgcatctttgtcttttttattttttacattctgcatgtgggcaaactgcgtttattcgacacctttctgttcgttattctgtttggtatcgttttttactttagcaacagccgtacagccttttacatttttattctgtgtgttctgaccattcattttaatcaagtgtttaatgttctgaataatacctttgttaaactgctgattaacaatagcattatctttatcaccatttttagcatttggagcgcaatctattatcaagattactattcttatctggaaccgattaataaaattttaagcaaacgtatttattttgcaaacgaagcctataagtctttaggcttcgagttctacccgcgcaatatcaagtggtggatcgaggagagcgactggcatattatcgacaatggctatgtttatttatttatcagcggcggtttactggtgggcaacttatttatcttttctattacttggctgatgtatcgtctgaataaatttaatttaagcaacgaggccattttactgatgtttagcatgctgtatttactgagcgagagccattttatcaatattttttataacatcccgattttactgctggcaatttttattaataaaaccaatattgtgcgttatttagagtgtaaaaaatga

SEQ ID NO: 9 - DNA, 97 bp, Artificial Sequence
  Ec_SL/Kan_fw
caaattccggttaaaaaaagaccgcttgtttgagagtgataatcgcaaacaagcggtcttttttgatcaaaatattattacacgtcttgagcgattg

SEQ ID NO: 10 - DNA, 91 bp, Artificial Sequence
  Ec_SL/Kan_rev
gatgaagagcaaagattgggagataatgtgagaaatctttagattcaaactaagctgagaagaaaaaggtccatatgaatatcctccttag

SEQ ID NO: 11 - DNA, 82 bp, Artificial Sequence
  E.c._5_DELTArfb fw
gtaatgttaatgaaagcatataagaaattttcaaatgaataaagaaactgtttcagttattattacacgtcttgagcgattg

SEQ ID NO: 12 - DNA, 85 bp, Artificial Sequence
  E.c._5_DELTArfb rev
gagcatgtaatcttctgataaaaatcatttgtacgatattttcagttacatactatgcgtaggtccatatgaatatcctccttag

SEQ ID NO: 13 - DNA, 83 bp, Artificial Sequence
  SL1344_DELTArfb fw
gagcaattaatttttattggcaaattaaataccacattaaatacgccttatggaatagaaaaattacacgtcttgagcgattg

SEQ ID NO: 14 - DNA, 83 bp, Artificial Sequence
  SL1344_DELTArfb rev
gcgttcagattttacgcaggctaatttatacaattattattcagtacttctcggtaagcggtccatatgaatatcctccttag

SEQ ID NO: 15 - DNA, 92 bp, Artificial Sequence
  Ec_SL/Kan_fw_elo
cagggctagcgctaattaccaatttattgtttagcttaggaatttttttaggttagttgcaaattccggttaaaaaaagaccgcttgtttga

SEQ ID NO: 16 - DNA, 92 bp, Artificial Sequence
  Ec_SL/Kan_rev_elo
caatattagcttatgtattatattagaaggcctacagataagcaaaaaatattattgatgaagagcaaagattgggagataatgtgagaaat

SEQ ID NO: 17 - DNA, 22 bp, Artificial Sequence
  BamHI-Fw KanR-Fw
cgggaattcaagcttggatccc

SEQ ID NO: 18 - DNA, 22 bp, Artificial Sequence
  XhoI 3′rspU- Rev
gacgctagcatatgagctcgag

SEQ ID NO: 19 - DNA, 30 bp, Artificial Sequence
  BamHI-SL-gnd Fw ext
gtttcatcagtaatgggacagaaaggtacc

SEQ ID NO: 20 - DNA, 40 bp, Artificial Sequence
  XhoI-SL-GalF Rev ext
cacactcgagcaattgaccggtttttctattccataaggc

SEQ ID NO: 21 - DNA, 46 bp, Artificial Sequence
  5′ NdeI_wzy
caggtaccatatgaactccttagtatatagaatagatattagaaca

SEQ ID NO: 22 - DNA, 40 bp, Artificial Sequence
  3′ EcoRI_wzy
cttatcagaattcattttttacattccaaatagcgtacaa

SEQ ID NO: 23 - DNA, 31 bp, Artificial Sequence
  3′ EcoRI_pEC415fw
gtaccgagctcgaattcttgaagacgaaagg

SEQ ID NO: 24 - DNA, 34 bp, Artificial Sequence
  5′ NdeI_pEC415rev
cactgcaatcgcgatagctgtctttttcatatgt

SEQ ID NO: 25 - DNA, 61 bp, Artificial Sequence
  3′-gne-cat_overlap
gaataggaactaaggaggatattcatatggaccttaagcgtaatctggaacatcgtatggg

SEQ ID NO: 26 - DNA, 80 bp, Artificial Sequence
  5′-gne_Sl1344rfaL
attgctcaaattggtatcattaccggttttctgctggcgctaagaaatagataatgaaaattcttattagcggtggtgca

SEQ ID NO: 27 - DNA, 80 bp, Artificial Sequence
  3′-cat_Sl1344rfaL
aaaaactggtttgataagtgattgagtcctgatgatggaaaacgcgctgataccgtaattgtgtaggctggagctgcttc

SEQ ID NO: 28 - DNA, 80 bp, Artificial Sequence
  5′-elo-gne_Sl1344rfaL
ttttatctttcgtcggtttttatatcgttcgtggcaattttgaacaggtcgatattgctcaaattggtatcattaccggt

SEQ ID NO: 29 - DNA, 80 bp, Artificial Sequence
  3′-elo-cat_Sl1344rfaL
tttcaaaatacagttgggaaaatgtagcgcagcgtttcgaggaacaaatgaaaaactggtttgataagtgattgagtcct

SEQ ID NO: 30 - DNA, 33 bp, Artificial Sequence
  5′-cat-P2new
ggtccatatgaatatcctccttagttcctattc

SEQ ID NO: 31 - DNA, 58 bp, Artificial Sequence
  5′ gne overlap_wzy (co)
cccatacgatgttccagattacgcttaatgaattctttagtgtatcgcattgacatcc

SEQ ID NO: 32 - DNA, 68 bp, Artificial Sequence
  3′ wzy (co)-cat_overlap
gaataggaactaaggaggatattcatatggacctcattttttacactctaaataacgcacaatattgg

SEQ ID NO: 33 - DNA, 58 bp, Artificial Sequence
  3′ gne_wzy (co) overlap
ggatgtcaatgcgatacactaaagaattcattaagcgtaatctggaacatcgtatggg

SEQ ID NO: 34 - DNA, 51 bp, Artificial Sequence
  5′ XmaI-XhoI-APXIIIne-HIS-fw
aaaaaacccgggctcgagatggatgtaactaaaaatggtttgcaatatggg

SEQ ID NO: 35 - DNA, 38 bp, Artificial Sequence
  3′ APXIIIne-HIS-HindIII-rv
aaaaaaaagcttttagtggtgatgatgatggtgatggt

SEQ ID NO: 36 - DNA, 72 bp, Artificial Sequence
  FW_pliCint
ctaattagtaaccacttttaagcatggttaatcctattttgaaaaagcaaaatccctggtgttttcaaaata

SEQ ID NO: 37 - DNA, 71 bp, Artificial Sequence
  REV_pagCint
gattcactctgaaaaattttcctggaattaatcacaatgtcaggtcgatattgctcaaattggtatcatta

SEQ ID NO: 38 - DNA, 76 bp, Artificial Sequence
  eloFW_pliCint
cgtaacgttaaagaatatgtgaatcactaccgtagtataatggctaattagtaaccacttttaagcatggttaatc

SEQ ID NO: 39 - DNA, 79 bp, Artificial Sequence
  eloREV_pagCint
gataagcaggaaggaaaatctggtgtaaataacgccagatctcacaagattcactctgaaaaattttcctggaattaat

SEQ ID NO: 40 - DNA, 32 bp, Artificial Sequence
  FW_rfaK_rfaL
ctatttatatggcgctatcatcagggaaacag

SEQ ID NO: 41 - DNA, 34 bp, Artificial Sequence
  REV_rfaL_rfaK
gacagtataattaatgatattaaccgtgcgcttg

SEQ ID NO: 42 - DNA, 13750 bp, Artificial Sequence
  APP2 O-antigen biosynthesis cluster integrated located on pDOC plasmid
tttctgtcccattactgatgaaacacggcttatttgtttatcgccacaaagacccgattgaattacgcccaattttaggacaaatggcagggctagcgctaattaccaatttattgtttagcttaggaatttttttaggttagttgcaaattccggttaaaaaaagaccgcttgtttgagagtgataatcgcaaacaagcggtcttttttgatcaaaatatttcaaatgctaaaggatgaaccgcaaccgcaggttgagctagcattcgggttttgcaccacaaaacgagagccgtccaaaccttcggtataatctaccgtaccaccgattaaatattgcaggctcataggatcaaccactaaaccgacgttttggttttcaatggttaaatcgccttcattgatttggtcgtcgaaggtaaaaccgtattggaaaccgctacaaccgccacccgtaatatacactcgtaaacgaagattcgggttatcctcgccctcaattaaactcttaactttcttcgccgctgcatcggtaaagataagaggaatttgaatatcgtccattttttcttctcagcttagtttgaatctaaagatttctcacattatctcccaatctttgctcttcatcaataatattttttgcttatctgtaggccttctaatataatacataagctaatattggctattattttgagagtaatattatgcttgaacaagcatcaaaccaaactaacgaggaaatcgatctgattgaattaattcgcgtgctttggaagaaaaaattattaattgctattgttacctttatttttactgcattggcagcagtttatgcttttaccgcgaaagagaaatggacatctcaagcagaggtaattgcgcctagagtgacagatatttccgaatatttatcgttacgaaaagaatataatttaattatgggttctgagttcaaagaaaatgatattcgtaatgaactaaatgagcttttttctcgttatgtgctttcttatgacgaagcaatctctttctttaagaccacagatacttataaaaaacttgcagaaaaagaaaatgaagcaggtttacaaagagcagcttctgaatttacaacggaatcattgaaggtgataaaaccggatgcgaaaaaagaccttaatgctttaggcagtaagattgctatttcatctgagactgctttatctgcacaaacagagttaaatgattttattcgtcatattagtgatatttcttttaattttagtaaaaatgaatttatttattgggtaaaagagagcatatctagcctaaattacgagaaagaagtgatagagcaagatcagagtattcaacgaaaagttcagattcaaaacttagaagccgcacttgatatggcgaaaaaagcaggaattaaagagtatagttctgcattgtcatcaaatagttctgtggctaatctcgcggtaagtgatacaaaaattccgttatcagattctaaattagcagatggaacctatttatttatgcttggtgagaaaaatctacaagcacaattagatattgcgaaaaccaaggaaattgtttattcgccaagatattatcagattcaagagcaacttttaaaattaaatactttattacctaaagtagagaaagtaactgggcaaacttatagttatgtatcttcgccgacatatcctgttatcaaagatgcacctaaaaaaggaattattttagttataggtttcttggtaggattgttactgagttcattcactgttttgatttctgtattagtgagaaataagaagagctaattatgaggtatgaagaggaatattaacctctttatgtgccttttgatgatccaatttactgaagtgcttagactatttaggtaataaaatatagggataataatgaagcataagatattacattttagccaagtgcttggcggtgtcggtcgatatttagagctatatgataagtacatcaataaagattcatttgaaaatatatatatattacctataggtgattgggaggcggcagaagctcaggataaacggtatatattaaatattgaacaatccttttcaccaattaaattgatctctaatgttataaaaattagaaatatcttaaaaaaagaaaagccagatatcttttatctacatagtacttttgctggcgttattgggcggttagctgctattggtatgaggtgtaaagtaatttataaccctcatggttggtcatttaagatgaatgtatctaggctaaaacaaactttttataagattatcgaaggtggtttagttttcttaactgataagtttgttttaatttcgaaatcggagtatgaggcggcacgttcaattggtgtttcagagaagaaatgttgtctcatatataacggtattgaaacgataaaaaaaacagatatagcaatcattcctaaattagatgataaatatatcattggaatgataggacgtattagtgagcaaaaaaatccaatgttttttgcccagtttgccaaagagattattaaacaataccctaatacttatttcattttggttggtgacggtgagcaacgagaatcgttagaagactatttagaacgtaataacttgaatgatgttttttatattacgggttgggtaactaatcccgaaagctacttaaatttatttgatcaagcagtattattctcaaaatgggaagggctatgtctttcagtctgtgagtatatgttatatgaaaagcctatattagtaagtaatattggtggtattaacgatcttattcagaatgaagttaatggttttactattgttgaaggtgatcttaaggatgcggttaacaaatctaatagattaagaaatgagcctaaaactgtagctaagtttattgaagcctcaaacatacttattcaagagaaatttaatgctcaaaaaatggtaaatagtttagaaaaactttttatcaaattatcagagaataaataatgaaagaaaaatttagttgcattgttgtttgttataaccccgataattcggtgcttgacaatctcaaaaactatattagttatgtgggaaaagtaatcgtagttgataattcagatgtggataattctcaattattttcctcactttcagaatacttaatttatataccattgtataaaaatgtgggtattgcctatgcactaaatataggagtagaaaagtccaaagaattaggatatgaatatatcattactatggatcaagatagttcttttgctactaatctagtggatgtatattcacattatataagtaattatcctatagatcagataggagcattatccccagtttatattacggacaggggatttaatcgaacaagtaaagaagaatttaaacaaataaaaattactatgcaatcaggttctatgttctttactgataaatttgatgtaatcggtcgctttgataatgatctgttcttagatgtagttgattgggaatatttctttagaatttatacgttaggatataaaacgattcaatgtaataaagcaatgctgaaacatgctccagcggaaacgctaacgttatttaaaataaaaggaaaaacaattggtgttggagtcgcttctccattaaggtattactatcaaattagaaatctactttggtgtgttttacataaaaagagtttttttatgataaaaacaatagcttataaatttattaagattctatttttgtttaataataaaaagcaatatttatcatttgcttatatggctattaaagacgcctttaataatcgtttaggggcatatgatacactttatttagagaaatctcgtaatgaaaaatgatttaccattaattagtattattattcctatctataacgtgaagccttatcttgaaaaatgtgtaaatagtgtattatcacaatcatatcctaatcttgaaattattctagttgatgacggtgcaactgatggttctgctcaagtgtgtgatgatttttctgaaaagtatgcaaatattcaggtaattcataagaaaaatggtgggctatcctcagcaagaaatgctggaattgaagctatgaagggagaatacgtatttttcttagatagtgatgactggattgctaatgatgcaatttctcaattatatgatgatatggtggaatataatgcagatataacagggattagtttttatcaagcatattcagacggtaatttagtattaaatacacatcttattgaaaaacaaatgctttcaaagaaagaggctttacgtactttcctatttaataattaccttactccttgttcctgtggaaaactttataaagcaagtctatggaaagatataagatttccggagggacgattatttgaagatcagcttactacttataaagttatcgagttagcaaatacaattatttttaatcctgctgcaaagtatttttattttaaaagaataggatctatcggtcattctgctttttctgaaaaaacatatgacctttatgaggctgttaatgaacaatataatgaaataactaagcatcatcctgatattgaatctgatttggcggttgctaaaattacttgggaaattgtatttattaatatgatgctcaattcaaattattcagatcaagcgatagttgataaaacacgagtttttgcaagaaaacgtattttagatgtagtgaaatgtgagtttatccctaatttacgaaaatttcagattactttatttgcatataattttagtttatataaagttttatatgcaagatataaaaagaaaaatccattatcttaattattgattttatcggttgttacaatgatacctaaaaaaattcattattgttggtttggtggaaatccattacctaaaagtgtgaaaaaatgtattaaatcttggaaaaaatactgtccagattacgagattattgagtggaatgaatctaattacaatgtgcataagaacctttttataaaagaagcttatgagaaaaaaaagtttgcatttgtttcagattatgctcgtttagatgtggttcattctgaaggtgggatttatttagatactgatgttgagttgataaaacctatagatgatttattagctcatagttgttttttagcatctgaatctattgatgatgttaatacagggctaggttttggggctgaaaaaggacattggtttatcgcagaaaatatgagtgtctatgaaaatatgtactttaatatggaaaatattattacctgtgtagagattactactaaattattaatagaaagaggtttttctgctagtgataaaattcaaaatatagatgatattttcatttatccaactgagtatttttgcccattaaattataaaacccacgagttgcatataacacagaatacttattctatacatcactatgatgcaacttggcaaagccctcttatgaaatttaaaacaaaaattaagtatatattgtgtttagccggaataataaaatgaactccttagtatatagaatagatattagaacacttattttttctattttttattttacttttttagtatcggattttttattattagctcaagatggcactattacaaaagatatcatcaaatgggttaaattattctcattattgccattgctcttattaatatttaaattgcctttgaatctcttgattttaggtttttttactataatgataagtgctttttattctatttatacgggagattcgtttttattatatatatgtttgctgatgtctttttcttataaagttaattttaactttttattcaagataggattatatcttacttcaattctagttgttctaatactaacttatttcttttttgaatattttctgattggtgacagtcattttgtatatgatgcgacctattggtttaaacgttatacatttaattttgataatcctaatgcatttcctatgagaatattcgttttttttatattttatatattgcatgtaggtaagctgcgactttttgatacatttctatttgttatactatttggaatagttttctatttttcaaattctagaactgcattttatatttttattttgtgtgtccttactattcattttaaccaagtttttaatgtgctaaataatacttttgttaaattactaattaataattcaattatatttataactattttttcaatttggtcggctatatattatcaagattattattcctatttagaaccgattaacaaaattttatctaaaagaatatactttgctaatgaggcttataagagtttaggatttgaattttaccctaggaatattaaatggtggatagaagaatctgattggcatattatagataatggatatgtatatttatttatttctggtggtcttttagtaggaaatttatttatattttctataacttggcttatgtatagactaaataaatttaacctaagtaatgaggcaatattattaatgttttctatgttatatcttttatctgagagtcattttataaatatattttacaatatacctattttattattagctattttcattaataaaactaatattgtacgctatttggaatgtaaaaaatgaataaaaaccttgtaaataatagtattatgagttttttgcttacaatatctaactttatttttccattaattacttttacttatgcggcaagaattttgcaacctgataatatgggaaagtttgcattttctctatcggttgtagattatctatctctatttgctacatttggtgttgtaggttatggtgttagagcttgtgcagaagtaagaaacaataaagaagaactaactaaaacggtacaagaaattttatttattaatatttttttagctattattgcctatcttgtgatatttcttctaattagctatcagcatgcatttagagaagatactttgttattcttaattatgtcttcttgtattatctttaatgtgataggaatagaatggttatataaaagtctcgatgaatatagatacattacagtaagaagtattctattaaaaataatttcattaataatgattttatgttttgttaaagaaaaggatgattatccactttttgcattgttttttgttctaccaatttgtctatcttcgttgttaaatattataaattcaagaaaaatattgctttttaaattatttaaacttgatttatcaaagcatataaaaccaatgtttgttttatttttagtgacattatcttatacattatatgctaatgttaatgatgtgctattagctactgtaactaatacagaacaagttggttactatagtgttgctttcaaaataaaagctgcattattagctttcattactagtacaagtatggtttttttacctcgattaacagagtatattaaaaataatcaagatattgaatttattgacttattaagaaagtcttttgatctggttttttttctagctgtgccaataacattatttttctttttatacgctaaagaaacaatatttttattgtttggtgagaaatataataagtcaagtttattattgcaaaccatgatatggtctgttttttttggtggtttaaataatatattaagtgtacaaatgttattgcctttaaaaaaagataatcagttcttaatttctattttaagtggtggatgtatatctttagttgtgaattttatcttcttgagggagcttcaatcattaagtacatcaatttcagttctagttgcagaagttgttatactgattattcaattagttattctaagaaaatatattgtaagaatttttaataatttaaatcctttaaaggtgataatgtcggtttttttttctatatggtttgttaatttaatttatgccaattttattgctctaggtaatagtttcttagagtatattatttctatttttatattttcattattttatgtgtttttacttttttttagtaaagaaagatttgttcatgatgtgtttttttatataaggagtaaatttgattaatttattaattagtattctagctaaaattctttctaggatttctaaactgattttgaatataaaaaaacggaaggaatacaaacgagttggctctatagttgattcaaagaatatagatttgagttttatttgtggtaactattgtagagtagggagagatactgtaattgagaaaaatgttattatggggagattatcttacattaattcagatatgggaaaaacatatattggtagtaatgtaaagattggtagtttatgctcaatttcctcaggtgtaataattgctcctgtaaatcattacctaaattatgtgacaacgcacccattactttataattcctattatagtagcattttaaatattaattctaatctgttatctcaacaagaattagatgcaaatgtatcaacagtgattggtaatgatgtatggattggagctaatgtgattataaagagaggagtaactataggagatggagcggttattggtgcaggtagtattataacaaaagatattccttcttatgcagtagtagcaggagttccagctaaaattattaaatatcgtttttcaaaagatgtaatagaaagcctgaaagatagtaagaatgtttgggaattatctacctcagaattagaagagaatttttctcatttatatgatgttgagaaatatcttaatagatttaagttgtaggattaatttttagtctaggattttagtatgagtaagaaaaatatagttgcacaaactttattactttgcttagatttattactaattagtatggcaatctttttagctgtatttattagaaataatattttaccgaatattatgttatttgagcctgtatcatatatagagtatctagtatacccatttccttatgtaatcattgttacattgtttatgtggtttgggctatatacaagaagatatgatttatggcaggagtcattatttattataaaagtatgttttatttcttttattattatctttgcaacattagcattgggtaagaatatagaatattattctagagctgttttattattatctcttttcttatcagtgatatttttaccaataggtcgttattttttgaaaaaaagcttgtttagactgggtctttgggaaaggaaagtaaagtttattggcaatttaaataagaatgaaattgggatttttaattctcctcatgtaggatatgtgttatctaaagatgatacatatgatgttatatttatatctagtggtgataagagtgtatcagaattaaatgatttaattgaaagtaataaattattgaatcgtgaggttctatttatccctgtgttaaatcaatatgattttactcaatctgttttgtacaataattttagtacaaggctaaatctatttacgttagaaaataaattacttggaaagcaaaataaaattttgaagtatttactagattatgtactagtattatctactttacctttttggggggggctgattttacttattagtataaaattaaaattagaagatcctaaagggaaaatatttttcttacaaaagagattaggtcaagagggtaagatattctattgttataaatttagaacaatggtttcagaccagagctttatgcaacaatggcttattgataatccagaagaaagagattattacgctgtgtatcataagtatattaatgatcctagaattactaaattcggacattttttgcgaagaacatctttagatgagttaccccaattatttaatgtacttaaaggggatatgagtttagttggaaatagaccttatatggttgaggaacaacaaaaaatgaaagatgctgccagtattattttgatgtcaaaaccaggagtaacaggtttatggcaagtaagtgggcggagtgacgtttcatttgaagaacgtttacaaattgattcttggtatattaaaaattggtctatttggaatgatattgttattttattcaaaacagttggtgttgtattaagaaaagatggagcatcttagtaataatgtaattacattaaattattatagatagggattattatgaaaaaaattttagtcaccggtggtgcaggttttattggctctgcggttgtacgtcatattataaatgatacacaagatagtgttgtaaatgttgataaacttacctatgcgggtaatttagaatcgttattaatggtagaaaatagccctcgttacgtatttgagcaagtagatatttgtaatcgtgcggaacttgatcgcgtatttgcccaacatcagcctgatgcagttatgcacttagccgcagaaagccatgttgaccgttcaatcgatgggccggctgcttttatcgaaacaaatattgtcggtacttacactttgctcgaagctgctcgctattattggaatagtttagatgctgataaaaaatcattattccgttttcatcatatttctacggatgaggtatatggtgatttggaaggtacagaagatttgtttacggaaacgacgccgtattctccgtctagcccatattcggcttctaaagcgtcaagtgatcatttagtccgtgcttggcttcgtacttatggattacctacgattgtgaccaattgttcgaataactatggtccgttccattttcctgaaaaattaattcctttaatgattttaaatgctttagagggtaaaccattaccagtttatggtaatgggcaacaaatccgtgactggttatttgtagaagatcatgctagagcattatacaaagtggtaacggaaggtaaggtgggagaaacttataatataggtggacataatgaaaaagctaatattgatgttgttcgtactatttgtagtttattagaagagcttgtaccaaataaaccggcgggtgtgcataaatatgaggatttaattacctacgttacagatcgtccagggcatgatgttcgttatgcaattgatgcaacaaaaattggacgagaattaggttggaagccacaagaaacatttgaaacaggtattcgtaaaacagtcgaatggtatttaaataatacagagtggtggagtcgtgtattagacggttcttacaatcgtgagcgtttaggttcaaattaatattattacaagcgatccaatttttaataaggtttacaatatgaaaggtattattcttgcaggtggctcaggtactcgtctttacccgattactcgtggcgtgtcaaaacagctcttaccggtatacgataaaccaatgatttattatcctttatcagtacttatgcttgcaggtatccgagaagtcttaattattacaacaccggaggataatgagagctttaaacgtttattaggcgacggttctgatttcggtatccaactttcctatgctattcaacctagtccagatggcttagctcaagcatttttaattggtgaagagtttatcggtcaggacagtgtatgtttggttctaggtgataatatcttctacggtcagcattttactcaatctttacaagaggctgtaaaatcggtagaaacgaaaggtgcgactgtatttggttatcaagtgaaagatccggaacgttttggtgtggtagagtttgatgacaatttccgtgcattgtctattgaggaaaaaccgattcaacccaaatctaattgggcggtaaccgggttatatttctatgataaccgagtagtagaatttgcaaaacaagtaaaaccctctgcacgtggcgaattagagattaccactcttaatgagatgtatcttaatgatggttcacttaatgtacaattattagggcgaggctttgcttggttagataccggcacacatgatagcttacatgatgcggcagcatttgtgaaaacagtacaaaatctacagaatttacaggtagcatgcttagaggaaattgcctatcgtaacggttggttatcacttgagcaacttgaagcattaacaaaaccgatggcgaaaaatgaatacggtcaatatttgttacgtttaacaaaaggaacaaaataatggcacgtttcttaattacgggagcgaagggacaggttggatattgtcttactaagcaattacagagcaaagcagatgtcttagcagtagatcgtgatgagcttgatataacaaatcgtgatgctgtatttaaagttgtcagagagtttcatcctgatgttattattaatgctgccgcacatactgctgtagatcgggctgagagtgaaatcgaactatcggaagcgattaacgtgaaaggcccacaatatcttgcagaagcagccaatgagattgatgcaatcattttacatatttcaacggattatgtctttgaagggacaggttctggagaatataaagaaaatgatgaacctaatccacaaggcgtatacggcaaaacaaaacttgccggagagatagcagttcaacaggcaaataaaaggcatatcattttgcgtactgcttgggtatttggtgaacatggtaataactttgttaaaacgatgctccgtttagcaaaagaaagagaatctttgggaattgtgagtgatcaatttggcggacctacctatgcaggggatattgcgagtagcctgattcatatagcaaatatcattcttaatagtaagatagatgtatttggtgtttaccattttactggcaagccttatgtaagttgggccgattttgctaagaaaatttttgatgaagctgtttcgcaaaaggtattagaaaaagcaccgcttgttaattttattgctacaagtaattatccaacatcagcaaaacgaccggcaaattctcgcttagatttaactaaaattgatgaggtttttggtattaaaccgagtaattggcaacaagcattaaaaaatattaaggcatatgcgtaatgaagattattgaaacaaatattccggatgtaaagcttttagaacctcaagtatttggtgatgaacgcggtttttttatggaaatttttcgagatgaatggttcagacaatatgtcgctgatcgtactttcgttcaagaaaatcattcaaaatctattaagggagttttgagaggcttacattatcaaactgaaaatacacaaggcaagttagtgcgtgtagtgcaggggtctgtgtttgatgtagcggtagatttacgtaaaagttctccgacttttggacaatgggttggggaagtattatccgctgaaaataaacgtcaactttgggtccctgaaggatttgctcacggtttttatgtattgacagaaaccgctgaatttacctataaatgcacagattactataatccaaaagcggaacattcattgatttggaatgatccgacagtagcgattaattggaatcttggtggcgcgcctagtttatcagcaaaggatttagctggtaaggtgttaaatgaagctgttttatttgaatagtaaattctctatttactttttatcttgactacgatataattggatacctttttttagttctatgtcgccaaaaattgtgtgcgactttatttaaacatatatttcctgaggt

SEQ ID NO: 43 - DNA, 22933 bp, Artificial Sequence
  pDOC_E.c.5_Drfb::KanR_APP2 LPS(cod.opt.)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataatcggcattttcttttgcgtttttatttgttaactgttaattgtccttgttcaaggatgctgtctttgacaacagatgttttcttgcctttgatgttcagcaggaagctaggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacagataccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcgaactcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagctagagtcgatcttcgccagcagggcgaggatcgtggcatcaccgaaccgcgccgtgcgcgggtcgtcggtgagccagagtttcagcaggccgcccaggcggcccaggtcgccattgatgcgggccagctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaatcttctctcatccgccaaaacagccaagctcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctcattaccctgttatccctactagcaggtagctgcttagttcaccgttattccactcggtaaaggtctgcgcaagttcttcgttggtgaggttcagaccacctttaagcagagaataggcttcagcaatcagctgcatatcgccgtattcaataccgttatgaaccatcttcacatagtgacctgcgccatcggcaccaatataggtaacgcatggctcaccgtcttcagccactgcggcaattttagtcaggatcggcgcaacaagttcataggcttctttctgcccaccaggcataatggaaggacctttcagcgcaccttcttcaccaccggagacaccggtaccgataaagttaaagccttcggcagaaagctcacggttacgacgaatggtgtcctggaagaaggtattaccaccatcaatgatgatgtcacctttatcgaggtatggcttgagggaatcaatagcagaatcagtgcctgcacctgctttcaccattaacaggatacgacgaggcatttccagagattcaacaaactctttcaccgtataataaggaaccagtttcttgcctggattttcggcaatcacttcttccgtcttttcgcgggaacggttgaaaatagagacggtataaccacggctttcgatgttgagcgcaaggttacgccccatcactgccataccaactacgccgatctgttgctttgacattgtttactcctgtcagagatttgtaacttacttaattatattaattgagcatgtaatcttctgataaaaatcatttgtacgatattttcagttacatactatgcgtatcgggaattcaagcttggatcccgggtacctttctgtcccattactgatgaaacacggcttatttgtttatcgccacaaagacccgattgaattacgcccaattttaggacaaatggcagggctagcgctaattaccaatttattgtttagcttaggaatttttttaggttagttgcaaattccggttaaaaaaagaccgcttgtttgagagtgataatcgcaaacaagcggtcttttttgatcaaaatattattacacgtcttgagcgattgtgtaggctggagctgcttcgaagttcctatactttctagagaataggaacttcggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccagcttcaaaagcgctctgaagttcctatactttctagagaataggaacttcggaataggaactaaggaggatattcatatggacctttttcttctcagcttagtttgaatctaaagatttctcacattatctcccaatctttgctcttcatcaataatattttttgcttatctgtaggccttctaatataatacataagctaatattggctattattttgagagtaatattatgctggaacaagctagcaatcaaaccaacgaagaaattgatctgattgagctgattcgcgtgctgtggaagaagaagctgctgattgccatcgttaccttcatctttacagctttagccgcagtttatgcctttacagcaaaagagaaatggaccagtcaagctgaagtgattgccccgcgcgtgaccgacattagtgaatatttatctttacgtaaagaatacaatctgattatgggcagtgaatttaaagaaaatgatattcgcaatgaattaaacgaattattcagccgctacgtgctgagctatgatgaagccatctccttttttaaaaccaccgatacctacaagaagctggccgagaaagaaaacgaggctggtctgcagcgcgcagccagtgaattcaccaccgaatctttaaaggtgattaagccggacgccaagaaagatttaaatgctttaggcagcaagatcgccattagcagcgaaaccgctttaagtgcccagaccgagctgaacgactttattcgccacatcagcgatattagcttcaattttagcaaaaatgaatttatttattgggtgaaggagagcatcagctctttaaactatgaaaaagaagtgatcgaacaagatcagagcatccagcgcaaagttcagatccagaatctggaggccgcactggacatggccaaaaaggccggcatcaaagagtacagcagcgcactgagcagcaacagcagcgtggcaaatttagcagtgagcgacaccaagattccgctgagcgacagtaaactggcagatggcacctatttattcatgctgggcgagaagaatttacaagcccaactggatatcgccaagaccaaagagatcgtgtacagcccgcgctattaccagatccaagaacagctgctgaagctgaatactttactgccgaaagttgagaaggtgaccggccagacatatagttacgtgagcagcccgacctatccggtgatcaaagacgccccgaagaaaggcatcattctggttatcggctttttagtgggtttactgctgagcagctttaccgtgctgatcagcgtgctggtgcgcaacaaaaagagttaattatgaggtatgaagaggaatattaacctctttatgtgccttttgatgatccaatttactgaagtgcttagactatttaggtaataaaatatagggataataatgaaacataagattttacattttagtcaagttctgggcggtgtgggccgctatctggaactgtatgacaaatacatcaacaaagatagctttgaaaacatttatattttaccgattggtgattgggaagccgcagaagcccaagataaacgttacattctgaatattgaacagagctttagcccgattaaactgattagtaatgttattaagattcgtaacattctgaaaaaagaaaaaccggatatcttttatttacacagcaccttcgccggtgttattggccgtttagcagccattggcatgcgctgcaaagtgatctacaacccgcatggctggagcttcaagatgaatgtgagccgtttaaagcagaccttctataagatcattgagggcggtttagtgtttttaacagataaattcgtgctgatcagcaagagcgaatacgaagcagcccgcagcatcggcgttagcgagaaaaaatgctgtttaatttacaatggcattgaaaccattaaaaagaccgatattgcaattattccgaagctggatgataaatatatcattggcatgattggccgcatcagcgagcagaaaaacccgatgtttttcgcccagttcgccaaggagatcattaagcagtacccgaacacctactttattttagttggcgatggtgagcagcgcgagagtctggaggattatttagaacgcaataacttaaacgacgtgttttatatcaccggctgggtgaccaacccggagagctatctgaatctgttcgaccaagctgttctgttcagtaaatgggaaggtttatgtttaagcgtgtgcgagtatatgctgtacgagaagccgattttagtgagcaacattggtggcatcaacgatttaatccagaacgaggttaacggcttcaccatcgttgagggcgatttaaaggatgccgtgaacaagagtaaccgtttacgcaatgaaccgaaaaccgtggccaagttcatcgaagccagcaacattttaattcaagaaaagttcaacgcacagaaaatggtgaatagcttagaaaaactgttcatcaaactgagcgaaaataaataatgaaggaaaaatttagttgcatcgtggtgtgttacaacccggacaatagcgtgctggataatttaaaaaactatattagttatgtgggtaaagtgattgtggtggataacagtgatgtggacaacagccagctgttcagctctttaagcgagtatctgatctacatcccgctgtacaaaaacgtgggcatcgcctatgctttaaacatcggtgtggagaagagcaaggaactgggttatgagtatattattaccatggaccaagatagcagcttcgccacaaatctggtggatgtgtacagccattacatcagcaactacccgatcgatcagattggcgctttaagcccggtgtatattaccgaccgcggtttcaaccgtaccagcaaagaagaatttaaacagattaagatcaccatgcagagcggcagcatgttcttcaccgacaaattcgatgtgatcggccgctttgacaacgatttatttttagacgtggtggactgggaatactttttccgcatttatactttaggttataaaacaattcagtgcaataaagccatgctgaaacacgccccggccgaaactttaactttatttaaaattaaaggtaaaaccattggtgtgggcgtggcaagcccgctgcgctattactatcagattcgcaatctgctgtggtgcgtgctgcacaagaaaagcttcttcatgattaagaccattgcctataagtttatcaagattctgtttctgtttaataataaaaaacagtatttaagcttcgcatacatggccatcaaggacgccttcaataaccgtttaggcgcctatgatacactgtatctggagaaaagccgtaatgaaaaatgatctgccgctgatcagcatcatcatcccgatctataacgtgaaaccgtatttagaaaagtgcgtgaacagcgtgctgagccagagctatccgaatctggagattattctggtggatgacggtgccaccgatggcagcgcacaagtttgcgatgattttagcgaaaaatatgcaaatattcaagttattcataagaaaaatggtggtttaagcagcgcacgtaatgccggtattgaggccatgaaaggcgagtacgtgttctttctggatagcgacgactggatcgcaaatgacgccatcagccagctgtacgatgatatggtggagtacaacgccgacatcaccggcatcagcttttaccaagcttatagcgacggtaatttagtgctgaacacccatctgatcgagaagcagatgctgagcaagaaagaggcactgcgtacctttttattcaataattatttaaccccgtgtagctgcggcaagctgtataaagcctctttatggaaggacatccgctttccggaaggtcgtttatttgaagatcagctgaccacctataaagttatcgaactggccaacaccatcatcttcaatccggccgccaaatacttttatttcaaacgtatcggcagcatcggccacagcgccttcagcgagaaaacctatgatttatatgaggcagtgaatgaacagtacaacgagatcaccaaacaccacccggatatcgagagtgatctggccgtggccaaaattacttgggaaattgtgtttattaatatgatgctgaacagtaactacagcgatcaagctatcgtggacaaaacccgcgtgtttgcacgcaaacgtattttagatgtggtgaagtgcgagttcatcccgaatttacgcaagtttcagatcactttatttgcctacaatttcagtctgtataaagttctgtatgcccgctataagaagaaaaatccgctgagttaattattgattttatcggttgttacaatgattccgaagaaaattcattattgctggttcggcggcaatccgctgccgaaaagtgtgaagaagtgcattaaaagttggaaaaaatattgtccggattatgaaattattgaatggaatgagagcaattacaatgtgcataaaaatttatttattaaagaggcctacgagaagaagaagttcgccttcgtgagcgattacgcccgtttagatgtggtgcacagtgaaggtggcatctatctggacaccgatgtggagctgatcaaaccgatcgatgatttactggcccatagctgctttctggccagcgaaagcatcgatgacgtgaataccggtttaggctttggtgccgaaaaaggccactggttcatcgccgagaacatgagcgtgtatgaaaatatgtactttaatatggaaaatattatcacttgtgtggagatcaccaccaaactgctgatcgaacgcggctttagcgccagcgataaaattcagaatattgatgatatttttatttatccgaccgaatatttttgcccgctgaactacaaaacccacgaactgcacatcacccagaacacctacagcatccaccactatgatgccacttggcagagcccgctgatgaaattcaaaaccaagatcaagtacattctgtgtttagccggcattattaaatgaattctttagtgtatcgcattgacatccgcactttaatttttagcatcttttattttacctttctggtgagcgattttttactgctggcccaagatggcacaatcaccaaggacatcatcaagtgggtgaagctgttttctttactgccgctgctgctgctgatcttcaagctgccgctgaatttactgattctgggcttttttaccattatgattagtgccttctacagcatctataccggtgacagctttttactgtacatctgtttactgatgagctttagctacaaagttaattttaattttttatttaagattggtttatatctgaccagtattttagtggtgctgattctgacatattttttctttgagtactttctgatcggcgacagccactttgtgtacgacgccacctactggttcaaacgctacacctttaactttgataacccgaacgcatttccgatgcgcatctttgtcttttttattttttacattctgcatgtgggcaaactgcgtttattcgacacctttctgttcgttattctgtttggtatcgttttttactttagcaacagccgtacagccttttacatttttattctgtgtgttctgaccattcattttaatcaagtgtttaatgttctgaataatacctttgttaaactgctgattaacaatagcattatctttatcaccatttttagcatttggagcgcaatctattatcaagattactattcttatctggaaccgattaataaaattttaagcaaacgtatttattttgcaaacgaagcctataagtctttaggcttcgagttctacccgcgcaatatcaagtggtggatcgaggagagcgactggcatattatcgacaatggctatgtttatttatttatcagcggcggtttactggtgggcaacttatttatcttttctattacttggctgatgtatcgtctgaataaatttaatttaagcaacgaggccattttactgatgtttagcatgctgtatttactgagcgagagccattttatcaatattttttataacatcccgattttactgctggcaatttttattaataaaaccaatattgtgcgttatttagagtgtaaaaaatgaataagaatctggtgaataatagcattatgagctttttactgaccatcagcaacttcatcttcccgctgatcaccttcacctacgccgcacgtattctgcagccggacaacatgggtaagttcgcctttagcttaagcgtggttgattatttatctttatttgccacctttggtgtggtgggttatggcgtgcgtgcttgtgccgaagttcgcaataataaggaagaactgaccaaaacagtgcaagaaattctgtttattaatatctttttagcaattattgcatatctggttatttttctgctgatcagttatcagcatgcctttcgcgaggacacactgctgtttctgatcatgagtagctgcatcattttcaacgttatcggcattgagtggctgtataaatctttagatgagtaccgctacatcaccgtgcgcagcattttactgaagattattagcttaatcatgattctgtgctttgtgaaggagaaagacgattatccgctgttcgctttattctttgtgctgccgatctgtttaagcagcttactgaacattattaacagccgcaaaattttactgtttaaactgtttaagctggacttaagcaaacatattaaaccgatgtttgtgctgtttctggttactttaagttacactttatacgccaacgtgaatgatgtgctgctggccacagtgaccaacaccgagcaagttggctactatagtgtggcatttaaaatcaaggccgctttactggcatttatcaccagcaccagcatggtgtttctgccgcgcttaaccgagtatatcaaaaacaatcaagatatcgaatttattgatctgttacgtaaaagctttgatctggtgttctttttagccgtgcctattactttattcttttttctgtatgccaaagagaccatctttttactgtttggtgaaaaatataacaagagctctttactgctgcagaccatgatctggagcgttttcttcggcggtttaaataacattttaagcgttcagatgctgctgccgctgaaaaaggacaatcagtttttaatcagtattttaagcggcggctgcatttctttagtggtgaatttcatctttttacgcgaattacagagtctgagtaccagtatcagcgttctggtggccgaagtggtgattttaatcatccagctggtgattttacgcaagtacatcgttcgtatcttcaataatttaaatccgctgaaagttattatgagcgtcttttttagcatttggtttgtgaatttaatctatgccaactttatcgctttaggcaacagctttctggagtatattattagtatttttatcttctctttattctatgtgtttctgctgtttttcagcaaagaacgctttgtgcatgatgtgttcttttatattcgcagcaaatttgattaatctgctgatcagcattctggccaaaattttaagccgcattagcaaactgattttaaatatcaagaagcgtaaagagtacaagcgcgtgggcagtattgtggatagcaaaaatattgatttaagctttatctgcggcaattattgccgcgtgggccgtgataccgtgattgagaagaacgtgatcatgggccgtttaagctacatcaacagcgacatgggcaagacctatatcggcagcaatgtgaagatcggctctttatgtagcatcagcagcggcgtgatcattgccccggtgaaccactatttaaactatgtgaccacccacccgctgctgtataacagctactacagcagcattctgaacatcaacagcaatttactgagccagcaagaactggacgcaaacgtgagcaccgtgattggcaatgatgtgtggatcggtgccaacgtgatcatcaaacgcggcgtgaccattggcgatggtgcagtgatcggcgctggtagcattatcaccaaagacatcccgagttacgcagtggttgccggcgtgccggccaaaatcatcaaatatcgctttagcaaagatgtgatcgagtctttaaaggacagcaagaatgtgtgggaactgagcaccagcgaactggaggaaaactttagccatttatacgacgtggaaaaatatctgaaccgttttaaactgtaagattaatttttagtctaggattttagtatgagtaaaaagaacatcgtggcacagactttactgctgtgtctggatttactgctgatcagcatggccatctttctggcagtgtttattcgtaataatattttaccgaacatcatgctgttcgagccggtgagctatatcgagtatttagtttatcctttcccgtatgttattattgtgactttattcatgtggtttggtttatacacacgtcgctacgatctgtggcaagaatctttatttatcatcaaagtgtgctttattagttttattatcatttttgcaactttagccttaggtaaaaatattgaatactacagccgtgccgtgctgctgctgtctttatttttaagcgtgatttttctgccgatcggccgttattttctgaagaaatctctgtttcgtttaggtttatgggaacgcaaagtgaaattcattggcaatctgaataaaaacgaaattggcatcttcaacagcccgcacgtgggttacgtgctgagcaaggacgacacctacgacgtgatcttcatcagcagcggcgataagagcgttagcgaactgaacgatttaatcgagagcaataaactgctgaaccgcgaggttctgttcatcccggttctgaaccagtatgacttcacccagagtgttctgtacaataatttcagcacccgtttaaatttatttacactggagaacaaattactgggcaaacagaataaaattttaaagtatttactggactacgttctggtgctgagtacactgccgttctggggcggtctgattttactgattagcattaagctgaaactggaagatccgaaaggcaaaattttctttttacagaagcgtctgggccaagaaggcaaaatcttctattgttataagtttcgtaccatggtgagcgaccaaagcttcatgcagcagtggctgatcgataacccggaggaacgcgactactatgccgtgtaccataagtatattaatgacccgcgcatcacaaaattcggccattttctgcgccgtaccagtctggatgaactgccgcagctgtttaatgtgctgaagggcgatatgtctttagttggcaatcgcccgtatatggttgaagagcagcagaagatgaaggacgccgccagcatcattctgatgagtaaaccgggcgttaccggtttatggcaagttagtggtcgtagcgatgtgagctttgaagagcgtttacagattgacagctggtatatcaaaaattggagcatttggaacgatattgttattctgttcaagaccgtgggcgtggtgctgcgtaaagatggcgccagttaataataatgtaattacattaaattattatagatagggattattatgaagaaaattctggttactggtggcgctggttttattggcagtgccgtggttcgccatatcatcaacgatacccaagatagcgtggtgaacgtggataaactgacatacgccggcaatctggagtctttactgatggtggaaaatagcccgcgctacgtgttcgaacaagttgacatttgcaatcgcgccgaactggaccgtgtttttgcccagcatcagccggatgccgttatgcatctggccgcagaaagccacgttgatcgcagcatcgatggcccggccgccttcatcgagaccaatatcgtgggcacatatactttactggaggccgcccgctattattggaatagtctggacgccgacaaaaagtctttatttcgcttccaccacattagcaccgatgaggtgtatggcgatttagaaggcaccgaggatttatttaccgaaaccaccccgtatagcccgagcagcccgtacagcgcaagcaaagcaagcagcgatcatctggtgcgcgcttggctgcgcacatatggtttaccgaccatcgtgaccaactgcagcaacaactacggcccgttccattttccggagaaactgatcccgctgatgattttaaatgctttagaaggtaaaccgctgccggtgtatggtaacggccagcagattcgtgattggctgttcgtggaggatcacgcccgtgctttatataaggttgtgaccgaaggcaaagtgggcgagacctacaatattggtggccacaacgagaaggccaacatcgacgttgtgcgcacaatttgctctttactggaggaactggttccgaataaaccggccggcgtgcataagtatgaagatttaatcacatatgtgaccgaccgccccggtcacgatgttcgttacgccattgatgccaccaagatcggtcgcgaactgggttggaaacctcaagaaaccttcgaaaccggcatccgtaaaaccgtggaatggtatttaaacaataccgagtggtggagccgtgtgctggatggtagctacaatcgcgaacgtttaggcagcaactaatattattacaagcgatccaatttttaataaggtttacaatatgaaaggcattattctggccggcggtagcggtacccgtttatatccgattacacgcggtgtgagcaaacagctgctgccggtgtatgataagccgatgatctattatccgttaagcgtgctgatgctggccggcatccgtgaggtgctgattattaccaccccggaggacaacgagagctttaaacgtctgctgggcgatggcagcgatttcggcattcagctgagttacgccattcaaccgagcccggatggtctggcacaagcttttctgatcggtgaagagttcatcggccaagatagcgtgtgtttagtgctgggcgacaacattttttacggtcagcatttcacccagagtctgcaagaggccgttaagagcgttgagaccaaaggtgccaccgtgtttggctaccaagttaaagatccggaacgctttggcgtggtggagtttgacgataacttccgcgctttaagtatcgaggagaaaccgatccagcctaaaagcaactgggccgtgaccggtctgtacttctacgacaaccgtgtggtggaattcgccaaacaagttaagccgagtgcacgcggcgagttagagattaccactttaaacgaaatgtatttaaacgatggcagtctgaacgtgcagctgctgggccgcggttttgcatggctggataccggtacccacgatagtctgcacgacgccgcagcctttgtgaaaaccgttcagaatttacagaatctgcaagttgcttgtttagaagaaatcgcctatcgtaacggctggctgagcttagagcagctggaggctttaaccaaaccgatggcaaagaacgagtatggccagtatctgctgcgtttaaccaaaggcaccaaataatggcacgttttttaatcaccggcgcaaaaggccaagttggttattgtttaaccaagcagctgcagagcaaagccgatgttctggccgtggatcgcgatgaactggacatcacaaaccgcgatgccgtgtttaaagtggtgcgcgaattccacccggacgtgattatcaatgccgccgcccataccgcagtggatcgtgcagaaagcgagatcgaactgagcgaagccatcaacgttaagggtccgcagtatctggccgaggcagcaaacgagatcgacgccatcattttacacattagcacagactacgtgttcgagggcaccggcagcggcgaatataaagagaatgatgaaccgaacccgcaaggtgtgtacggcaaaaccaaactggccggcgaaatcgcagttcagcaagctaacaagcgccatatcattctgcgcaccgcttgggttttcggcgaacacggcaacaacttcgtgaaaacaatgctgcgtttagccaaagaacgcgagagcttaggcattgtgagcgatcagttcggtggtccgacctatgccggtgacatcgccagctctttaattcatattgccaacatcatcttaaacagtaaaattgatgtgttcggcgtgtaccatttcaccggtaagccgtatgtgagctgggccgatttcgccaaaaagatcttcgacgaggccgttagccagaaggttctggaaaaagccccgctggtgaatttcatcgccaccagcaactatccgaccagcgccaaacgcccggcaaacagccgtttagatttaaccaaaatcgacgaggtgtttggcatcaagccgagcaattggcagcaagctttaaagaatatcaaagcctatgcctaatgaaaattatcgaaaccaacatcccggatgtgaaactgctggaaccgcaagtttttggcgacgagcgcggctttttcatggagatcttccgcgacgagtggtttcgccagtacgtggcagatcgcacctttgttcaagaaaaccacagcaagagcatcaagggtgtgctgcgcggtctgcactatcagaccgaaaacacccaaggtaaactggtgcgtgtggtgcaaggtagcgtgtttgacgtggccgtggatctgcgcaaaagcagcccgacctttggtcagtgggtgggtgaagtgctgagcgccgaaaataaacgtcagctgtgggtgccggaaggcttcgcccatggtttctatgtgctgaccgagaccgcagagtttacctacaagtgcaccgactactataacccgaaagccgagcattctttaatctggaacgatccgaccgtggccattaactggaatctgggtggtgccccgtctttaagtgccaaagatctggccggcaaagtgctgaacgaagcagtgctgtttgaataataaattctctatttactttttatcttgactacgatataattggatacctttttttagttctatgtcgccaaaaattgtgtgcgactttatttaaacatatatttcctgaggtgatggcatttcatatctcgagctcatatgctagcgtcggatatgaatatcctccttagttcctattccgaagcagctccagccaattgaccggtaataactgaaacagtttctttattcatttgaaaatttcttatatgctttcattaacattacttaagcacgctaccgcccctggcttaacagctaccagtgcactaattaaaaagttatgttgcaaagagcatactcagctcatgtaaaaacattatatccagtattcattatctgattattcaaaaggagaaacaatacctctgaaaaaaaaatcgctagcatattgattttatgcccattacctctatgattaattaaaaaactcactaacagaaatattcattatactatacttctacaacaagaagtttactgctactataaatttctcggctgccgttacgcccaatcagatttttattcacttaaaattatctcaatacatttgcggaacttcgccccttctttcaggttgcgcaatccatacttcacaaacgcctgcatatagcccatttttttaccgcagtcgtagctgtctccagtcatcagcatggcgtcaactgactgttttttcgccagcccggcaatggcatcagtcagctgaatacgcccccatgcaccaggctgagtgcattcaagttccggccaaatatcggcagaaagcacatagcgaccaacggccatgatgtctgagtccagcgtctgcggctgatccggtttttcgataaattcaacaatgcggctgactttgccttcacgatccagcggctctttggtctgaataacggagtattcagaaaggtcacccggcatacgttttgccagcacctggctacggcccgtttcattgaaacgcgcaatcatggcagcaaggttgtagcgcagcgggtcggcactggcgtcgtcgatcacaacatctggcagtaccacgacaaatgggttgtcaccaatggcgggtcgtgcacacaaaatggagtgacctaaacctaaaggttcgccctgacgcacgttcataatagtcacgcccggcgggcagatagattgcacttccgccagtagttgacgcttcacgcgctgctcaaggagagattctaattcataagaggtgtcgaagtggttttcgaccgcgttcttggacgcgtgagttaccaggaggatttctttgatccctgcagccacaatctcgtcaacaatgtactgaatcattggcttgtcgacgatcggtagcatctctttgggaatcgccttagtggcaggcaacatatgcatcccaagacctgctaccggtataactgcttttaaattcgtcatatcgattaccctgttatccctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 44 - DNA, 23019 bp, Artificial Sequence
  pDOC_SL1344_Drfb_KanR::APP2 LPS(cod.opt.)
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagggataacagggtaatcgatatgttatggtgccgactaagacgtaatgtagagcgtgccatcattatccctggcagcagagtaattcatgctggcgaaaacaagctaaagagctataattcagcaaccattttacaggtggaagaaacaatgatgaatttgaaagcagttataccggtagcgggtttgggtatgcatatgttgcctgccaccaaggcaatcccaaaagagatgctaccgatcgtcgacaagccaatgattcagtacattgtcgatgagattgtggctgcagggatcaaagaaatcgtgctggtgactcacgcgtctaaaaacgccgttgagaaccacttcgacacctcttatgaacttgaatcacttcttgagcagcgcgttaagcgtcagcttttggcggaagtgcaatctatctgcccaccgggcgtgacgattatgaacgttcgccaggcgcagccgttagggctggggcattctattctgtgcgcgcgtccggtcgtgggcgataaccctttcattgtggtactcccggatattattatcgatgatgctaccgccgatccgctgcgctataaccttgcggcgatggtggcgcgtttcaatgaaacgggtcgcagccaggtgctggcgaagcgcatgaaaggtgatttatcggagtattccgttatccagacgaaagaacctctggataatgaaggcaaagtcagccggattgtggagtttatcgaaaaaccggatcagccgcagacgctggattccgatttgatggcggtaggccgttatgtgctttcagccgacatctgggcggaactggaaagaaccgaaccgggcgcctggggccgcatccagctcaccgatgccattgctgaactggcgaaaaaacagtcggttgacgcgatgctaatgacgggtgacagctatgactgcggtaaaaaaatgggctacatgcaggcatttgtgaagtacgggctgcgcaacctgaaagaaggagccaagttccgtaagagcatagagcagcttttgcatgaataagtattaacaaccgtgataaatggttggtgataaacataataacggcagtgaacattcgaagcggcaagttggctgaaacgagtgttgactgccgttttagttttgtataaagggcttaagtaacaaggggttatctggagcattttaatgctgattttataagattaatccttgtttccggatgcaattaataagacaattagcgtttaagttttagtgagctttgccctgctgggcgaggtttgcaacaagtcgatatgtacgcagtgcactggtagctgatgagccaggggcggtagcgtgtgtaacgacttgagcaattaatttttattggcaaattaaataccacattaaatacgccttatggaatagaaaaaccggtcaattgctcgagatatgaaatgccatcacctcaggaaatatatgtttaaataaagtcgcacacaatttttggcgacatagaactaaaaaaaggtatccaattatatcgtagtcaagataaaaagtaaatagagaatttattattcaaacagcactgcttcgttcagcactttgccggccagatctttggcacttaaagacggggcaccacccagattccagttaatggccacggtcggatcgttccagattaaagaatgctcggctttcgggttatagtagtcggtgcacttgtaggtaaactctgcggtctcggtcagcacatagaaaccatgggcgaagccttccggcacccacagctgacgtttattttcggcgctcagcacttcacccacccactgaccaaaggtcgggctgcttttgcgcagatccacggccacgtcaaacacgctaccttgcaccacacgcaccagtttaccttgggtgttttcggtctgatagtgcagaccgcgcagcacacccttgatgctcttgctgtggttttcttgaacaaaggtgcgatctgccacgtactggcgaaaccactcgtcgcggaagatctccatgaaaaagccgcgctcgtcgccaaaaacttgcggttccagcagtttcacatccgggatgttggtttcgataattttcattaggcataggctttgatattctttaaagcttgctgccaattgctcggcttgatgccaaacacctcgtcgattttggttaaatctaaacggctgtttgccgggcgtttggcgctggtcggatagttgctggtggcgatgaaattcaccagcggggctttttccagaaccttctggctaacggcctcgtcgaagatctttttggcgaaatcggcccagctcacatacggcttaccggtgaaatggtacacgccgaacacatcaattttactgtttaagatgatgttggcaatatgaattaaagagctggcgatgtcaccggcataggtcggaccaccgaactgatcgctcacaatgcctaagctctcgcgttctttggctaaacgcagcattgttttcacgaagttgttgccgtgttcgccgaaaacccaagcggtgcgcagaatgatatggcgcttgttagcttgctgaactgcgatttcgccggccagtttggttttgccgtacacaccttgcgggttcggttcatcattctctttatattcgccgctgccggtgccctcgaacacgtagtctgtgctaatgtgtaaaatgatggcgtcgatctcgtttgctgcctcggccagatactgcggacccttaacgttgatggcttcgctcagttcgatctcgctttctgcacgatccactgcggtatgggcggcggcattgataatcacgtccgggtggaattcgcgcaccactttaaacacggcatcgcggtttgtgatgtccagttcatcgcgatccacggccagaacatcggctttgctctgcagctgcttggttaaacaataaccaacttggccttttgcgccggtgattaaaaaacgtgccattatttggtgcctttggttaaacgcagcagatactggccatactcgttctttgccatcggtttggttaaagcctccagctgctctaagctcagccagccgttacgataggcgatttcttctaaacaagcaacttgcagattctgtaaattctgaacggttttcacaaaggctgcggcgtcgtgcagactatcgtgggtaccggtatccagccatgcaaaaccgcggcccagcagctgcacgttcagactgccatcgtttaaatacatttcgtttaaagtggtaatctctaactcgccgcgtgcactcggcttaacttgtttggcgaattccaccacacggttgtcgtagaagtacagaccggtcacggcccagttgcttttaggctggatcggtttctcctcgatacttaaagcgcggaagttatcgtcaaactccaccacgccaaagcgttccggatctttaacttggtagccaaacacggtggcacctttggtctcaacgctcttaacggcctcttgcagactctgggtgaaatgctgaccgtaaaaaatgttgtcgcccagcactaaacacacgctatcttggccgatgaactcttcaccgatcagaaaagcttgtgccagaccatccgggctcggttgaatggcgtaactcagctgaatgccgaaatcgctgccatcgcccagcagacgtttaaagctctcgttgtcctccggggtggtaataatcagcacctcacggatgccggccagcatcagcacgcttaacggataatagatcatcggcttatcatacaccggcagcagctgtttgctcacaccgcgtgtaatcggatataaacgggtaccgctaccgccggccagaataatgcctttcatattgtaaaccttattaaaaattggatcgcttgtaataatattagttgctgcctaaacgttcgcgattgtagctaccatccagcacacggctccaccactcggtattgtttaaataccattccacggttttacggatgccggtttcgaaggtttcttgaggtttccaacccagttcgcgaccgatcttggtggcatcaatggcgtaacgaacatcgtgaccggggcggtcggtcacatatgtgattaaatcttcatacttatgcacgccggccggtttattcggaaccagttcctccagtaaagagcaaattgtgcgcacaacgtcgatgttggccttctcgttgtggccaccaatattgtaggtctcgcccactttgccttcggtcacaaccttatataaagcacgggcgtgatcctccacgaacagccaatcacgaatctgctggccgttaccatacaccggcagcggtttaccttctaaagcatttaaaatcatcagcgggatcagtttctccggaaaatggaacgggccgtagttgttgctgcagttggtcacgatggtcggtaaaccatatgtgcgcagccaagcgcgcaccagatgatcgctgcttgctttgcttgcgctgtacgggctgctcgggctatacggggtggtttcggtaaataaatcctcggtgccttctaaatcgccatacacctcatcggtgctaatgtggtggaagcgaaataaagactttttgtcggcgtccagactattccaataatagcgggcggcctccagtaaagtatatgtgcccacgatattggtctcgatgaaggcggccgggccatcgatgctgcgatcaacgtggctttctgcggccagatgcataacggcatccggctgatgctgggcaaaaacacggtccagttcggcgcgattgcaaatgtcaacttgttcgaacacgtagcgcgggctattttccaccatcagtaaagactccagattgccggcgtatgtcagtttatccacgttcaccacgctatcttgggtatcgttgatgatatggcgaaccacggcactgccaataaaaccagcgccaccagtaaccagaattttcttcataataatccctatctataataatttaatgtaattacattattattaactggcgccatctttacgcagcaccacgcccacggtcttgaacagaataacaatatcgttccaaatgctccaatttttgatataccagctgtcaatctgtaaacgctcttcaaagctcacatcgctacgaccactaacttgccataaaccggtaacgcccggtttactcatcagaatgatgctggcggcgtccttcatcttctgctgctcttcaaccatatacgggcgattgccaactaaagacatatcgcccttcagcacattaaacagctgcggcagttcatccagactggtacggcgcagaaaatggccgaattttgtgatgcgcgggtcattaatatacttatggtacacggcatagtagtcgcgttcctccgggttatcgatcagccactgctgcatgaagctttggtcgctcaccatggtacgaaacttataacaatagaagattttgccttcttggcccagacgcttctgtaaaaagaaaattttgcctttcggatcttccagtttcagcttaatgctaatcagtaaaatcagaccgccccagaacggcagtgtactcagcaccagaacgtagtccagtaaatactttaaaattttattctgtttgcccagtaatttgttctccagtgtaaataaatttaaacgggtgctgaaattattgtacagaacactctgggtgaagtcatactggttcagaaccgggatgaacagaacctcgcggttcagcagtttattgctctcgattaaatcgttcagttcgctaacgctcttatcgccgctgctgatgaagatcacgtcgtaggtgtcgtccttgctcagcacgtaacccacgtgcgggctgttgaagatgccaatttcgtttttattcagattgccaatgaatttcactttgcgttcccataaacctaaacgaaacagagatttcttcagaaaataacggccgatcggcagaaaaatcacgcttaaaaataaagacagcagcagcacggcacggctgtagtattcaatatttttacctaaggctaaagttgcaaaaatgataataaaactaataaagcacactttgatgataaataaagattcttgccacagatcgtagcgacgtgtgtataaaccaaaccacatgaataaagtcacaataataacatacgggaaaggataaactaaatactcgatatagctcaccggctcgaacagcatgatgttcggtaaaatattattacgaataaacactgccagaaagatggccatgctgatcagcagtaaatccagacacagcagtaaagtctgtgccacgatgttctttttactcatactaaaatcctagactaaaaattaatcttacagtttaaaacggttcagatatttttccacgtcgtataaatggctaaagttttcctccagttcgctggtgctcagttcccacacattcttgctgtcctttaaagactcgatcacatctttgctaaagcgatatttgatgattttggccggcacgccggcaaccactgcgtaactcgggatgtctttggtgataatgctaccagcgccgatcactgcaccatcgccaatggtcacgccgcgtttgatgatcacgttggcaccgatccacacatcattgccaatcacggtgctcacgtttgcgtccagttcttgctggctcagtaaattgctgttgatgttcagaatgctgctgtagtagctgttatacagcagcgggtgggtggtcacatagtttaaatagtggttcaccggggcaatgatcacgccgctgctgatgctacataaagagccgatcttcacattgctgccgatataggtcttgcccatgtcgctgttgatgtagcttaaacggcccatgatcacgttcttctcaatcacggtatcacggcccacgcggcaataattgccgcagataaagcttaaatcaatatttttgctatccacaatactgcccacgcgcttgtactctttacgcttcttgatatttaaaatcagtttgctaatgcggcttaaaattttggccagaatgctgatcagcagattaatcaaatttgctgcgaatataaaagaacacatcatgcacaaagcgttctttgctgaaaaacagcagaaacacatagaataaagagaagataaaaatactaataatatactccagaaagctgttgcctaaagcgataaagttggcatagattaaattcacaaaccaaatgctaaaaaagacgctcataataactttcagcggatttaaattattgaagatacgaacgatgtacttgcgtaaaatcaccagctggatgattaaaatcaccacttcggccaccagaacgctgatactggtactcagactctgtaattcgcgtaaaaagatgaaattcaccactaaagaaatgcagccgccgcttaaaatactgattaaaaactgattgtcctttttcagcggcagcagcatctgaacgcttaaaatgttatttaaaccgccgaagaaaacgctccagatcatggtctgcagcagtaaagagctcttgttatatttttcaccaaacagtaaaaagatggtctctttggcatacagaaaaaagaataaagtaataggcacggctaaaaagaacaccagatcaaagcttttacgtaacagatcaataaattcgatatcttgattgtttttgatatactcggttaagcgcggcagaaacaccatgctggtgctggtgataaatgccagtaaagcggccttgattttaaatgccacactatagtagccaacttgctcggtgttggtcactgtggccagcagcacatcattcacgttggcgtataaagtgtaacttaaagtaaccagaaacagcacaaacatcggtttaatatgtttgcttaagtccagcttaaacagtttaaacagtaaaattttgcggctgttaataatgttcagtaagctgcttaaacagatcggcagcacaaagaataaagcgaacagcggataatcgtctttctccttcacaaagcacagaatcatgattaagctaataatcttcagtaaaatgctgcgcacggtgatgtagcggtactcatctaaagatttatacagccactcaatgccgataacgttgaaaatgatgcagctactcatgatcagaaacagcagtgtgtcctcgcgaaaggcatgctgataactgatcagcagaaaaataaccagatatgcaataattgctaaaaagatattaataaacagaatttcttgcactgttttggtcagttcttccttattattgcgaacttcggcacaagcacgcacgccataacccaccacaccaaaggtggcaaataaagataaataatcaaccacgcttaagctaaaggcgaacttacccatgttgtccggctgcagaatacgtgcggcgtaggtgaaggtgatcagcgggaagatgaagttgctgatggtcagtaaaaagctcataatgctattattcaccagattcttattcattttttacactctaaataacgcacaatattggttttattaataaaaattgccagcagtaaaatcgggatgttataaaaaatattgataaaatggctctcgctcagtaaatacagcatgctaaacatcagtaaaatggcctcgttgcttaaattaaatttattcagacgatacatcagccaagtaatagaaaagataaataagttgcccaccagtaaaccgccgctgataaataaataaacatagccattgtcgataatatgccagtcgctctcctcgatccaccacttgatattgcgcgggtagaactcgaagcctaaagacttataggcttcgtttgcaaaataaatacgtttgcttaaaattttattaatcggttccagataagaatagtaatcttgataatagattgcgctccaaatgctaaaaatggtgataaagataatgctattgttaatcagcagtttaacaaaggtattattcagaacattaaacacttgattaaaatgaatggtcagaacacacagaataaaaatgtaaaaggctgtacggctgttgctaaagtaaaaaacgataccaaacagaataacgaacagaaaggtgtcgaataaacgcagtttgcccacatgcagaatgtaaaaaataaaaaagacaaagatgcgcatcggaaatgcgttcgggttatcaaagttaaaggtgtagcgtttgaaccagtaggtggcgtcgtacacaaagtggctgtcgccgatcagaaagtactcaaagaaaaaatatgtcagaatcagcaccactaaaatactggtcagatataaaccaatcttaaataaaaaattaaaattaactttgtagctaaagctcatcagtaaacagatgtacagtaaaaagctgtcaccggtatagatgctgtagaaggcactaatcataatggtaaaaaagcccagaatcagtaaattcagcggcagcttgaagatcagcagcagcagcggcagtaaagaaaacagcttcacccacttgatgatgtccttggtgattgtgccatcttgggccagcagtaaaaaatcgctcaccagaaaggtaaaataaaagatgctaaaaattaaagtgcggatgtcaatgcgatacactaaagaattcatttaataatgccggctaaacacagaatgtacttgatcttggttttgaatttcatcagcgggctctgccaagtggcatcatagtggtggatgctgtaggtgttctgggtgatgtgcagttcgtgggttttgtagttcagcgggcaaaaatattcggtcggataaataaaaatatcatcaatattctgaattttatcgctggcgctaaagccgcgttcgatcagcagtttggtggtgatctccacacaagtgataatattttccatattaaagtacatattttcatacacgctcatgttctcggcgatgaaccagtggcctttttcggcaccaaagcctaaaccggtattcacgtcatcgatgctttcgctggccagaaagcagctatgggccagtaaatcatcgatcggtttgatcagctccacatcggtgtccagatagatgccaccttcactgtgcaccacatctaaacgggcgtaatcgctcacgaaggcgaacttcttcttctcgtaggcctctttaataaataaatttttatgcacattgtaattgctctcattccattcaataatttcataatccggacaatattttttccaacttttaatgcacttcttcacacttttcggcagcggattgccgccgaaccagcaataatgaattttcttcggaatcattgtaacaaccgataaaatcaataattaactcagcggatttttcttcttatagcgggcatacagaactttatacagactgaaattgtaggcaaataaagtgatctgaaacttgcgtaaattcgggatgaactcgcacttcaccacatctaaaatacgtttgcgtgcaaacacgcgggttttgtccacgatagcttgatcgctgtagttactgttcagcatcatattaataaacacaatttcccaagtaattttggccacggccagatcactctcgatatccgggtggtgtttggtgatctcgttgtactgttcattcactgcctcatataaatcataggttttctcgctgaaggcgctgtggccgatgctgccgatacgtttgaaataaaagtatttggcggccggattgaagatgatggtgttggccagttcgataactttataggtggtcagctgatcttcaaataaacgaccttccggaaagcggatgtccttccataaagaggctttatacagcttgccgcagctacacggggttaaataattattgaataaaaaggtacgcagtgcctctttcttgctcagcatctgcttctcgatcagatgggtgttcagcactaaattaccgtcgctataagcttggtaaaagctgatgccggtgatgtcggcgttgtactccaccatatcatcgtacagctggctgatggcgtcatttgcgatccagtcgtcgctatccagaaagaacacgtactcgcctttcatggcctcaataccggcattacgtgcgctgcttaaaccaccatttttcttatgaataacttgaatatttgcatatttttcgctaaaatcatcgcaaacttgtgcgctgccatcggtggcaccgtcatccaccagaataatctccagattcggatagctctggctcagcacgctgttcacgcacttttctaaatacggtttcacgttatagatcgggatgatgatgctgatcagcggcagatcatttttcattacggcttttctccagatacagtgtatcataggcgcctaaacggttattgaaggcgtccttgatggccatgtatgcgaagcttaaatactgttttttattattaaacagaaacagaatcttgataaacttataggcaatggtcttaatcatgaagaagcttttcttgtgcagcacgcaccacagcagattgcgaatctgatagtaatagcgcagcgggcttgccacgcccacaccaatggttttacctttaattttaaataaagttaaagtttcggccggggcgtgtttcagcatggctttattgcactgaattgttttataacctaaagtataaatgcggaaaaagtattcccagtccaccacgtctaaaaataaatcgttgtcaaagcggccgatcacatcgaatttgtcggtgaagaacatgctgccgctctgcatggtgatcttaatctgtttaaattcttctttgctggtacggttgaaaccgcggtcggtaatatacaccgggcttaaagcgccaatctgatcgatcgggtagttgctgatgtaatggctgtacacatccaccagatttgtggcgaagctgctatcttggtccatggtaataatatactcataacccagttccttgctcttctccacaccgatgtttaaagcataggcgatgcccacgtttttgtacagcgggatgtagatcagatactcgcttaaagagctgaacagctggctgttgtccacatcactgttatccaccacaatcactttacccacataactaatatagttttttaaattatccagcacgctattgtccgggttgtaacacaccacgatgcaactaaatttttccttcattatttattttcgctcagtttgatgaacagtttttctaagctattcaccattttctgtgcgttgaacttttcttgaattaaaatgttgctggcttcgatgaacttggccacggttttcggttcattgcgtaaacggttactcttgttcacggcatcctttaaatcgccctcaacgatggtgaagccgttaacctcgttctggattaaatcgttgatgccaccaatgttgctcactaaaatcggcttctcgtacagcatatactcgcacacgcttaaacataaaccttcccatttactgaacagaacagcttggtcgaacagattcagatagctctccgggttggtcacccagccggtgatataaaacacgtcgtttaagttattgcgttctaaataatcctccagactctcgcgctgctcaccatcgccaactaaaataaagtaggtgttcgggtactgcttaatgatctccttggcgaactgggcgaaaaacatcgggtttttctgctcgctgatgcggccaatcatgccaatgatatatttatcatccagcttcggaataattgcaatatcggtctttttaatggtttcaatgccattgtaaattaaacagcattttttctcgctaacgccgatgctgcgggctgcttcgtattcgctcttgctgatcagcacgaatttatctgttaaaaacactaaaccgccctcaatgatcttatagaaggtctgctttaaacggctcacattcatcttgaagctccagccatgcgggttgtagatcactttgcagcgcatgccaatggctgctaaacggccaataacaccggcgaaggtgctgtgtaaataaaagatatccggtttttcttttttcagaatgttacgaatcttaataacattactaatcagtttaatcgggctaaagctctgttcaatattcagaatgtaacgtttatcttgggcttctgcggcttcccaatcaccaatcggtaaaatataaatgttttcaaagctatctttgttgatgtatttgtcatacagttccagatagcggcccacaccgcccagaacttgactaaaatgtaaaatcttatgtttcattattatccctatattttattacctaaatagtctaagcacttcagtaaattggatcatcaaaaggcacataaagaggttaatattcctcttcatacctcataattaactctttttgttgcgcaccagcacgctgatcagcacggtaaagctgctcagcagtaaacccactaaaaagccgataaccagaatgatgcctttcttcggggcgtctttgatcaccggataggtcgggctgctcacgtaactatatgtctggccggtcaccttctcaactttcggcagtaaagtattcagcttcagcagctgttcttggatctggtaatagcgcgggctgtacacgatctctttggtcttggcgatatccagttgggcttgtaaattcttctcgcccagcatgaataaataggtgccatctgccagtttactgtcgctcagcggaatcttggtgtcgctcactgctaaatttgccacgctgctgttgctgctcagtgcgctgctgtactctttgatgccggcctttttggccatgtccagtgcggcctccagattctggatctgaactttgcgctggatgctctgatcttgttcgatcacttctttttcatagtttaaagagctgatgctctccttcacccaataaataaattcatttttgctaaaattgaagctaatatcgctgatgtggcgaataaagtcgttcagctcggtctgggcacttaaagcggtttcgctgctaatggcgatcttgctgcctaaagcatttaaatctttcttggcgtccggcttaatcacctttaaagattcggtggtgaattcactggctgcgcgctgcagaccagcctcgttttctttctcggccagcttcttgtaggtatcggtggttttaaaaaaggagatggcttcatcatagctcagcacgtagcggctgaataattcgtttaattcattgcgaatatcattttctttaaattcactgcccataatcagattgtattctttacgtaaagataaatattcactaatgtcggtcacgcgcggggcaatcacttcagcttgactggtccatttctcttttgctgtaaaggcataaactgcggctaaagctgtaaagatgaaggtaacgatggcaatcagcagcttcttcttccacagcacgcgaatcagctcaatcagatcaatttcttcgttggtttgattgctagcttgttccagcataatattactctcaaaataatagccaatattagcttatgtattatattagaaggcctacagataagcaaaaaatattattgatgaagagcaaagattgggagataatgtgagaaatctttagattcaaactaagctgagaagaaaaaggtccatatgaatatcctccttagttcctattccgaagttcctattctctagaaagtataggaacttcagagcgcttttgaagctggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaggggatcttgaagttcctattccgaagttcctattctctagaaagtataggaacttcgaagcagctccagcctacacaatcgctcaagacgtgtaataatattttgatcaaaaaagaccgcttgtttgcgattatcactctcaaacaagcggtctttttttaaccggaatttgcaactaacctaaaaaaattcctaagctaaacaataaattggtaattagcgctagccctgccatttgtcctaaaattgggcgtaattcaatcgggtctttgtggcgataaacaaataagccgtgtttcatcagtaatgggacagaaaggtacccgggatccaagcttgaattcccgagcttaccgagaagtactgaataataattgtataaattagcctgcgtaaaatctgaacgcatcaatcgctaccttaatatcatacctttgagttaacatactattcacctttaacctgccatgaccgtttgtggcagggtttccacacctgacaggagtatgtaatgtccaagcaacagatcggcgtcgtcggtatggcagtgatggggcgcaacctcgcgctcaacatcgaaagccgtggttataccgtctccgttttcaaccgctcccgtgaaaagaccgaagaagtgattgccgagaatcccggcaaaaagctggtgccttattacacggtgaaagagtttgttgaatccctcgaaacgcctcgtcgtatcctgttaatggtgaaagcgggcgcaggtactgatgcagctatcgattcgctgaaaccgtatctggaaaaaggcgatatcattattgatggcggtaacaccttcttccaggacacaatccgtcgcaatcgcgagctgtctgcggaaggttttaactttatcggtaccggtgtttccggtggtgaagagggcgcgctgaaagggccatctatcatgcctggcggtcagaaagatgcctatgaactggtggcgccgatcctgacgaagattgctgctgtggcagaagatggcgaaccgtgcgtgacctatatcggcgccgatggtgctggtcactacgtcaagatggtccacaatggtattgaatatggcgatatgcagcttatcgctgaagcttactccctgctgaaaggcggcctgaatctcagcaatgctagtagggataacagggtaatgagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgcatgcgcctgatgcggtattttctccttacgcatatcgacatccgccctcaccgccaggaacgcaaccgcagcctcatcacgccggcgcttcttggccgcgcgggattcaacccactcggccagctcgtcggtgtagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgactctagctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgagttcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtatctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgcctagcttcctgctgaacatcaaaggcaagaaaacatctgttgtcaaagacagcatccttgaacaaggacaattaacagttaacaaataaaaacgcaaaagaaaatgccgattatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 45 - DNA, 1414 bp, Artificial Sequence
  synthesized chloramphenicol resistance cassette flanked on the 3′ side by kanamycin promoter
attacacgtcttgagcgattgtgtaggctggagctgcttcgaagttcctatactttctagagaataggaacttcggaataggaacttcatttaaatggcgcgccttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaaggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcctaaggaggatattcatatggacc

SEQ ID NO: 46 - DNA, 17796 bp, Artificial Sequence
  codon optimized APP8 rfb cluster with cat/kP promoter and flanking hom.rec sites for SL1344 rfb integration (for pDOC)
attaccctgttatccctactagcattgctgagattcaggccgcctttcagcagggagtaagcttcagcgataagctgcatatcgccatattcaataccattgtggaccatcttgacgtagtgaccagcaccatcggcgccgatataggtcacgcacggttcgccatcttctgccacagcagcaatcttcgtcaggatcggcgccaccagttcataggcatctttctgaccgccaggcatgatagatggccctttcagcgcgccctcttcaccaccggaaacaccggtaccgataaagttaaaaccttccgcagacagctcgcgattgcgacggattgtgtcctggaagaaggtgttaccgccatcaataatgatatcgcctttttccagatacggtttcagcgaatcgatagctgcatcagtacctgcgcccgctttcaccattaacaggatacgacgaggcgtttcgagggattcaacaaactctttcaccgtgtaataaggcaccagctttttgccgggattctcggcaatcacttcttcggtcttttcacgggagcggttgaaaacggagacggtataaccacggctttcgatgttgagcgcgaggttgcgccccatcactgccataccgacgacgccgatctgttgcttggacattacatactcctgtcaggtgtggaaaccctgccacaaacggtcatggcaggttaaaggtgaatagtatgttaactcaaaggtatgatattaaggtagcgattgatgcgttcagattttacgcaggctaatttatacaattattattcagtacttctcggtaagctcgggaattcaagcttggatcccgggtacctttctgtcccattactgatgaaacacggcttatttgtttatcgccacaaagacccgattgaattacgcccaattttaggacaaatggcagggctagcgctaattaccaatttattgtttagcttaggaatttttttaggttagttgcaaattccggttaaaaaaagaccgcttgtttgagagtgataatcgcaaacaagcggtcttttttgatcaaaatatattacacgtcttgagcgattgtgtaggctggagctgcttcgaagttcctatactttctagagaataggaacttcggaataggaacttcatttaaatggcgcgccttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaaggaataggaacttcaagatcccctcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcctaaggaggatattcatatggaccttttttattctcagtttgaatctaaaaattctaggtattatcccccaatattaactctgcatcaataacattctttgcttatctataggctttctaatatactatgcagtcaatatatttttgttatttatttttgagaaaaccttatggcgacgccagagcaagtgagcaaccagaccaacgaggagattgacctcatcgagctggttcgcgtgctgtggaagaaaaagctcctcatcgccatcgtgacgtgcatcttcaccgccctcgcggccgtttatgccttcaccgccaaagagaagtggacgagccagaccgaagttatcgcgccacgtgtgaccgacattagcgaatatctgagtctgcgcaaggagtacaacctcatcatcggcagcgagttcaaagaaaacgaaatccgcaacgagctcagcgagctcttcagtcgctatgtgctgagttacgacgaagccatcgccttcttcaagacgacggacacgtacaagaagctggccgaaaccgagaatgaagtgggcctccagcgcgcggttgccgaattcacgacggagagtctgaaggtgatcaagccagacgccaagaaagacccgaatgcgctgggcagtaagattgcgatcagcttcgataccgcgctgagtgcccagaccacgctgaacgacttcatctgccacatcagcgacaccagtttcaacttcagtaagaatgagttcatttattggattaaggagagcattagcagtctgaattacgagaaagaggttatcgaacaagatcagagcatccaacgcaaggtgcagatccagaatctggagacggccctcgatatggcgaagaaggcgggcatcaaagagtacagcagcgccctcagcagtaatagtagcgtggccaatctggcggtgagcgataccaaaatcccgctgagcgatagcaagctggcggacggtacctatctgttcatgctgggcgagaagaatctgcaagcgcagctcgacatcgcgaaaacgaaggagatcgtgtacagcccgcgctactaccagatccaagaacagctgctgaagctgaataccctcctcccgaaagtggaaaaggttaccggccagagcttcagctatatcagcagcccagagctcccgattaagcgcgattggccaaagcgctttattctgctcctcattggcgcggtgattggtggcgttctgagcagtctgtgggtgatcggcaaacaaatcttcggccagaaataattatgaacaaggacatcaagattctgatcgcgacgcataagcagcacttcatgccgagcgacgaaatgtatctgccgctccacgtgggcaagctgggtaaagccgatctgggttaccaaggcgacgacagcggcgacaacatcagcatcaaaaacccaaatttttgcgagctgaccggtctgtactgggcgtggaaaaatctgccgaacgattatctgggtctgatccattatcgccgcttcttcagcgtgaagaaccgcgcggaacgcaaaaacaatccgctggagacgctgtatctgaccaacgaagaagccaaccagctgctgagtcagtacgatgtgatcgttccgagcaagcgcaactactacatcgagacgctgtacagccattacgccaatacgctgcacgccgaacatctggacgttacccgcgaaatcatcgcggaaaagtgcagcgagtacctcgccagctttgacgcggtgatcaaacagcgcagcggctacatgttcaacatgttcatcatgagcaaagcgctggtgaacgactactgcagctggctgttcccgattctgtttgaactggagaagcgtatcccaacggaccagtacagcgccttccatgcccgtttctacggccgcgtgagcgaactgctgttcaacgtgtggctgaaacagtacagccagagcaacccactgaaggtgaaggccatcccgtttgtgtatggcgagaagatcaactggctgaagaagggtaccgcgtttctggttgcgaaattcttcggcaaaaaatatgagaaaagcttctaagttattatagggacaaataaatgaagcgcattctggtttacggcatgaccgacaacttcggtggcatggaggcctacattcataacatctatcagcatctggataaaacccaaatccagtttgacttcgtgtgtgacttcccgaaaatgacgctgagcgactactatctggacaacggttgcaagatccacttcatcccgccgaaaaaccaaggtctgtttaagagtctgtgggcgatgtggaaagtgatcaaggagaacaactatgatgttatctatttcaacatcatgaacgcgggctacgtgctcaacatgctgccggcctttctgctgggtaagaagatcatcgcgcacagccacaatgcggacaccgacaaaaagaagctgcactacggtctgcgtctgctgctgaacatcgtgacgaagatcaagctcgcgtgcagtaaggaggccggtttcttcatgttcggcaaggaagaaaatttcagcatcattaataatgcgatcaacctcgaccgctatctgtatagcgaggagaaataccgcgacctccgccacaaactgggctggggcgataagaaggttattctgtacgtggcccgcatgaatcaccagaagaatccgctgttcgcgctgtatatcatgcgcgaactgaagcagagcatgccgaatgccgttctggtgtacgtgggtacgggtgagctgaaggaacaagttcagcagtacattctggacaacaacctcgacaacgtgattctgctgggtctgcgcaacgatgtgaacgagctcatgatcgcggccgatctgtttattctgccgagtctgtttgagggtctgccgattgttgccgttgaggcgcaagccgcgggtctcccaatcattctgagcgaaaacatcagcatcgaggcgaaactcgtgaacagcacctacttcctcccgattaacgacgtttttctgtgggttaataaaatcaaaaagattctggagatcagtggcaacaagcgctttagtgaccagctggcgctgagcaaagcgggttacaacatcgagagcgtggtgaagaacatccagaagattctcgtgaattaaggtttggtatgaataacaccaagatcagtctgatcttcgcgtgctataacgtgagccagtatctggacaatctgttccagctgctgacgaaccagccgtaccaaaatattgaaatcattttcgtggaggactgtgccacggacgatacgaaggcgaaactccagagcttcaacgatccgcgcgtgaagctgctgtgcaacgagaaaaatatcggtgcggccgaaagccgtaaccgtggcatccagatcgttaccggcgaatacatctggttcccggatccagacgatctgtttgacgaactgctgctgaccaaggtgaacaccatcatccagaaaaaccgcccggatgtgatcagcatcggcatgcaagaacgctacgagatcaacggcaagacggactacacgaaggacatcatcagccgctacgacggcctcattaccggcgacttcaccgatgttttcgtggatctggaggaaagctttctgtttggctacacgaataacaagttttacaaggccaacatcatccataagtaccgcattctgaacgagcaccaagcgctgaaggaagatttcgaattcaatatcaaggtgtttaaacaagttagtaacttctatctgctgaatgaaccgctctatttctacatgaagcgcaacaacggcagtctgaccagcaaattcgtgccggactatttccgcatccacatgcagaccctcgccagcttcaaaagtctgatcgaggtgaaggccaccatcaacgacaacgtgaaccgtctgctggtgaaccgcttcgttcgctactgcctcagcgcgatcgagcgcaacagcagtctgaaaagcggcatgagctttctggagcagaaccaatggattaaggaaaatatctttaatcaagaaaaatacaacgagtatctgctgctgagcgatctggtgaacaagaaacagaagctgttttactttctgatcaagtatcgcatcggttttctgctcgtgacggccgcgaacatcgtgaaactggtgaaggcgaagttcccgattctgttcgtgaagctgaagggttaattaactggattttaaaatgaagaagtaccagatcgtggagctgagtaccgaacacaaccatgcgggcagcaaggccgtgcaagatgtgtatgagatcgcgctcagcatgggttacaaggcgaatgtggttcgcacggccaccagtgtggatagtctgctggccaaaattctgcgccaagttatcttcttcatcgactggctgaagatctacttcagcatcgagagtaacagcatcgtgctgatccagaacccgtactaccacaaacagctcatccgtaactggattctgaatcgtctgaagcgcattaaaaaagtgaagtttatcagtctggttcacgacgtggaagagctgcgcaagagtctgtacaacaactactataaaaacgagttcgagaccatgctgagtctggcggacagcatcatcgtgcacaatgataagatgaaaagctttttcatcaaaaagggctacagcgaggacaaactcatcagtctgggcatcttcgactatctgcagaagagcgtggacaaaaagcgcgtgagcttcgaacgtgcgatcagcgtggcgggcaacctcgatatcaagaagagcagctatattgcgcagctcggcagcctcccggcgatcaaagcgcatctgtacggtccgaacttcgaacatagtctggaggcgttcccgaacatcgaataccacggtagcttcccggccacggaaatcccgcagaaactcgtgagcggttttggtctggtgtgggacggccagagcattgaaacgtgcaccggcgacttcggcgagtacctccagtacaataacccgcacaagctgagcctctatctgagcagtggcatgccggttgtgatctgggacaaagccgccgaggccgatttcgtgaagaaacacaacgtgggtctgtgcgtgagcagtctgagcgagctccaagacaagctcaacgtgatgaccgagcaagaatttgaagaaatggtgaacaacgtggaaaaacagaccgcgtgcctcatcagcggcgagtacaccaaaaaggcgatcagcgaggcggaacgtgtgatctaagaatgttcctctatctgctggtgttcagtctgctgctgattctgatcttcaatctgctcatcgtgaatctggactacatgcacccgagcatcctctttgttgtgccatttctggtgtttggcgtgacgagcattctgggcgaggaggcgtataagatcatcttccacgaggagacgctgctggtgatcgttagcagcgcgctgatcttcaccttcatcacgctgctgagccagaccgtgtacaaaagcaaagagaatctgaacttcccgctgaccgagatcatcatcagtaagaaagtgacgctgttttttattgtgttcttcatcgtgacccagctggcgttcatcaagtatctggaggccattagtctggcccacttcggttacagcggcagtctgggtgagatgatcagtctgtacgacgtgatgacgaagttctggaccgagatcttcagcgaactcaacgtgccgatcccgctgctctaccgtatcggcaatccaatcacgcaaggcttcggctatctgattgtgtatattttcatccacaactacgttgccaccaagcgcatcgataagctgcatctgctgatcattctgctgctgtgtctgaacatcattctcaacggcagccgcagtccgatcttccgcatcgttacgatgatgctgatcaccttttatgtgctgtataacaagcagaacaacgtgcgtcgcggcaacatcaagtttctgctgaagagtctgctgatcgtgatcttcagcggcaccttcttcattgcgctgctgagtctgatgggccgtgaaaacgatctggacatgttccattacatttttatctacgttggtgcgccgctggtgaacctcgataactatctggcgtttcgtccggatggtagctacgccaccatctttggcgagcaaacgtttcgcggtctgtacgcctatatcgcgaagatcatcagcgatgagagtctgatcttcccgacgatcgatcagttcacgttcagcaacaacggtctggagatcggtaacgtgtataccaccttctatagcttcatctacgatttcgagtacgtgggcttcatcccgctgattctgattatcgcgctgtactacgtgttcacgtatcagcgcctcaagacgcgcgccatcaagaccaataaagtgcatttcagtctgttcatctatgcctacctcttcaacgacctcatcatgctggccttcagtaatcgcttctacaccacggtgctggacatcggcttcatcaagattgttatcttcagctatatctgccacctcctctttgtgcaccgcagcaagatcaaaggcaccgtatgaacgttaaaagtgtgaaatttaatttcattatgaatctgattctgaccgttagcaactttctgttcccgctggtgacgttcccatacgttagtcgcattctgcagccagaaggtaccggtaaagtggcctttgcgattagcgtggttagctacttcagcatcttcgcgagtctgggtgtggccacctatggcgttcgtgcgtgtgcgcaagttcgcgacaataaagatctgctgagtcgtacggtgcatgagctgctgttcatcaacatcatcgccacgatcattgtgtacgtttgctttctgctggtggtggcgtttaccccacgctttagcgcggaaaaagagctgttctgggcgacgagcatctttattctgttcaccatcattggcatcgagtggctctacaagggtctggagaagtaccagtacatcacgatccgcacgatcatcttcaagctcattgcgctggtgctcgtgtttgtgttcatcaagacgaaggatgactacgtgatcttcgccgtgatcagtgtgtttgcgatcgttggcagcggcatcttcaacctctttaacagtcgcaagctgattaactaccatctgtacgaggattacgagttccgcaagcatttcaagccaatgtttctgctgtttctcacgacgctcagcatcgccatctacaccagtgtggatgaagcgattctgggtctgctgacgagtccgcaagatgtgggctactataacgcggccatgaaggttaagggcattctgtttacgctgatcaccagtctgggcattgtgctgctgccgcgtctgagctattatgttgagaacaatatgacggatgaattccatgccgccctcaagaagagcatgaacttcatcatcgtgatcgccgttccagtggtgatcttcttcatgctgttcgccaaggagattattctgctgctggccggcgaaagttatatcaacgccattctgccgctgcagattattgtgtgggcgctgctgctcagcgccattaccaacattctgggcatccagattctgctgccgctcaagaaggataaagagctgctgatcagcgtgctgctcgcggccattgtggacattgtggccaatctgattctggttccgcaactcgccagcgttggtaccgccatcagcgttgtgatggccgaactcaccgtgctggtggtgcagctggttatcctccgcaagtacatctggatcctcttcagcaatctccagttcgtgcgcatcggtctgagcatcgttttcagcatcgtgctgagcctcagcatctatcagtggaacatcacgaacagcatcatgctcacgtttctgatcatgggcttcatcttcttcacgacctacttcattctgctgctgattctgaaggagaacttcatgatgtacgtgtaccagaccatccagcacaagattctgaaataaattatatagtgttatcacataacgtatccttggagaatagaaatgaaatatgattatctgatcgtgggcgccggtctgtttggcagcatctttgcgcgcgaggccaccaagcgtggcaagaaatgtctggttatcgagaagcgcgatcacatcggtggcaactgctacacgcagaacgtggaaggcatcaacgttcacaaatacggtgcgcacatcttccacaccagcaacaaggtggtttgggactacatccagcagttcgccgagttcaatcgctttaccaacagcccggtggcccgctataaggacgaactgtacagcctcccgttcaacatgctcaccttcaacaagatgtggggcgttatcacgccgcaagaagccgaagcgaaaatcaaggagcagatcgcgaaggagaacatcacggatccgaagaatctcgaggagcaagccatcagtctggttggtcgcgatatctacgagaagctcatcaagggctataccgagaagcagtggggccgtaagtgtacggagctgccagccttcatcatcaagcgtctgccagttcgctacacgtacgacaacaactacttctacgacacctatcaaggcatcccgatcggtggctacaccggcatctttgaacgcatgctcgagggcatcgaggtgaaactgggcgttgacttcttcgcggaacgcgaacattacgagagtctggccgagaagatcgtgttcaccggtatgattgacgaatattttggttaccagttcggcaaactggaataccgcagtctgcgcttcgacaacgaagtgctgaacatcccgaactaccaaggcaatgcggtggtgaactatacggaagccgaggtgccatatacgcgcatcatcgagcataagcatttcgagtacggcacccagccgaaaaccgtgatcacgcgcgaacacagcaaggagtacgaagaaggcgacgagccgtattacccgatcaacgacgcccgcaacaacgaactgtacgccaagtacaaggcgctggccgacgcgaccccaaacgttattttcggtggccgtctggcccagtataagtacttcgacatgcacaatatcatcgccgaggcgctggagtgcgttaaggtgcacttttaatataagggagtaacgctatgaataagatcatcgcgaagatcagtctgatcctcgtggatatcgtggccatcttcgttagcattctgatcgccgtgagtctgcgtaaaattctgggtctgctcttcacgctgccggagatcgactacagctacatcttcttcgcgtatgtgtatctgattctgattctgatgatgacgtacctcggcgcgtataccaaacgctacgacttttggcacgaaagccgtctgatcgtgcgcggcagctttctcagtctgctgattctgctgagtgccctcgcgctgggccaaaacgcggaatactatagccgcagcacgctcgtgctgatctttctctgctgcgccatcgtgctgccgatcgccaagattttcaccaaaaaaattctgttcaaactgggtatctggcagctgccggcgaaggtgatcagcgagaacgaccagttcaaaaacgagctcttcgaagaccagtatctgggctatgtgaaggcgaaacacagcgagcacaagattatcttcatcgacggcgcgaatctgggcaaagatcgtctgaaccagatcatcgaggacaacatcaagaatagccgtgagatcatcttcaccccggttctgaatggctacgacttcagccatagctacatttataacatcttcaacacgcgcaccaacattttcacgctggagaacgagctgctgagcaaaagcaaccgcatcttcaaactgctgatggactatattctggtgctgggtagtgccgtgttctgggtgccggtgctggtgctcatcgcgttctggatcaagaaggaggatccgaaaggcgaggtgttctttctgcagcgtcgcctcggcgtgaatggcaaggaattcatgtgctacaaattccgcagcatgtacagcgaccagagcttcatgcaagaatggctggagaaaaatccggaggaggccgcgtactaccgcatctaccataagtatatgaacgatccgcgcatcaccaaaatcggcgcgttcctccgcaaaaccagtctggacgaactgccgcagctgatcaacgtgctgcgtggtgagatgagtctcgttggtccgcgcccgtacatggttatcgagaagaaggacatcggcaaaaaagccccactggtgctcgcggttaagccgggcattacgggcatgtggcaagttagcggccgcagtgatgtgaacttcgacagccgcgtggagatggatgtgtggtatatgaaaaattggagtctgtggaatgacatcgtgattctgatcaaaacggtgcaagccgtgttcaagcgcgacggtgcctattaaagtatgatcaccagcatccagtacctccgtggcatcgccgcgctgttcgtggtgctgttccacatgaagtggatgctcaacaatgtgtacgtggagaagaacctcggcgacatcttcttcatcagcggcaacttcggcgtggatctgttcttcgtgatcagcggcttcgtgatctgtctgagcacggaacgcgaaacgctgcacccggtgaaggagtttttcatccgccgcttcttccgcatctacccactgctgctgctgagcgtttgcaccatctacattctgggcgacttcaagatccacgagctgatcctcagcatgatcccaatccatctggactacagcagcccgagcccggtgttcggctacaacattctggttagcgcgtggaccatcacctacgagattagcttctacatcatcctcgtgctgagtctgatgatcaaccatcgcttccgctgcgaactgaccattctgttctaattatcattaatatagtttcaaactattattattttggtgaatatagcctatcactagatagagagataccccttgataaaaggggacatttttttgttatgttctcatcatcaatgttattaacatttatttatgggattttaatatatataaaattacaaattttatgaaaagcattatcatcctcgacaagtacttcctctacagcattctgctggtggtgatcagcttcgtgttcatcaaacacccgatcttcgacggccacggtgtgctgaaatggggctttctgagcttcatcattctgctgattctgctcatcatcgagaacacctacggcatcgccaaaagcaactttctgttctggctgggcgaaatcagctacagtctgtatctgacgcacatcattatcctcgaattcattctgaagcacatcaccccggagatctggaacaacccgaatctgggcatgagcaagatcctcttctacctcgccatcagcatcagcttcagctatctggtgtatctgctggtggagaagccgttcatcaacctcggcaagaagctgatcacgaagctgtaaatattaatggatgattttatgaagtcacgaaatctcgaacctacaaaaacgcatctgatctatttagatatactaaatatttttgcttgcattgctgtacttttttacatcacaatggtattgtacattggtataacgtaaatgaattggcttggaaacaagccttattttttgaagtggctttttattgggctgttcctattttctttatgctcaccggcgccacgctgttcgaataccgcaaccgctacagcacgaagcagtttttcatcaagcgcatccagcgcgccgtgttcccgtttctgagctgcagcctcattctgctgggctatagcttttacagcggcatgatcgaggcctttagcatccgcgacagcatcagtgccatcttcaacaccaaggacatcccgttcattgaaatctattggttttttatccatctctttagtctgtacatggtgatcccggtgctcagtctgctgaaagataactaccgcattctgtgctatattgtgggcgccatgtttctgacccacagtctgtttccggtgatctttgacttcttcaagctgcactacaactggagcatcattttcccgatggcgggctacagcatctatctggttctgggctatctgctgagtaaggtgaaactggaaaagaaatatcagatcatcatttacattctgggcattctgagcgtgctgctccgctacttttatacctacgtgagcagtctggaggccaaccagctcgatcgcacgctgttcagctacatgcaattccacaccgtgtttctggcggtggcgatcttcattttcgtgaaggaattcttcagcggtgtgaaactgttcaacgccaaggtgctggcggtgttcagcagctgtagtctgggcatctatctgatccacaagctcgtgatggactacgaactcaagtttctgggcatcagcgaggacaatctctactggcgctttttcggcgccttcatgacgtacggcgcgtgcctcgtgatcgtgctgtttgttaagcgcatcccgtatctgcgcgccatctttccgtaaagatattataaatatgaaaattctgatcaccggtggcgccggttttatcggcagcgccgtgatccgctatatcatccagcatacccaagatagcgtggtgaatgtggacaaactgacctacgccggcaatctggcgagtctggaaagcgtgagcaatagcagccgctaccactttgagcaagcggatatttgcgacagcacccgcatcagtcagatcttctgcaagtaccagccggatgttgtgatgcatctggccgccgagagccacgttgatcgcagcattgatggtccggcggcgttcatgcagacgaacatcatcggcacctataccctcctcgaagccagccgccagtattggctcagtctgccgctggaacgcaagcaaaccttccgcttccagcacatcagtacggacgaggtgtatggcgatctcaacgatagcaacgagctgttcagcgagaacacggcctatagcccgagcagcccatatagcgccagcaaggccgccagcgatcatctcgttcgtgcgtggtttcgtacctatggtctgccgacgctggtgaccaactgcagcaataactatggcccgttccagttcccggagaaactgatcccgctgatgattctgaacgccattagtggcaaaccgctgccgatctatggcaatggtctgcagatccgcgactggctgttcgttgaagaccacgccatcgcgctgtatcaagttctctgtcgcggcaaagtgggcgaaacgtacaacatcggtggccacaatgagaagaccaatatcgaggtggtgcaagcgatctgccgtctgctggacgaactggtgccgaataaaccgagcggcatcgagcagtatgaagaactcgtgacctacgtggccgatcgcccgggccatgatgttcgctacgccatcgacgcgagcaaaatcgagaatcagctgggttggacgccgaaagaaaccttcgaaagcggtctccgcaagaccgtggagtggtatctgaataaccagaagtggtggcagagcgttctggatggcagttactgcggtgagcgtctgggtctgagtctgaaaagctactaagcggcaaatagtatttcagtggggatcattttggatccatataaaatagttggtctgtttttgttgaaattttagcgaaaattgttaaaaaataagtcgatttgcctcttattctcactgaatttaccctttactttaactatcattttctattccataaggcgtatttaatgtggtatttaatttgccaataaaaattaattgctcaagtcgttacacacgctaccgcccctggctcatcagctaccagtgcactgcgtacatatcgacttgttgcaaacctcgcccagcagggcaaagctcactaaaacttaaacgctaattgtcttattaattgcatccggaaacaaggattaatcttataaaatcagcattaaaatgctccagataaccccttgttacttaagccctttatacaaaactaaaacggcagtcaacactcgtttcagccaacttgccgcttcgaatgttcactgccgttattatgtttatcaccaaccatttatcacggttgttaatacttattcatgcaaaagctgctctatgctcttacggaacttggctccttctttcaggttgcgcagcccgtacttcacaaatgcctgcatgtagcccatttttttaccgcagtcatagctgtcacccgtcattagcatcgcgtcaaccgactgttttttcgccagttcagcaatggcatcggtgagctggatgcggccccaggcgcccggttcggttctttccagttccgcccagatgtcggctgaaagcacataacggcctaccgccatcaaatcggaatccagcgtctgcggctgatccggtttttcgataaactccacaatccggctgactttgccttcattatccagaggttctttcgtctggataacggaatactccgataaatcacctttcatgcgcttcgccagcacctggctgcgacccgtttcattgaaacgcgccaccatcgccgcaaggttatagcgcagcggatcggcggtagcatcatcgataataatatccgggagtaccacaatgaaagggttatcgcccacgaccggacgcgcgcacagaatagaatgccccagccctaacggctgcgcctggcgaacgttcataatcgtcacgcccggtgggcagatagattgcacttccgccaaaagctgacgcttaacgcgctgctcaagaagtgattcaagttcataagaggtgtcgaagtggttctcaacggcgtttttagacgcgtgagtcaccagcacgatttctttgatccctgcagccacaatctcatcgacaatgtactgaatcattggcttgtcgacgatcggtagcatctcttttgggattgccttggtggcaggcaacatatgcatacccaaacccgctaccggtataactgctttcaaattcatcattgtttcttccacctgtaaaatggttgctgaattatagctctttagcttgttttcgccagcatgaattactctgctgccagggataatgatggcacgctctacattacgtcttagtcggcaccataacatatcgattaccctgttatccct

SEQ ID NO: 47 - DNA, 1128 bp, Artificial Sequence
  truncated ApxII (N-terminal HIS10 tag ApxII(439-801aa)
atggggcaccaccatcaccatcatcatcaccaccatcaaggttatgattctcgtcatttagctgatttacaagacaatatgaagtttcttatcaatttaaataaagaacttcaggctgaacgcgtagtagctattacccaacaaagatgggataaccaaattggagacctagcggcaattagccgtagaacggataaaatttccagtggaaaagcttatgtggatgcttttgaggaggggcaacaccagtcctacgattcatccgtacagctagataacaaaaacggtattattaatattagtaatacaaatagaaagacacaaagtgttttattcagaactccattactaactccaggtgaagagaatcgggaacgtattcaggaaggtaaaaattcttatattacaaaattacatatacaaagagttgacagttggactgtaacagatggtgatgctagctcaagcgtagatttcactaatgtagtacaacgaatcgctgtgaaatttgatgatgcaggtaacattatagaatctaaagatactaaaattatcgcaaatttaggtgctggtaacgataatgtatttgttgggtcaagtactaccgttattgatggcggggacggacatgatcgagttcactacagtagaggagaatatggcgcattagttattgatgctacagccgagacagaaaaaggctcatattcagtaaaacgctatgtcggagacagtaaagcattacatgaaacaattgccacccacccaacaaatgttggtaatcgtgaagaaaaaattgaatatcgtcgtgaagatgatcgttttcatactggttatactgtgacggactcactcaaatcagttgaagagatcattggttcacaatttaatgatattttcaaaggaagccaatttgatgatgtgttccatggtggtaatggtgtagacactattgatggtaacgatggtgacgatcatttatttggtggcgcaggcgatgatgttatcgatggaggaaacggtaacaatttccttgttggaggaaccggtaatgatattatctcgggaggtaaagataatgatatttatgtccataaaacaggcgatggaaatgattctattacataa

SEQ ID NO: 48 - DNA, 693 bp, Artificial Sequence
  ApxIII(27-245aa)-HIS10 (synthesized)
gatgtaactaaaaatggtttgcaatatggggtgagtcaagcaaaattacaagcattagcagctggtaaagccgttcaaaagtacggtaataaattagttttagttattccaaaagagtatgacggaagtgttggtaacggtttctttgatttagtaaaagcagctgaggaattaggcattcaagttaaatatgttaaccgtaatgaattggaagttgcccataaaagtttaggtaccgcagaccaattcttgggtttaacagaacgtggacttactttatttgcaccgcaactagatcagttcttacaaaaacattcaaaaatttctaacgtagtgggcagttctactggtgatgcagtaagtaaacttgctaagagtcaaactattatttcaggaattcaatctgtattaggtactgtattagcaggtattaatcttaatgaagctattattagtggcggttcagagctcgaattagctgaagctggtgtttctttagcctctgagctcgttagtaatattgctaaaggtacaacaacaatagatgctttcactacacaaatccagaactttgggaaattagtggaaaatgctaaagggttaggtggtgttggccgccaattacagaatatttcaggttctgcattaagcaaaactggacaccaccatcaccatcatcatcaccaccattaa

SEQ ID NO: 49 - DNA, 4581 bp, Artificial Sequence
  rfaK(SL1344)-proD- ClyA-ApxI(628-845aa)-ApxII(612-801aa)-ApxIII(626-860aa)-HIS6-chlo ramphenicol resistance cassette-rfaL(SL1344)
cgatcgcaatttgtgttagatggcataacgggctatcacctcgcagaacctatgtcgagcgacagtataattaatgatattaaccgtgcgcttgctgataaggaacgccaccagattgccgaaaaagcaaaatccctggtgttttcaaaatacagttgggaaaatgtagcgcagcgtttcgaggaacaaatgaaaaactggtttgataagtgactcgagttctagagcacagctaacaccacgtcgtccctatctgctgccctaggtctatgagtggttgctggataactttacgggcatgcataaggctcgtataatatattcagggagaccacaacggtttccctctacaaataattttgtttaacttttactagagtcacacaggaaagtactagatgaccgaaatcgtggcggacaagaccgtggaggtggttaaaaacgccatcgaaacggcggatggcgccctcgatctgtataataaatatctcgaccaagttattccgtggcagaccttcgatgagacgatcaaggaactcagccgtttcaagcaagaatacagccaagccgccagtgtgctggtgggcgatatcaaaacgctgctgatggacagccaagataagtacttcgaggcgacccaaaccgtgtatgaatggtgcggtgttgccacgcaactgctggccgcgtacattctgctgttcgatgagtataacgagaagaaggcgagcgcccagaaggacattctgatcaaggtgctcgacgacggcatcacgaagctgaacgaagcgcagaagagtctgctcgttagtagccagagcttcaataacgccagtggcaagctcctcgcgctggacagtcagctgacgaacgactttagcgagaaaagcagttacttccagagccaagttgacaaaatccgcaaggaggcgtacgccggtgccgccgcgggtgttgtggccggtccgtttggcctcattatcagctacagcatcgcggccggcgtggtggaaggcaaactgatcccggagctgaaaaataaactgaaaagcgttcagaatttcttcaccacgctgagcaacacggtgaagcaagcgaacaaggacatcgacgcggcgaaactgaagctgaccacggagatcgcggccatcggcgaaatcaagaccgaaacggaaaccacgcgcttctatgtggactacgatgatctgatgctcagtctgctgaaggaagcggcgaagaaaatgattaacacgtgcaacgagtaccagaagcgccatggtaagaagacgctcttcgaggtgccggaggtgtacgcgggtaatggccacgacgtggcgtactacgataagacggacaccggttacctcaccttcgatggtcagagtgcccaaaaggcgggcgaatatatcgttaccaaggaactgaaggcggacgttaaggtgctcaaagaggtggtgaaaacccaagatattagcgttggcaagcgcagcgagaaactggaataccgcgactatgagctgagcccattcgaactcggcaacggtatccgcgccaaagatgagctgcatagcgtggaagaaattatcggcagcaaccgcaaggataagttcttcggcagtcgcttcacggatatcttccatggcgcgaaaggcgacgacgaaatctatggcaatgacggtcacgacattctgtacggcgatgacggcaacgacgttattcatggtggtgacggtaatgaccatctggttggcggcaatggtaatgatcgcctcatcggcggcaaaggtaataatttcctcaacggcggcgatggtgatgatgaactgcaagttttcgagggtcagtacaacgttctgctcggtggtgcgggtaacgacattctgtatggcagcgatggcacgaatctgttcgatggcggtgtgggtaacgacaagatttacggcggtctcggcaaagatatcaatctcggtgcgggcaatgacaacgtgtttgtgggcagcagtaccacggtgattgatggtggtgatggccatgatcgcgttcattacagccgcggcgaatacggcgccctcgttattgacgccaccgcggaaacggagaagggtagctatagcgtgaagcgttacgtgggcgatagcaaagccctccacgaaaccatcgcgacccatcagaccaatgtgggcaaccgcgaagagaaaatcgagtaccgccgcgaggatgatcgcttccatacgggctacacggtgaccgatagtctgaaaagtgtggaggaaatcatcggtagccagtttaatgatatcttcaagggcagccaattcgacgacgtgttccacggcggcaacggtgtggataccatcgatggcaatgatggtgacgaccatctcttcggcggtgcgggcgatgatgttatcgatggcggtaacggcaataactttctggttggcggtaccggcaacgacatcattagtggcggcaaggacaacgacatctacgttcacaaaacgggcgacggtaacgacagcatcaccgatagcggtggccaagataaactggcgcatctgggtaacggcaacgataaagtgtttctggccgcgggtagtgcggagattcatgcgggtgagggtcacgatgtggtgtactatgataagaccgataccggtctgctggtgatcgatggtacgaaagcgacggaacaaggccgctacagcgttacgcgcgagctgagtggcgcgaccaaaattctgcgtgaggtgattaaaaatcagaaaagcgcggttggcaaacgcgaagagacgctggaataccgtgactacgaactgacccagagcggcaacagcaatctgaaagcccacgatgaactgcatagtgtggaggagattattggcagcaatcagcgtgacgagttcaaaggcagcaagttccgcgacatcttccacggcgccgacggtgacgatctgctgaacggtaatgacggcgatgacattctgtacggcgacaaaggcaatgatgagctgcgcggcgacaacggtaatgatcagctgtatggcggtgagggtgatgataaactgctgggcggtaatggcaacaattatctgagtggtggcgacggcaacgatgagctgcaagttctgggcaacggctttaacgtgctgcgcggcggtaaaggcgatgataagctgtacggtagcagtggtagcgatctgctggatggcggcgagggcaatgattatctggaaggcggtgatggcagtgactttcaccatcaccaccatcactaactaaatatattttaggtcacctctcaaatcgtttgcctgataccgctccaattacacgtcttgagcgattgtgtaggctggagctgcttcgaagttcctatactttctagagaataggaacttcggaataggaacttcatttaaatggcgcgccttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgtaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacaactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtaggcgcgccgaagttcctatactttctagagaataggaacttcggaataggaactaaggaggatattcatatggacccagcgcgtttttttatctatttcttagcgccagcagaaaaccggtaatgataccaatttgagcaatatcgacctgttcaaaattgccacgaacgatataaaaaccgacgaaagataaaaatagcaagagatgagcattgtaggggcttatctctactttcctgagggtagagctggctgtttccctgatgatagcgccatataaataggcggccgc

SEQ ID NO: 50 - protein, 365 aa, Artificial Sequence
  amino acid sequence of rfb (APP2) cluster gene kanE
MKHKILHFSQVLGGVGRYLELYDKYINKDSFENIYILPIGDWEAAEAQDKRYILNIEQSFSPIKLISNVIKIRNILKKEKPDIFYLHSTFAGVIGRLAAIGMRCKVIYNPHGWSFKMNVSRLKQTFYKIIEGGLVFLTDKFVLISKSEYEAARSIGVSEKKCCLIYNGIETIKKTDIAIIPKLDDKYIIGMIGRISEQKNPMFFAQFAKEIIKQYPNTYFILVGDGEQRESLEDYLERNNLNDVFYITGWVTNPESYLNLFDQAVLFSKWEGLCLSVCEYMLYEKPILVSNIGGINDLIQNEVNGFTIVEGDLKDAVNKSNRLRNEPKTVAKFIEASNILIQEKFNAQKMVNSLEKLFIKLSENK

SEQ ID NO: 51 - protein, 288 aa, Artificial Sequence
  amino acid sequence of rfb (APP2) cluster gene cpsP
MKEKFSCIVVCYNPDNSVLDNLKNYISYVGKVIVVDNSDVDNSQLFSSLSEYLIYIPLYKNVGIAYALNIGVEKSKELGYEYIITMDQDSSFATNLVDVYSHYISNYPIDQIGALSPVYITDRGFNRTSKEEFKQIKITMQSGSMFFTDKFDVIGRFDNDLFLDVVDWEYFFRIYTLGYKTIQCNKAMLKHAPAETLTLFKIKGKTIGVGVASPLRYYYQIRNLLWCVLHKKSFFMIKTIAYKFIKILFLFNNKKQYLSFAYMAIKDAFNNRLGAYDTLYLEKSRNEK

SEQ ID NO: 52 - protein, 328 aa, Artificial Sequence
  amino acid sequence of rfb (APP2) cluster gene epsJ
MKNDLPLISIIIPIYNVKPYLEKCVNSVLSQSYPNLEIILVDDGATDGSAQVCDDFSEKYANIQVIHKKNGGLSSARNAGIEAMKGEYVFFLDSDDWIANDAISQLYDDMVEYNADITGISFYQAYSDGNLVLNTHLIEKQMLSKKEALRTFLFNNYLTPCSCGKLYKASLWKDIRFPEGRLFEDQLTTYKVIELANTIIFNPAAKYFYFKRIGSIGHSAFSEKTYDLYEAVNEQYNEITKHHPDIESDLAVAKITWEIVFINMMLNSNYSDQAIVDKTRVFARKRILDVVKCEFIPNLRKFQITLFAYNFSLYKVLYARYKKKNPLS

SEQ ID NO: 53 - protein, 225 aa, Artificial Sequence
  amino acid sequence of rfb (APP2) cluster gene setA
MIPKKIHYCWFGGNPLPKSVKKCIKSWKKYCPDYEIIEWNESNYNVHKNLFIKEAYEKKKFAFVSDYARLDVVHSEGGIYLDTDVELIKPIDDLLAHSCFLASESIDDVNTGLGFGAEKGHWFIAENMSVYENMYFNMENIITCVEITTKLLIERGFSASDKIQNIDDIFIYPTEYFCPLNYKTHELHITQNTYSIHHYDATWQSPLMKFKTKIKYILCLAGIIK

SEQ ID NO: 54 - protein, 378 aa, Artificial Sequence
  amino acid sequence of rfb (APP2) cluster gene Hypothetical protein
MNSLVYRIDIRTLIFSIFYFTFLVSDFLLLAQDGTITKDIIKWVKLFSLLPLLLLIFKLPLNLLILGFFTIMISAFYSIYTGDSFLLYICLLMSFSYKVNFNFLFKIGLYLTSILVVLILTYFFFEYFLIGDSHFVYDATYWFKRYTFNFDNPNAFPMRIFVFFIFYILHVGKLRLFDTFLFVILFGIVFYFSNSRTAFYIFILCVLTIHFNQVFNVLNNTFVKLLINNSIIFITIFSIWSAIYYQDYYSYLEPINKILSKRIYFANEAYKSLGFEFYPRNIKWWIEESDWHIIDNGYVYLFISGGLLVGNLFIFSITWLMYRLNKFNLSNEAILLMFSMLYLLSESHFINIFYNIPILLLAIFINKTNIVRYLECKK

SEQ ID NO: 55 - protein, 481 aa, Artificial Sequence
  amino acid sequence of rfb (APP2) cluster gene rfbX
MNKNLVNNSIMSFLLTISNFIFPLITFTYAARILQPDNMGKFAFSLSVVDYLSLFATFGVVGYGVRACAEVRNNKEELTKTVQEILFINIFLAIIAYLVIFLLISYQHAFREDTLLFLIMSSCIIFNVIGIEWLYKSLDEYRYITVRSILLKIISLIMILCFVKEKDDYPLFALFFVLPICLSSLLNIINSRKILLFKLFKLDLSKHIKPMFVLFLVTLSYTLYANVNDVLLATVTNTEQVGYYSVAFKIKAALLAFITSTSMVFLPRLTEYIKNNQDIEFIDLLRKSFDLVFFLAVPITLFFFLYAKETIFLLFGEKYNKSSLLLQTMIWSVFFGGLNNILSVQMLLPLKKDNQFLISILSGGCISLVVNFIFLRELQSLSTSISVLVAEVVILIIQLVILRKYIVRIFNNLNPLKVIMSVFFSIWFVNLIYANFIALGNSFLEYIISIFIFSLFYVFLLFFSKERFVHDVFFYIRSKFD

SEQ ID NO: 56 - protein, 236 aa, Artificial Sequence
  amino acid sequence of rfb (APP2) cluster gene vatD
LINLLISILAKILSRISKLILNIKKRKEYKRVGSIVDSKNIDLSFICGNYCRVGRDTVIEKNVIMGRLSYINSDMGKTYIGSNVKIGSLCSISSGVIIAPVNHYLNYVTTHPLLYNSYYSSILNINSNLLSQQELDANVSTVIGNDVWIGANVIIKRGVTIGDGAVIGAGSIITKDIPSYAVVAGVPAKIIKYRFSKDVIESLKDSKNVWELSTSELEENFSHLYDVEKYLNRFKL

SEQ ID NO: 57 - protein, 440 aa, Artificial Sequence
  amino acid sequence of rfb (APP2) cluster gene pglC
MSKKNIVAQTLLLCLDLLLISMAIFLAVFIRNNILPNIMLFEPVSYIEYLVYPFPYVIIVTLFMWFGLYTRRYDLWQESLFIIKVCFISFIIIFATLALGKNIEYYSRAVLLLSLFLSVIFLPIGRYFLKKSLFRLGLWERKVKFIGNLNKNEIGIFNSPHVGYVLSKDDTYDVIFISSGDKSVSELNDLIESNKLLNREVLFIPVLNQYDFTQSVLYNNFSTRLNLFTLENKLLGKQNKILKYLLDYVLVLSTLPFWGGLILLISIKLKLEDPKGKIFFLQKRLGQEGKIFYCYKFRTMVSDQSFMQQWLIDNPEERDYYAVYHKYINDPRITKFGHFLRRTSLDELPQLFNVLKGDMSLVGNRPYMVEEQQKMKDAASIILMSKPGVTGLWQVSGRSDVSFEERLQIDSWYIKNWSIWNDIVILFKTVGVVLRKDGAS

SEQ ID NO: 58 - protein, 354 aa, Artificial Sequence
  amino acid sequence of rfb (APP2) cluster gene rffG
MKKILVTGGAGFIGSAVVRHIINDTQDSVVNVDKLTYAGNLESLLMVENSPRYVFEQVDICNRAELDRVFAQHQPDAVMHLAAESHVDRSIDGPAAFIETNIVGTYTLLEAARYYWNSLDADKKSLFRFHHISTDEVYGDLEGTEDLFTETTPYSPSSPYSASKASSDHLVRAWLRTYGLPTIVTNCSNNYGPFHFPEKLIPLMILNALEGKPLPVYGNGQQIRDWLFVEDHARALYKVVTEGKVGETYNIGGHNEKANIDVVRTICSLLEELVPNKPAGVHKYEDLITYVTDRPGHDVRYAIDATKIGRELGWKPQETFETGIRKTVEWYLNNTEWWSRVLDGSYNRERLGSN

SEQ ID NO: 59 - protein, 292 aa, Artificial Sequence
  amino acid sequence of rfb (APP2) cluster gene rmlA2
MKGIILAGGSGTRLYPITRGVSKQLLPVYDKPMIYYPLSVLMLAGIREVLIITTPEDNESFKRLLGDGSDFGIQLSYAIQPSPDGLAQAFLIGEEFIGQDSVCLVLGDNIFYGQHFTQSLQEAVKSVETKGATVFGYQVKDPERFGVVEFDDNFRALSIEEKPIQPKSNWAVTGLYFYDNRVVEFAKQVKPSARGELEITTLNEMYLNDGSLNVQLLGRGFAWLDTGTHDSLHDAAAFVKTVQNLQNLQVACLEEIAYRNGWLSLEQLEALTKPMAKNEYGQYLLRLTKGTK

SEQ ID NO: 60 - protein, 291 aa, Artificial Sequence
  amino acid sequence of rfb (APP2) cluster gene rmlD
MARFLITGAKGQVGYCLTKQLQSKADVLAVDRDELDITNRDAVFKVVREFHPDVIINAAAHTAVDRAESEIELSEAINVKGPQYLAEAANEIDAIILHISTDYVFEGTGSGEYKENDEPNPQGVYGKTKLAGEIAVQQANKRHIILRTAWVFGEHGNNFVKTMLRLAKERESLGIVSDQFGGPTYAGDIASSLIHIANIILNSKIDVFGVYHFTGKPYVSWADFAKKIFDEAVSQKVLEKAPLVNFIATSNYPTSAKRPANSRLDLTKIDEVFGIKPSNWQQALKNIKAYA

SEQ ID NO: 61 - protein, 180 aa, Artificial Sequence
  amino acid sequence of rfb (APP2) cluster gene rfbC
MKIIETNIPDVKLLEPQVFGDERGFFMEIFRDEWFRQYVADRTFVQENHSKSIKGVLRGLHYQTENTQGKLVRVVQGSVFDVAVDLRKSSPTFGQWVGEVLSAENKRQLWVPEGFAHGFYVLTETAEFTYKCTDYYNPKAEHSLIWNDPTVAINWNLGGAPSLSAKDLAGKVLNEAVLFE

SEQ ID NO: 62 - protein, 267 aa, Artificial Sequence
  amino acid sequence rfb APP8 DUF4422 domain- containing protein
MNKDIKILIATHKQHFMPSDEMYLPLHVGKLGKADLGYQGDDSGDNISIKNPNFCELTGLYWAWKNLPNDYLGLIHYRRFFSVKNRAERKNNPLETLYLTNEEANQLLSQYDVIVPSKRNYYIETLYSHYANTLHAEHLDVTREIIAEKCSEYLASFDAVIKQRSGYMFNMFIMSKALVNDYCSWLFPILFELEKRIPTDQYSAFHARFYGRVSELLFNVWLKQYSQSNPLKVKAIPFVYGEKINWLKKGTAFLVAKFFGKKYEKSF

SEQ ID NO: 63 - protein, 355 aa, Artificial Sequence
  amino acid sequence rfb APP8 Glycosyltransferase family 1 protein
MKRILVYGMTDNFGGMEAYIHNIYQHLDKTQIQFDFVCDFPKMTLSDYYLDNGCKIHFIPPKNQGLFKSLWAMWKVIKENNYDVIYFNIMNAGYVLNMLPAFLLGKKIIAHSHNADTDKKKLHYGLRLLLNIVTKIKLACSKEAGFFMFGKEENFSIINNAINLDRYLYSEEKYRDLRHKLGWGDKKVILYVARMNHQKNPLFALYIMRELKQSMPNAVLVYVGTGELKEQVQQYILDNNLDNVILLGLRNDVNELMIAADLFILPSLFEGLPIVAVEAQAAGLPIILSENISIEAKLVNSTYFLPINDVFLWVNKIKKILEISGNKRFSDQLALSKAGYNIESVVKNIQKILVN

SEQ ID NO: 64 - protein, 350 aa, Artificial Sequence
  amino acid sequence rfb APP8 Glycosyltransferase family 2 protein
MNNTKISLIFACYNVSQYLDNLFQLLTNQPYQNIEIIFVEDCATDDTKAKLQSFNDPRVKLLCNEKNIGAAESRNRGIQIVTGEYIWFPDPDDLFDELLLTKVNTIIQKNRPDVISIGMQERYEINGKTDYTKDIISRYDGLITGDFTDVFVDLEESFLFGYTNNKFYKANIIHKYRILNEHQALKEDFEFNIKVFKQVSNFYLLNEPLYFYMKRNNGSLTSKFVPDYFRIHMQTLASFKSLIEVKATINDNVNRLLVNRFVRYCLSAIERNSSLKSGMSFLEQNQWIKENIFNQEKYNEYLLLSDLVNKKQKLFYFLIKYRIGFLLVTAANIVKLVKAKFPILFVKLKG

SEQ ID NO: 65 - protein, 349 aa, Artificial Sequence
  amino acid sequence rfb APP8 Beta-1,6- galactofuranosyltransferase
MKKYQIVELSTEHNHAGSKAVQDVYEIALSMGYKANVVRTATSVDSLLAKILRQVIFFIDWLKIYFSIESNSIVLIQNPYYHKQLIRNWILNRLKRIKKVKFISLVHDVEELRKSLYNNYYKNEFETMLSLADSIIVHNDKMKSFFIKKGYSEDKLISLGIFDYLQKSVDKKRVSFERAISVAGNLDIKKSSYIAQLGSLPAIKAHLYGPNFEHSLEAFPNIEYHGSFPATEIPQKLVSGFGLVWDGQSIETCTGDFGEYLQYNNPHKLSLYLSSGMPVVIWDKAAEADFVKKHNVGLCVSSLSELQDKLNVMTEQEFEEMVNNVEKQTACLISGEYTKKAISEAERVI

SEQ ID NO: 66 - protein, 443 aa, Artificial Sequence
  amino acid sequence rfb APP8 Oligosaccharide repeat unit polymerase
MFLYLLVFSLLLILIFNLLIVNLDYMHPSILFVVPFLVFGVTSILGEEAYKIIFHEETLLVIVSSALIFTFITLLSQTVYKSKENLNFPLTEIIISKKVTLFFIVFFIVTQLAFIKYLEAISLAHFGYSGSLGEMISLYDVMTKFWTEIFSELNVPIPLLYRIGNPITQGFGYLIVYIFIHNYVATKRIDKLHLLIILLLCLNIILNGSRSPIFRIVTMMLITFYVLYNKQNNVRRGNIKFLLKSLLIVIFSGTFFIALLSLMGRENDLDMFHYIFIYVGAPLVNLDNYLAFRPDGSYATIFGEQTFRGLYAYIAKIISDESLIFPTIDQFTFSNNGLEIGNVYTTFYSFIYDFEYVGFIPLILIIALYYVFTYQRLKTRAIKTNKVHFSLFIYAYLFNDLIMLAFSNRFYTTVLDIGFIKIVIFSYICHLLFVHRSKIKGTV

SEQ ID NO: 67 - protein, 483 aa, Artificial Sequence
  amino acid sequence rfb APP8 Flippase
MNVKSVKFNFIMNLILTVSNFLFPLVTFPYVSRILQPEGTGKVAFAISVVSYFSIFASLGVATYGVRACAQVRDNKDLLSRTVHELLFINIIATIIVYVCFLLVVAFTPRFSAEKELFWATSIFILFTIIGIEWLYKGLEKYQYITIRTIIFKLIALVLVFVFIKTKDDYVIFAVISVFAIVGSGIFNLFNSRKLINYHLYEDYEFRKHFKPMFLLFLTTLSIAIYTSVDEAILGLLTSPQDVGYYNAAMKVKGILFTLITSLGIVLLPRLSYYVENNMTDEFHAALKKSMNFIIVIAVPVVIFFMLFAKEIILLLAGESYINAILPLQIIVWALLLSAITNILGIQILLPLKKDKELLISVLLAAIVDIVANLILVPQLASVGTAISVVMAELTVLVVQLVILRKYIWILFSNLQFVRIGLSIVFSIVLSLSIYQWNITNSIMLTFLIMGFIFFTTYFILLLILKENFMMYVYQTIQHKILK

SEQ ID NO: 68 - protein, 365 aa, Artificial Sequence
  amino acid sequence rfb APP8 UDP-galactopyranose mutase
MKYDYLIVGAGLFGSIFAREATKRGKKCLVIEKRDHIGGNCYTQNVEGINVHKYGAHIFHTSNKVVWDYIQQFAEFNRFTNSPVARYKDELYSLPFNMLTFNKMWGVITPQEAEAKIKEQIAKENITDPKNLEEQAISLVGRDIYEKLIKGYTEKQWGRKCTELPAFIIKRLPVRYTYDNNYFYDTYQGIPIGGYTGIFERMLEGIEVKLGVDFFAEREHYESLAEKIVFTGMIDEYFGYQFGKLEYRSLRFDNEVLNIPNYQGNAVVNYTEAEVPYTRIIEHKHFEYGTQPKTVITREHSKEYEEGDEPYYPINDARNNELYAKYKALADATPNVIFGGRLAQYKYFDMHNIIAEALECVKVHF

SEQ ID NO: 69 - protein, 436 aa, Artificial Sequence
  amino acid sequence rfb APP8 UDP-phopshate galactose phosphotransferase
MNKIIAKISLILVDIVAIFVSILIAVSLRKILGLLFTLPEIDYSYIFFAYVYLILILMMTYLGAYTKRYDFWHESRLIVRGSFLSLLILLSALALGQNAEYYSRSTLVLIFLCCAIVLPIAKIFTKKILFKLGIWQLPAKVISENDQFKNELFEDQYLGYVKAKHSEHKIIFIDGANLGKDRLNQIIEDNIKNSREIIFTPVLNGYDFSHSYIYNIFNTRTNIFTLENELLSKSNRIFKLLMDYILVLGSAVFWVPVLVLIAFWIKKEDPKGEVFFLQRRLGVNGKEFMCYKFRSMYSDQSFMQEWLEKNPEEAAYYRIYHKYMNDPRITKIGAFLRKTSLDELPQLINVLRGEMSLVGPRPYMVIEKKDIGKKAPLVLAVKPGITGMWQVSGRSDVNFDSRVEMDVWYMKNWSLWNDIVILIKTVQAVFKRDGAY

SEQ ID NO: 70 - protein, 159 aa, Artificial Sequence
  amino acid sequence rfb APP8 Acyltransferase
MITSIQYLRGIAALFVVLFHMKWMLNNVYVEKNLGDIFFISGNFGVDLFFVISGFVICLSTERETLHPVKEFFIRRFFRIYPLLLLSVCTIYILGDFKIHELILSMIPIHLDYSSPSPVFGYNILVSAWTITYEISFYIILVLSLMINHRFRCELTILF

SEQ ID NO: 71 - protein, 137 aa, Artificial Sequence
  amino acid sequence rfb APP8 Acyltransferase
MKSIIILDKYFLYSILLVVISFVFIKHPIFDGHGVLKWGFLSFIILLILLIIENTYGIAKSNFLFWLGEISYSLYLTHIIILEFILKHITPEIWNNPNLGMSKILFYLAISISFSYLVYLLVEKPFINLGKKLITKL

SEQ ID NO: 72 - protein, 357 aa, Artificial Sequence
  amino acid sequence rfb APP8 dTDP-glucose 4,6-dehydratase
MKILITGGAGFIGSAVIRYIIQHTQDSVVNVDKLTYAGNLASLESVSNSSRYHFEQADICDSTRISQIFCKYQPDVVMHLAAESHVDRSIDGPAAFMQTNIIGTYTLLEASRQYWLSLPLERKQTFRFQHISTDEVYGDLNDSNELFSENTAYSPSSPYSASKAASDHLVRAWFRTYGLPTLVTNCSNNYGPFQFPEKLIPLMILNAISGKPLPIYGNGLQIRDWLFVEDHAIALYQVLCRGKVGETYNIGGHNEKTNIEVVQAICRLLDELVPNKPSGIEQYEELVTYVADRPGHDVRYAIDASKIENQLGWTPKETFESGLRKTVEWYLNNQKWWQSVLDGSYCGERLGLSLKSY